SEQUENCE LISTING (77 sequences)

SEQ ID NO: 1 - DNA, 30 bp, Artificial Sequence
  PCR primer hPDI/UP1
agcgctgacgcccccgaggaggaggaccac

SEQ ID NO: 2 - DNA, 42 bp, Artificial Sequence
  PCR primer hPDI/LP-PacI
ccttaattaattacagttcatcatgcacagctttctgatcat

SEQ ID NO: 3 - DNA, 47 bp, Artificial Sequence
  PCR primer PB248
atgaattcaggccatatcggccattgtttactgtgcgcccacagtag

SEQ ID NO: 4 - DNA, 35 bp, Artificial Sequence
  PCR primer PB249
atgtttaaacgtgaggattactggtgatgaaagac

SEQ ID NO: 5 - DNA, 34 bp, Artificial Sequence
  PCR primer PB250
agactagtctatttggagacattgacggatccac

SEQ ID NO: 6 - DNA, 46 bp, Artificial Sequence
  PCR primer PB251
atctcgagaggccatgcaggccaaccacaagatgaatcaaattttg

SEQ ID NO: 7 - DNA, 34 bp, Artificial Sequence
  PCR primer PpPDI/UPi-1
ggtgaggttgaggtcccaagtgactatcaaggtc

SEQ ID NO: 8 - DNA, 34 bp, Artificial Sequence
  PCR primer PpPDI/LPi-1
gaccttgatagtcacttgggacctcaacctcacc

SEQ ID NO: 9 - DNA, 34 bp, Artificial Sequence
  PCR primer PpPDI/UPi-2
cgccaatgatgaggatgcctcttcaaaggttgtg

SEQ ID NO: 10 - DNA, 34 bp, Artificial Sequence
  PCR primer PpPDI/LPi-2
cacaacctttgaagaggcatcctcatcattggcg

SEQ ID NO: 11 - DNA, 24 bp, Artificial Sequence
  PCR primer PpPDI-5′/UP
ggcgattgcattcgcgactgtatc

SEQ ID NO: 12 - DNA, 23 bp, Artificial Sequence
  PCR primer hPDI-3′/LP
cctagagagcggtggccaagatg

SEQ ID NO: 13 - DNA, 25 bp, Artificial Sequence
  PCR primer hPDI/UP
gtggccacaccagggggcatggaac

SEQ ID NO: 14 - DNA, 23 bp, Artificial Sequence
  PCR primer hPDI-3′/LP
cctagagagcggtggccaagatg

SEQ ID NO: 15 - DNA, 37 bp, Artificial Sequence
  PCR primer hGRP94/UP1
agcgctgacgatgaagttgatgtggatggtacagtag

SEQ ID NO: 16 - DNA, 38 bp, Artificial Sequence
  PCR primer hGRP94/LP1
ggccggccttacaattcatcatgttcagctgtagattc

SEQ ID NO: 17 - DNA, 24 bp, Artificial Sequence
  PCR primer PMT1-KO1
tgaacccatctgtaaatagaatgc

SEQ ID NO: 18 - DNA, 45 bp, Artificial Sequence
  PCR primer PMT1-KO2
gtgtcacctaaatcgtatgtgcccatttactggaagctgctaacc

SEQ ID NO: 19 - DNA, 45 bp, Artificial Sequence
  PCR primer PMT1-KO3
ctccctatagtgagtcgtattcatcattgtactttggtatattgg

SEQ ID NO: 20 - DNA, 24 bp, Artificial Sequence
  PCR primer PMT1-KO4
tatttgtacctgcgtcctgtttgc

SEQ ID NO: 21 - DNA, 21 bp, Artificial Sequence
  PCR primer PR29
cacatacgatttaggtgacac

SEQ ID NO: 22 - DNA, 21 bp, Artificial Sequence
  PCR primer PR32
aatacgactcactatagggag

SEQ ID NO: 23 - DNA, 24 bp, Artificial Sequence
  PCR primer PMT4-KO1
tgctctccgcgtgcaatagaaact

SEQ ID NO: 24 - DNA, 45 bp, Artificial Sequence
  PCR primer PMT4-KO2
ctccctatagtgagtcgtattcacagtgtaccatctttcatctcc

SEQ ID NO: 25 - DNA, 45 bp, Artificial Sequence
  PCR primer PMT4-KO3
gtgtcacctaaatcgtatgtgaacctaactctaattcttcaaagc

SEQ ID NO: 26 - DNA, 24 bp, Artificial Sequence
  PCR primer PMT4-KO4
actagggtatataattcccaaggt

SEQ ID NO: 27 - DNA, 57 bp, Artificial Sequence
  Encodes Saccharomyces cerevisiae mating factor pre-signal peptide
atgagattcccatccatcttcactgctgttttgttcgctgcttcttctgctttggct

SEQ ID NO: 28 - protein, 19 aa, Artificial Sequence
  Saccharomyces cerevisiae mating factor pre-signal peptide
MRFPSIFTAVLFAASSALA

SEQ ID NO: 29 - DNA, 1353 bp, Artificial Sequence
  Encodes anti-Her2 Heavy chain (VH + IgG1 constant region)
gaggttcagttggttgaatctggaggaggattggttcaacctggtggttctttgagattgtcctgtgctgcttccggtttcaacatcaaggacacttacatccactgggttagacaagctccaggaaagggattggagtgggttgctagaatctacccaactaacggttacacaagatacgctgactccgttaagggaagattcactatctctgctgacacttccaagaacactgcttacttgcagatgaactccttgagagctgaggatactgctgtttactactgttccagatggggtggtgatggtttctacgctatggactactggggtcaaggaactttggttactgtttcctccgcttctactaagggaccatctgttttcccattggctccatcttctaagtctacttccggtggtactgctgctttgggatgtttggttaaagactacttcccagagccagttactgtttcttggaactccggtgctttgacttctggtgttcacactttcccagctgttttgcaatcttccggtttgtactctttgtcctccgttgttactgttccatcctcttccttgggtactcagacttacatctgtaacgttaaccacaagccatccaacactaaggttgacaagaaggttgagccaaagtcctgtgacaagactcatacttgtccaccatgtccagctccagaattgttgggtggtccttccgtttttttgttcccaccaaagccaaaggacactttgatgatctccagaactccagaggttacatgtgttgttgttgacgtttctcacgaggacccagaggttaagttcaactggtacgttgacggtgttgaagttcacaacgctaagactaagccaagagaggagcagtacaactccacttacagagttgtttccgttttgactgttttgcaccaggattggttgaacggaaaggagtacaagtgtaaggtttccaacaaggctttgccagctccaatcgaaaagactatctccaaggctaagggtcaaccaagagagccacaggtttacactttgccaccatccagagatgagttgactaagaaccaggtttccttgacttgtttggttaagggattctacccatccgacattgctgttgaatgggagtctaacggtcaaccagagaacaactacaagactactccacctgttttggactctgacggttcctttttcttgtactccaagttgactgttgacaagtccagatggcaacagggtaacgttttctcctgttccgttatgcatgaggctttgcacaaccactacactcaaaagtccttgtctttgtcccctggtaagtaa

SEQ ID NO: 30 - protein, 450 aa, Artificial Sequence
  Anti-Her2 Heavy chain (VH + IgG1 constant region)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 645 bp, Artificial Sequence
  Encodes anti-Her2 light chain (VL + Kappa constant region)
gacatccaaatgactcaatccccatcttctttgtctgcttccgttggtgacagagttactatcacttgtagagcttcccaggacgttaatactgctgttgcttggtatcaacagaagccaggaaaggctccaaagttgttgatctactccgcttccttcttgtactctggtgttccatccagattctctggttccagatccggtactgacttcactttgactatctcctccttgcaaccagaagatttcgctacttactactgtcagcagcactacactactccaccaactttcggacagggtactaaggttgagatcaagagaactgttgctgctccatccgttttcattttcccaccatccgacgaacagttgaagtctggtacagcttccgttgtttgtttgttgaacaacttctacccaagagaggctaaggttcagtggaaggttgacaacgctttgcaatccggtaactcccaagaatccgttactgagcaagactctaaggactccacttactccttgtcctccactttgactttgtccaaggctgattacgagaagcacaaggtttacgcttgtgaggttacacatcagggtttgtcctccccagttactaagtccttcaacagaggagagtgttaa

SEQ ID NO: 32 - protein, 213 aa, Artificial Sequence
  Anti-Her2 light chain (VL + Kappa constant region)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLNNFYPREAKVQWKVDNALQSGNSQESVTEQDSKDSTYSLSSTLTLSKADYEKHKVYACEVTHQGLSSPVTKSFNRGEC

SEQ ID NO: 33 - DNA, 60 bp, Artificial Sequence
  Encodes alpha amylase signal peptide (from Aspergillus niger  -amylase)
atggttgcttggtggtccttgttcttgtacggattgcaagttgctgctccagctttggct

SEQ ID NO: 34 - protein, 20 aa, Artificial Sequence
  Alpha amylase signal peptide (from Aspergillus niger  -amylase)
MVAWWSLFLYGLQVAAPALA

SEQ ID NO: 35 - DNA, 397 bp, Artificial Sequence
  Encodes anti-CD20 Light chain Variable Region
gagatcgttttgacacagtccccagctactttgtctttgtccccaggtgaaagagctacattgtcctgtagagcttcccaatctgtttcctcctacttggcttggtatcaacaaaagccaggacaggctccaagattgttgatctacgacgcttccaatagagctactggtatcccagctagattctctggttctggttccggtactgacttcactttgactatctcttccttggaaccagaggacttcgctgtttactactgtcagcagagatccaattggccattgactttcggtggtggtactaaggttgagatcaagcgtacggttgctgctccttccgttttcattttcccaccatccgacgaacaattgaagtctggtacccaattcgccc

SEQ ID NO: 36 - protein, 132 aa, Artificial Sequence
  Anti-CD20 Light chain Variable Region
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTQFA

SEQ ID NO: 37 - DNA, 445 bp, Artificial Sequence
  Encodes anti-CD20 Heavy chain Variable Region
gctgttcagctggttgaatctggtggtggattggttcaacctggtagatccttgagattgtcctgtgctgcttccggttttactttcggtgactacactatgcactgggttagacaagctccaggaaagggattggaatgggtttccggtatttcttggaactccggttccattggttacgctgattccgttaagggaagattcactatctccagagacaacgctaagaactccttgtacttgcagatgaactccttgagagctgaggatactgctttgtactactgtactaaggacaaccaatacggttctggttccacttacggattgggagtttggggacagggaactttggttactgtctcgagtgcttctactaagggaccatccgtttttccattggctccatcctctaagtctacttccggtggtacccaattcgccc

SEQ ID NO: 38 - protein, 148 aa, Artificial Sequence
  Anti-CD20 Heavy chain Variable Region
AVQLVESGGGLVQPGRSLRLSCAASGFTFGDYTMHWVRQAPGKGLEWVSGISWNSGSIGYADSVKGRFTISRDNAKNSLYLQMNSLRAEDTALYYCTKDNQYGSGSTYGLGVWGQGTLVTVSSASTKGPSVFPLAPSSKSTSGGTQFA

SEQ ID NO: 39 - DNA, 1476 bp, Artificial Sequence
  Encodes human PDI without leader
gacgcccccgaggaggaggaccacgtcttggtgctgcggaaaagcaacttcgcggaggcgctggcggcccacaagtacccgccggtggagttccatgccccctggtgtggccactgcaaggctctggcccctgagtatgccaaagccgctgggaagctgaaggcagaaggttccgagatcaggttggccaaggtggacgccacggaggagtctgacctagcccagcagtacggcgtgcgcggctatcccaccatcaagttcttcaggaatggagacacggcttcccccaaggaatatacagctggcagagaggctgatgacatcgtgaactggctgaagaagcgcacgggcccggctgccaccaccctgcctgacggcgcagctgcagagtccttggtggagtccagcgaggtggccgtcatcggcttcttcaaggacgtggagtcggactctgccaagcagtttttgcaggcagcagaggccatcgatgacataccatttgggatcacttccaacagtgacgtgttctccaaataccagctcgacaaagatggggttgtcctctttaagaagtttgatgaaggccggaacaactttgaaggggaggtcaccaaggagaacctgctggactttatcaaacacaaccagctgccccttgtcatcgagttcaccgagcagacagccccgaagatttttggaggtgaaatcaagactcacatcctgctgttcttgcccaagagtgtgtctgactatgacggcaaactgagcaacttcaaaacagcagccgagagcttcaagggcaagatcctgttcatcttcatcgacagcgaccacaccgacaaccagcgcatcctcgagttctttggcctgaagaaggaagagtgcccggccgtgcgcctcatcaccttggaggaggagatgaccaagtacaagcccgaatcggaggagctgacggcagagaggatcacagagttctgccaccgcttcctggagggcaaaatcaagccccacctgatgagccaggagctgccggaggactgggacaagcagcctgtcaaggtgcttgttgggaagaactttgaagacgtggcttttgatgagaaaaaaaacgtctttgtggagttctatgccccatggtgtggtcactgcaaacagttggctcccatttgggataaactgggagagacgtacaaggaccatgagaacatcgtcatcgccaagatggactcgactgccaacgaggtggaggccgtcaaagtgcacggcttccccacactcgggttctttcctgccagtgccgacaggacggtcattgattacaacggggaacgcacgctggatggttttaagaaattcctagagagcggtggccaagatggggcaggggatgttgacgacctcgaggacctcgaagaagcagaggagccagacatggaggaagacgatgaccagaaagctgtgaaagatgaactgtaa

SEQ ID NO: 40 - protein, 491 aa, Artificial Sequence
  human PDI without leader
DAPEEEDHVLVLRKSNFAEALAAHKYPPVEFHAPWCGHCKALAPEYAKAAGKLKAEGSEIRLAKVDATEESDLAQQYGVRGYPTIKFFRNGDTASPKEYTAGREADDIVNWLKKRTGPAATTLPDGAAAESLVESSEVAVIGFFKDVESDSAKQFLQAAEAIDDIPFGITSNSDVFSKYQLDKDGVVLFKKFDEGRNNFEGEVTKENLLDFIKHNQLPLVIEFTEQTAPKIFGGEIKTHILLFLPKSVSDYDGKLSNFKTAAESFKGKILFIFIDSDHTDNQRILEFFGLKKEECPAVRLITLEEEMTKYKPESEELTAERITEFCHRFLEGKIKPHLMSQELPEDWDKQPVKVLVGKNFEDVAFDEKKNVFVEFYAPWCGHCKQLAPIWDKLGETYKDHENIVIAKMDSTANEVEAVKVHGFPTLGFFPASADRTVIDYNGERTLDGFKKFLESGGQDGAGDVDDLEDLEEAEEPDMEEDDDQKAVHDEL

SEQ ID NO: 41 - DNA, 1554 bp, Pichia pastoris
  Pichia pastoris PDI1 Gene
atgcaattcaactggaatattaaaactgtggcaagtattttgtccgctctcacactagcacaagcaagtgatcaggaggctattgctccagaggactctcatgtcgtcaaattgactgaagccacttttgagtctttcatcaccagtaatcctcacgttttggcagagttttttgccccttggtgtggtcactgtaagaagttgggccctgaacttgtttctgctgccgagatcttaaaggacaatgagcaggttaagattgctcaaattgattgtacggaggagaaggaattatgtcaaggctacgaaattaaagggtatcctactttgaaggtgttccatggtgaggttgaggtcccaagtgactatcaaggtcaaagacagagccaaagcattgtcagctatatgctaaagcagagtttaccccctgtcagtgaaatcaatgcaaccaaagatttagacgacacaatcgccgaggcaaaagagcccgtgattgtgcaagtactaccggaagatgcatccaacttggaatctaacaccacattttacggagttgccggtactctcagagagaaattcacttttgtctccactaagtctactgattatgccaaaaaatacactagcgactcgactcctgcctatttgcttgtcagacctggcgaggaacctagtgtttactctggtgaggagttagatgagactcatttggtgcactggattgatattgagtccaaacctctatttggagacattgacggatccaccttcaaatcatatgctgaagctaacatccctttagcctactatttctatgagaacgaagaacaacgtgctgctgctgccgatattattaaaccttttgctaaagagcaacgtggcaaaattaactttgttggcttagatgccgttaaattcggtaagcatgccaagaacttaaacatggatgaagagaaactccctctatttgtcattcatgatttggtgagcaacaagaagtttggagttcctcaagaccaagaattgacgaacaaagatgtgaccgagctgattgagaaattcatcgcaggagaggcagaaccaattgtgaaatcagagccaattccagaaattcaagaagagaaagtcttcaagctagtcggaaaggcccacgatgaagttgtcttcgatgaatctaaagatgttctagtcaagtactacgccccttggtgtggtcactgtaagagaatggctcctgcttatgaggaattggctactctttacgccaatgatgaggatgcctcttcaaaggttgtgattgcaaaacttgatcacactttgaacgatgtcgacaacgttgatattcaaggttatcctactttgatcctttatccagctggtgataaatccaatcctcaactgtatgatggatctcgtgacctagaatcattggctgagtttgtaaaggagagaggaacccacaaagtggatgccctagcactcagaccagtcgaggaagaaaaggaagctgaagaagaagctgaaagtgaggcagacgctcacgacgagctttaa

SEQ ID NO: 42 - protein, 1554 aa, Pichia pastoris
ATGCAATTCAACTGGAATATTAAAACTGTGGCAAGTATTTTGTCCGCTCTCACACTAGCACAAGCAAGTGATCAGGAGGCTATTGCTCCAGAGGACTCTCATGTCGTCAAATTGACTGAAGCCACTTTTGAGTCTTTCATCACCAGTAATCCTCACGTTTTGGCAGAGTTTTTTGCCCCTTGGTGTGGTCACTGTAAGAAGTTGGGCCCTGAACTTGTTTCTGCTGCCGAGATCTTAAAGGACAATGAGCAGGTTAAGATTGCTCAAATTGATTGTACGGAGGAGAAGGAATTATGTCAAGGCTACGAAATTAAAGGGTATCCTACTTTGAAGGTGTTCCATGGTGAGGTTGAGGTCCCAAGTGACTATCAAGGTCAAAGACAGAGCCAAAGCATTGTCAGCTATATGCTAAAGCAGAGTTTACCCCCTGTCAGTGAAATCAATGCAACCAAAGATTTAGACGACACAATCGCCGAGGCAAAAGAGCCCGTGATTGTGCAAGTACTACCGGAAGATGCATCCAACTTGGAATCTAACACCACATTTTACGGAGTTGCCGGTACTCTCAGAGAGAAATTCACTTTTGTCTCCACTAAGTCTACTGATTATGCCAAAAAATACACTAGCGACTCGACTCCTGCCTATTTGCTTGTCAGACCTGGCGAGGAACCTAGTGTTTACTCTGGTGAGGAGTTAGATGAGACTCATTTGGTGCACTGGATTGATATTGAGTCCAAACCTCTATTTGGAGACATTGACGGATCCACCTTCAAATCATATGCTGAAGCTAACATCCCTTTAGCCTACTATTTCTATGAGAACGAAGAACAACGTGCTGCTGCTGCCGATATTATTAAACCTTTTGCTAAAGAGCAACGTGGCAAAATTAACTTTGTTGGCTTAGATGCCGTTAAATTCGGTAAGCATGCCAAGAACTTAAACATGGATGAAGAGAAACTCCCTCTATTTGTCATTCATGATTTGGTGAGCAACAAGAAGTTTGGAGTTCCTCAAGACCAAGAATTGACGAACAAAGATGTGACCGAGCTGATTGAGAAATTCATCGCAGGAGAGGCAGAACCAATTGTGAAATCAGAGCCAATTCCAGAAATTCAAGAAGAGAAAGTCTTCAAGCTAGTCGGAAAGGCCCACGATGAAGTTGTCTTCGATGAATCTAAAGATGTTCTAGTCAAGTACTACGCCCCTTGGTGTGGTCACTGTAAGAGAATGGCTCCTGCTTATGAGGAATTGGCTACTCTTTACGCCAATGATGAGGATGCCTCTTCAAAGGTTGTGATTGCAAAACTTGATCACACTTTGAACGATGTCGACAACGTTGATATTCAAGGTTATCCTACTTTGATCCTTTATCCAGCTGGTGATAAATCCAATCCTCAACTGTATGATGGATCTCGTGACCTAGAATCATTGGCTGAGTTTGTAAAGGAGAGAGGAACCCACAAAGTGGATGCCCTAGCACTCAGACCAGTCGAGGAAGAAAAGGAAGCTGAAGAAGAAGCTGAAAGTGAGGCAGACGCTCACGACGAGCTTTAA

SEQ ID NO: 43 - DNA, 1337 bp, Artificial Sequence
  Encodes human ERO1alpha without leader
gaagaacaaccaccagagactgctgctcagagatgcttctgtcaggtttccggttacttggacgactgtacttgtgacgttgagactatcgacagattcaacaactacagattgttcccaagattgcagaagttgttggagtccgactacttcagatactacaaggttaacttgaagagaccatgtccattctggaacgacatttcccagtgtggtagaagagactgtgctgttaagccatgtcaatccgacgaagttccagacggtattaagtccgcttcctacaagtactctgaagaggctaacaacttgatcgaagagtgtgagcaagctgaaagattgggtgctgttgacgaatctttgtccgagagactcagaaggctgttttgcagtggactaagcacgatgattcctccgacaacttctgtgaagctgacgacattcaatctccagaggctgagtacgttgacttgttgttgaacccagagagatacactggttacaagggtccagacgcttggaagatttggaacgttatctacgaagagaactgtttcaagccacagactatcaagagaccattgaacccattggcttccggacagggaacttctgaagagaacactttctactcttggttggagggtttgtgtgttgagaagagagctttctacagattgatctccggattgcacgcttctatcaacgttcacttgtccgctagatacttgttgcaagagacttggttggaaaagaagtggggtcacaacattactgagttccagcagagattcgacggtattttgactgaaggtgaaggtccaagaagattgaagaacttgtactttttgtacttgatcgagttgagagctttgtccaaggttttgccattcttcgagagaccagacttccaattgttcactggtaacaagatccaggacgaagagaacaagatgttgttgttggagattttgcacgagatcaagtcctttccattgcacttcgacgagaactcatttttcgctggtgacaagaaagaagctcacaagttgaaagaggacttcagattgcacttcagaaatatctccagaatcatggactgtgttggttgtttcaagtgtagattgtggggtaagttgcagactcaaggattgggtactgctttgaagattttgttctccgagaagttgatcgctaacatgcctgaatctggtccatcttacgagttccacttgactagacaagagatcgtttccttgttcaacgctttcggtagaatctccacttccgttaaagagttggagaacttcagaaacttgttgcagaacatccactaa

SEQ ID NO: 44 - protein, 445 aa, Artificial Sequence
  human ERO1alpha without leader
EEQPPETAAQRCFCQVSGYLDDCTCDVETIDRFNNYRLFPRLQKLLESDYFRYYKVNLKRPCPFWNDISQCGRRDCAVKPCQSDEVPDGIKSASYKYSEEANNLIEECEQAERLGAVDESLSEETQKAVLQWTKHDDSSDNFCEADDIQSPEAEYVDLLLNPERYTGYKGPDAWKIWNVIYEENCFKPQTIKRPLNPLASGQGTSEENTFYSWLEGLCVEKRAFYRLISGLHASINVHLSARYLLQETWLEKKWGHNITEFQQRFDGILTEGEGPRRLKNLYFLYLIELRALSKVLPFFERPDFQLFTGNKIQDEENKMLLLEILHEIKSFPLHFDENSFFAGDKKEAHKLKEDFRLHFRNISRIMDCVGCFKCRLWGKLQTQGLGTALKILFSEKLIANMPESGPSYEFHLTRQEIVSLFNAFGRISTSVKELENFRNLLQNIH

SEQ ID NO: 45 - DNA, 2349 bp, Artificial Sequence
  Encodes human GRP94 without leader
gatgatgaagttgacgttgacggtactgttgaagaggacttgggaaagtctagagagggttccagaactgacgacgaagttgttcagagagaggaagaggctattcagttggacggattgaacgcttcccaaatcagagagttgagagagaagtccgagaagttcgctttccaagctgaggttaacagaatgatgaaattgattatcaactccttgtacaagaacaaagagattttcttgagagagttgatctctaacgcttctgacgctttggacaagatcagattgatctccttgactgacgaaaacgctttgtccggtaacgaagagttgactgttaagatcaagtgtgacaaagagaagaacttgttgcacgttactgacactggtgttggaatgactagagaagagttggttaagaacttgggtactatcgctaagtctggtacttccgagttcttgaacaagatgactgaggctcaagaagatggtcaatccacttccgagttgattggtcagttcggtgttggtttctactccgctttcttggttgctgacaaggttatcgttacttccaagcacaacaacgacactcaacacatttgggaatccgattccaacgagttctccgttattgctgacccaagaggtaacactttgggtagaggtactactatcactttggttttgaaagaagaggcttccgactacttggagttggacactatcaagaacttggttaagaagtactcccagttcatcaacttcccaatctatgtttggtcctccaagactgagactgttgaggaaccaatggaagaagaagaggctgctaaagaagagaaagaggaatctgacgacgaggctgctgttgaagaagaggaagaagaaaagaagccaaagactaagaaggttgaaaagactgtttgggactgggagcttatgaacgacatcaagccaatttggcagagaccatccaaagaggttgaggaggacgagtacaaggctttctacaagtccttctccaaagaatccgatgacccaatggcttacatccacttcactgctgagggtgaagttactttcaagtccatcttgttcgttccaacttctgctccaagaggattgttcgacgagtacggttctaagaagtccgactacatcaaactttatgttagaagagttttcatcactgacgacttccacgatatgatgccaaagtacttgaacttcgttaagggtgttgttgattccgatgacttgccattgaacgtttccagagagactttgcagcagcacaagttgttgaaggttatcagaaagaaacttgttagaaagactttggacatgatcaagaagatcgctgacgacaagtacaacgacactttctggaaagagttcggaactaacatcaagttgggtgttattgaggaccactccaacagaactagattggctaagttgttgagattccagtcctctcatcacccaactgacatcacttccttggaccagtacgttgagagaatgaaagagaagcaggacaaaatctacttcatggctggttcctctagaaaagaggctgaatcctccccattcgttgagagattgttgaagaagggttacgaggttatctacttgactgagccagttgacgagtactgtatccaggctttgccagagtttgacggaaagagattccagaacgttgctaaagagggtgttaagttcgacgaatccgaaaagactaaagaatccagagaggctgttgagaaagagttcgagccattgttgaactggatgaaggacaaggctttgaaggacaagatcgagaaggctgttgtttcccagagattgactgaatccccatgtgctttggttgcttcccaatacggatggagtggtaacatggaaagaatcatgaaggctcaggcttaccaaactggaaaggacatctccactaactactacgcttcccagaagaaaactttcgagatcaacccaagacacccattgatcagagacatgttgagaagaatcaaagaggacgaggacgacaagactgttttggatttggctgttgttttgttcgagactgctactttgagatccggttacttgttgccagacactaaggcttacggtgacagaatcgagagaatgttgagattgtccttgaacattgacccagacgctaaggttgaagaagaaccagaagaagagccagaggaaactgctgaagatactactgaggacactgaacaagacgaggacgaagagatggatgttggtactgacgaagaggaagagacagcaaaggaatccactgctgaacacgacgagttgtaa

SEQ ID NO: 46 - protein, 782 aa, Artificial Sequence
  human GRP94 without leader
DDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEHDEL

SEQ ID NO: 47 - DNA, 8448 bp, Pichia pastoris
  CDS (3016)...(5382): Encodes PMT1
actttttcaattcctcagggtactccgttggaattctgtacttagcagcatactgatctttgaccacccaaggagcaccagatctttgcgatctagtcaacgtcaacttgagaaaagttttcacgtaccacttagtgaacgcattcctatcacgggaaacttgattttcgttcacggttacttctccatcagagtttgagaggccaacgcgataagagcagtatccttcacgtacggtaccatcaggtaaggtgatgggagcaaaccgtgccttttctctgatgatccctttatatctgttagatccagcacttttaacattcactagatccccaggaaaaaattctttcttgaagtgtaaatacacgtcatcgactaattgatctagtctgggtatgagactgaattgcacatatctcaaaattggttctctgacaggttctggaaacttattttcaaccgcttgcatttcctcctgttcgtacttgagggcctcaaagaaatcaaacgagctgtttccagtgatttcacacgcaaacttcttttggctataatagtccatcctatcaaggtactcatcgtagttcaaaaaccactcgccagtctgtggaatgtaccaaatttccgtgtctagatcatcaggaagctgttgtggagggacaacttccacctgctttcttttgaagagaaccatggtgtttggggattagaagaaaacaaatatttgagcggaacttgcgaaaaaacgcccctagcgaatgcaagctagacatgtcaggaagataaaattgataccgcagaagcaggggtagttggggagggcaatcaagtacgttcacagagcatggctgcgttatcaactgactattttatggcgtggtttagaagagagagtatcaattaggcgtcaactgggaccattatgattagacgttgtaggtagatgcaggtgaaaaatggacagacgtaggcaacaaacacaaactgtcgggtaacctttaacagtattcaattccaggtgtttcaagacagccttagatactagcaagcttccagggaaaccctattactcatgctcccactgttggaactcacaaccaagaggctacatgtatgcgtatgcatacaggtactgctcagtgataaatttatttcgcgagatcgtactccagaaactttcatgtaagccttcctacttcgctctgcccactatgttagccagaaaggtattagctagacaatgtctggtggtagccaggctttgtgcgggtagatttgcctcctcattatgcgggtgcagttgtagaggtttgatgaggccaccaaaatttaacagttccaaatctctttcgagatcgatgacctcatcgtccctgtttgagtctccaaattgtccttcctgtggtgtggttctccaaacagaacatccagacaaagatgggtattgtctactgcccaaaggtgaaaggaaagttaaaaattatcaaaatgaactaaaagaaaagctttttttgaatgtgaaaagggaagaacttgccgacagactgggccatgaggtggactctgaatcactgattatacccaaggaaatgtaccaaaagccccgtaccccgaaacgactggtttgtcagagatgcttcaaatcgcaaaactattccttgatcgaccattccattcgtgaagaaaatcccgaacacaagatcctggatgagatcccttcaaacgccaatatcgtccacgttttatctgctgttgattttcctcttggtctcagcaaggaactggtaaacagatttaaacccactcagattacgtacgttattacaaagtctgacgtgttcttccccgataagctaggtctccaacggacgggagctgcttattttgaagacagcttggtaaagcttgtcggtgcagatcctagaaaggtagtattggtctcaggaaaaagaaattggggcctcaaacagctgctatccactttacccagaggtcccaattactttctgggaatgacgaacaccggaaaatcaaccctaatacgatccatcgttggtaaggattactcaaagaagcagacagagaatggcccgggtgtctctcaccttccttcattcacaagaaaacccatgaagttcaaaatggacaacaacagtcttgaactcgtagatctccctggatacactgctccaaatggaggtgtttacaagtatcttaaggaagagaactaccgagacattttgaacgttaaacagttaaagccattgacatccctcaaggcatacacagaaacgttgccttcgaagccaaaactattcaatggtgtgcgagtaatatgcattggtggtttagtgtacattcggcccccaaagggtgtagtgctgaaacagtttagtctcgtcaaccttccatccttcatgtactcgtcgctaaaaaaggccaccagtgtaatccaagcgcccccacaagccttggtgaattgcagcgtcgtcaaggaggacagtccagatgaactggtaagatatgtgatccctccattttatggtttaattgacctggtcattcaaggtgttggatttatcaagcttctgcccactggagctcggaacaccagagaactgatagaaatttttgccccaaaagacatccagctcatggtgcgtgattccatcctcaaatacgtctacaagacccatgccgaacacgactcaaccaataatctcctgcataaaaagaacataaaagccagaggccaaaccatactacgaagactacccaaaaagcctgtattcacaaagctttttcccgtaccagccaacgtaccgtctcatgaactgctcaccatggtgacgggaaaggacgacctagccgaggaagacaaagaataccgctacgatatccagtatcccaacagatactgggatgaaaccatctgtaaatagaatgcttatgtaatcaagcactttctgaaattccttagagtttcgcgtgtctccccgtcaaaaatcgcgtctcatgtgccagatatttctcccgcaaaacgtaacacgttgttctgtttcccttttgacaatgagtaaaacaagtcctcaagaggtgccagaaaacactactgagcttaaaatctcaaaaggagagctccgtccttttattgtgacctctccatctcctcaattgagcaagtctcgttctgtgacttcaaccaaggagaagctgatattggctagtttgttcatatttgcaatggtcatcaggttccacaacgtcgcccaccctgacagcgttgtgtttgatgaagttcactttggggggtttgccagaaagtacattttgggaacctttttcatggatgttcatccgccattggccaagctattatttgctggtgttggcagtcttggtggatacgatggagagtttgagttcaagaaaattggtgacgaattcccagagaatgttccttatgtgctcatgagatatcttccctctggtatgggagttggaacatgtattatgttgtatttgactctgagagcttctggttgtcaaccaatagtctgtgctctgacaaccgctcttttgatcattgagaatgctaatgttacaatctccagattcattttgctggattcgccaatgctgttttttattgcttcaacagtttactctttcaagaaatttcaaattcaggaaccgtttaccttccaatggtacaagacccttattgctactggtgtttctttagggttagcagcttccagtaaatgggttggtttgttcaccgttgcctggattggattgataacaatttgggacttatggttcatcattggtgatttgactgtttctgtaaagaaaattttcggccattttatcaccagagctgtagctttcttagtcgtccccactctgatctacctcactttctttgccatccatttgcaagtcttaaccaaggaaggtgatggtggtgctttcatgtcttccgtcttcagatcgaccttagaaggtaatgctgttccaaaacagtcgctggccaacgttggtttgggctctttagtcactatccgtcatttgaacaccagaggtggttacttacactctcacaatcatctttacgagggtggttctggtcaacagcaggtcaccttgtacccacacattgattctaataatcaatggattgtacaggattacaacgcgactgaggagccaactgaatttgttccattgaaagacggtgtcaaaatcagattaaaccacaaattgacttcccgaagattgcactctcataacctcagacctcctgtgactgaacaagattggcaaaatgaggtatctgcttatggacatgagggctttggcggtgatgccaatgatgactttgttgtggagattgccaaggatctttcaactactgaagaagctaaggaaaacgttagggccattcaaactgtttttagattgagacatgcgatgactggttgttacttgttctcccacgaagtcaagcttcccaagtgggcatatgagcaacaagaggttacttgtgctactcaaggtatcaaaccactatcttactggtacgttgagaccaacgaaaacccattcttggataaagaggttgatgaaatagttagctatcctgttccgactttctttcaaaaggttgccgagctacacgccagaatgtggaagatcaacaagggcttaactgatcatcatgtctatgaatccagtccagattcttggcccttcctgctcagaggtataagctactggtcaaaaaatcactcacaaatttatttcataggtaatgctgtcacttggtggacagtcaccgcaagtattgctttgttctctgtctttttggttttctctattctgagatggcaaagaggttttgggttcagcgttgacccaactgtgttcaacttcaatgttcaaatgcttcattacatcctaggatgggtactgcattacttgccatctttccttatggcccgtcagctatttttgcaccactatctaccatcattgtactttggtatattggctctcggacatgtgtttgagattattcactcttatgtcttcaaaaacaaacaggttgtgtcttactccatattcgttctcttttttgccgttgcgctttctttcttccaaagatattctccattgatctatgcaggacgatggaccaaggaccaatgcaacgaatccaagatactcaagtgggactttgactgtaacaccttccccagtcacacatctcagtatgaaatatgggcatcccctgtacaaacttccactcctaaagaaggaacccactcagaatctaccgtcggagaacctgacgttgagaagctgggagagacagtctaagctgtgtttatatagccctgtacgtaaaatctatgacacaagtttatggttatttgtcttatgtaagcaatatttggattgatgtctcgagaccatcaactccatcactgataagttgatcggatttgtatttctgtcccctatttactaattccctttccagaaatagatcatgaatgaggcagaatataagtgccaaagatgccggctgccgttgaccatagacggatctctggaagaccttagcatatcacaggccaatcttttgacgggacgaaatgggaactttacaaagaacacaatccccttggaggatgccgtggaagaagatttacccaaggtgcctcagagccgacttaacctctttaaagaggtctaccagaagatggatcacgattttaccaatgccagagatgaatttgttgtgttgaacaagcacaatgataacagcgacgtcaatgtggagtatgattacgaagaaaacaacactatcagtcgtagaatcaacacaatgacgaatatcttcaatatcctcagcaacaagtacgaaattgattttccggtttgctacgaatgcgccacattgctgatggaggaattgaagaatgagtacgaaagggtcaatgctgataaagaagtttacgcaaagtttctatccaagcttcgcaaacaggacgcaggtacaaatatgaaagaaagaactgctcaactactggagcaattggagaaaactaagcaagaagagagagataaagaaaagaagctccaaggcctatatgatgaaagagatagtttggaaaaggtattagcttctttagagaatgaaatggaacagttgaatattgaagagcagcaaatttttgaattagagaacaaatatgaatatgagttaatggagttcaagaatgagcaaagcagaatggaagcaatgtatgaggatggtttgacgcaattagataatttaagaaaagtgaacgtctttaatgacgctttcaatatctcgcatgatggtcaattcggcactataaatgggctcaggttgggcacgttagacagtaagagggtttcttggtatgaaataaatgctgcgttgggtcaagttgttttgttactcttcacgttattgagcagacttgagcttgagctcaaacattacaagatttttcccattggctcgacttccaagattgaataccaagttgacccagattccaaacctgttactattaactgcttttcttcgggagaacagttactggataagctttttcattctaataaactagatcctgctatgaacgcaatcctagaaatcactattcaaattgcagatcatttcacaaaacaagatccaacaaacgaattgccctacaaaatggagaacgaaacaatatcaaacttgaatatcaaaccttccaaacgtaaatccaacgaggaatggactttggcatgcaaacatctgttgaccaatctcaaatggataattgccttcagtagttcaacgtgaactagtgtattaaaaaaaagaaacagaaactttattggattataaaactatttatcaagttcaaattaacatagcgacgaagagaccagctgcggctaagactgaactacctagtaccgcttgggcaccgttaccagtttctgtacctgtgccagtggtaccagtaccagtaccggttccagtgccagttcctgtgcctgtgcctgtgcctgtgccggttccggttccagtgcctgtgcctgtgcccgtgcctgttgcagtggtattagtgaaacctcctgtgccagttgcagtggtattagtaaatcctcctgttcctgtggtgtttgtgagtcctccagtttcggtcaagtttccaggaacactaacatcaggggttgaagtgatctctggtggcaccgtggggactgtgacattgacatcatttgtgaagattggctccaactcagttgtagccttaacaacgcttaatgcgagagttgcaccgatcaaacttttgaattgcattttacttttgttacttctaaaatgagatgaggaaagaaagaagagagaagtggaagcactgaaagtgtggtgttatatctgaaaaattcattaccaatcaaaacgtcagacgatgatatgtctaagcccgtgcagaaacgtctagatcttttcaaacgtaaagtacttccccttttggcacatcgtggacttgctattccaaatatagacggggaccttttttagagtatccccgggcgcctcgaattctggggtatttttttgctatagcatgaattggcaatagggattggggacaacgtgtttgacagaagacgtgtgtgtcctgccaaaaaggggtaaaggtgcatttgccaaggcctgtgaatgatctgaacactagaggaaagcaagaaggctgtgtcgtagtctgtattggctgtgttgtcgctgtgtcggttgcttcaaaactttattcgagtccggtacgcgtcaatgggtatttttcaaaaagtttctaactccctcaatcaactttggttttggccggatatggcatgccagaaaaggaagttttactcctggcgatgatgtttacaaatcaagcttagagggagtaaccaatgcagataagtttgcgatggcgctgatctttatgctctcaacaccttctcgactattcagggtcatttcgtggctttgtatttcgggcacaactgatcaccgaggatcaatgaaattttcatgcacatcactgatccagtttctgtcgaatttgcaattccagttgattgcaggacccgcgttctgcctacacattttctcgtgattgtggaagtaattctaattgacagtcgatcaccacaatgacaatcttagttgaccttagattccagtggaatgcagttgaattgtcttttcgtttaattagaggagagtaacggaccagggctcctttattgtatataataattataatttttttcactatttcaccttttcgcttggaatataaaattctaattataattcaacaggaaatattgtccaaaccacatgaagttgtcatg

SEQ ID NO: 48 - protein, 789 aa, Pichia pastoris
MCQIFLPQNVTRCSVSLLTMSKTSPQEVPENTTELKISKGELRPFIVTSPSPQLSKSRSVTSTKEKLILASLFIFAMVIRFHNVAHPDSVVFDEVHFGGFARKYILGTFFMDVHPPLAKLLFAGVGSLGGYDGEFEFKKIGDEFPENVPYVLMRYLPSGMGVGTCIMLYLTLRASGCQPIVCALTTALLIIENANVTISRFILLDSPMLFFIASTVYSFKKFQIQEPFTFQWYKTLIATGVSLGLAASSKWVGLFTVAWIGLITIWDLWFIIGDLTVSVKKIFGHFITRAVAFLVVPTLIYLTFFAIHLQVLTKEGDGGAFMSSVFRSTLEGNAVPKQSLANVGLGSLVTIRHLNTRGGYLHSHNHLYEGGSGQQQVTLYPHIDSNNQWIVQDYNATEEPTEFVPLKDGVKIRLNHKLTSRRLHSHNLRPPVTEQDWQNEVSAYGHEGFGGDANDDFVVEIAKDLSTTEEAKENVRAIQTVFRLRHAMTGCYLFSHEVKLPKWAYEQQEVTCATQGIKPLSYWYVETNENPFLDKEVDEIVSYPVPTFFQKVAELHARMWKINKGLTDHHVYESSPDSWPFLLRGISYWSKNHSQIYFIGNAVTWWTVTASIALFSVFLVFSILRWQRGFGFSVDPTVFNFNVQMLHYILGWVLHYLPSFLMARQLFLHHYLPSLYFGILALGHVFEIIHSYVFKNKQVVSYSIFVLFFAVALSFFQRYSPLIYAGRWTKDQCNESKILKWDFDCNTFPSHTSQYEIWASPVQTSTPKEGTHSESTVGEPDVEKLGETV

SEQ ID NO: 49 - DNA, 8400 bp, Pichia pastoris
  CDS (3169)...(5391): Encodes PMT4
tagtaaagaaatcttgcagtttaattcttcctcttgtgtttttagcgatgagacatcggcactcagagttaagtttgcttgcatctgctctgataacttttgctgtgactctgttgcaatgcttttggtaacggtcaattcgtctatggtttgttgatactttgactttaaggcagtaatattgtcctgtagtttatcattatatgcttccaatgttttgacctttgatgaaatgttttttcgattaacagttagttcatcgaaggagagctccaactctgatacttgcattcttaaattatttataatggtatccttaacttctagtgatttcgagtggcttgcctgggcactcttaagttcttttctcaactgtgctatggatggctcaagcactagaatttgtttctctgaatcgaataattttatttctagcttctgagcaagctcacaggcgcttactttttcggaagttagaaactttgcttcgttattcatggcagacagttctattcttaattgcttattttctttcctaacttccaaaatctccgattccaggggttcatatctacgggaggaaacctgattgcatgacttttcgaacgttttttgatcagaaagttgacagattgtgccatcagttgacgagacagcctcaaactgagttgcttccatgttgcacaaattatcattgaattcagccacttctttctccaaatctccgtttaccagctccttcttctttcctgaagcaatagatgatgatcgatgaatatagtcctctttcaatgggttttggatctctttgtcccattgacaagaagctatgctccttgaatccttcattgacattgggtatgaaattttgctaccatctacctttgcactaatttctgtgggcgaattgtgtgttttcagtagatcttcaagtgctttcttttcgttttctatcttcataagagatattctcaatttatttacggtgtcagtggcagacaaattgtttaatgaaacggtatccagagactcctgctccaggtactctgactgagctacagggaagggtttcttgtcgtgtatggagaacttctgctcaagttgggctttcaaggaattcacttggtgatgcaaaagctcattttcctcattcaaagaagtattcatatttttaagttcctccagctcaaacgtactgcggccttccaaagtgcagattttatccttaagactcaatttctcattcttcaaactttccatctctctattgagcgtttcaacctggtcagttttcaacttgagttcttctgaaaatttgatacttgaacttttagcaagggaagcttcatcaaaagtatcttgtagcttggtttctaaagaccagttagaatcaagtagcctttgctgctcttgttccaactttttggaaaaaattgcctggtgagtgatcttttcagcgagatcctcattttctttaactttttgcttcaatagagatttgagtttttgattatcagagttcagcttccgacattcgactaaaaggttgtcactaatacctgttaaaaaatctattttgttcgtcttttttttgcttggcgactttagaggtaatgcaggaagggaaggatgaaattctgactcctggtgccgatttctcagctttctcggaagtgggggtagctgaaatggtacattgtggttcgtatcaccaagatccatttttatgtctttgtccattagaaaattcaagaatctttcaaaaaaaatagaaacagaagatttagtaaacttaggtgaggtgatataaacctaattgcctgttttattttgatcatgtatgtaaattgtgaaaggtaaatacgcgaaacttatgtatgtattgcaaagatgcacaagacacacaaggattaatgggctatttgctctacattcgcaaaaaatagccagcatttattttttgaatggatactcaataagcccatccctacgcttccatatcttttttttctttttggtagtaacatgctccacgaatacctcttcacaagtagattttttaaatgagcggataaagcgggggtcccatagttcactagcaactcctaagtctttgcagcatctcattaaagcattgctcttacagccttcagtagcagtaggaattcccttctctgaaaaaaaatcttgctctccgcgtgcaatagaaactagtcggccctgtacaattaaagcatactccctggttaaagtacctcctccgaacttgctcttgttgatcaaagtttctgaccttggggccagtccccagccaccagggccaaacgctttattgaggatacgacgatacttaatctctggaagataaagtagtccatctggtgtgatttcgacatcttcgttgctaattggttgacataatatgttactactttcattactgaaggagcaaatacctagtccatggaacgaatccgaccaattgattccatcgccacttgtattagagattggggtgtcgtttaactgtgaagttccaaacaaaattgataaactgctctcgttcttagcttggccactttttggagtctcaatagtagcgttttggctctcgtgaattttctgcacagagtcggatgaagaaggtgcaaatgcttctagcattgtagagtcgaccacatagaacctttttaaagagttatgaaaataactcttggtagggccaaatacaacccgatatcgtcttagcataagagctgcttctttggaatatcgtttcttgtaagtaattacgtgttggctaaacacttagaagtcagtcgcgcatgcggccaaaaacagactagggatagaagatgaactgacaaaaacatcaagaaggtgaagacattcattctatgaaaactagtttttatataaaattatggtctgcatttagagagcaatgatgtaatcaaacatcaataagtgcttgtcgcatcaatatttaataggtaatcatggagtattctagtctaccgccttaaaaaaagctcactcgatctagtgcagcttgattgtgtacttcaatagtattccaacgaccttaacatcttaacaccatgtaaatttaagatccacgtatacgatacaatttctttcaatatcaattctcgttcaagccaactgatgataaaatcaagaaagagatcgagaaaagtttctttgaacactgaaaaggagctgaaaaatagccatatttctcttggagatgaaagatggtacactgtgggtcttctcttggtgacaatcacagctttctgtactcgattctatgctatcaactatccagatgaggttgtttttgacgaagttcatttcggaaaatttgctagctactatctagagcgtacttatttttttgatctgcaccctccgtttgccaagctcctgattgcgtttgtcggctttttagctgggtacaatggtgagttcaagtttacaactattggtgaatcttatatcaaaaacgaggttccctacgtagtttacagatcattgagcgctgtgcaaggatctttaacggtgccaattgtttatttgtgtctcaaagaatgcggatatacagttttgacttgtgtttttggtgcatgtatcatattgtttgatggggcccacgttgctgagactagactaatcttgctggatgccacgttgatttttttcgtttcattgtccatctatagctatatcaaattcacaaaacaaagatcagaaccattcggccaaaagtggtggaagtggctgttctttacaggggtgtctttatcttgcgtcataagtaccaagtatgtgggggtgttcacctatcttacaataggctgtggtgtcctgtttgacttatggagtttactggattataaaaagggacattccttggcatatgttggtaaacactttgctgcacgatttttccttctaatactggtccctttcttgatatatctcaattggttttatgttcatttcgctattctaagcaagtctggcccaggagacagttttatgagctctgaattccaggagactctcggagattctcctcttgcagctttcgcaaaggaagttcactttaacgacataatcacaataaagcataaagagactgatgccatgttgcactcacacttggcaaactaccccctccgttacgaggacgggagggtatcatctcaaggtcaacaagttacagcatactctggagaggacccaaacaataattggcagattatttctcccgaaggacttactggcgttgtaactcagggcgatgtcgttagactgagacacgttgggacagatggctatctactgacgcatgatgttgcgtctcctttctatccaactaacgaggagtttactgtagtgggacaggagaaagctactcaacgctggaacgaaacactttttagaattgatccctatgacaagaagaaaacccgtcctttgaagtcgaaagcttcatttttcaaactcattcatgttcctacggttgtggccatgtggactcataatgaccagcttcttcctgattggggtttcaaccaacaagaagtcaatggtaataagaagcttgctgatgaatcaaacttatgggttgtagacaatatcgtcgatattgcagaggacgatccaaggaaacactacgttccaaaggaagtgaaaaatttgccatttttgaccaagtggttggaattacaaagacttatgtttattcagaataacaagttgagctcagatcatccatttgcgtctgaccctatatcttggcctttttcacttagtggggtttcattttggacaaacaacgagtcacgcaaacagatctattttgtcggaaatattcctggatggtggatggaggttgcagcattgggatcctttctaggactcgtgtttgcagatcagttcacgagaagaagaaacagtcttgttttgaccaatagcgccaggtctcggttatacaataatttggggttcttctttgtaggctggtgttgtcattacctaccctttttcctaatgagccgtcaaaaatttttgcaccattacttacctgcacatttaatagcagccatgttcactgctggtttcttggaatttatttttactgacaacagaactgaagaattcaaggatcagaaaacttcatgtgaacctaactctaattcttcaaagccgaaagagcaattgattctgtggttaagtttctcgtcctttgtcgctttgctactaagcatcattgtttggactttcttcttttttgctcctctaacatatggtaatactgcgctttcggcggaggaggttcagcagcgacaatggttagatatgaagctccaattcgccaagtaagagtatacaatgtgtagttcaacgcaaaggaaattctaactttctgtgcaatctggtgacaatttctaaataactatcacaattggaagaagagattatcccaaatcttatcaaaaaatcgatgattgccagtgcacaattaggcttgaatttttcttgcagcaacgaagagattacttcagtgatgttcattagcctgaaatcttcactttcgtggtctatcggattaggaattagaccttgtttcatcggcaggtcgtatatgtattccacttctggttgaataaaatcttcgggtggtttgtttctgaacatatatgagatggctcccactggactgatatattgcgaaacatagtcctcattcaaccctgcctcctcgtaacattctttcaggcaagtttgcaaagtgccattaggatattccaagcctcctgccacagtattatctaacataccgggaaatgttggtttgtgtctgcttctcctaggtatccaaagttgaatactgttaggatcggcagaattttgcaaatatccattgatatgaactccataagtaacaactcccaaaatattagaaaaagccctttccaccaacatgtacatcttatggttatcgcagtaaactgcaaaaagctcatttctccaaccgctaagggtttcaaagagacgctgatctctccaacgctgagctatctttgcaaacatctgcgttcttttattttcggtatccagactaggaattatcttgacttcgtgtttttcattatttactatcacagcctgtgtttcgaactcaaattgttttgccaccttgggaattatataccctagtaagatcccatcatgcgataagaatttatacacagatacttcaaattcatgaaaagatggctcatctttatgaggaacagaatcaacagatctgactagatcaatatatggcattggttgattttattcaatggttatctatctcaaacatgctataaaaataaggtaattcctttatggtgttagggtgttatagtttttgcgtagaaaataattgtcatcatttttgggcaacctatgaaacaactactcagagaagttgagacatctcttttgacaaatgaaaccgaaatatcccctgcccttaagctattaattactcagttaaataaatcaacccatgaagataaatcaacagaaagaaaaacgttttggctagcattagacaatttaaggcaaaaaatcggtctacaatcccaatcacatgtccttttctttctacatctttttgaagagctagctccaactttagaaaatgagaaaatatttttaacctggattacttcttttttgaagttagcaattaatagtgcaggggtaccacattgtgtggtgaacgagtcaaggagaattataatgaatttattattgccctcaaaagctacaaacaccgaatacaatttgttaaagaattctgctgcaggcattcaattacttgtgcaagtgtatttgctaaaaactgatttagttgttgattccacttctagtagtccccaggagtatgaagagagggttagattcataaagaaaaactgcagggatttactacaaggtcttgatttaaataatcaagtactagaggctatcagcaaagaatttacggatcctcactaccgcttcgagtgcttcgtacttttgtcctcattaatgtcgtcatcagccttgttgtaccagataatgcaaacaactttgtggcataatatacttttgtctatattgatagataaaagtaacagtgtggttgagtcaggaatcaaggttctcagtatggttttgccccacgtctgtgatgtaatagcggattatctaccgaccattatggcgattttaagtaaaggtctggggggtgttgaaattgatgatgagtcaccattaccatcaaattggaaagtattgaatgatcaggatcctgaaattattggtccagcatttgttagctataaacaactgttcactgtattatacggcctgttccctcttagtttaacatcatttattcgcagtccatctacatatatcgactctaacaagattatagacgatctcaagcttcagttgcttgaaactaaagtgaagtcaaagtgtcaggacttgctaaagtgttttattgttcatccaaattattttatatattcttcccaggaggaagaaatttttgatacttcaaggtgggacaaaatgcactccccgaacgagatagcagcattttgttatcaattggaattccgtgggacatcgaaggagaatgcctttgatatgagggtagatgaccttttggaaggtcatcgatatctatatttgaaagatatgaaggatgcgcagaaagagagggctaaaaaatgtgaaaattctattatctcactcgaaagttcatctgatagtaagtcagtttcacaatacgacgaagactcgacgaaagaaaccacttgcaggcatgtttcgttttatttaagagagatccttttggcaaaaaatgaattggacttcacgctacatatcaatcaggtacttggagccgagtgtgagcttttgaaaaaaaaattgaacgaaatggataccctacgagatcaaaacaggtttttagctgacataaacgaaggtttacgaatacagcaatctaaggcgagtgagcaaattacggaattgctcaaagaaaaagagcgttctcaaaatgatttcaactctctggttactcatatgcttaaacaatctaacgaattaaaagaaagggagtcgaaactagtcgagattcatcaatcaaatgatgcagagataggagatttaaattataggttggaaaaactgtgcaaccttatacaacccaaagaattagaagtggaactgctcaagaagaagttgcgtgtagcatcgatccttttttcgcaagataaatcaaaatcttcaagcaagacatctctagcacatttgcaccaggcaggcgacgcaact

SEQ ID NO: 50 - protein, 741 aa, Pichia pastoris
MIKSRKRSRKVSLNTEKELKNSHISLGDERWYTVGLLLVTITAFCTRFYAINYPDEVVFDEVHFGKFASYYLERTYFFDLHPPFAKLLIAFVGFLAGYNGEFKFTTIGESYIKNEVPYVVYRSLSAVQGSLTVPIVYLCLKECGYTVLTCVFGACIILFDGAHVAETRLILLDATLIFFVSLSIYSYIKFTKQRSEPFGQKWWKWLFFTGVSLSCVISTKYVGVFTYLTIGCGVLFDLWSLLDYKKGHSLAYVGKHFAARFFLLILVPFLIYLNWFYVHFAILSKSGPGDSFMSSEFQETLGDSPLAAFAKEVHFNDIITIKHKETDAMLHSHLANYPLRYEDGRVSSQGQQVTAYSGEDPNNNWQIISPEGLTGVVTQGDVVRLRHVGTDGYLLTHDVASPFYPTNEEFTVVGQEKATQRWNETLFRIDPYDKKKTRPLKSKASFFKLIHVPTVVAMWTHNDQLLPDWGFNQQEVNGNKKLADESNLWVVDNIVDIAEDDPRKHYVPKEVKNLPFLTKWLELQRLMFIQNNKLSSDHPFASDPISWPFSLSGVSFWTNNESRKQIYFVGNIPGWWMEVAALGSFLGLVFADQFTRRRNSLVLTNSARSRLYNNLGFFFVGWCCHYLPFFLMSRQKFLHHYLPAHLIAAMFTAGFLEFIFTDNRTEEFKDQKTSCEPNSNSSKPKEQLILWLSFSSFVALLLSIIVWTFFFFAPLTYGNTALSAEEVQQRQWLDMKLQFAK

SEQ ID NO: 51 - DNA, 1380 bp, Artificial Sequence
  Encodes anti-DKK1 Heavy chain (VH + IgG2m4) with alpha-amylase leader
acgatggtcgcttggtggtctttgtttctgtacggtcttcaggtcgctgcacctgctttggctgaggttcagttggttcaatctggtgctgaggttaagaaacctggtgcttccgttaaggtttcctgtaaggcttccggttacactttcactgactactacatccactgggttagacaagctccaggtcaaggattggaatggatgggatggattcactctaactccggtgctactacttacgctcagaagttccaggctagagttactatgtccagagacacttcttcttccactgcttacatggaattgtccagattggaatccgatgacactgctatgtacttttgttccagagaggactactggggacagggaactttggttactgtttcctccgcttctactaaagggccctctgtttttccattggctccatgttctagatccacttccgaatccactgctgctttgggatgtttggttaaggactacttcccagagccagttactgtttcttggaactccggtgctttgacttctggtgttcacactttcccagctgttttgcaatcttccggtttgtactccttgtcctccgttgttactgttacttcctccaacttcggtactcagacttacacttgtaacgttgaccacaagccatccaacactaaggttgacaagactgttgagagaaagtgttgtgttgagtgtccaccatgtccagctccaccagttgctggtccatccgtttttttgttcccaccaaagccaaaggacactttgatgatctccagaactccagaggttacatgtgttgttgttgacgtttcccaagaggacccagaggttcaattcaactggtacgttgacggtgttgaagttcacaacgctaagactaagccaagagaagagcagttcaactccactttcagagttgtttccgttttgactgttttgcaccaggattggttgaacggtaaagaatacaagtgtaaggtttccaacaagggattgccatcctccatcgaaaagactatctccaagactaagggacaaccaagagagccacaggtttacactttgccaccatccagagaagagatgactaagaaccaggtttccttgacttgtttggttaaaggattctacccatccgacattgctgttgagtgggaatctaacggtcaaccagagaacaactacaagactactccaccaatgttggattctgacggttccttcttcttgtactccaagttgactgttgacaagtccagatggcaacagggtaacgttttctcctgttccgttatgcatgaggctttgcacaaccactacactcaaaagtccttgtctttgtcccctggtaagtaa

SEQ ID NO: 52 - protein, 438 aa, Artificial Sequence
  anti-DKK1 Heavy chain (VH + IgG2m4)
EVQLVQSGAEVKKPGASVKVSCKASGYTFTDYYIHWVRQAPGQGLEWMGWIHSNSGATTYAQKFQARVTMSRDTSSSTAYMELSRLESDDTAMYFCSREDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 53 - DNA, 717 bp, Artificial Sequence
  Encodes anti-DKK1 Light chain (VL + lambda constant regions) with alpha-amylase leader
acgatggtcgcttggtggtctttgtttctgtacggtcttcaggtcgctgcacctgctttggctcagtccgttttgacacaaccaccatctgtttctggtgctccaggacagagagttactatctcctgtactggttcctcttccaacattggtgctggttacgatgttcactggtatcaacagttgccaggtactgctccaaagttgttgatctacggttactccaacagaccatctggtgttccagacagattctctggttctaagtctggtgcttctgcttccttggctatcactggattgagaccagatgacgaggctgactactactgtcaatcctacgacaactccttgtcctcttacgttttcggtggtggtactcagttgactgttttgtcccagccaaaggctaatccaactgttactttgttcccaccatcttccgaagaactgcaggctaataaggctactttggtttgtttgatctccgacttctacccaggtgctgttactgttgcttggaaggctgatggttctccagttaaggctggtgttgagactactaagccatccaagcagtccaataacaagtacgctgctagctcttacttgtccttgacaccagaacaatggaagtcccacagatcctactcttgtcaggttacacacgagggttctactgttgaaaagactgttgctccaactgagtgttcctaa

SEQ ID NO: 54 - protein, 217 aa, Artificial Sequence
  aanti-DKK1 Light chain (VL + lambda constant regions)
QSVLTQPPSVSGAPGQRVTISCTGSSSNIGAGYDVHWYQQLPGTAPKLLIYGYSNRPSGVPDRFSGSKSGASASLAITGLRPDDEADYYCQSYDNSLSSYVFGGGTQLTVLSQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 55 - DNA, 1908 bp, Artificial Sequence
  encodes human BIP
gaggaagaggacaagaaagaggatgttggtactgttgtcggtatcgacttgggtactacctactcctgtgtcggtgttttcaagaacggtagagtggagattatcgccaacgaccagggtaacagaattactccatcctacgttgcttttaccccagaaggagagagattgatcggagacgctgctaagaaccaattgacctccaacccagagaacactgttttcgacgccaagagactgattggtagaacttggaacgacccatccgttcaacaagacatcaagttcttgcccttcaaggtcgtcgagaagaaaaccaagccatacatccaggttgacatcggtggtggtcaaactaagactttcgctccagaggaaatctccgctatggtcctgactaagatgaaagagactgccgaggcttacttgggtaaaaaggttacccacgctgttgttactgttccagcttacttcaacgacgctcagagacaagctactaaggacgctggtactatcgctggactgaacgtgatgagaatcatcaacgagccaactgctgctgctattgcctacggattggacaagagagagggagagaagaacatcttggttttcgacttgggtggtggtactttcgacgtttccttgttgaccatcgacaacggtgttttcgaagttgttgctaccaacggtgatactcacttgggtggagaggacttcgatcagagagtgatggaacacttcatcaagctgtacaagaagaaaaccggaaaggacgttagaaaggacaacagagccgttcagaagttgagaagagaggttgagaaggctaaggctttgtcctcccaacaccaagctagaatcgagatcgaatccttctacgagggtgaagatttctccgagaccttgactagagccaagttcgaagagctgaacatggacctgttcagatccactatgaagccagttcagaaggttttggaggattccgacttgaagaagtccgacatcgacgagattgttttggttggtggttccaccagaatcccaaagatccagcagctggtcaaagagttcttcaacggtaaagagccatccagaggtattaacccagatgaggctgttgcttacggtgctgctgttcaagctggtgttttgtctggtgaccaggacactggtgacttggttttgttgcatgtttgcccattgactttgggtatcgagactgttggtggtgttatgaccaagttgatcccatccaacactgttgttcccaccaagaactcccaaattttctccactgcttccgacaaccagccaaccgttactattaaggtctacgaaggtgaaagaccattgaccaaggacaaccacttgttgggaactttcgacttgactggtattccacctgctccaagaggtgttccacaaatcgaggttaccttcgagatcgacgtcaacggtatcttgagagttactgccgaggataagggaaccggtaacaagaacaagatcaccatcaccaacgaccaaaacagattgacccccgaagagatcgaaagaatggtcaacgatgctgagaagttcgccgaagaggataagaagctgaaagagagaatcgacaccagaaacgagttggaatcctacgcttactccttgaagaaccagatcggtgacaaagaaaagttgggtggaaagctgtcatccgaagataaagaaactatggaaaaggccgtcgaagaaaagattgagtggctggaatctcaccaagatgctgacatcgaggacttcaaggccaagaagaaagagttggaagagatcgtccagccaatcatttctaagttgtacggttctgctggtccaccaccaactggtgaagaagatactgccgagcacgacgagttgtag

SEQ ID NO: 56 - protein, 635 aa, Artificial Sequence
  human BIP without leader
EEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLHVCPLTLGIETVGGVMTKLIPSNTVVPTKNSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEHDEL

SEQ ID NO: 57 - DNA, 1896 bp, Artificial Sequence
  Encodes chimeric BIP
gacgatgtcgaatcttatggaacagtgattggtatcgatttgggtaccacgtactcttgtgtcggtgtgatgaagtcgggtcgtgtagaaattcttgctaatgaccaaggtaacagaatcactccttcctacgttagtttcactgaagacgagagactggttggtgatgctgctaagaacttagctgcttctaacccaaaaaacaccatctttgatattaagagattgatcggtatgaagtatgatgccccagaggtccaaagagacttgaagcgtcttccttacactgtcaagagcaagaacggccaacctgtcgtttctgtcgagtacaagggtgaggagaagtctttcactcctgaggagatttccgccatggtcttgggtaagatgaagttgatcgctgaggactacttaggaaagaaagtcactcatgctgtcgttaccgttccagcctacttcaacgacgctcaacgtcaagccactaaggatgccggtctcatcgccggtttgactgttctgagaattgtgaacgagcctaccgccgctgcccttgcttacggtttggacaagactggtgaggaaagacagatcatcgtctacgacttgggtggaggaaccttcgatgtttctctgctttctattgagggtggtgctttcgaggttcttgctaccgccggtgacacccacttgggtggtgaggactttgactacagagttgttcgccacttcgttaagattttcaagaagaagcataacattgacatcagcaacaatgataaggctttaggtaagctgaagagagaggtcgaaaaggccaagcgtactttgtcttcccagatgactaccagaattgagattgactctttcgtcgacggtatcgacttctctgagcaactgtctagagctaagtttgaggagatcaacattgaattattcaagaagacactgaaaccagttgaacaagtcctcaaagacgctggtgtcaagaaatctgaaattgatgacattgtcttggttggtggttctaccagaattccaaaggttcaacaattattggaggattactttgacggaaagaaggcttctaagggaattaacccagatgaagctgtcgcatacggtgctgctgttcaggctggtgttttgtctggtgatcaagatacaggtgacctggtactgcttgatgtatgtccccttacacttggtattgaaactgtgggaggtgtcatgaccaaactgattccaaggaacacagtggtgcctaccaagaagtctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctcaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaacatgatgagttgtag

SEQ ID NO: 58 - protein, 631 aa, Artificial Sequence
  chimeric BIP
DDVESYGTVIGIDLGTTYSCVGVMKSGRVEILANDQGNRITPSYVSFTEDERLVGDAAKNLAASNPKNTIFDIKRLIGMKYDAPEVQRDLKRLPYTVKSKNGQPVVSVEYKGEEKSFTPEEISAMVLGKMKLIAEDYLGKKVTHAVVTVPAYFNDAQRQATKDAGLIAGLTVLRIVNEPTAAALAYGLDKTGEERQIIVYDLGGGTFDVSLLSIEGGAFEVLATAGDTHLGGEDFDYRVVRHFVKIFKKKHNIDISNNDKALGKLKREVEKAKRTLSSQMTTRIEIDSFVDGIDFSEQLSRAKFEEINIELFKKTLKPVEQVLKDAGVKKSEIDDIVLVGGSTRIPKVQQLLEDYFDGKKASKGINPDEAVAYGAAVQAGVLSGDQDTGDLVLLDVCPLTLGIETVGGVMTKLIPRNTVVPTKKSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEHDEL

SEQ ID NO: 59 - DNA, 254 bp, Artificial Sequence
  Pichia pastoris PDI1 promoter
aacacgaacactgtaaatagaataaaagaaaacttggatagtagaacttcaatgtagtgtttctattgtcttacgcggctctttagattgcaatccccagaatggaatcgtccatctttctcaacccactcaaagataatctaccagacatacctacgccctccatcccagcaccacgtcgcgatcacccctaaaacttcaataattgaacacgtactgatttccaaaccttcttcttcttcctatctataaga

SEQ ID NO: 60 - DNA, 2775 bp, Pichia pastoris
atgacagctaatgaaaatccttttgagaatgagctgacaggatcttctgaatctgccccccctgcattggaatcgaagactggagagtctcttaagtattgcaaatataccgtggatcaggtcatagaagagtttcaaacggatggtctcaaaggattgtgcaattcccaggacatcgtatatcggaggtctgttcatgggccaaatgaaatggaagtcgaagaggaagagagtcttttttcgaaattcttgtcaagtttctacagcgatccattgattctgttactgatgggttccgctgtgattagctttttgatgtctaacattgatgatgcgatatctatcactatggcaattacgatcgttgtcacagttggatttgttcaagagtatcgatccgagaaatcattggaggcattgaacaagttagtccctgccgaagctcatctaactaggaatgggaacactgaaactgttcttgctgccaacctagtcccaggagacttggtggatttttcggttggtgacagaattccggctgatgtgagaattattcacgcttcccacttgagtatcgacgagagcaacctaactggtgaaaatgaaccagtttctaaagacagcaaacctgttgaaagtgatgacccaaacattcccttgaacagccgttcatgtattgggtatatgggcactttagttcgtgatggtaatggcaaaggtattgtcatcggaacagccaaaaacacagcttttggctctgttttcgaaatgatgagctctattgagaaaccaaagactcctcttcaacaggctatggataaacttggtaaggatttgtctgctttttccttcggaatcatcggccttatttgcttggttggtgtttttcaaggtagaccctggttggaaatgttccagatctctgtatccttggctgttgctgcgattccagaaggtcttcctattattgtgactgtgactcttgctcttggtgtgttgcgtatggctaaacagagggccatcgtcaaaagactgcctagtgttgaaactttgggatccgtcaatgttatctgtagtgataagacgggaacattgacccaaaatcatatgaccgttaacagattatggactgtggatatgggcgatgaattcttgaaaattgaacaaggggagtcctatgccaattatctcaaacccgatacgctaaaagttctgcaaactggtaatatagtcaacaatgccaaatattcaaatgaaaaggaaaaatacctcggaaacccaactgatattgcaattattgaatctttagaaaaatttgatttgcaggacattagagcaacaaaggaaagaatgttggagattccattttcttcgtccaagaaatatcaggccgtcagtgttcactctggagacaaaagcaaatctgaaatttttgttaaaggcgctctgaacaaagttttggaaagatgttcaagatattacaatgctgaaggtatcgccactccactcacagatgaaattagaagaaaatccttgcaaatggccgatacgttagcatcttcaggattgagaatactgtcgtttgcttacgacaaaggcaattttgaagaaactggcgatggaccatcggatatgatcttttgtggtcttttaggtatgaacgatcctcctagaccatctgtaagtaaatcaattttgaaattcatgagaggtggggttcacattattatgattacaggagattcagaatccacggccgtagccgttgccaaacaggtcggaatggtaattgacaattcaaaatatgctgtcctcagtggagacgatatagatgctatgagtacagagcaactgtctcaggcgatctcacattgttctgtatttgcccggactactccaaaacataaggtgtccattgtaagagcactacaggccagaggagatattgttgcaatgactggtgacggtgtcaatgatgccccagctctaaaactggccgacatcggaattgccatgggtaatatggggaccgatgttgccaaagaggcagccgacatggttttgactgatgatgacttttctacaatcttatctgcaatccaggagggtaaaggtattttctacaacatccagaactttttaacgttccaactttctacttcaattgctgctctttcgttaattgctctgagtactgctttcaacctgccaaatccattgaatgccatgcagattttgtggatcaatattatcatggatggacctccagctcagtctttgggtgttgagccagttgataaagctgtgatgaacaaaccaccaagaaagcgaaatgataaaattctgacaggtaaggtgattcaaagggtagtacaaagtagttttatcattgtttgtggtactctgtacgtatacatgcatgagatcaaagataatgaggtcacagcaagagacactacgatgacctttacatgctttgtattctttgacatgttcaacgcattaacgacaagacaccattctaaaagtattgcagaacttggatggaataatactatgttcaacttttccgttgcagcttctattttgggtcaactaggagctatttacattccatttttgcagtctattttccagactgaacctctgagcctcaaagatttggtccatttattgttgttatcgagttcagtatggattgtagacgagcttcgaaaactctacgtcaggagacgtgacgcatccccatacaatggatacagcatggctgtttga

SEQ ID NO: 61 - protein, 924 aa, Pichia pastoris
MTANENPFENELTGSSESAPPALESKTGESLKYCKYTVDQVIEEFQTDGLKGLCNSQDIVYRRSVHGPNEMEVEEEESLFSKFLSSFYSDPLILLLMGSAVISFLMSNIDDAISITMAITIVVTVGFVQEYRSEKSLEALNKLVPAEAHLTRNGNTETVLAANLVPGDLVDFSVGDRIPADVRIIHASHLSIDESNLTGENEPVSKDSKPVESDDPNIPLNSRSCIGYMGTLVRDGNGKGIVIGTAKNTAFGSVFEMMSSIEKPKTPLQQAMDKLGKDLSAFSFGIIGLICLVGVFQGRPWLEMFQISVSLAVAAIPEGLPIIVTVTLALGVLRMAKQRAIVKRLPSVETLGSVNVICSDKTGTLTQNHMTVNRLWTVDMGDEFLKIEQGESYANYLKPDTLKVLQTGNIVNNAKYSNEKEKYLGNPTDIAIIESLEKFDLQDIRATKERMLEIPFSSSKKYQAVSVHSGDKSKSEIFVKGALNKVLERCSRYYNAEGIATPLTDEIRRKSLQMADTLASSGLRILSFAYDKGNFEETGDGPSDMIFCGLLGMNDPPRPSVSKSILKFMRGGVHIIMITGDSESTAVAVAKQVGMVIDNSKYAVLSGDDIDAMSTEQLSQAISHCSVFARTTPKHKVSIVRALQARGDIVAMTGDGVNDAPALKLADIGIAMGNMGTDVAKEAADMVLTDDDFSTILSAIQEGKGIFYNIQNFLTFQLSTSIAALSLIALSTAFNLPNPLNAMQILWINIIMDGPPAQSLGVEPVDKAVMNKPPRKRNDKILTGKVIQRVVQSSFIIVCGTLYVYMHEIKDNEVTARDTTMTFTCFVFFDMFNALTTRHHSKSIAELGWNNTMFNFSVAASILGQLGAIYIPFLQSIFQTEPLSLKDLVHLLLLSSSVWIVDELRKLYVRRRDASPYNGYSMAV

SEQ ID NO: 62 - DNA, 3186 bp, Artificial Sequence
  Arabidopsis thalian DNA encoding ECA1 codon-optimized for Pichia expression
atgggaaagggttccgaggacctggttaagaaagaatccctgaactccactccagttaactctgacactttcccagcttgggctaaggatgttgctgagtgcgaagagcacttcgttgtttccagagagaagggtttgtcctccgacgaagtcttgaagagacaccaaatctacggactgaacgagttggaaaagccagagggaacctccatcttcaagctgatcttggagcagttcaacgacacccttgtcagaattttgttggctgccgctgttatttccttcgtcctggctttttttgatggtgacgagggtggtgaaatgggtatcactgccttcgttgagcctttggtcatcttcctgatcttgatcgttaacgccatcgttggtatctggcaagagactaacgctgaaaaggctttggaggccttgaaagagattcaatcccagcaggctaccgttatgagagatggtactaaggtttcctccttgccagctaaagaattggttccaggtgacatcgttgagctgagagttggtgataaggttccagccgacatgagagttgttgctttgatctcctccaccttgagagttgaacaaggttccctgactggtgaatctgaggctgtttccaagactactaagcacgttgacgagaacgctgacatccagggtaaaaagtgcatggttttcgccggtactaccgttgttaacggtaactgcatctgtttggtcactgacactggaatgaacaccgagatcggtagagttcactcccaaatccaagaagctgctcaacacgaagaggacaccccattgaagaagaagctgaacgagttcggagaggtcttgaccatgatcatcggattgatctgtgccctggtctggttgatcaacgtcaagtacttcttgtcctgggaatacgttgatggatggccaagaaacttcaagttctccttcgagaagtgcacctactacttcgagatcgctgttgctttggctgttgctgctattccagagggattgccagctgttatcaccacttgcttggccttgggtactagaaagatggctcagaagaacgcccttgttagaaagttgccatccgttgagactttgggttgtactaccgtcatctgttccgacaagactggtactttgactaccaaccagatggccgtttccaaattggttgccatgggttccagaatcggtactctgagatccttcaacgtcgagggaacttcttttgacccaagagatggaaagattgaggactggccaatgggtagaatggacgccaacttgcagatgattgctaagatcgccgctatctgtaacgacgctaacgttgagcaatccgaccaacagttcgtttccagaggaatgccaactgaggctgccttgaaggttttggtcgagaagatgggtttcccagaaggattgaacgaggcttcttccgatggtgacgtcttgagatgttgcagactgtggagtgagttggagcagagaatcgctactttggagttcgacagagatagaaagtccatgggtgtcatggttgattcttcctccggtaacaagttgttgttggtcaaaggagcagttgaaaacgttttggagagatccacccacattcaattgctggacggttccaagagagaattggaccagtactccagagacttgatcttgcagtccttgagagacatgtccttgtccgccttgagatgtttgggtttcgcttactctgacgttccatccgatttcgctacttacgatggttctgaggatcatccagctcaccaacagttgctgaacccatccaactactcctccatcgaatccaacctgatcttcgttggtttcgtcggtcttagagacccaccaagaaaagaagttagacaggccatcgctgattgtagaaccgccggtatcagagttatggtcatcaccggagataacaagtccactgccgaggctatttgtagagagatcggagttttcgaggctgacgaggacatttcttccagatccctgaccggtattgagttcatggacgtccaagaccagaagaaccacttgagacagaccggtggtttgttgttctccagagccgaaccaaagcacaagcaagagattgtcagactgctgaaagaggacggagaagttgttgctatgaccggtgatggtgttaatgacgccccagctttgaagttggctgacatcggtgttgctatgggaatttccggtactgaagttgctaaggaagcctccgatatggttttggctgacgacaacttttcaactatcgttgctgctgtcggagaaggtagaagtatctacaacaacatgaaagcctttatcagatacatgatttcctccaacatcggtgaagttgcctccattttcttgactgctgccttgggtattcctgagggaatgatcccagttcagttgttgtgggttaacttggttactgacggtccacctgctactgctttgggtttcaacccaccagacaaagacattatgaagaagccaccaagaagatccgacgattccttgatcaccgcctggatcttgttcagatacatggtcatcggtctttatgttggtgttgccaccgtcggtgttttcatcatctggtacacccactcttccttcatgggtattgacttgtctcaagatggtcattctttggtttcctactcccaattggctcattggggacaatgttcttcctgggagggtttcaaggtttccccattcactgctggttcccagactttctccttcgattccaacccatgtgactacttccagcagggaaagatcaaggcttccaccttgtctttgtccgttttggtcgccattgagatgttcaactccctgaacgctttgtctgaggacggttccttggttactatgccaccttgggtgaacccatggttgttgttggctatggctgtttccttcggattgcacttcgtcatcctgtacgttccattcttggcccaggttttcggtattgttccactgtccttgaacgagtggttgttggtcttggccgtttctttgccagttatcctgatcgacgaggttttgaagttcgttggtagatgcacctctggttacagatactccccaagaactctgtccaccaagcagaaagaagagtaa

SEQ ID NO: 63 - protein, 1061 aa, Arabidopsis thaliana
MGKGSEDLVKKESLNSTPVNSDTFPAWAKDVAECEEHFVVSREKGLSSDEVLKRHQIYGLNELEKPEGTSIFKLILEQFNDTLVRILLAAAVISFVLAFFDGDEGGEMGITAFVEPLVIFLILIVNAIVGIWQETNAEKALEALKEIQSQQATVMRDGTKVSSLPAKELVPGDIVELRVGDKVPADMRVVALISSTLRVEQGSLTGESEAVSKTTKHVDENADIQGKKCMVFAGTTVVNGNCICLVTDTGMNTEIGRVHSQIQEAAQHEEDTPLKKKLNEFGEVLTMIIGLICALVWLINVKYFLSWEYVDGWPRNFKFSFEKCTYYFEIAVALAVAAIPEGLPAVITTCLALGTRKMAQKNALVRKLPSVETLGCTTVICSDKTGTLTTNQMAVSKLVAMGSRIGTLRSFNVEGTSFDPRDGKIEDWPMGRMDANLQMIAKIAAICNDANVEQSDQQFVSRGMPTEAALKVLVEKMGFPEGLNEASSDGDVLRCCRLWSELEQRIATLEFDRDRKSMGVMVDSSSGNKLLLVKGAVENVLERSTHIQLLDGSKRELDQYSRDLILQSLRDMSLSALRCLGFAYSDVPSDFATYDGSEDHPAHQQLLNPSNYSSIESNLIFVGFVGLRDPPRKEVRQAIADCRTAGIRVMVITGDNKSTAEAICREIGVFEADEDISSRSLTGIEFMDVQDQKNHLRQTGGLLFSRAEPKHKQEIVRLLKEDGEVVAMTGDGVNDAPALKLADIGVAMGISGTEVAKEASDMVLADDNFSTIVAAVGEGRSIYNNMKAFIRYMISSNIGEVASIFLTAALGIPEGMIPVQLLWVNLVTDGPPATALGFNPPDKDIMKKPPRRSDDSLITAWILFRYMVIGLYVGVATVGVFIIWYTHSSFMGIDLSQDGHSLVSYSQLAHWGQCSSWEGFKVSPFTAGSQTFSFDSNPCDYFQQGKIKASTLSLSVLVAIEMFNSLNALSEDGSLVTMPPWVNPWLLLAMAVSFGLHFVILYVPFLAQVFGIVPLSLNEWLLVLAVSLPVILIDEVLKFVGRCTSGYRYSPRTLSTKQKEE

SEQ ID NO: 64 - DNA, 39 bp, Artificial Sequence
  PpPMR1/UP PCR primer
gaattcatgacagctaatgaaaatccttttgagaatgag

SEQ ID NO: 65 - DNA, 37 bp, Artificial Sequence
  PpPMR1/LP PCR primer
ggccggcctcaaacagccatgctgtatccattgtatg

SEQ ID NO: 66 - DNA, 25 bp, Artificial Sequence
  5′AOX1 PCR primer
gcgactggttccaattgacaagctt

SEQ ID NO: 67 - DNA, 30 bp, Artificial Sequence
  PpPMR1/cLP PCR primer
ggttgctctcgtcgatactcaagtgggaag

SEQ ID NO: 68 - DNA, 30 bp, Artificial Sequence
  AtECA1/cLP PCR primer
gtcggctggaaccttatcaccaactctcag

SEQ ID NO: 69 - DNA, 1314 bp, Homo sapiens
atgagatttccttcaatttttactgctgttttattcgcagcatcctccgcattagcttacccatacgacgtcccagactacgcttacccatacgacgtcccagactacgctgagcccgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggcttaaggaggaggaagaagacaagaaacgcaaagaggaggaggaggcagaggacaaggaggatgatgaggacaaagatgaggatgaggaggatgaggaggacaaggaggaagatgaggaggaagatgtccccggccaggcccatgacgagctgtag

SEQ ID NO: 70 - protein, 437 aa, Artificial Sequence
  Human calreticulin (hCRT)-protein with Saccharomyces cerevisiae mating factor pre-signal peptide leader
MRFPSIFTAVLFAASSALAYPYDVPDYAYPYDVPDYAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAHDEL

SEQ ID NO: 71 - DNA, 1512 bp, Homo sapiens
atgcaattcaactggaacatcaagactgttgcttccatcttgtccgctttgactttggctcaagcttctgacgttttggagttgactgacgacaacttcgagtccagaatttctgacactggttccgctggattgatgttggttgagttcttcgctccatggtgtggtcattgtaagagattggctccagaatacgaagctgctgctactagattgaagggtatcgttccattggctaaggttgactgtactgctaacactaacacttgtaacaagtacggtgtttccggttacccaactttgaagatcttcagagatggtgaagaagctggagcttacgacggtccaagaactgctgacggtatcgtttcccacttgaagaagcaagctggtccagcttctgttccattgagaactgaggaggagttcaagaagttcatctccgacaaggacgcttctatcgttggtttcttcgacgattctttctctgaagctcactccgaattcttgaaggctgcttccaacttgagagacaactacagattcgctcacactaacgttgagtccttggttaacgagtacgacgataacggtgaaggtatcatcttgttcagaccatcccacttgactaacaagttcgaggacaagacagttgcttacactgagcagaagatgacttccggaaagatcaagaagtttatccaagagaacatcttcggtatctgtccacacatgactgaggacaacaaggacttgattcagggaaaggacttgttgatcgcttactacgacgttgactacgagaagaacgctaagggttccaactactggagaaacagagttatgatggttgctaagaagttcttggacgctggtcacaagttgaacttcgctgttgcttctagaaagactttctcccacgagttgtctgatttcggattggaatccactgctggagagattccagttgttgctatcagaactgctaagggagagaagttcgttatgcaagaggagttctccagagatggaaaggctttggagagattcttgcaggattacttcgacggtaacttgaagagatacttgaagtccgagccaattccagaatctaacgacggtccagttaaagttgttgttgctgagaacttcgacgagatcgttaacaacgagaacaaggacgttttgatcgagttttacgctccttggtgtggacactgtaaaaacttggagccaaagtacaaggaattgggtgaaaagttgtccaaggacccaaacatcgttatcgctaagatggacgctactgctaacgatgttccatccccatacgaagttagaggtttcccaactatctacttctccccagctaacaagaagttgaacccaaagaagtacgagggaggtagagaattgtccgacttcatctcctacttgcagagagaggctactaatccaccagttatccaagaggagaagccaaagaagaagaagaaagctcacgacgagttgtag

SEQ ID NO: 72 - protein, 503 aa, Homo sapiens
MQFNWNIKTVASILSALTLAQASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAHDEL

SEQ ID NO: 73 - DNA, 55 bp, Artificial Sequence
  hCRT-BstZ17I-HA/UP PCR primer
gtatacccatacgacgtcccagactacgctgagcccgccgtctacttcaaggagc

SEQ ID NO: 74 - DNA, 45 bp, Artificial Sequence
  hCRT-PacI/LP PCR primer
ttaattaactacagctcgtcatgggcctggccggggacatcttcc

SEQ ID NO: 75 - protein, 7 aa, Artificial Sequence
  Synthetic peptide that binds CRT
KLGFFKR

SEQ ID NO: 76 - DNA, 1068 bp, Artificial Sequence
  Encodes human ERdj3 with Saccharomyces cerevisiae mating factor pre-signal peptide leader
atgagatttccttcaatttttactgctgttttattcgcagcatcctccgcattagctggtagagacttctacaagattttgggtgttccaagatccgcttccatcaaggacatcaagaaggcttacagaaagttggctttgcaattgcacccagacagaaacccagatgacccacaagctcaagagaagttccaagacttgggtgctgcttacgaagttttgtccgattccgagaagagaaagcagtacgacacttacggtgaagaaggattgaaggacggtcaccaatcttctcacggtgacatcttctcccactttttcggtgacttcggtttcatgttcggtggtactccaagacaacaggacagaaacatcccaagaggttccgacattatcgttgacttggaggttacattggaagaggtttacgctggtaacttcgttgaagttgttagaaacaagccagttgctagacaagctccaggtaaaagaaagtgtaactgtagacaagagatgagaactactcagttgggtcctggtagattccaaatgacacaggaagttgtttgcgacgagtgtccaaacgttaagttggttaacgaagagagaactttggaggttgagatcgagccaggtgttagagatggaatggaatacccattcatcggtgaaggtgaaccacatgttgatggtgaacctggtgacttgagattcagaatcaaagttgttaagcacccaatcttcgagagaagaggtgacgacttgtacactaacgttactatttccttggttgaatccttggttggtttcgagatggacatcactcatttggacggtcacaaggttcacatttccagagacaagatcactagaccaggtgctaagttgtggaagaagggtgaaggattgccaaacttcgacaacaacaacatcaagggatctttgatcatcactttcgacgttgacttcccaaaagagcagttgactgaagaagctagagagggtatcaagcagttgttgaagcaaggttccgttcagaaggtttacaacggattgcagggatactaa

SEQ ID NO: 77 - protein, 355 aa, Artificial Sequence
  human ERdj3 with Saccharomyces cerevisiae mating factor pre-signal peptide leader
MRFPSIFTAVLFAASSALAGRDFYKILGVPRSASIKDIKKAYRKLALQLHPDRNPDDPQAQEKFQDLGAAYEVLSDSEKRKQYDTYGEEGLKDGHQSSHGDIFSHFFGDFGFMFGGTPRQQDRNIPRGSDIIVDLEVTLEEVYAGNFVEVVRNKPVARQAPGKRKCNCRQEMRTTQLGPGRFQMTQEVVCDECPNVKLVNEERTLEVEIEPGVRDGMEYPFIGEGEPHVDGEPGDLRFRIKVVKHPIFERRGDDLYTNVTISLVESLVGFEMDITHLDGHKVHISRDKITRPGAKLWKKGEGLPNFDNNNIKGSLIITFDVDFPKEQLTEEAREGIKQLLKQGSVQKVYNGLQGY

(19) United States
(12) Patent Application Publication
(10) Pub. No.: US 2011/0312032 A1
(43) Pub. Date: Dec. 22, 2011

(54) VECTORS AND YEAST STRAINS FOR PROTEIN PRODUCTION

(76) Inventors: Byung-Kwon Choi, Norwich, VT (US); Piotr Bobrowicz, Hanover, NH (US); W. James Cook, Hanover, NH (US); Elena E. Brevnova, Lebanon, NH (US)

(21) Appl. No.: 13/207,848

(22) Filed: Aug. 11, 2011

Related U.S. Application Data
(63) Continuation of application No. 12/863,468, filed on Jul. 19, 2010.
(60) Provisional application No. 61/066,409, filed on Feb. 20, 2008.
(60) Provisional application No. 61/188,723, filed on Aug. 12, 2008.

(51) Int. Cl. C12P 21/00; C12N 9/00; C12N 1/19

(52) U.S. Cl. 435/69.4; 435/254.23; 435/69.1; 435/69.6; 435/69.7; 435/183

(57) ABSTRACT

Lower eukaryote host cells in which the function of at least one endogenous gene encoding a chaperone protein, such as a Protein Disulphide Isomerase (PDI), has been reduced or eliminated and at least one mammalian homolog of the chaperone protein is expressed are described. In particular aspects, the host cells further include a deletion or disruption of one or more O-protein mannosyltransferase genes, and/or overexpression of an endogenous or exogenous Ca2⁺ATPase. These host cells are useful for producing recombinant glycoproteins in large amounts and for producing recombinant glycoproteins that have reduced O-glycosylation.

### CROSS REFERENCE TO RELATED APPLICATIONS

This patent application is a continuation of U.S. Ser. No. 12/863,468 filed 19 Jul. 2010, which in turn is a National Phase entry of PCT International Application No. PCT/US2009/033507 filed 9 Feb. 2009 and which claims benefit of U.S. Provisional Application No. 61/066,409, filed 20 Feb. 2008, and U.S. Provisional Application No. 61/188,723, filed 12 Aug. 2008.

### REFERENCE TO SEQUENCE LISTING SUBMITTED ELECTRONICALLY

The sequence listing of the present application is submitted electronically via EFS-Web as an ASCII formatted sequence listing with a file name “MRLBIO22395USCNT-SEQTXT-10AUG2011.txt”, creation date of Aug. 10, 2011 and a size of 163 KB. This sequence listing submitted via EFS-Web is part of the specification and is herein incorporated by reference in its entirety.

### BACKGROUND OF THE INVENTION

(1) Field of the Invention

The present invention relates to use of chaperone genes to improve protein production in recombinant expression systems. In general, recombinant lower eukaryote host cells comprise a nucleic acid encoding a heterologous chaperone protein and a deletion or disruption of the gene encoding the endogenous chaperone protein. These host cells are useful for producing recombinant glycoproteins in large amounts and for producing recombinant glycoproteins that have reduced O-glycosylation.

(2) Description of Related Art

Molecular chaperones play a critical role in the folding and secretion of proteins, and in particular, for the folding and secretion of antibodies. In lower eukaryotes, and particularly in yeast, Protein Disulfide Isomerase (PDI) is a chaperone protein, which functions to help create the disulphide bonds between multimeric proteins, such as those between antibody heavy and light chains. There have been past attempts to increase antibody expression levels in P. pastoris by overexpressing human PDI chaperone protein and/or overexpressing endogenous PDI. See for example, Wittrup et al., U.S. Pat. No. 5,772,245; Toyoshima et al., U.S. Pat. Nos. 5,700,678 and 5,874,247; Ng et al., U.S. Application Publication No. 2002/0068325; Toman et al., J. Biol. Chem. 275: 23303-23309 (2000); Keizer-Gunnink et al., Martix Biol. 19: 29-36 (2000); Vad et al., J. Biotechnol. 116: 251-260 (2005); Inana et al., Biotechnol. Bioengineer. 93: 771-778 (2005); Zhang et al., Biotechnol. Prog. 22: 1090-1095 (2006); Damasceno et al., Appl. Microbiol. Biotechnol. 74: 381-389 (2006); and, Huo et al., Protein express. Purif. 54: 234-239 (2007).

Protein disulfide isomerase (PDI) can produce a substantial increase or a substantial decrease in the recovery of disulfide-containing proteins, when compared with the uncatalyzed reaction; a high concentration of PDI in the endoplasmic reticulum (ER) is essential for the expression of disulfide-containing proteins (Puig and Gilbert, J. Biol. Chem., 269:7764-7771 (1994)). The action of PDI1 and its co-chaperones is shown in FIG. 2.

In Gunther et al., J. Biol. Chem., 268:7728-7732 (1993) the Trg1/Pdi1 gene of Saccharomyces cerevisiae was replaced by a murine gene of the protein disulfide isomerase family. It was found that two unglycosylated mammalian proteins PDI and ERp72 were capable of replacing at least some of the critical functions of Trg1, even though the three proteins diverged considerably in the sequences surrounding the thioredoxin-related domains; whereas ERp61 was inactive.

Development of further protein expression systems for yeasts and filamentous fungi, such as Pichia pastoris, based on improved vectors and host cell lines in which effective chaperone proteins would facilitate development of genetically enhanced yeast strains for the recombinant production of proteins, and in particular, for recombinant production of antibodies.

The present invention provides improved methods and materials for the production of recombinant proteins using auxiliary genes and chaperone proteins. In one embodiment, genetic engineering to humanize the chaperone pathway resulted in improved yield of recombinant antibody produced in Pichia pastoris cells.

As described herein, there are many attributes of the methods and materials of the present invention which provide unobvious advantages for such expression processes over prior known expression processes.

### BRIEF SUMMARY OF THE INVENTION

The present inventors have found that expression of recombinant proteins in a recombinant host cell can be improved by replacing one or more of the endogenous chaperone proteins in the recombinant host cell with one or more heterologous chaperone proteins. In general, it has been found that expression of a recombinant protein can be increased when the gene encoding an endogenous chaperone protein is replaced with a heterologous gene from the same or similar species as that of the recombinant protein to be produced in the host cell encoding a homolog of the endogenous chaperone protein. For example, the function of an endogenous gene encoding a chaperone protein can be reduced or eliminated in a lower eukaryotic host cell and a heterologous gene encoding a mammalian chaperone protein is introduced into the host cell. In general, the mammalian chaperone is selected to be from the same species as the recombinant protein that is to be produced by the host cell. The lower eukaryotic host cell that expresses the mammalian chaperone protein but not its endogenous chaperone protein is able to produce active, correctly folded recombinant proteins in high amounts. This is an improvement in productivity compared to production of the recombinant protein in lower eukaryotic host cells that retain the endogenous PDI gene.

The present inventors have also found that by improving protein expression as described herein provides the further advantage that healthy, viable recombinant host cells that have a deletion or disruption of one or more of its endogenous protein O-mannosyltransferases (PMT) genes can be constructed. Deleting or disrupting one or more of the PMT genes in a lower eukaryotic cell results in a reduction in the amount of O-glycosylation of recombinant proteins produced in the cell. However, when PMT deletions are made in lower eukaryotic host cells that further include a deletion in one or genes encoding mannosyltransferases and express the endogenous chaperone proteins, the resulting cells often proved to be non-viable or low-producing cells, rendering them inappropriate for commercial use.

Thus, in certain aspects, the present invention provides lower eukaryotic host cells, in which the function of at least one endogenous gene encoding a chaperone protein has been reduced or eliminated, and a nucleic acid molecule encoding at least one mammalian homolog of the chaperone protein is expressed in the host cell. In further aspects, the lower eukaryotic host cell is a yeast or filamentous fungi host cell.

In further still aspects, the function of the endogeneous gene encoding the chaperone protein Protein Disulphide Isomerase (PDI) is disrupted or deleted such that the endogenous PDI1 is no longer present in the host cell and a nucleic acid molecule encoding a mammalian PDI protein is introduced into the host cell and expressed in the host cell. In one embodiment, the mammalian PDI protein is of the same species as that of the recombinant proteins to be expressed in the host cell and that the nucleic acid molecule encoding the mammalian PDI be integrated into the genome of the host cell. For example, when the recombinant protein is expressed from a human gene introduced into the host cell, it is preferable that the gene encoding the PDI be of human origin as well. In further embodiments, the nucleic acid molecule for expressing the PDI comprises regulatory elements, such as promoter and transcription termination sequences, which are functional in the host cell, operably linked to an open reading frame encoding the mammalian PDI protein. In other embodiments, the endogenous PDI gene is replaced with a nucleic acid molecule encoding a mammalian PDI gene. This can be accomplished by homologous recombination or a single substitution event in which the endogenous PDI1 gene is looped out by the mammalian PDI gene, comprising overlapping sequences on both ends.

In further aspects, the lower eukaryotic host cells of the invention are further transformed with a recombinant vector comprising regulatory nucleotide sequences derived from lower eukaryotic host cells and a coding sequence encoding a selected mammalian protein to be produced by the above host cells. In certain aspects, the selected mammalian protein is a therapeutic protein, and may be a glycoprotein, such as an antibody.

The present invention also provides lower eukaryotic host cells, such as yeast and filamentous fungal host cells, wherein, in addition to replacing the genes encoding one or more of the endogenous chaperone proteins as described above, the function of at least one endogenous gene encoding a protein O-mannosyltransferase (PMT) protein is reduced, disrupted, or deleted. In particular embodiments, the function of at least one endogenous PMT gene selected from the group consisting of the PMT1 and PMT4 genes is reduced, disrupted, or deleted.

In further embodiments, the host cell may be a yeast or filamentous fungal host cell, such as a Pichia pastoris cell, in which the endogenous Pichia pastoris PDI1 has been replaced with a mammalian PDI and the host cell further expresses a vector comprising regulatory nucleotide sequences derived from or functional in Pichia pastoris cells operably linked with an open reading frame encoding a human therapeutic glycoprotein, such as an antibody, which is introduced into the host cell. The host cell is then further be engineered to reduce or eliminate the function of at least one endogenous Pichia pastoris gene encoding a protein O-mannosyltransferase (PMT) protein selected from the group consisting of PMT1 and PMT4 to provide a host cell that is capable of making recombinant proteins having reduced O-glycosylation compared to host cells that have functional PMT genes. In further aspects, the host cells are further contacted with one or more inhibitors of PMT gene expression or PMT protein function.

In further aspects, the present invention comprises recombinant host cells, such as non-human eukaryotic host cells, lower eukaryotic host cells, and yeast and filamentous fungal host cells, with improved characteristics for production of recombinant glycoproteins, glycoproteins of mammalian origin including human proteins. The recombinant host cells of the present invention have been modified by reduction or elimination of the function of at least one endogenous gene encoding a chaperone protein. Reduction or elimination of the function of endogenous genes can be accomplished by any method known in the art, and can be accomplished by alteration of the genetic locus of the endogenous gene, for example, by mutation, insertion or deletion of genetic sequences sufficient to reduce or eliminate the function of the endogenous gene. The chaperone proteins whose function may be reduced or eliminated include, but are not limited to, PDI. In one embodiment, the endogenous gene encoding PDI is either deleted or altered in a manner which reduces or eliminates its function.

In further aspects, the function of the chaperone protein is reduced or eliminated and is then replaced, for example, by transforming the host cell with at least one non-endogenous gene which encodes a homolog of the chaperone protein which has been disrupted or deleted. In further aspects, the host cells are transformed to express at least one foreign gene encoding a human or mammalian homolog of the chaperone protein which has been disrupted or deleted. In further aspects, the foreign gene encodes a homolog from the same species as, or a species closely related to, the species of origin of the recombinant glycoprotein to be produced using the host cell.

In particular aspects, the function of the endogenous chaperone protein PDI1 is reduced or eliminated, and the host cell is transformed to express a homolog of PDI which originates from the same species as, or a species closely related to, the species of origin of the recombinant protein to be produced using the host cell. For example, in a Pichia pastoris expression system for expression of mammalian proteins, the Pichia pastoris host cell is modified to reduce or eliminate the function of the endogenous PDI1 gene, and the host cell is transformed with a nucleic acid molecule which encodes a mammalian PDI gene.

The present invention also provides methods for increasing the productivity of recombinant human or mammalian glycoproteins in a non-human eukaryotic host cell, lower eukaryotic host cell, or a yeast or filamentous fungal host cell. The methods of the present invention comprise the step of reducing or eliminating the function of at least one endogenous gene encoding a chaperone protein. Generally, the method further comprises transforming the host cell with at least one heterogeneous gene which encodes a homolog of the chaperone protein in which the function has been reduced or eliminated. The heterogeneous genes comprise foreign genes encoding human or mammalian homologs of the chaperone proteins in which the functions have been reduced or eliminated. In further aspects, the foreign gene encodes a homolog from the same species as, or a species closely related to, the species of origin of the recombinant glycoprotein to be produced using the host cell. In many aspects, the chaperone proteins whose function may be reduced or eliminated include PDI.

Thus, further provide are methods for producing a recombinant protein in the host cells disclosed herein, for example, in one embodiment, the method comprises providing a lower eukaryotic host cell in which the function of at least one endogenous gene encoding a chaperone protein has been disrupted or deleted and a nucleic acid molecule encoding at least one mammalian homolog of the endogenous chaperone protein is expressed in the host cell: introducing a nucleic acid molecule into the host cell encoding the recombinant protein: and growing the host cell under conditions suitable for producing the recombinant protein. In another embodiment, the method comprises providing a lower eukaryotic host cell in which the function of (i) at least one endogenous gene encoding a chaperone protein; and (ii) at least one endogenous gene encoding a protein O-mannosyltransferase (PMT) protein; have been reduced, disrupted, or deleted; and a nucleic acid molecule encoding at least one mammalian homolog of the chaperone protein is expressed in the host cell; introducing a nucleic acid molecule into the host cell encoding the recombinant protein: and growing the host cell under conditions suitable for producing the recombinant protein. In another embodiment, the method comprises providing lower eukaryotic host cell in which the function of the endogenous gene encoding a chaperone protein PDI; and at least one endogenous gene encoding a protein O-mannosyltransferase-1 (PMT1) or PMT4 protein; have been reduced, disrupted, or deleted; and a nucleic acid molecule encoding at least one mammalian homolog of the chaperone protein PDI is expressed in the host cell; introducing a nucleic acid molecule into the host cell encoding the recombinant protein: and growing the host cell under conditions suitable for producing the recombinant protein.

It has further been found that overexpressing an Ca²⁺ ATPase in the above host cells herein effects a decrease in O-glycan occupancy. It has also been found that overexpressing a calreticulin and an ERp57 protein in the above host cells also effected a reduction in O-glycan occupancy. Thus, in further embodiments of the above host cells, the host cell further includes one or more nucleic acid molecules encoding one or more exogenous or endogenous Ca²⁺ ATPases operably linked to a heterologous promoter. In further embodiments, the Ca²⁺ ATPase is the Ca²⁺ ATPase encoded by the Pichia pastoris PMR1 gene or the Arabidopsis thaliana ECA1 gene. In further embodiments, the host cells further include one or more nucleic acid molecules encoding a calreticulin and/or an ERp57. Other Ca²⁺ ATPases that are suitable include but are not limited to human SERCA2b protein (ATP2A2 ATPase, Ca^{++ transporting, cardiac muscle, slow twitch} 2) and the Pichia pastoris COD1 protein (homologue of Saccharomyces cerevisiae SPF1). Other proteins that are suitable include but are not limited to human UGGT (UDP-glucose:glycoprotein glucosyltransferase) protein and human ERp27 protein.

Thus, the present invention provides a lower eukaryote host cell in which the function of at least one endogenous gene encoding a chaperone protein has been disrupted or deleted and a nucleic acid molecule encoding at least one mammalian homolog of the endogenous chaperone protein is expressed in the host cell.

In a further embodiments, the chaperone protein that is disrupted is a Protein Disulphide Isomerase (PDI) and in further embodiments, the mammalian homolog is a human PDI.

In general, the lower eukaryote host cell further includes a nucleic acid molecule encoding a recombinant protein, which in particular aspects, is a glycoprotein, which in further aspects is an antibody or fragment thereof such as Fc or Fab.

In further embodiments, the function of at least one endogenous gene encoding a protein O-mannosyltransferase (PMT) protein has been reduced, disrupted, or deleted. In particular aspects, the PMT protein is selected from the group consisting of PMT1 and PMT4. Thus, the host cell can further include reduction, disruption, or deletion of the PMT1 or PMT4 alone or reduction, disruption, or deletion of both the PMT1 and PMT4. Thus, further provided is a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and a nucleic acid molecule encoding at least one mammalian homolog of the chaperone protein is expressed in the host cell.

In further embodiments, the host cell further includes a nucleic acid molecule encoding an endogenous or heterologous Ca²⁺ ATPase. In particular aspects, the Ca2+ ATP is selected from the group consisting of the Pichia pastoris PMR1 and the Arabidopsis thaliana ECA1. Thus, further provided is a lower eukaryote host cell in which the function of (a) at least one endogenous gene encoding a chaperone protein has been reduced, disrupted, or deleted; and nucleic acid molecules encoding at least one mammalian homolog of the chaperone protein and at least one Ca²⁺ ATPase are expressed in the host cell. Further provided is a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein and at least one Ca²⁺ ATPase are expressed in the host cell.

In further still aspects, the host cell further includes a nucleic acid molecule encoding the human ERp57 chaparone protein or a nucleic acid molecule encoding a calreticulin (CRT) protein, or both. In particular aspects, the calreticulin protein is the human CRT and the ERp57 is the human ERp57. Thus, further provided is a lower eukaryote host cell in which the function of (a) at least one endogenous gene encoding a chaperone protein has been reduced, disrupted, or deleted; and nucleic acid molecules encoding at least one mammalian homolog of the chaperone protein and at least one of CRT or ERp57 are expressed in the host cell. Further provided is a lower eukaryote host cell in which the function of (a) at least one endogenous gene encoding a chaperone protein has been reduced, disrupted, or deleted; and nucleic acid molecules encoding at least one mammalian homolog of the chaperone protein, at least one of CRT or ERp57, and at least one Ca²⁺ ATPase are expressed in the host cell. Further provided is a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein, at least one of CRT or ERp57, and at least one Ca²⁺ ATPase are expressed in the host cell.

In further aspects of the above host cells, the host cell is selected from the group consisting of Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stipitis, Pichia methanolica, Pichia sp., Saccharomyces cerevisiae, Saccharomyces sp., Schizosacchromyces pombe, Schizosacchroyces sp. Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., any Schizosacchroyces sp., Hansenula polymorpha, any Kluyveromyces sp., Candida albicans, any Aspergillus sp., Trichoderma reesei, Chrysosporium lucknowense, any Fusarium sp. and Neurospora crass.

Further embodiments include methods for producing recombinant proteins in yields higher than is obtainable in host cells that are not modified as disclosed herein and for producing recombinant proteins that have reduced O-glycosylation or O-glycan occupancy compared to recombinant glycoproteins that do not include the genetic modifications disclosed herein. Recombinant proteins include proteins and glycoproteins of therapeutic relevance, including antibodies and fragments thereof.

Thus, provided is a method for producing a recombinant protein comprising: (a) providing a lower eukaryote host cell in which the function of at least one endogenous gene encoding a chaperone protein has been disrupted or deleted and a nucleic acid molecule encoding at least one mammalian homolog of the endogenous chaperone protein is expressed in the host cell; (b) introducing a nucleic acid molecule into the host cell encoding the recombinant protein: and (c) growing the host cell under conditions suitable for producing the recombinant protein.

Further provided is a method for producing a recombinant protein comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and a nucleic acid molecule encoding at least one mammalian homolog of the chaperone protein is expressed in the host cell; (b) introducing a nucleic acid molecule into the host cell encoding the recombinant protein: and (c) growing the host cell under conditions suitable for producing the recombinant protein.

Further provided is a method for producing a recombinant protein comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein and at least one Ca²⁺ ATPase are expressed in the host cell; (b) introducing a nucleic acid molecule into the host cell encoding the recombinant protein: and (c) growing the host cell under conditions suitable for producing the recombinant protein.

Further provided is a method for producing a recombinant protein comprising: (a) providing a lower eukaryote host cell in which the function of (a) at least one endogenous gene encoding a chaperone protein has been reduced, disrupted, or deleted; and nucleic acid molecules encoding at least one mammalian homolog of the chaperone protein and at least one of CRT or ERp57 are expressed in the host cell; (b) introducing a nucleic acid molecule into the host cell encoding the recombinant protein: and (c) growing the host cell under conditions suitable for producing the recombinant protein.

Further provided is a method for producing a recombinant protein comprising: (a) providing a lower eukaryote host cell in which the function of (a) at least one endogenous gene encoding a chaperone protein has been reduced, disrupted, or deleted; and nucleic acid molecules encoding at least one mammalian homolog of the chaperone protein, at least one of CRT or ERp57, and at least one Ca²⁺ ATPase are expressed in the host cell; (b) introducing a nucleic acid molecule into the host cell encoding the recombinant protein: and (c) growing the host cell under conditions suitable for producing the recombinant protein.

Further provided is a method for producing a recombinant protein comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein, at least one of CRT or ERp57, and at least one Ca²⁺ ATPase are expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of at least one endogenous gene encoding a chaperone protein has been disrupted or deleted and a nucleic acid molecule encoding at least one mammalian homolog of the endogenous chaperone protein is expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and a nucleic acid molecule encoding at least one mammalian homolog of the chaperone protein is expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein and at least one Ca²⁺ ATPase are expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of (a) at least one endogenous gene encoding a chaperone protein has been reduced, disrupted, or deleted; and nucleic acid molecules encoding at least one mammalian homolog of the chaperone protein and at least one of CRT or ERp57 are expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of (a) at least one endogenous gene encoding a chaperone protein has been reduced, disrupted, or deleted; and nucleic acid molecules encoding at least one mammalian homolog of the chaperone protein, at least one of CRT or ERp57, and at least one Ca²⁺ ATPase are expressed in the host cell; (b) introducing a nucleic acid molecule into the host cell encoding the recombinant protein: and (c) growing the host cell under conditions suitable for producing the recombinant protein.

Further provided is a method for producing a recombinant protein with reduced O-glycosylation or O-glycan occupancy comprising: (a) providing a lower eukaryote host cell in which the function of (a) at least one endogenous gene encoding a chaperone protein; and (b) at least one endogenous gene encoding a protein O-mannosyltransferase (PMT) protein; have been reduced, disrupted, or deleted; and nucleic acid molecules encoding at least one mammalian homolog of the chaperone protein, at least one of CRT or ERp57, and at least one Ca²⁺ ATPase are expressed in the host cell; (b) introducing a nucleic acid molecule into the host cell encoding the recombinant protein: and (c) growing the host cell under conditions suitable for producing the recombinant protein.

In further aspects of the above methods, the host cell is selected from the group consisting of Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stipitis, Pichia methanolica, Pichia sp., Saccharomyces cerevisiae, Saccharomyces sp., Schizosacchromyces pombe, Schizosacchroyces sp. Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., any Schizosacchromyces sp., Hansenula polymorpha, any Kluyveromyces sp., Candida albicans, any Aspergillus sp., Trichoderma reesei, Chrysosporium lucknowense, any Fusarium sp. and Neurospora crassa.

Further provided are recombinant proteins produced by the host cells disclosed herein.

In particular embodiments, any one of the aforementioned host cells can further include genetic modifications that enable the host cells to produce glycoproteins have predominantly particular N-glycan structures thereon or particular mixtures of N-glycan structures thereon. For example, the host cells have been genetically engineered to produce N-glycans having a Man₃GlcNAc₂ or Man₅GlcNAc₂ core structure, which in particular aspects include one or more additional sugars such as GlcNAc, Galactose, or sialic acid on the non-reducing end, and optionally fucose on the GlcNAc at the reducing end. Thus, the N-glycans include both bi-antennary and multi-antennary glycoforms and glycoforms that are bisected. Examples of N-glycans include but are not limited to Man₈GlcNAc₂, Man₇GlcNAc₂, Man₆GlcNAc₂, Man₅GlcNAc₂, GlcNAcMan₅GlcNAc₂, GalGlcNAcMan₅GlcNAc₂, NANAGalGlcNAcMan₅GlcNAc₂, Man₃GlcNAc₂, GlcNAc₍₁₋₄₎Man₃GlcNAc₂, Gal₍₁₋₄₎GlcNAc₍₁₋₄₎Man₃GlcNAc₂, NANA₍₁₋₄₎Gal₍₁₋₄₎GlcNAc₍₁₋₄₎Man₃GlcNAc₂.

### Definitions

Unless otherwise defined herein, scientific and technical terms and phrases used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

As used herein, the terms “N-glycan” and “glycoform” are used interchangeably and refer to an N-linked oligosaccharide, e.g., one that is attached by an asparagine-N-acetylglucosamine linkage to an asparagine residue of a polypeptide. N-linked glycoproteins contain an N-acetylglucosamine residue linked to the amide nitrogen of an asparagine residue in the protein. The predominant sugars found on glycoproteins are glucose, galactose, mannose, fucose, N-acetylgalactosamine (GalNAc), N-acetylglucosamine (GlcNAc) and sialic acid (e.g., N-acetyl-neuraminic acid (NANA)). The processing of the sugar groups occurs cotranslationally in the lumen of the ER and continues in the Golgi apparatus for N-linked glycoproteins.

N-glycans have a common pentasaccharide core of Man₃GlcNAc₂ (“Man” refers to mannose; “Glc” refers to glucose; and “NAc” refers to N-acetyl; GlcNAc refers to N-acetylglucosamine) N-glycans differ with respect to the number of branches (antennae) comprising peripheral sugars (e.g., GlcNAc, galactose, fucose and sialic acid) that are added to the Man₃GlcNAc₂ (“Man3”) core structure which is also referred to as the “trimannose core”, the “pentasaccharide core” or the “paucimannose core”. N-glycans are classified according to their branched constituents (e.g., high mannose, complex or hybrid). A “high mannose” type N-glycan has five or more mannose residues. A “complex” type N-glycan typically has at least one GlcNAc attached to the 1,3 mannose arm and at least one GlcNAc attached to the 1,6 mannose arm of a “trimannose” core. Complex N-glycans may also have galactose (“Gal”) or N-acetylgalactosamine (“GalNAc”) residues that are optionally modified with sialic acid or derivatives (e.g., “NANA” or “NeuAc”, where “Neu” refers to neuraminic acid and “Ac” refers to acetyl). Complex N-glycans may also have intrachain substitutions comprising “bisecting” GlcNAc and core fucose (“Fuc”). Complex N-glycans may also have multiple antennae on the “trimannose core,” often referred to as “multiple antennary glycans.” A “hybrid” N-glycan has at least one GlcNAc on the terminal of the 1,3 mannose arm of the trimannose core and zero or more mannoses on the 1,6 mannose arm of the trimannose core. The various N-glycans are also referred to as “glycoforms.”

Abbreviations used herein are of common usage in the art, see, e.g., abbreviations of sugars, above. Other common abbreviations include “PNGase”, or “glycanase” or “glucosidase” which all refer to peptide N-glycosidase F (EC 3.2.2.18).

The term “vector” as used herein is intended to refer to a nucleic acid molecule capable of transporting another nucleic acid molecule to which it has been linked. One type of vector is a “plasmid vector”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”).

As used herein, the term “sequence of interest” or “gene of interest” refers to a nucleic acid sequence, typically encoding a protein, that is not normally produced in the host cell. The methods disclosed herein allow efficient expression of one or more sequences of interest or genes of interest stably integrated into a host cell genome. Non-limiting examples of sequences of interest include sequences encoding one or more polypeptides having an enzymatic activity, e.g., an enzyme which affects N-glycan synthesis in a host such as mannosyltransferases, N-acetylglucosaminyltransferases, UDP-N-acetylglucosamine transporters, galactosyltransferases, UDP-N-acetylgalactosyltransferase, sialyltransferases and fucosyltransferases.

The term “marker sequence” or “marker gene” refers to a nucleic acid sequence capable of expressing an activity that allows either positive or negative selection for the presence or absence of the sequence within a host cell. For example, the Pichia pastoris URA5 gene is a marker gene because its presence can be selected for by the ability of cells containing the gene to grow in the absence of uracil. Its presence can also be selected against by the inability of cells containing the gene to grow in the presence of 5-FOA. Marker sequences or genes do not necessarily need to display both positive and negative selectability. Non-limiting examples of marker sequences or genes from Pichia pastoris include ADE1, ARG4, HIS4 and URA3. For antibiotic resistance marker genes, kanamycin, neomycin, geneticin (or G418), paromomycin and hygromycin resistance genes are commonly used to allow for growth in the presence of these antibiotics.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “eukaryotic” refers to a nucleated cell or organism, and includes insect cells, plant cells, mammalian cells, animal cells and lower eukaryotic cells.

The term “lower eukaryotic cells” includes yeast and filamentous fungi. Yeast and filamentous fungi include, but are not limited to: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stipitis, Pichia methanolica, Pichia sp., Saccharomyces cerevisiae, Saccharomyces sp., Schizosacchromyces pombe, Schizosacchroyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Pichia sp., any Saccharomyces sp., any Schizosacchromyces sp., Hansenula polymorpha, any Kluyveromyces sp., Candida albicans, any Aspergillus sp., Trichoderma reesei, Chrysosporium lucknowense, any Fusarium sp. and Neurospora crassa.

The function of a gene encoding a protein is said to be ‘reduced’ when that gene has been modified, for example, by deletion, insertion, mutation or substitution of one or more nucleotides, such that the modified gene encodes a protein which has at least 20% to 50% lower activity, in particular aspects, at least 40% lower activity or at least 50% lower activity, when measured in a standard assay, as compared to the protein encoded by the corresponding gene without such modification. The function of a gene encoding a protein is said to be ‘eliminated’ when the gene has been modified, for example, by deletion, insertion, mutation or substitution of one or more nucleotides, such that the modified gene encodes a protein which has at least 90% to 99% lower activity, in particular aspects, at least 95% lower activity or at least 99% lower activity, when measured in a standard assay, as compared to the protein encoded by the corresponding gene without such modification.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting in any manner.

### BRIEF DESCRIPTION OF THE DRAWINGS

FIG. 1 illustrates representative results from deep-well plate screening where human anti-DKK1 antibody is produced in Pichia pastoris host cells in which the endogenous PDI1 gene is expressed (Panel A), both in the presence of the endogenous PDI1 gene and the human PDI gene (Panel B), and in a cell line expressing the human PDI gene and in which the endogenous PDI1 gene function has been knocked out (Panel C).

FIG. 2 illustrates the action of human PDI and its co-chaperones in thiol-redox reactions in the endoplasmic reticulum.

FIGS. 3A, 3B, and 3C show the genealogy of yeast strains described in the examples for illustrating the invention.

FIGS. 4A and 4B shows representative results from shakeflask (A) and 0.5 L bioreactor (B) expression studies in which human anti-Her2 antibody was produced in Pichia pastoris strains in which the human PDI gene (hPDI) replaced the endogenous PDI1 and strains in which the human PDI replaced the endogenous PDI1 and the PMT1 gene is disrupted (hPDI+Δpmt1). Antibodies were recovered and resolved by polyacrylamide gel electrophoresis on non-reducing and reducing polyacrylamide gels. Lanes 1-2 shows antibodies produced from two clones produced from transformation of strain yGLY2696 with plasmid vector pGLY2988 encoding the anti-Her2 antibody and lanes 3-6 shows the antibodies produced from four clones produced from transformation of strain yGLY2696 in which the PMT1 gene was deleted and with plasmid vector pGLY2988 encoding the anti-Her2 antibody.

FIG. 5 shows representative results from a shakeflask expression study in which human anti-DKK1 antibody was produced in Pichia pastoris strains in which the human PDI (hPDI) gene replaced the endogenous PDI1 and strains in which the human PDI replaced the endogenous PDI1 and the PMT1 gene disrupted (hPDI+Δpmt1). Antibodies were recovered and resolved by polyacrylamide gel electrophoresis on non-reducing and reducing polyacrylamide gels. Lanes 1 and 3 shows antibodies produced from two clones produced from transformation of strains yGLY2696 and yGLY2690 with plasmid vector pGLY2260 encoding the anti-DKK1 antibody and lanes 2 and 4 shows the antibodies produced from two clones produced from transformation of strains yGLY2696 and yGLY2690 in which the PMT1 gene was deleted with plasmid vector pGLY2260 encoding the anti-DKK1 antibody.

FIG. 6 shows results from a 0.5 L bioreactor expression study where human anti-Her2 antibody is produced in Pichia pastoris strains in which the human PDI gene (hPDI) replaced the endogenous PDI1, strains in which the human PDI replaced the endogenous PDI1 and the PMT4 gene disrupted (hPDI+Δpmt4), and strains that express only the endogenous PDI1 but in which the PMT4 gene is disrupted (PpPDI+Δpmt4). Antibodies were recovered and resolved by polyacrylamide gel electrophoresis on non-reducing polyacrylamide gels. Lanes 1 and 2 shows antibodies produced from two clones from transformation of strain yGLY24-1 with plasmid vector pGLY2988 encoding the anti-Her2 antibody and lanes 3-5 show anti-Her2 antibodies produced from three clones produced from transformation of strain yGLY2690 in which the PMT4 gene was deleted.

FIG. 7 shows results from a shakeflask expression study where human anti-CD20 antibody is produced in Pichia pastoris strains in which the human PDI replaced the endogenous PDI1 and the PMT4 gene is disrupted (hPDI+Δpmt4) and strains that express only the endogenous PDI1 but in which the PMT4 gene is disrupted (PpPDI+Δpmt4). Antibodies were recovered and resolved by polyacrylamide gel electrophoresis on non-reducing and reducing polyacrylamide gels. Lane 1 shows antibodies produced from strain yGLY24-1 transformed with plasmid vector pGLY3200 encoding the anti-CD20 antibody; lanes 2-7 show anti-CD20 antibodies produced from six clones produced from transformation of strain yGLY2690 in which the PMT4 gene was deleted.

FIG. 8 illustrates the construction of plasmid vector pGLY642 encoding the human PDI (hPDI) and targeting the Pichia pastoris PDI1 locus.

FIG. 9 illustrates the construction of plasmid vector pGLY2232 encoding the human ERO1α (hERO1α) and targeting the Pichia pastoris PrB1 locus.

FIG. 10 illustrates the construction of plasmid vector pGLY2233 encoding the human GRP94 and targeting the Pichia pastoris PEP4 locus.

FIG. 11 illustrates the construction of plasmid vector pGFI207t encoding the T. reesei α-1,2 mannosidase (TrMNS1) and mouse α-1,2 mannosidase IA (FB53) and targeting the Pichia pastoris PRO locus.

FIG. 12 illustrates the construction of plasmid vector pGLY1162 encoding the T. reesei α-1,2 mannosidase (TrMNS1) and targeting the Pichia pastoris PRO locus.

FIG. 13 is maps of plasmid vector pGLY2260 and 2261 encoding the anti-DKK1 antibody heavy chain (GFI710H) and light chain (GFI710L) or two light chains (GFI710L) and targeting the Pichia pastoris TRP2 locus.

FIG. 14 is a map of plasmid vector pGLY2012 encoding the anti-ADDL antibody heavy chain (Hc) and light chain (Lc) and targeting the Pichia pastoris TRP2 locus.

FIG. 15 is a map of plasmid vector pGLY2988 encoding the anti-HER2 antibody (anti-HER2) heavy chain (Hc) and light chain (Lc) and targeting the Pichia pastoris TRP2 locus.

FIG. 16 is a map of plasmid vector pGLY3200 encoding the anti-CD20 antibody heavy chain (Hc) and light chain (Lc) and targeting the Pichia pastoris TRP2 locus.

FIG. 17 is a map of plasmid vector pGLY3822 encoding the Pichia pastoris PMR1 and targeting the Pichia pastoris URA6 locus.

FIG. 18 is a map of plasmid vector pGLY3827 encoding the Arabidopsis thaliana ECA1 (AtECA1) and targeting the Pichia pastoris URA6 locus.

FIG. 19 is a map of plasmid vector pGLY1234 encoding the human CRT (hCRT) and human ERp57 (hERp57) and targeting the Pichia pastoris HIS3 locus.

### DETAILED DESCRIPTION OF THE INVENTION

Molecular chaperones play a critical role in the folding and secretion of antibodies. One chaperone protein in particular, Protein Disulfide Isomerase (PDI), functions to catalyze inter and intra disulphide bond formation that link the antibody heavy and light chains. Protein disulfide isomerase (PDI) can produce a substantial increase or a substantial decrease in the recovery of disulfide-containing proteins, when compared with the uncatalyzed reaction; a high concentration of PDI in the endoplasmic reticulum (ER) is essential for the expression of disulfide-containing proteins [Puig and Gilbert, J. Biol. Chem., 269:7764-7771 (1994)]. Past attempts to increase antibody expression levels in Pichia pastoris by overexpressing human PDI chaperone protein and/or overexpressing endogenous PDI1 have been with limited success. We have undertaken humanization of the chaperone pathway in Pichia pastoris to explore the possibility of antibody yield improvement through direct genetic engineering.

We have found in a Pichia pastoris model that replacement of the yeast gene encoding the endogenous PDI1 protein with an expression cassette encoding a heterologous PDI protein resulted in approximately a five-fold improvement in the yield of recombinant human antibody produced by the recombinant yeast cells as compared to the yield produced by recombinant yeast cells that expressed only the endogenous PDI1 protein and about a three-fold increase in yield compared to the yield produced by recombinant yeast cells that co-expressed the heterologous PDI protein with the endogenous PDI1 protein.

Without being limited to any scientific theory of the mechanism of the invention, it is believed that heterologous recombinant proteins may interact more efficiently with heterologous chaperone proteins than host cell chaperone proteins in the course of their folding and assembly along the secretory pathway. In the case of co-expression, the heterologous chaperone protein may compete with the endogenous chaperone protein for its substrate, i.e., heterologous recombinant proteins. It is further believed that the heterologous PDI protein and recombinant protein be from the same species. Therefore, replacement of the gene encoding the endogenous chaperone protein with an expression cassette encoding a heterologous chaperone may be a better means for producing recombinant host cells for producing recombinant proteins that merely co-expressing the heterologous chaperone protein with the endogenous chaperone protein.

In addition, further improvements in recombinant protein yield may be obtained by overexpressing in the recombinant host cell the heterologous PDI protein and an additional heterologous co-chaperone proteins, such as ERO1α and or the GRP94 proteins. In further aspects, the recombinant host cell can further overexpress FAD, FLC1, and ERp44 proteins. Since these genes are related in function, it may be desirable to include the nucleic acid molecules that encode these genes in a single vector, which transformed into the host cell. Expression of the proteins may be effected by operably linking the nucleic acid molecules encoding the proteins to a heterologous or homologous promoter. In particular aspects, when the host cell is Pichia pastoris, expression of one or more of the heterologous co-chaperone proteins may be effected by a homologous promoter such as the KAR2 promoter or a promoter from another ER-specific gene. In further aspects, all of the heterologous chaperone proteins and recombinant protein be from the same species.

As exemplified in the Examples using Pichia pastoris as a model, the methods disclosed herein are particularly useful in the production of recombinant human glycoproteins, including antibodies, from lower eukaryotic host cells, such as yeast and filamentous fungi. For example, secretion of recombinant proteins from Pichia pastoris proceeds more efficiently as the folding and assembly of the protein of interest is assisted by human PDI, and optionally including other mammalian-derived chaperone proteins, such as ERO1α and GRP94, thereby improving yield. As exemplified in the Examples, the methods herein will especially benefit antibody production in which the heavy and light chains must be properly assembled through disulphide bonds in order to achieve activity.

Thus, there methods herein provide significant advantages with respect to addressing the problem of low productivity in the secretion of recombinant antibodies from lower eukaryotic host cells, and in particular yeast and filamentous fungi, for example, Pichia pastoris. In the past, yeast, human or mouse chaperone proteins were overexpressed with limited success while the present invention demonstrates that improved productivity of correctly folded and secreted heterologous proteins, such as antibodies, can be obtained through replacement of the host cells' endogenous chaperone proteins with heterologous chaperone proteins. The overexpression of mammalian-derived chaperone proteins, combined with the deletion of the endogenous gene encoding a protein homolog unexpectedly results in improved productivity of glycoproteins, compared with overexpression of the mammalian-derived protein alone.

We further found that host cells, transformed with nucleic acid molecules encoding one or more chaperone genes as described above, can be further genetically manipulated to improve other characteristics of the recombinant proteins produced therefrom. This is especially true in the case of recombinant mammalian glycoprotein production from lower eukaryotic host cells such as yeast or filamentous fungi.

For example, lower eukaryotic cells such as Saccharomyces cerevisiae, Candida albicans, and Pichia pastoris, contain a family of genes known as protein O-mannosyltransferases (PMTs) involved in the transfer of mannose to seryl and threonyl residues of secretory proteins. We found that Pichia pastoris cell lines, which have been genetically altered to express one or more humanized or chimeric chaperone genes, are better able to tolerate deletion of one or more PMT genes, with little or no effect on cell growth or protein expression. PMT genes which may be deleted include PMT1, PMT2, PMT4, PMT5, and PMT6. In general, Pichia pastoris host cells in which both the OCH1 gene and the PMT gene is deleted either grow poorly or not at all. Deletion or functional knockout of the OCH1 gene is necessary for constructing recombinant Pichia pastoris host cells that can make human glycoproteins that have human-like N-glycans. Because it is desirable to produce human glycoproteins that have no or reduced O-glycosylation, there has been a need to find means for reducing O-glycosylation in recombinant Pichia pastoris host cells that are also capable of producing human glycoproteins with human-like N-glycans. We found that Pichia pastoris host cells containing one or more chaperone genes as disclosed herein can be further genetically altered to contain a deletion or functional knockout of the OCH1 gene and a deletion or functional knockout of one or more PMT genes, such as PMT1, PMT4, PMT5, and/or PMT6. These recombinant cells are viable and produce human glycoproteins with human-like N-glycans in high yield and with reduced O-glycosylation. In addition, a further reduction in O-glycosylation was achieved by growing the cells in the presence of a PMT protein inhibitor.

As exemplified in the Examples, we demonstrate that the methods disclosed herein are particularly useful in the production of recombinant human glycoproteins, including antibodies, from lower eukaryotic host cells, such as yeast and filamentous fungi with improved properties, since the host cells of the present invention exhibit tolerance to chemical PMT protein inhibitors and/or deletion of PMT genes. The Examples show that the recombinant proteins have reduced O-glycosylation occupancy and length of O-glycans compared with prior lower eukaryotic expression systems. As exemplified in the Examples, the methods herein will especially benefit antibody production in which the heavy and light chains must be properly assembled through disulphide bonds in order to achieve activity and the antibodies must have reduced or no O-glycosylation.

We have further found that over-expression of Pichia pastoris Golgi Ca²⁺ ATPase (PpPMR1) or Arabidopsis thaliana ER Ca²⁺ ATPase (AtECA1) effected about a 2-fold reduction in O-glycan occupancy compared to the above strains wherein the endogenous PDI1 had been replaced with the human PDI but which did not express either Ca²⁺ ATPase. Thus, in further embodiments, any one of the host cells disclosed herein can further include one or more nucleic acid molecules encoding an endogenous or exogenous Golgi or ER Ca²⁺ ATPase, wherein the Ca²⁺ ATPase is operably linked to a heterologous promoter. These host cells can be used to produce glycoproteins with reduced O-glycosylation.

Calreticulin (CRT) is a multifunctional protein that acts as a major Ca(2+)-binding (storage) protein in the lumen of the endoplasmic reticulum. It is also found in the nucleus, suggesting that it may have a role in transcription regulation. Calreticulin binds to the synthetic peptide KLGFFKR (SEQ ID NO:75), which is almost identical to an amino acid sequence in the DNA-binding domain of the superfamily of nuclear receptors. Calreticulin binds to antibodies in certain sera of systemic lupus and Sjogren patients which contain anti-Ro/SSA antibodies, it is highly conserved among species, and it is located in the endoplasmic and sarcoplasmic reticulum where it may bind calcium. Calreticulin binds to misfolded proteins and prevents them from being exported from the Endoplasmic reticulum to the Golgi apparatus.

ERp57 is a chaperone protein of the endoplasmic reticulum that interacts with lectin chaperones calreticulin and calnexin to modulate folding of newly synthesized glycoproteins. The protein was once thought to be a phospholipase; however, it has been demonstrated that the protein actually has protein disulfide isomerase activity. Thus, the ERp57 is a lumenal protein of the endoplasmic reticulum (ER) and a member of the protein disulfide isomerase (PDI) family. It is thought that complexes of lectins and this protein mediate protein folding by promoting formation of disulfide bonds in their glycoprotein substrates. In contrast to archetypal PDI, ERp57 interacts specifically with newly synthesized glycoproteins.

We have further found that over-expression of the human CRT and human ERp57 in Pichia pastoris effected about a one-third reduction in O-glycan occupancy compared to strains wherein the endogenous PDI1 had been replaced with the human PDI but which did not express the hCRT and hERp57. Thus, in further embodiments, any one of the host cells herein can further include one or more nucleic acid molecules encoding a calreticulin and an ERp57 protein, each operably linked to a heterologous promoter. These host cells can be used to produce glycoproteins with reduced O-glycosylation.

Thus, the methods herein provide significant advantages with respect to addressing the problem of low productivity in the secretion of recombinant antibodies from lower eukaryotic host cells, and in particular yeast and filamentous fungi, for example, Pichia pastoris. In the past, yeast, human or mouse chaperone proteins were overexpressed with limited success while the present invention demonstrates that improved productivity of correctly folded and secreted heterologous proteins, such as antibodies, can be obtained through replacement of the host cells' endogenous chaperone proteins with heterologous chaperone proteins. The overexpression of mammalian-derived chaperone proteins, combined with the deletion of the endogenous gene encoding a protein homolog unexpectedly results in improved productivity of glycoproteins, compared with overexpression of the mammalian-derived protein alone.

Therefore, the present invention provides methods for increasing production of an overexpressed gene product present in a lower eukaryote host cell, which includes expressing a heterologous chaperone protein in the host cell in place of an endogenous chaperone protein and thereby increasing production of the overexpressed gene product. Also provided is a method of increasing production of an overexpressed gene product from a host cell by disrupting or deleting a gene encoding an endogenous chaperone protein and expressing a nucleic acid molecule encoding a heterologous chaperone protein encoded in an expression vector present in or provided to the host cell, thereby increasing the production of the overexpressed gene product. Further provided is a method for increasing production of overexpressed gene products from a host cell, which comprises expressing at least one heterologous chaperone protein in the host cell in place of the endogenous chaperone protein. In the present context, an overexpressed gene product is one which is expressed at levels greater than normal endogenous expression for that gene product.

In one embodiment, the method comprises deleting or disrupting expression of an endogenous chaperone protein and effecting the expression of one or more heterologous chaperone proteins and an overexpressed gene product in a host cell, and cultivating said host cell under conditions suitable for secretion of the overexpressed gene product. The expression of the chaperone protein and the overexpressed gene product can be effected by inducing expression of a nucleic acid molecule encoding the chaperone protein and a nucleic acid molecule encoding the overexpressed gene product wherein said nucleic acid molecules are present in a host cell.

In another embodiment, the expression of the heterologous chaperone protein and the overexpressed gene product are effected by introducing a first nucleic acid molecule encoding a heterologous chaperone protein and a second nucleic acid molecule encoding a gene product to be overexpressed into a host cell in which expression of at least one gene encoding an endogenous chaperone protein has been disrupted or deleted under conditions suitable for expression of the first and second nucleic acid molecules. In further aspects, one or both of said first and second nucleic acid molecules are present in expression vectors. In further aspects, one or both of said first and second nucleic acid molecules are present in expression/integration vectors. In a further embodiment, expression of the heterologous chaperone protein is effected by inducing expression of the nucleic acid molecule encoding the chaperone protein wherein the nucleic acid molecule into a host cell in which the gene encoding the endogenous chaperone protein has been deleted or disrupted. Expression of the second protein is effected by inducing expression of a nucleic acid molecule encoding the gene product to be overexpressed by introducing a nucleic acid molecule encoding said second gene product into the host cell.

The present invention further provides methods for increasing production of an overexpressed gene product present in a lower eukaryote host cell with reduced O-glycosylation, which includes expressing a heterologous chaperone protein in the host cell in place of an endogenous chaperone protein and wherein the host cell has had one or more genes in the protein O-mannosyltransferase (PMT) family disrupted or deleted, thereby increasing production of the overexpressed gene product with reduced O-glycosylation. Also provided is a method of increasing production of an overexpressed gene product with reduced O-glycosylation from a host cell by disrupting or deleting a gene encoding an endogenous chaperone protein and a gene encoding a PMT and expressing a nucleic acid molecule encoding a heterologous chaperone protein encoded in an expression vector present in or provided to the host cell, thereby increasing the production of the overexpressed gene product. Further provided is a method for increasing production of overexpressed gene products with reduced O-glycosylation from a host cell, which comprises expressing at least one heterologous chaperone protein in the host cell in place of the endogenous chaperone protein and wherein at least one PMT gene has been disrupted or deleted. In one embodiment, the method comprises deleting or disrupting expression of at least one endogenous chaperone protein and at least one PMT gene and effecting the expression of one or more heterologous chaperone proteins and an overexpressed gene product in a host cell, and cultivating said host cell under conditions suitable for secretion of the overexpressed gene product with reduced O-glycosylation. The expression of the chaperone protein and the overexpressed gene product can be effected by inducing expression of a nucleic acid molecule encoding the chaperone protein and a nucleic acid molecule encoding the overexpressed gene product wherein said nucleic acid molecules are present in a host cell.

In another embodiment, the expression of the heterologous chaperone protein and the overexpressed gene product are effected by introducing a first nucleic acid molecule encoding a heterologous chaperone protein and a second nucleic acid molecule encoding a gene product to be overexpressed into a host cell in which expression of at least one gene encoding an endogenous chaperone protein and at least one PMT gene have been disrupted or deleted under conditions suitable for expression of the first and second nucleic acid molecules. In further aspects, one or both of said first and second nucleic acid molecules are present in expression vectors. In further aspects, one or both of said first and second nucleic acid molecules are present in expression/integration vectors. In a further embodiment, expression of the heterologous chaperone protein is effected by inducing expression of the nucleic acid molecule encoding the chaperone protein wherein the nucleic acid molecule into a host cell in which the gene encoding the endogenous chaperone protein has been deleted or disrupted. Expression of the second protein is effected by inducing expression of a nucleic acid molecule encoding the gene product to be overexpressed by introducing a nucleic acid molecule encoding said second gene product into the host cell.

In a further aspect of any one of the above embodiments, the heterologous chaperone protein corresponds in species or class to the endogenous chaperone protein. For example, if the host cell is a yeast cell and the endogenous chaperone protein is a protein disulfide isomerase (PDI) then the corresponding heterologous PDI can be a mammalian PDI. In further still aspects of any one of the above embodiments, the heterologous chaperone proteins expressed in a particular host cell are from the same species as the species for the overexpressed gene product. For example, if the overexpressed gene product is a human protein then the heterologous chaperone proteins are human chaperone proteins; or if the overexpressed gene product is a bovine protein then the heterologous chaperone protein is a bovine chaperone protein.

Chaperone proteins include any chaperone protein which can facilitate or increase the secretion of proteins. In particular, members of the protein disulfide isomerase and heat shock 70 (hsp70) families of proteins are contemplated. An uncapitalized “hsp70” is used herein to designate the heat shock protein 70 family of proteins which share structural and functional similarity and whose expression are generally induced by stress. To distinguish the hsp70 family of proteins from the single heat shock protein of a species which has a molecular weight of about 70,000, and which has an art-recognized name of heat shock protein-70, a capitalized HSP70 is used herein. Accordingly, each member of the hsp70 family of proteins from a given species has structural similarity to the HSP70 protein from that species.

The present invention is directed to any chaperone protein having the capability to stimulate secretion of an overexpressed gene product. The members of the hsp70 family of proteins are known to be structurally homologous and include yeast hsp70 proteins such as KAR2, HSP70, BiP, SSA1-4, SSB1, SSC1 and SSD1 gene products and eukaryotic hsp70 proteins such as HSP68, HSP72, HSP73, HSC70, clathrin uncoating ATPase, IgG heavy chain binding protein (BiP), glucose-regulated proteins 75, 78 and 80 (GRP75, GRP78 and GRP80) and the like. Moreover, according to the present invention any hsp70 chaperone protein having sufficient homology to the yeast KAR2 or mammalian BiP polypeptide sequence can be used in the present methods to stimulate secretion of an overexpressed gene product. Members of the PDI family are also structurally homologous, and any PDI which can be used according to the present method is contemplated herein. In particular, mammalian (including human) and yeast PDI, prolyl-4-hydroxylase β-subunit, ERp57, ERp29, ERp72, GSBP, ERO1α, GRP94, GRP170, BiP, and T3BP and yeast EUG1 are contemplated. Because many therapeutic proteins for use in human are of human origin, a particular aspect of the methods herein is that the heterologous chaperone protein is of human origin. In further still embodiments, the preferred heterologous chaperone protein is a PDI protein, particularly a PDI protein of human origin.

Attempts to increase expression levels of heterologous human proteins in yeast cell lines by overexpressing human BiP, using constitutive promoters such as GAPDH, have been largely unsuccessful. Knockouts of Pichia pastoris KAR2, the homolog of human BiP, have been harmful to cells. The limitations of the prior art can be overcome by constructing a chimeric BiP gene, in which the human ATPase domain is replaced by the ATPase domain of Pichia pastoris KAR2, fused to the human BiP peptide binding domain, under the control of the KAR2, or other ER-specific promoter from Pichia pastoris. Further improvements in yield may be obtained by combining the replacement of the endogenous PDI1 gene, as described above, with the use of chimeric BiP and human ERdj3.

In further aspects, the overexpressed gene product is a secreted gene product. Procedures for observing whether an overexpressed gene product is secreted are readily available to the skilled artisan. For example, Goeddel, (Ed.) 1990, Gene Expression Technology, Methods in Enzymology, Vol 185, Academic Press, and Sambrook et al. 1989, Molecular Cloning: A Laboratory Manual, Vols. 1-3, Cold Spring Harbor Press, N.Y., provide procedures for detecting secreted gene products.

To secrete an overexpressed gene product the host cell is cultivated under conditions sufficient for secretion of the overexpressed gene product. Such conditions include temperature, nutrient and cell density conditions that permit secretion by the cell. Moreover, such conditions are conditions under which the cell can perform basic cellular functions of transcription, translation and passage of proteins from one cellular compartment to another and are known to the skilled artisan.

Moreover, as is known to the skilled artisan a secreted gene product can be detected in the culture medium used to maintain or grow the present host cells. The culture medium can be separated from the host cells by known procedures, for example, centrifugation or filtration. The overexpressed gene product can then be detected in the cell-free culture medium by taking advantage of known properties characteristic of the overexpressed gene product. Such properties can include the distinct immunological, enzymatic or physical properties of the overexpressed gene product. For example, if an overexpressed gene product has a unique enzyme activity an assay for that activity can be performed on the culture medium used by the host cells. Moreover, when antibodies reactive against a given overexpressed gene product are available, such antibodies can be used to detect the gene product in any known immunological assay (See Harlowe, et al., 1988, Antibodies: A Laboratory Manual, Cold Spring Harbor Laboratory Press)

In addition, a secreted gene product can be a fusion protein wherein the gene product includes a heterologous signal or leader peptide that facilitates the secretion of the gene product. Secretion signal peptides are discrete amino acid sequences, which cause the host cell to direct a gene product through internal and external cellular membranes and into the extracellular environment. Secretion signal peptides are present at the N-terminus of a nascent polypeptide gene product targeted for secretion. Additional eukaryotic secretion signals can also be present along the polypeptide chain of the gene product in the form of carbohydrates attached to specific amino acids, i.e. glycosylation secretion signals.

N-terminal signal peptides include a hydrophobic domain of about 10 to about 30 amino acids which can be preceded by a short charged domain of about two to about 10 amino acids. Moreover, the signal peptide is present at the N-terminus of gene products destined for secretion. In general, the particular sequence of a signal sequence is not critical but signal sequences are rich in hydrophobic amino acids such as alanine (Ala), valine (Val), leucine (Leu), isoleucine (Ile), proline (Pro), phenylalanine (Phe), tryptophan (Trp), methionine (Met) and the like.

Many signal peptides are known (Michaelis et al., Ann. Rev. Microbiol. 36: 425 (1982). For example, the yeast acid phosphatase, yeast invertase, and the yeast α-factor signal peptides have been attached to heterologous polypeptide coding regions and used successfully for secretion of the heterologous polypeptide (See for example, Sato et al. Gene 83: 355-365 (1989); Chang et al. Mol. Cell. Biol. 6: 1812-1819 (1986); and Brake et al. Proc. Natl. Acad. Sci. USA 81: 4642-4646 (1984). Therefore, the skilled artisan can readily design or obtain a nucleic acid molecule which encodes a coding region for an overexpressed gene product which also has a signal peptide at the 5′-end.

Examples of overexpressed gene products which are preferably secreted by the present methods include mammalian gene products such as enzymes, cytokines, growth factors, hormones, vaccines, antibodies and the like. More particularly, overexpressed gene products include but are not limited to gene products such as erythropoietin, insulin, somatotropin, growth hormone releasing factor, platelet derived growth factor, epidermal growth factor, transforming growth factor α, transforming growth factor β, epidermal growth factor, fibroblast growth factor, nerve growth factor, insulin-like growth factor I, insulin-like growth factor II, clotting Factor VIII, superoxide dismutase, α-interferon, γ-interferon, interleukin-1, interleukin-2, interleukin-3, interleukin-4, interleukin-5, interleukin-6, granulocyte colony stimulating factor, multi-lineage colony stimulating activity, granulocyte-macrophage stimulating factor, macrophage colony stimulating factor, T cell growth factor, lymphotoxin, immunoglobulins, antibodies, and the like. Further included are fusion proteins, including but not limited to, peptides and polypeptides fused to the constant region of an immunoglobulin or antibody. Particularly useful overexpressed gene products are human gene products.

The terms “antibody”, “antibodies”, and “immunoglobulin(s)” encompass any recombinant monoclonal antibody produced by recombinant DNA technology and further is meant to include humanized and chimeric antibodies.

The present methods can readily be adapted to enhance secretion of any overexpressed gene product which can be used as a vaccine. Overexpressed gene products which can be used as vaccines include any structural, membrane-associated, membrane-bound or secreted gene product of a mammalian pathogen. Mammalian pathogens include viruses, bacteria, single-celled or multi-celled parasites which can infect or attack a mammal For example, viral vaccines can include vaccines against viruses such as human immunodeficiency virus (HIV), R. rickettsii, vaccinia, Shigella, poliovirus, adenovirus, influenza, hepatitis A, hepatitis B, dengue virus, Japanese B encephalitis, Varicella zoster, cytomegalovirus, hepatitis A, rotavirus, as well as vaccines against viral diseases like Lyme disease, measles, yellow fever, mumps, rabies, herpes, influenza, parainfluenza and the like. Bacterial vaccines can include vaccines against bacteria such as Vibrio cholerae, Salmonella typhi, Bordetella pertussis, Streptococcus pneumoniae, Hemophilus influenza, Clostridium tetani, Corynebacterium diphtheriae, Mycobacterium leprae, Neisseria gonorrhoeae, Neisseria meningitidis, Coccidioides immitis, and the like.

In general, the overexpressed gene products and the heterologous chaperone proteins of the present invention are expressed recombinantly, that is, by placing a nucleic acid molecule encoding a gene product or a chaperone protein into an expression vector. Such an expression vector minimally contains a sequence which effects expression of the gene product or the heterologous chaperone protein when the sequence is operably linked to a nucleic acid molecule encoding the gene product or the chaperone protein. Such an expression vector can also contain additional elements like origins of replication, selectable markers, transcription or termination signals, centromeres, autonomous replication sequences, and the like.

According to the present invention, first and second nucleic acid molecules encoding an overexpressed gene product and a heterologous chaperone protein, respectively, can be placed within expression vectors to permit regulated expression of the overexpressed gene product and/or the heterologous chaperone protein. While the heterologous chaperone protein and the overexpressed gene product can be encoded in the same expression vector, the heterologous chaperone protein is preferably encoded in an expression vector which is separate from the vector encoding the overexpressed gene product. Placement of nucleic acid molecules encoding the heterologous chaperone protein and the overexpressed gene product in separate expression vectors can increase the amount of secreted overexpressed gene product.

As used herein, an expression vector can be a replicable or a non-replicable expression vector. A replicable expression vector can replicate either independently of host cell chromosomal DNA or because such a vector has integrated into host cell chromosomal DNA. Upon integration into host cell chromosomal DNA such an expression vector can lose some structural elements but retains the nucleic acid molecule encoding the gene product or the chaperone protein and a segment which can effect expression of the gene product or the heterologous chaperone protein. Therefore, the expression vectors of the present invention can be chromosomally integrating or chromosomally nonintegrating expression vectors.

In a further embodiment, one or more heterologous chaperone proteins are overexpressed in a host cell by introduction of integrating or nonintegrating expression vectors into the host cell. Following introduction of at least one expression vector encoding at least one chaperone protein, the gene product is then overexpressed by inducing expression of an endogenous gene encoding the gene product, or by introducing into the host cell an expression vector encoding the gene product. In another embodiment, cell lines are established which constitutively or inducibly express at least one heterologous chaperone protein. An expression vector encoding the gene product to be overexpressed is introduced into such cell lines to achieve increased secretion of the overexpressed gene product.

The present expression vectors can be replicable in one host cell type, e.g., Escherichia coli, and undergo little or no replication in another host cell type, e.g., a eukaryotic host cell, so long as an expression vector permits expression of the heterologous chaperone proteins or overexpressed gene products and thereby facilitates secretion of such gene products in a selected host cell type.

Expression vectors as described herein include DNA or RNA molecules engineered for controlled expression of a desired gene, that is, a gene encoding the present chaperone proteins or a overexpressed gene product. Such vectors also encode nucleic acid molecule segments which are operably linked to nucleic acid molecules encoding the present chaperone polypeptides or the present overexpressed gene products. Operably linked in this context means that such segments can effect expression of nucleic acid molecules encoding chaperone protein or overexpressed gene products. These nucleic acid sequences include promoters, enhancers, upstream control elements, transcription factors or repressor binding sites, termination signals and other elements which can control gene expression in the contemplated host cell. Preferably the vectors are vectors, bacteriophages, cosmids, or viruses.

Expression vectors of the present invention function in yeast or mammalian cells. Yeast vectors can include the yeast 2μ circle and derivatives thereof, yeast vectors encoding yeast autonomous replication sequences, yeast minichromosomes, any yeast integrating vector and the like. A comprehensive listing of many types of yeast vectors is provided in Parent et al. (Yeast 1: 83-138 (1985)).

Elements or nucleic acid sequences capable of effecting expression of a gene product include promoters, enhancer elements, upstream activating sequences, transcription termination signals and polyadenylation sites. All such promoter and transcriptional regulatory elements, singly or in combination, are contemplated for use in the present expression vectors. Moreover, genetically-engineered and mutated regulatory sequences are also contemplated herein.

Promoters are DNA sequence elements for controlling gene expression. In particular, promoters specify transcription initiation sites and can include a TATA box and upstream promoter elements. The promoters selected are those which would be expected to be operable in the particular host system selected. For example, yeast promoters are used in the present expression vectors when a yeast host cell such as Saccharomyces cerevisiae, Kluyveromyces lactis, or Pichia pastoris is used whereas fungal promoters would be used in host cells such as Aspergillus niger, Neurospora crassa, or Tricoderma reesei. Examples of yeast promoters include but are not limited to the GAPDH, AOX1, GAL1, PGK, GAP, TPI, CYC1, ADH2, PHO5, CUP1, MFα1, PMA1, PDI, TEF, and GUT1 promoters. Romanos et al. (Yeast 8: 423-488 (1992)) provide a review of yeast promoters and expression vectors.

The promoters that are operably linked to the nucleic acid molecules disclosed herein can be constitutive promoters or inducible promoters. Inducible promoters, that is, promoters which direct transcription at an increased or decreased rate upon binding of a transcription factor. Transcription factors as used herein include any factor that can bind to a regulatory or control region of a promoter an thereby affect transcription. The synthesis or the promoter binding ability of a transcription factor within the host cell can be controlled by exposing the host to an inducer or removing an inducer from the host cell medium. Accordingly to regulate expression of an inducible promoter, an inducer is added or removed from the growth medium of the host cell. Such inducers can include sugars, phosphate, alcohol, metal ions, hormones, heat, cold and the like. For example, commonly used inducers in yeast are glucose, galactose, and the like.

Transcription termination sequences that are selected are those that are operable in the particular host cell selected. For example, yeast transcription termination sequences are used in the present expression vectors when a yeast host cell such as Saccharomyces cerevisiae, Kluyveromyces lactis, or Pichia pastoris is used whereas fungal transcription termination sequences would be used in host cells such as Aspergillus niger, Neurospora crassa, or Tricoderma reesei. Transcription termination sequences include but are not limited to the Saccharomyces cerevisiae CYC transcription termination sequence (ScCYC TT), the Pichia pastoris ALG3 transcription termination sequence (ALG3 TT), and Pichia pastoris PMA1 transcription termination sequence (PpPMA1 TT).

The expression vectors of the present invention can also encode selectable markers. Selectable markers are genetic functions that confer an identifiable trait upon a host cell so that cells transformed with a vector carrying the selectable marker can be distinguished from non-transformed cells. Inclusion of a selectable marker into a vector can also be used to ensure that genetic functions linked to the marker are retained in the host cell population. Such selectable markers can confer any easily identified dominant trait, e.g. drug resistance, the ability to synthesize or metabolize cellular nutrients and the like.

Yeast selectable markers include drug resistance markers and genetic functions which allow the yeast host cell to synthesize essential cellular nutrients, e.g. amino acids. Drug resistance markers which are commonly used in yeast include chloramphenicol, kanamycin, methotrexate, G418 (geneticin), Zeocin, and the like. Genetic functions which allow the yeast host cell to synthesize essential cellular nutrients are used with available yeast strains having auxotrophic mutations in the corresponding genomic function. Common yeast selectable markers provide genetic functions for synthesizing leucine (LEU2), tryptophan (TRP1 and TRP2), uracil (URA3, URA5, URA6), histidine (HIS3), lysine (LYS2), adenine (ADE1 or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al., J. Biol. Chem. 272:30061-066 (1997)). A number of suitable integration sites include those enumerated in U.S. Published application No. 20070072262 and include homologs to loci known for Saccharomyces cerevisiae and other yeast or fungi.

Therefore the present expression vectors can encode selectable markers which are useful for identifying and maintaining vector-containing host cells within a cell population present in culture. In some circumstances selectable markers can also be used to amplify the copy number of the expression vector. After inducing transcription from the present expression vectors to produce an RNA encoding an overexpressed gene product or a heterologous chaperone protein, the RNA is translated by cellular factors to produce the gene product or the heterologous chaperone protein.

In yeast and other eukaryotes, translation of a messenger RNA (mRNA) is initiated by ribosomal binding to the 5′ cap of the mRNA and migration of the ribosome along the mRNA to the first AUG start codon where polypeptide synthesis can begin. Expression in yeast and mammalian cells generally does not require specific number of nucleotides between a ribosomal-binding site and an initiation codon, as is sometimes required in prokaryotic expression systems. However, for expression in a yeast or a mammalian host cell, the first AUG codon in an mRNA is preferably the desired translational start codon.

Moreover, when expression is performed in a yeast host cell the presence of long untranslated leader sequences, e.g. longer than 50-100 nucleotides, can diminish translation of an mRNA. Yeast mRNA leader sequences have an average length of about 50 nucleotides, are rich in adenine, have little secondary structure and almost always use the first AUG for initiation. Since leader sequences which do not have these characteristics can decrease the efficiency of protein translation, yeast leader sequences are preferably used for expression of an overexpressed gene product or a chaperone protein in a yeast host cell. The sequences of many yeast leader sequences are known and are available to the skilled artisan, for example, by reference to Cigan et al. (Gene 59: 1-18 (1987)).

In addition to the promoter, the ribosomal-binding site and the position of the start codon, factors which can effect the level of expression obtained include the copy number of a replicable expression vector. The copy number of a vector is generally determined by the vector's origin of replication and any cis-acting control elements associated therewith. For example, an increase in copy number of a yeast episomal vector encoding a regulated centromere can be achieved by inducing transcription from a promoter which is closely juxtaposed to the centromere. Moreover, encoding the yeast FLP function in a yeast vector can also increase the copy number of the vector.

One skilled in the art can also readily design and make expression vectors which include the above-described sequences by combining DNA fragments from available vectors, by synthesizing nucleic acid molecules encoding such regulatory elements or by cloning and placing new regulatory elements into the present vectors. Methods for making expression vectors are well-known. Overexpressed DNA methods are found in any of the myriad of standard laboratory manuals on genetic engineering.

The expression vectors of the present invention can be made by ligating the heterologous chaperone protein coding regions in the proper orientation to the promoter and other sequence elements being used to control gene expression. After construction of the present expression vectors, such vectors are transformed into host cells where the overexpressed gene product and the heterologous chaperone protein can be expressed. Methods for transforming yeast and other lower eukaryotic cells with expression vectors are well known and readily available to the skilled artisan. For example, expression vectors can be transformed into yeast cells by any of several procedures including lithium acetate, spheroplast, electroporation, and similar procedures.

Yeast host cells which can be used with yeast replicable expression vectors include any wild type or mutant strain of yeast which is capable of secretion. Such strains can be derived from Saccharomyces cerevisiae, Hansenula polymorpha, Kluyveromyces lactis, Pichia pastoris, Schizosaccharomyces pombe, Yarrowia lipolytica, and related species of yeast. In general, useful mutant strains of yeast include strains which have a genetic deficiency that can be used in combination with a yeast vector encoding a selectable marker. Many types of yeast strains are available from the Yeast Genetics Stock Center (Donner Laboratory, University of California, Berkeley, Calif. 94720), the American Type Culture Collection (12301 Parklawn Drive, Rockville, Md. 20852, hereinafter ATCC), the National Collection of Yeast Cultures (Food Research Institute, Colney Lane, Norwich NR4 7UA, UK) and the Centraalbureau voor Schimmelcultures (Yeast Division, Julianalaan 67a, 2628 BC Delft, Netherlands).

In general, lower eukaryotes such as yeast are useful for expression of glycoproteins because they can be economically cultured, give high yields, and when appropriately modified are capable of suitable glycosylation. Yeast particularly offers established genetics allowing for rapid transformations, tested protein localization strategies and facile gene knock-out techniques. Suitable vectors have expression control sequences, such as promoters, including 3-phosphoglycerate kinase or other glycolytic enzymes, and an origin of replication, termination sequences and the like as desired.

Various yeasts, such as Kluyveromyces lactis, Pichia pastoris, Pichia methanolica, and Hansenula polymorpha are useful for cell culture because they are able to grow to high cell densities and secrete large quantities of recombinant protein. Likewise, filamentous fungi, such as Aspergillus niger, Fusarium sp, Neurospora crassa and others can be used to produce glycoproteins of the invention at an industrial scale.

Lower eukaryotes, particularly yeast, can be genetically modified so that they express glycoproteins in which the glycosylation pattern is human-like or humanized. Such can be achieved by eliminating selected endogenous glycosylation enzymes and/or supplying exogenous enzymes as described by Gerngross et al., US 20040018590. For example, a host cell can be selected or engineered to be depleted in 1,6-mannosyl transferase activities, which would otherwise add mannose residues onto the N-glycan on a glycoprotein.

In one embodiment, the host cell further includes an α1,2-mannosidase catalytic domain fused to a cellular targeting signal peptide not normally associated with the catalytic domain and selected to target the α1,2-mannosidase activity to the ER or Golgi apparatus of the host cell. Passage of a recombinant glycoprotein through the ER or Golgi apparatus of the host cell produces a recombinant glycoprotein comprising a Man₅GlcNAc₂ glycoform, for example, a recombinant glycoprotein composition comprising predominantly a Man₅GlcNAc₂ glycoform. For example, U.S. Pat. No. 7,029,872 and U.S. Published Patent Application Nos. 2004/0018590 and 2005/0170452 disclose lower eukaryote host cells capable of producing a glycoprotein comprising a Man₅GlcNAc₂ glycoform.

In a further embodiment, the immediately preceding host cell further includes a GlcNAc transferase I (GnT I) catalytic domain fused to a cellular targeting signal peptide not normally associated with the catalytic domain and selected to target GlcNAc transferase I activity to the ER or Golgi apparatus of the host cell. Passage of the recombinant glycoprotein through the ER or Golgi apparatus of the host cell produces a recombinant glycoprotein comprising a GlcNAcMan₅GlcNAc₂ glycoform, for example a recombinant glycoprotein composition comprising predominantly a GlcNAcMan₅GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAcMan₅GlcNAc₂ glycoform. The glycoprotein produced in the above cells can be treated in vitro with a hexaminidase to produce a recombinant glycoprotein comprising a Man₅GlcNAc₂ glycoform.

In a further embodiment, the immediately preceding host cell further includes a mannosidase II catalytic domain fused to a cellular targeting signal peptide not normally associated with the catalytic domain and selected to target mannosidase II activity to the ER or Golgi apparatus of the host cell. Passage of the recombinant glycoprotein through the ER or Golgi apparatus of the host cell produces a recombinant glycoprotein comprising a GlcNAcMan₃GlcNAc₂ glycoform, for example a recombinant glycoprotein composition comprising predominantly a GlcNAcMan₃GlcNAc₂ glycoform. U.S. Pat. No. 7,029,872 and U.S. Published Patent Application No. 2004/0230042 discloses lower eukaryote host cells that express mannosidase II enzymes and are capable of producing glycoproteins having predominantly a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform.

In a further embodiment, the immediately preceding host cell further includes GlcNAc transferase II (GnT II) catalytic domain fused to a cellular targeting signal peptide not normally associated with the catalytic domain and selected to target GlcNAc transferase II activity to the ER or Golgi apparatus of the host cell. Passage of the recombinant glycoprotein through the ER or Golgi apparatus of the host cell produces a recombinant glycoprotein comprising a GlcNAc₂Man₃GlcNAc₂ glycoform, for example a recombinant glycoprotein composition comprising predominantly a GlcNAc₂Man₃GlcNAc₂ glycoform. U.S. Pat. No. 7,029,872 and U.S. Published Patent Application Nos. 2004/0018590 and 2005/0170452 disclose lower eukaryote host cells capable of producing a glycoprotein comprising a GlcNAc₂Man₃GlcNAc₂ glycoform. The glycoprotein produced in the above cells can be treated in vitro with a hexaminidase to produce a recombinant glycoprotein comprising a Man₃GlcNAc₂ glycoform.

In a further embodiment, the immediately preceding host cell further includes a galactosyltransferase catalytic domain fused to a cellular targeting signal peptide not normally associated with the catalytic domain and selected to target galactosyltransferase activity to the ER or Golgi apparatus of the host cell. Passage of the recombinant glycoprotein through the ER or Golgi apparatus of the host cell produces a recombinant glycoprotein comprising a GalGlcNAc₂Man₃GlcNAc₂ or Gal₂GlcNAc₂Man₃GlcNAc₂ glycoform, or mixture thereof for example a recombinant glycoprotein composition comprising predominantly a GalGlcNAc₂Man₃GlcNAc₂ glycoform or Gal₂GlcNAc₂Man₃GlcNAc₂ glycoform or mixture thereof. U.S. Pat. No. 7,029,872 and U.S. Published Patent Application No. 2006/0040353 discloses lower eukaryote host cells capable of producing a glycoprotein comprising a Gal₂GlcNAc₂Man₃GlcNAc₂ glycoform. The glycoprotein produced in the above cells can be treated in vitro with a galactosidase to produce a recombinant glycoprotein comprising a GlcNAc₂Man₃GlcNAc₂ glycoform, for example a recombinant glycoprotein composition comprising predominantly a GlcNAc₂Man₃GlcNAc₂ glycoform.

In a further embodiment, the immediately preceding host cell further includes a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising predominantly a NANA₂Gal₂GlcNAc₂Man₃GlcNAc₂ glycoform or NANAGal₂GlcNAc₂Man₃GlcNAc₂ glycoform or mixture thereof. For lower eukaryote host cells such as yeast and filamentous fungi, it is useful that the host cell further include a means for providing CMP-sialic acid for transfer to the N-glycan. U.S. Published Patent Application No. 2005/0260729 discloses a method for genetically engineering lower eukaryotes to have a CMP-sialic acid synthesis pathway and U.S. Published Patent Application No. 2006/0286637 discloses a method for genetically engineering lower eukaryotes to produce sialylated glycoproteins. The glycoprotein produced in the above cells can be treated in vitro with a neuraminidase to produce a recombinant glycoprotein comprising predominantly a Gal₂GlcNAc₂Man₃GlcNAc₂ glycoform or GalGlcNAc₂Man₃GlcNAc₂ glycoform or mixture thereof.

Any one of the preceding host cells can further include one or more GlcNAc transferase selected from the group consisting of GnT III, GnT IV, GnT V, GnT VI, and GnT IX to produce glycoproteins having bisected (GnT III) and/or multiantennary (GnT IV, V, VI, and IX) N-glycan structures such as disclosed in U.S. Published Patent Application Nos. 2004/074458 and 2007/0037248.

In further embodiments, the host cell that produces glycoproteins that have predominantly GlcNAcMan₅GlcNAc₂ N-glycans further includes a galactosyltransferase catalytic domain fused to a cellular targeting signal peptide not normally associated with the catalytic domain and selected to target Galactosyltransferase activity to the ER or Golgi apparatus of the host cell. Passage of the recombinant glycoprotein through the ER or Golgi apparatus of the host cell produces a recombinant glycoprotein comprising predominantly the GalGlcNAcMan₅GlcNAc₂ glycoform.

In a further embodiment, the immediately preceding host cell that produced glycoproteins that have predominantly the predominantly the GalGlcNAcMan₅GlcNAc₂ N-glycans further includes a sialyltransferase catalytic domain fused to a cellular targeting signal peptide not normally associated with the catalytic domain and selected to target sialytransferase activity to the ER or Golgi apparatus of the host cell. Passage of the recombinant glycoprotein through the ER or Golgi apparatus of the host cell produces a recombinant glycoprotein comprising a NANAGalGlcNAcMan₅GlcNAc₂ glycoform.

Various of the preceding host cells further include one or more sugar transporters such as UDP-GlcNAc transporters (for example, Kluyveromyces lactis and Mus musculus UDP-GlcNAc transporters), UDP-galactose transporters (for example, Drosophila melanogaster UDP-galactose transporter), and CMP-sialic acid transporter (for example, human sialic acid transporter). Because lower eukaryote host cells such as yeast and filamentous fungi lack the above transporters, it is preferable that lower eukaryote host cells such as yeast and filamentous fungi be genetically engineered to include the above transporters.

In further embodiments of the above host cells, the host cells are further genetically engineered to eliminate glycoproteins having α-mannosidase-resistant N-glycans by deleting or disrupting the β-mannosyltransferase gene (BMT2) (See, U.S. Published Patent Application No. 2006/0211085) and glycoproteins having phosphomannose residues by deleting or disrupting one or both of the phosphomannosyl transferase genes PNO1 and MNN4B (See for example, U.S. Pat. Nos. 7,198,921 and 7,259,007). In further still embodiments of the above host cells, the host cells are further genetically modified to eliminate O-glycosylation of the glycoprotein by deleting or disrupting one or more of the protein O-mannosyltransferase (Dol-P-Man:Protein (Ser/Thr) Mannosyl Transferase genes) (PMTs) (See U.S. Pat. No. 5,714,377) or grown in the presence of i inhibitors such as Pmt-1, Pmti-2, and Pmti-3 as disclosed in Published International Application No. WO 2007061631, or both.

Thus, provided are host cells that have been genetically modified to produce glycoproteins wherein the predominant N-glycans thereon include but are not limited to Man₈GlcNAc₂, Man₇GlcNAc₂, Man₆GlcNAc₂, Man₅GlcNAc₂, GlcNAcMan₅GlcNAc₂, GalGlcNAcMan₅GlcNAc₂, NANAGalGlcNAcMan₅GlcNAc₂, Man₃GlcNAc₂, GlcNAc₍₁₋₄₎Man₃GlcNAc₂, Gal₍₁₋₄₎GlcNAc₍₁₋₄₎Man₃GlcNAc₂, NANA₍₁₋₄₎Gal₍₁₋₄₎GlcNAc₍₁₋₄₎Man₃GlcNAc₂. Further included are host cells that produce glycoproteins that have particular mixtures of the aforementioned N-glycans thereon.

In the following examples, heterologous human proteins are expressed in host cells of the species Pichia pastoris. These examples demonstrate the invention with respect to specific embodiments of the invention, and are not to be construed as limiting in any manner. The skilled artisan, having read the disclosure and examples herein, will recognize that numerous variants, modifications and improvements to the methods and materials described that are possible without deviating from the practice of the present invention.

### EXAMPLE 1

This example shows that expression of heterologous human proteins in Pichia pastoris was enhanced by using host cells in which the gene encoding the endogenous PDI1 has been inactivated and replaced with an expression cassette encoding the human PDI. The example further shows that these host cells produced recombinant antibodies that had reduced O-glycosylation.

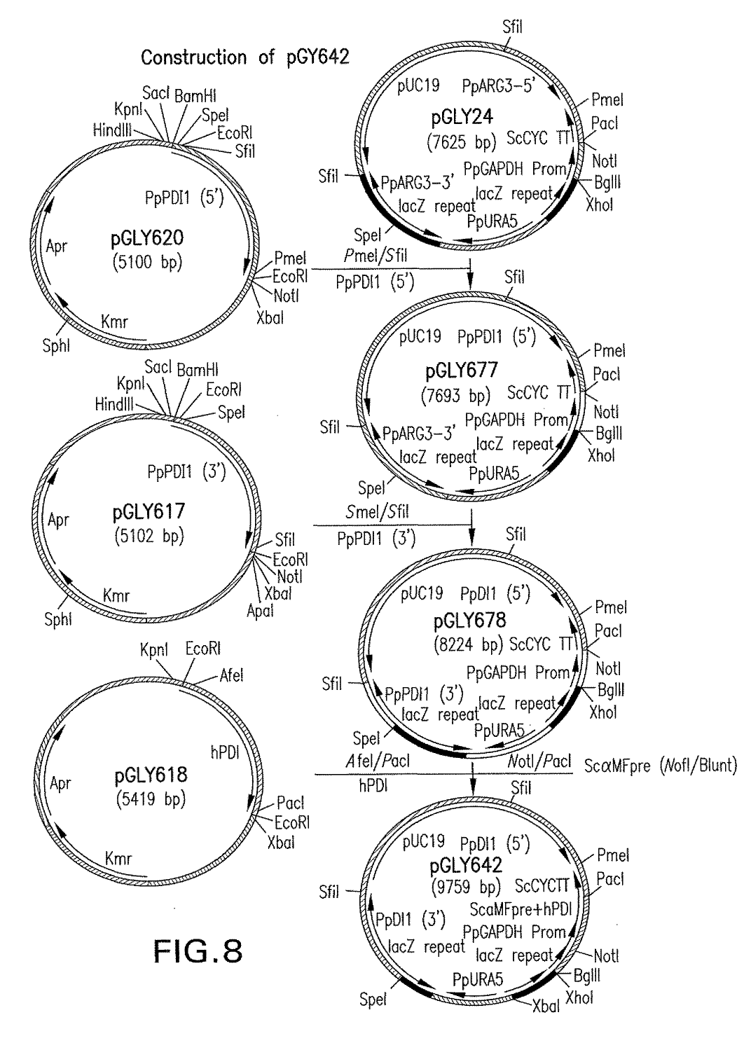

Construction of expression/integration plasmid vector pGLY642 comprising an expression cassette encoding the human PDI protein and nucleic acid molecules to target the plasmid vector to the Pichia pastoris PDI1 locus for replacement of the gene encoding the Pichia pastoris PDI1 with a nucleic acid molecule encoding the human PDI was as follows and is shown in FIG. 8. cDNA encoding the human PDI was amplified by PCR using the primers hPDI/UP1: 5′ AGCGCTGACGCCCCCGAGGAGGAGGACCAC 3′ (SEQ ID NO: 1) and hPDI/LP-PacI: 5′ CCTTAATTAATTACAGTTCATCATGCACAGCTTTCTGATCAT 3′ (SEQ ID NO: 2), Pfu turbo DNA polymerase (Stratagene, La Jolla, Calif.), and a human liver cDNA (BD Bioscience, San Jose, Calif.). The PCR conditions were 1 cycle of 95° C. for two minutes, 25 cycles of 95° C. for 20 seconds, 58° C. for 30 seconds, and 72° C. for 1.5 minutes, and followed by one cycle of 72° C. for 10 minutes. The resulting PCR product was cloned into plasmid vector pCR2.1 to make plasmid vector pGLY618. The nucleotide and amino acid sequences of the human PDI (SEQ ID NOs: 39 and 40, respectively) are shown in Table 11.

The nucleotide and amino acid sequences of the Pichia pastoris PDI1 (SEQ ID NOs:41 and 42, respectively) are shown in Table 11. Isolation of nucleic acid molecules comprising the Pichia pastoris PDI1 5′ and 3′ regions was performed by PCR amplification of the regions from Pichia pastoris genomic DNA. The 5′ region was amplified using primers PB248: 5′ ATGAATTCAGGCCATATCGGCCATTGTTTACTGTGCGCCCACAGT AG 3′ (SEQ ID NO: 3); PB249: 5′ ATGTTTAAACGTGAGGATTACTGGTGATGAAAGAC 3′ (SEQ ID NO: 4). The 3′ region was amplified using primers PB250: 5′ AGACTAGTCTATTTG GAGACATTGACGGATCCAC 3′ (SEQ ID NO: 5); PB251: 5′ ATCTCGAGAGGCCAT GCAGGCCAACCACAAGATGAATCAAATTTTG-3′ (SEQ ID NO: 6). Pichia pastoris strain NRRL-Y11430 genomic DNA was used for PCR amplification. The PCR conditions were one cycle of 95° C. for two minutes, 25 cycles of 95° C. for 30 seconds, 55° C. for 30 seconds, and 72° C. for 2.5 minutes, and followed by one cycle of 72° C. for 10 minutes. The resulting PCR fragments, PpPDI1 (5′) and PpPDI1 (3′), were separately cloned into plasmid vector pCR2.1 to make plasmid vectors pGLY620 and pGLY617, respectively. To construct pGLY678, DNA fragments PpARG3-5′ and PpARG-3′ of integration plasmid vector pGLY24, which targets the plasmid vector to Pichia pastoris ARG3 locus, were replaced with DNA fragments PpPDI (5′) and PpPDI (3′), respectively, which targets the plasmid vector pGLY678 to the PDI1 locus and disrupts expression of the PDI1 locus.

The nucleic acid molecule encoding the human PDI was then cloned into plasmid vector pGLY678 to produce plasmid vector pGLY642 in which the nucleic acid molecule encoding the human PDI was placed under the control of the Pichia pastoris GAPDH promoter (PpGAPDH). Expression/integration plasmid vector pGLY642 was constructed by ligating a nucleic acid molecule (SEQ ID NO: 27) encoding the Saccharomyces cerevisiae alpha mating factor pre-signal peptide (ScαMFpre-signal peptide (SEQ ID NO: 28) having a NotI restriction enzyme site at the 5′ end and a blunt 3′ end and the expression cassette comprising the nucleic acid molecule encoding the human PDI released from plasmid vector pGLY618 with AfeI and PacI to produce a nucleic acid molecule having a blunt 5′ end and a PacI site at the 3′ end into plasmid vector pGLY678 digested with NotI and PacI. The resulting integration/expression plasmid vector pGLY642 comprises an expression cassette encoding a human PDF ScαMFpre-signal peptide fusion protein operably linked to the Pichia pastoris promoter and nucleic acid molecule sequences to target the plasmid vector to the Pichia pastoris PDI1 locus for disruption of the PDI1 locus and integration of the expression cassette into the PDI1 locus. FIG. 8 illustrates the construction of plasmid vector pGLY642. The nucleotide and amino acid sequences of the ScαMFpre-signal peptide are shown in SEQ ID NOs: 27 and 28, respectively.

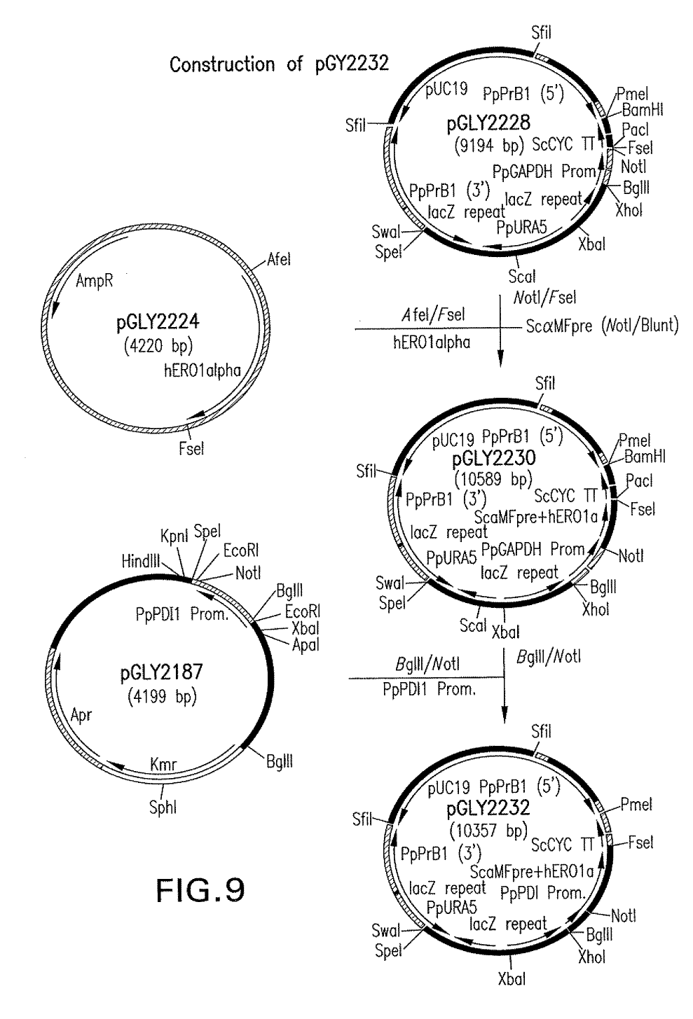

Construction of expression/integration vector pGLY2232 encoding the human ERO1α protein was as follows and is shown in FIG. 9. A nucleic acid molecule encoding the human ERO1α protein was synthesized by GeneArt AG (Regensburg, Germany) and used to construct plasmid vector pGLY2224. The nucleotide and amino acid sequences of the human ERO1α protein (SEQ ID NOs: 43 and 44, respectively) are shown in Table 11. The nucleic acid molecule encoding the human ERO1α protein was released from the plasmid vector using restriction enzymes AfeI and FseI and then ligated with a nucleic acid molecule encoding the ScαMPpre-signal peptide with 5′ NotI and 3′ blunt ends as above into plasmid vector pGLY2228 digested with NotI and FseI. Plasmid vector pGLY2228 also included nucleic acid molecules that included the 5′ and 3′ regions of the Pichia pastoris PRB1 gene (PpPRB1-5′ and PpPRB1-3′ regions, respectively). The resulting plasmid vector, pGLY2230 was digested with BglII and NotI and then ligated with a nucleic acid molecule containing the Pichia pastoris PDI1 promoter (PpPDI promoter) which had been obtained from plasmid vector pGLY2187 digested with BglII and NotI. The nucleotide sequence of the PpPDI promoter is 5′-AACACGAACACTGTAAAT AGAATAAAAGAAAACTTGGATAGTAGAACTTCAATGTAGTGTTTCTATTGTCTTACG CGGCTCTTTAGATTGCAATCCCCAGAATGGAATCGTCCATCTTTCTCAACCCACTCAA AGATAATCTACCAGACATACCTACGCCCTCCATCCCAGCACCACGTCGCGATCACCC CTAAAACTTCAATAATTGAACACGTACTGATTTCCAAACCTTCTTCTTCTTCCTATCT ATAAGA-3′ (SEQ ID NO: 59). The resulting plasmid vector, pGLY2232, is an expression/integration vector that contains an expression cassette that encodes the human ERO1α fusion protein under control of the Pichia pastoris PDI1 promoter and includes the 5′ and 3′ regions of the Pichia pastoris PRB1 gene to target the plasmid vector to the PRB1 locus of genome for disruption of the PRB1 locus and integration of the expression cassette into the PRB1 locus. FIG. 9 illustrates the construction of plasmid vector pGLY2232.

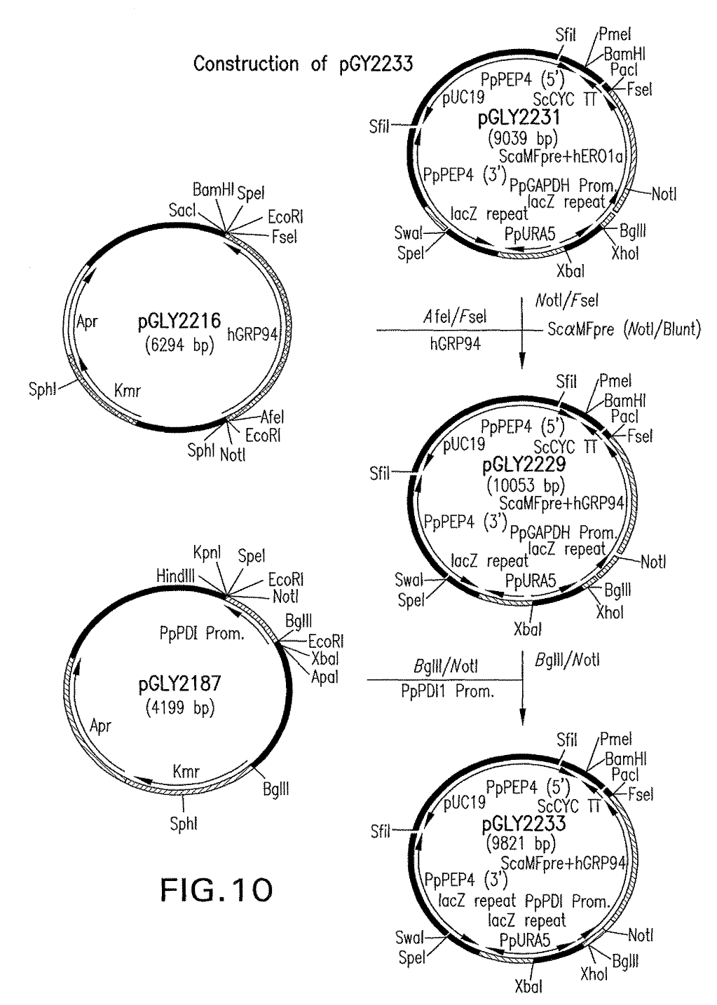

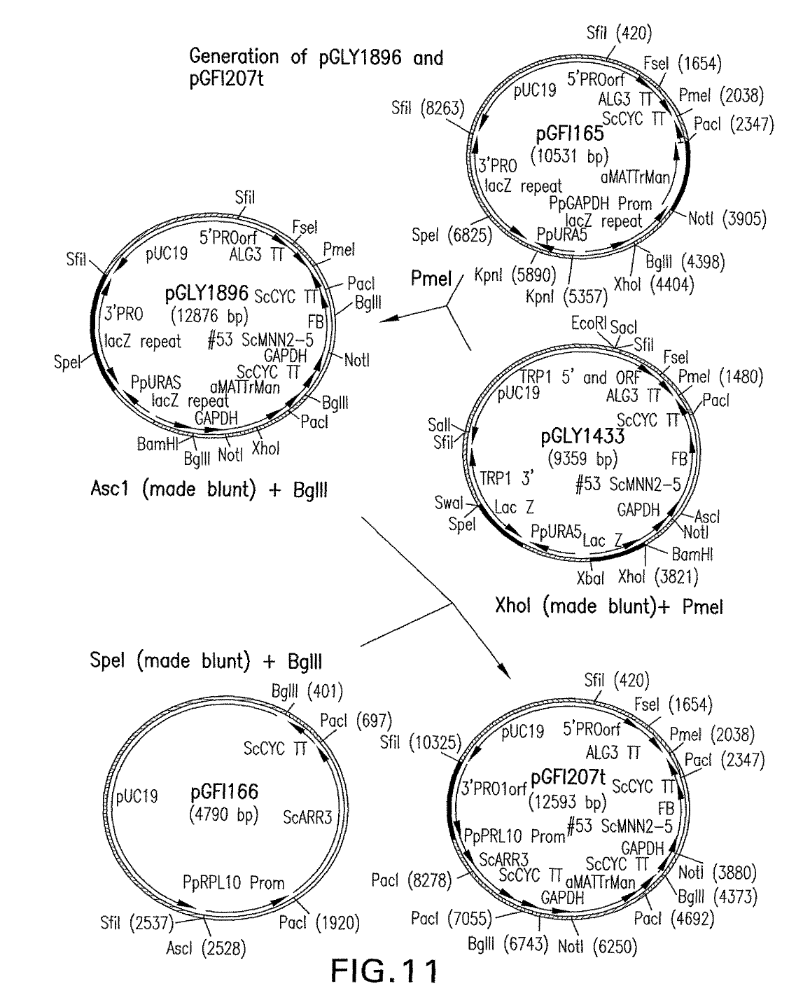

Construction of expression/integration vector pGLY2233 encoding the human GRP94 protein was as follows and is shown in FIG. 10. The human GRP94 was PCR amplified from human liver cDNA (BD Bioscience) with the primers hGRP94/UP1: 5′-AGCGC TGACGATGAAGTTGATGTGGATGGTACAGTAG-3′; (SEQ ID NO: 15); and hGRP94/LP1: 5′-GGCCG GCCTT ACAAT TCATC ATGTT CAGCT GTAGA TTC 3′; (SEQ ID NO: 16). The PCR conditions were one cycle of 95° C. for two minutes, 25 cycles of 95° C. for 20 seconds, 55° C. for 20 seconds, and 72° C. for 2.5 minutes, and followed by one cycle of 72° C. for 10 minutes. The PCR product was cloned into plasmid vector pCR2.1 to make plasmid vector pGLY2216. The nucleotide and amino acid sequences of the human GRP94 (SEQ ID NOs: 45 and 46, respectively) are shown in Table 11.

The nucleic acid molecule encoding the human GRP94 was released from plasmid vector pGLY2216 with AfeI and FseI. The nucleic acid molecule was then ligated to a nucleic acid molecule encoding the ScαMPpre-signal peptide having NotI and blunt ends as above and plasmid vector pGLY2231 digested with NotI and FseI carrying nucleic acid molecules comprising the Pichia pastoris PEP4 5′ and 3′ regions (PpPEP4-5′ and PpPEP4-3′ regions, respectively) to make plasmid vector pGLY2229. Plasmid vector pGLY2229 was digested with BglII and NotI and a DNA fragment containing the PpPDI1 promoter was removed from plasmid vector pGLY2187 with BglII and NotI and the DNA fragment ligated into pGLY2229 to make plasmid vector pGLY2233. Plasmid vector pGLY2233 encodes the human GRP94 fusion protein under control of the Pichia pastoris PDI promoter and includes the 5′ and 3′ regions of the Pichia pastoris PEP4 gene to target the plasmid vector to the PEP4 locus of genome for disruption of the PEP4 locus and integration of the expression cassette into the PEP4 locus. FIG. 10 illustrates the construction of plasmid vector pGLY2233.

Construction of plasmid vectors pGLY1162, pGLY1896, and pGFI207t was as follows. All Trichoderma reesei α-1,2-mannosidase expression plasmid vectors were derived from pGFI165, which encodes the T. reesei α-1,2-mannosidase catalytic domain (See published International Application No. WO2007061631) fused to S. cerevisiae αMATpre signal peptide herein expression is under the control of the Pichia pastoris GAP promoter and wherein integration of the plasmid vectors is targeted to the Pichia pastoris PRO1 locus and selection is using the Pichia pastoris URA5 gene. A map of plasmid vector pGFI165 is shown in FIG. 11.

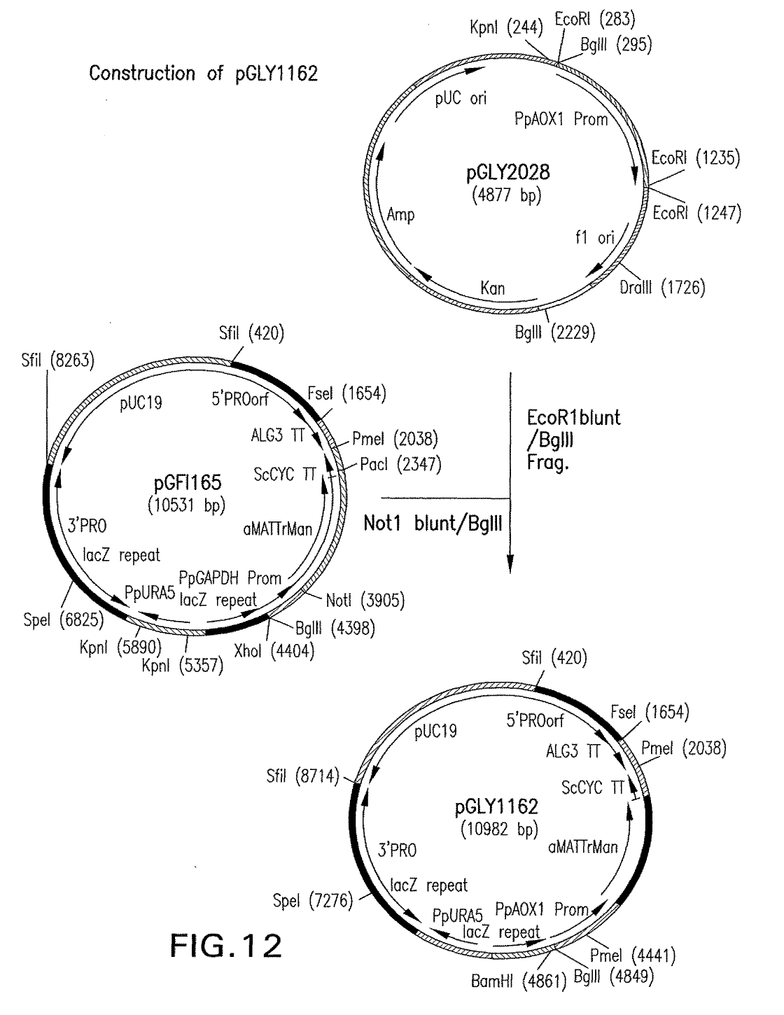

Plasmid vector pGLY1162 was made by replacing the GAP promoter in pGFI165 with the Pichia pastoris AOX1 (PpAOX1) promoter. This was accomplished by isolating the PpAOX1 promoter as an EcoRI (made blunt)-BglII fragment from pGLY2028, and inserting into pGFI165 that was digested with NotI (made blunt) and BglII. Integration of the plasmid vector is to the Pichia pastoris PRO1 locus and selection is using the Pichia pastoris URA5 gene. A map of plasmid vector pGLY1162 is shown in FIG. 12.

Plasmid vector pGLY1896 contains an expression cassette encoding the mouse α-1,2-mannosidase catalytic domain fused to the S. cerevisiae MNN2 membrane insertion leader peptide fusion protein (See Choi et al., Proc. Natl. Acad. Sci. USA 100: 5022 (2003)) inserted into plasmid vector pGFI165 (FIG. 12). This was accomplished by isolating the GAPp-ScMNN2-mouse MNSI expression cassette from pGLY1433 digested with XhoI (and the ends made blunt) and PmeI, and inserting the fragment into pGFI165 that digested with PmeI. Integration of the plasmid vector is to the Pichia pastoris PRO1 locus and selection is using the Pichia pastoris URA5 gene. A map of plasmid vector pGLY1896 is shown in FIG. 11.

Plasmid vector pGFI207t is similar to pGLY1896 except that the URA5 selection marker was replaced with the S. cerevisiae ARR3 (ScARR3) gene, which confers resistance to arsenite. This was accomplished by isolating the ScARR3 gene from pGFI166 digested with AscI and the AscI ends made blunt) and BglII, and inserting the fragment into pGLY1896 that digested with SpeI and the SpeI ends made blunt and BglII. Integration of the plasmid vector is to the Pichia pastoris PRO1 locus and selection is using the Saccharomyces cerevisiae ARR3 gene. A map of plasmid vector pGFI207t is shown in FIG. 11.

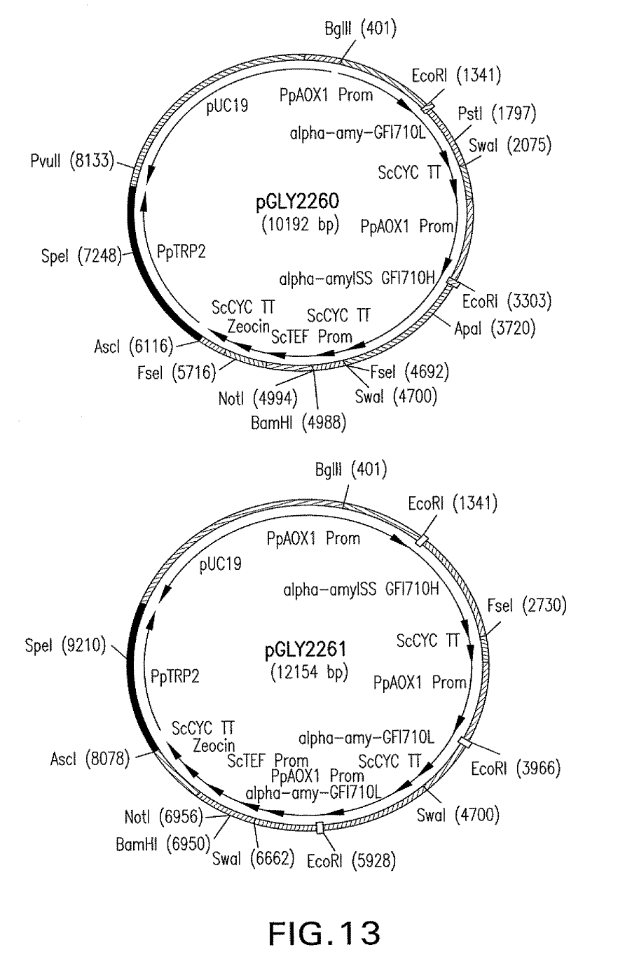

Construction of anti-DKK1 antibody expression/integration plasmid vectors pGLY2260 and pGLY2261 was as follows. Anti-DKK1 antibodies are antibodies that recognize Dickkopf protein 1, a ligand involved in the Wnt signaling pathway. To generate expression/integration plasmid vectors pGLY2260 and pGLY2261 encoding an anti-DKK1 antibody, codon-optimized nucleic acid molecules encoding heavy chain (HC; fusion protein containing VH+IgG₂m4) and light chain (LC; fusion protein containing VL+Lλ constant region) fusion proteins, each in frame with a nucleic acid molecule encoding an α-amylase (from Aspergillus niger) signal peptide were synthesized by GeneArt AG. The nucleotide and amino acid sequences for the α-amylase signal peptide are shown in SEQ ID NOs: 33 and 34. The nucleotide sequence of the HC is shown in SEQ ID NO: 51 and the amino acid sequence is shown in SEQ ID NO: 52. The nucleotide sequence of the LC is shown in SEQ ID NO: 53 and the amino acid sequence is shown in SEQ ID NO: 54. The IgG₂m4 isotype has been disclosed in U.S. Published Application No. 2007/0148167 and U.S. Published Application No. 2006/0228349. The nucleic acid molecules encoding the HC and LC fusion proteins were separately cloned using unique 5′-EcoRI and 3′-FseI sites into expression plasmid vector pGLY1508 to form plasmid vectors pGLY1278 and pGLY1274, respectively. These plasmid vectors contained the Zeocin-resistance marker and TRP2 integration sites and the Pichia pastoris AOX1 promoter operably linked to the nucleic acid molecules encoding the HC and LC fusion proteins. The LC fusion protein expression cassette was removed from pGLY1274 with BglII and BamHI and cloned into pGLY1278 digested with BglII to generate plasmid vector pGLY2260, which encodes the HC and LC fusion proteins and targets the expression cassettes to the TRP2 locus for integration of the expression cassettes into the TRP2 locus. The plasmid vector pGLY2261 contains an additional LC in plasmid vector pGLY2260. (FIG. 13).

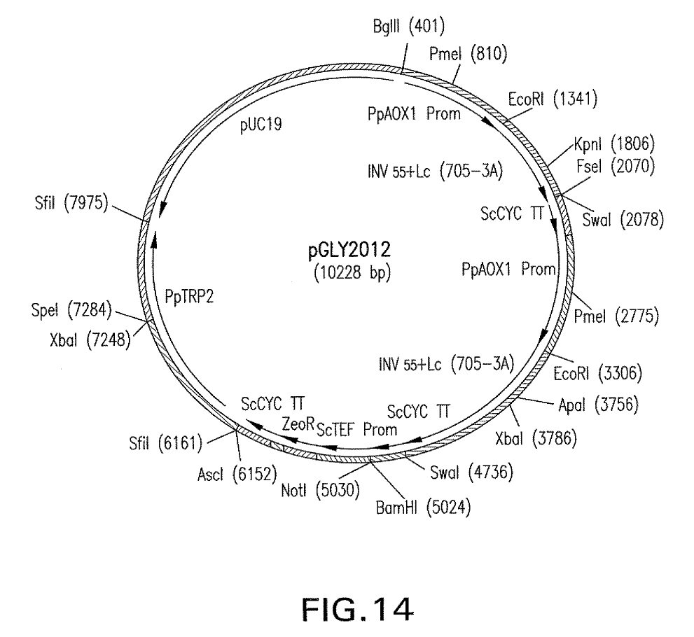

Construction of anti-ADDL antibody expression/integration plasmid vector pGLY2260 was as follows. Anti-ADDL antibodies are antibodies that recognize Aβ-derived diffusible ligands, see for example U.S. Published Application No. 20070081998. To generate expression/integration plasmid vector pGLY2012, codon-optimized nucleic acid molecules encoding heavy chain (HC; contained VH+IgG2m4) and light chain (LC; fusion protein containing VL+Lλ constant region) fusion proteins, each in frame with a nucleic acid molecule encoding Saccharomyces cerevisiae invertase signal peptide were synthesized by GeneArt AG. The nucleic acid molecules encoding the HC and LC fusion proteins were separately cloned using unique 5′-EcoRI and 3′-FseI sites into expression/integration plasmid vectors pGLY1508 and pGLY1261 to form pGLY2011 and pGLY2010, respectively, which contained the Zeocin-resistance marker and TRP2 integration sites and the Pichia pastoris AOX1 promoter operably linked to the nucleic acid molecules encoding the HC and LC fusion proteins. The HC expression cassette was removed from pGLY2011 with BglII and NotI and cloned into pGLY2010 digested with BamHI and NotI to generate pGLY2012, which encodes the HC and LC fusion proteins and targets the expression cassettes to the TRP2 locus for integration of the expression cassettes into the TRP2 locus (FIG. 14).

Yeast transformations with the above expression/integration vectors were as follows. Pichia pastoris strains were grown in 50 mL YPD media (yeast extract (1%), peptone (2%), dextrose (2%)) overnight to an OD of between about 0.2 to 6.0. After incubation on ice for 30 minutes, cells were pelleted by centrifugation at 2500-3000 rpm for 5 minutes. Media was removed and the cells washed three times with ice cold sterile 1M sorbitol before resuspension in 0.5 ml ice cold sterile 1M sorbitol. Ten μL linearized DNA (5-20 μg) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (24° C.) before plating the cells on selective media.

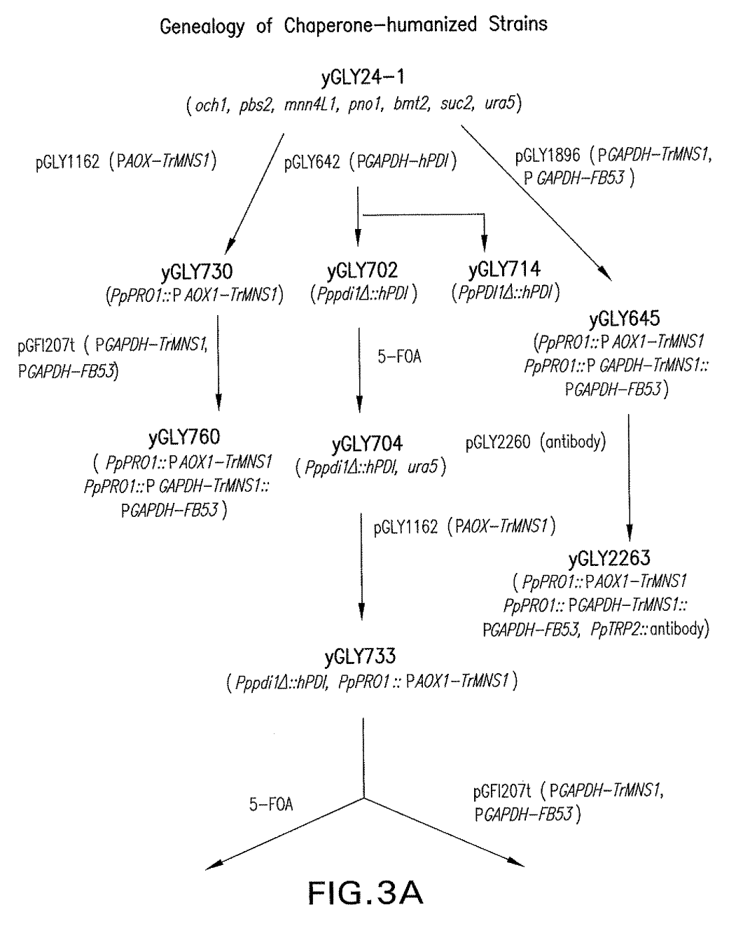

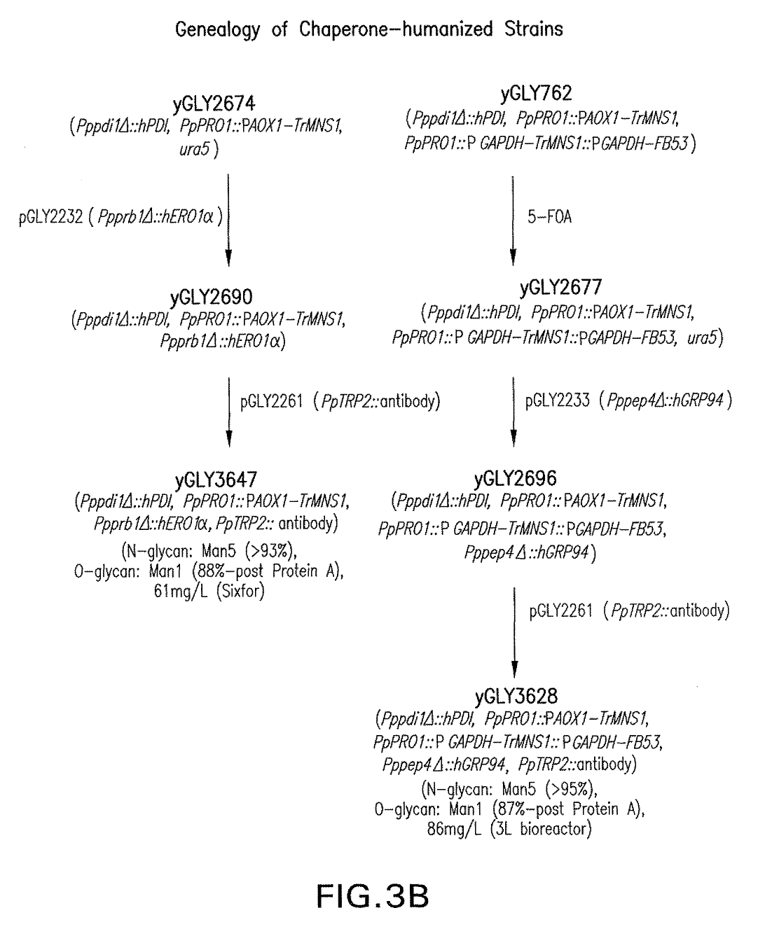

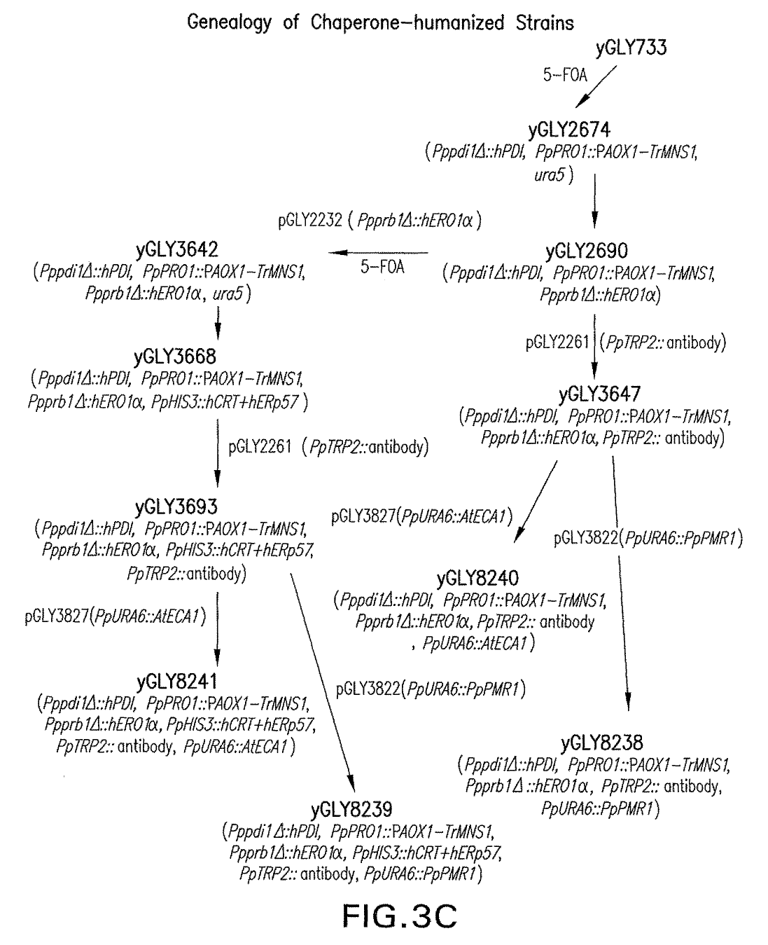

Generation of Cell Lines was as follows and is shown in FIG. 3. The strain yGLY24-1 (ura5Δ::MET1 och1Δ::lacZ bmt2Δ::lacZ/KlMNN2-2/mnn4L1Δ::lacZ/MmSLC35A3 pno1Δmnn4Δ::lacZ met16Δ::lacZ), was constructed using methods described earlier (See for example, Nett and Gerngross, Yeast 20:1279 (2003); Choi et al., Proc. Natl. Acad. Sci. USA 100:5022 (2003); Hamilton et al., Science 301:1244 (2003)). The BMT2 gene has been disclosed in Mille et al., J. Biol. Chem. 283: 9724-9736 (2008) and U.S. Published Application No. 20060211085. The PNO1 gene has been disclosed in U.S. Pat. No. 7,198,921 and the mnn4L1 gene (also referred to as mnn4b) has been disclosed in U.S. Pat. No. 7,259,007. The mnn4 refers to mnn4L2 or mnn4a. In the genotype, KlMNN2-2 is the Kluveromyces lactis GlcNAc transporter and MmSLC35A3 is the Mus musculus GlcNAc transporter. The URA5 deletion renders the yGLY24-1 strain auxotrophic for uracil (See U.S. Published application No. 2004/0229306) and was used to construct the humanized chaperone strains that follow. While the various expression cassettes were integrated into particular loci of the Pichia pastoris genome in the examples herein, it is understood that the operation of the invention is independent of the loci used for integration. Loci other than those disclosed herein can be used for integration of the expression cassettes. Suitable integration sites include those enumerated in U.S. Published application No. 20070072262 and include homologs to loci known for Saccharomyces cerevisiae and other yeast or fungi.

Control strain yGLY645 (PpPDI1) was constructed. Strain yGLY645 expresses both a Trichoderma Reesei mannosidase1 (TrMNS1) and a mouse mannosidase IA (MuMNS1A), each constitutively expressed under the control of a PpGAPDH promoter, with the native Pichia pastoris PDI1 locus intact. Strain yGLY645 was generated from strain yGLY24-1 by transforming yGLY24-1 with plasmid vector pGLY1896, which targeted the plasmid vector to the Proline 1 (PRO1) locus in the Pichia genome. Plasmid vector pGLY1896 contains expression cassettes encoding the Trichoderma Reesei mannosidase 1 (TrMNS 1) and the mouse mannosidase IA (FB53, MuMNS1A), each constitutively expressed under the control of a PpGAPDH promoter.

Strains yGLY702 and yGLY704 were generated in order to test the effectiveness of the human PDI1 expressed in Pichia pastoris cells in the absence of the endogenous Pichia pastoris PDI1 gene. Strains yGLY702 and yGLY704 (hPDI) were constructed as follows. Strain yGLY702 was generated by transforming yGLY24-1 with plasmid vector pGLY642 containing the expression cassette encoding the human PDI under control of the constitutive PpGAPDH promoter. Plasmid vector pGLY642 also contained an expression cassette encoding the Pichia pastoris URA5, which rendered strain yGLY702 prototrophic for uracil. The URA5 expression cassette was removed by counterselecting yGLY702 on 5-FOA plates to produce strain yGLY704 in which, so that the Pichia pastoris PDI1 gene has been stably replaced by the human PDI gene and the strain is auxotrophic for uracil.

The replacement of the Pichia pastoris PDI1 with the human PDI using plasmid vector pGLY642 was confirmed by colony PCR using the following primers specific to only the PpPDI1 ORF; PpPDI/UPi-1, 5′-GGTGAGGTTGAGGTCCCAAGTGACTATCAAGGTC-3′; (SEQ ID NO: 7); PpPDI/LPi-1, 5′-GACCTTGATAGTCACTTGGGACCTCAACCTCACC-3′; (SEQ ID NO: 8); PpPDI/UPi-2, 5′ CGCCAATGATGAGGATGCCTCTTCAAAGGT TGTG-3′; (SEQ ID NO: 9); and PpPDI/LPi-2, 5′-CACAACCTTTGAAGAGGCATCCTCATCATT GGCG-3′; (SEQ ID NO: 10). Thus, the absence of PCR product indicates the knockout of PpPDI1. The PCR conditions were one cycle of 95° C. for two minutes, 25 cycles of 95° C. for 20 seconds, 58° C. for 20 seconds, and 72° C. for one minute, and followed by one cycle of 72° C. for 10 minutes.

Additional PCR was used to confirm the double crossover of pGLY642 at the PpPDI1 locus using PCR primers; PpPDI-5′/UP, 5′-GGCGATTGCATTCGCGACTGTATC-3′; (SEQ ID NO: 11); and, hPDI-3′/LP 5′-CCTAGAGAGCGGTGGCCAAGATG-3′; (SEQ ID NO: 12). PpPDI-5′/UP primes the upstream region of PpPDI1 that is absent in PpPDI1 (5′) of pGY642 and hPDI-3′/LP primes human PDI ORF in pGLY642. The PCR conditions were one cycle of 95° C. for two minutes, 25 cycles of 95° C. for 20 seconds, 50° C. for 30 seconds, and 72° C. for 2.5 minutes, and followed by one cycle of 72° C. for 10 minutes.

The integration efficiency of a plasmid vector as a knockout (i.e., a double cross-over event) or as a ‘roll-in’ (i.e., a single integration of the plasmid vector into the genome, can be dependent upon a number of factors, including the number and length of homologous regions between vectors and the corresponding genes on host chromosomal DNA, selection markers, the role of the gene of interest, and the ability of the knocked-in gene to complement the endogenous function. The inventors found that in some instances pGLY642 was integrated as a double cross-over, resulting in replacement of the endogenous PpPDI gene with human PpPDI, while in other cases, the pGLY642 plasmid vector was integrated as a single integration, resulting in presence of both the endogenous PpPDI1 gene and a human PpPDI gene. In order to distinguish between these events, the inventors utilized PCR primers of Sequence ID Nos. 11 through 14, described herein. If the PpPDI gene has been retained after integration of the pGLY642 plasmid vector, PpPDI-5′/UP and hPDI-3′/LP, directed to the internal PpPDI coding sequence, will result in an amplification product and a corresponding band. In the event of a knockout or double cross-over, these primers will not result in any amplification product and no corresponding band will be visible.

The roll-in of pGLY642 was confirmed with the primers; PpPDI/UPi (SEQ ID NO: 7) and PpPDI/LPi-1 (SEQ ID NO: 8) encoding PpPDI1, and hPDI/UP, 5′-GTGGCCACACCAGGGGGCATGGAAC-3′; (SEQ ID NO: 13); and hPDI-3′/LP, 5′-CCTAGAGAGCGGTGGCCAAGATG-3′; (SEQ ID NO: 14); encoding human PDI. The PCR conditions were one cycle of 95° C. for two minutes, 25 cycles of 95° C. for 20 seconds, 58° C. for 20 seconds, and 72° C. for one minute, and followed by 1 cycle of 72° C. for 10 minutes for PpPDI1, and 1 cycle of 95° C. for two minutes, 25 cycles of 95° C. for 20 seconds, 50° C. for 30 seconds, and 72° C. for 2 5 minutes, and followed by one cycle of 72° C. for 10 minutes for human PDI.

Strain yGLY714 is a strain that contains both the Pichia pastoris PDI1 locus and expresses the human PDI and was a result of integration via a single crossover event. Strain yGLY714 was generated from strain yGLY24-1 by integrating plasmid vector pGLY642, which comprises the human PDI gene under constitutive regulatory control of the Pichia pastoris GAPDH promoter, into the PpPDI 5′UTR region in yGLY24-1. Integration of this vector does not disrupt expression of the Pichia pastoris PDI1 locus. Thus, in yGLY714, the human PDI is constitutively expressed in the presence of the Pichia pastoris endogenous PDI1.

Strain yGLY733 was generated by transforming with plasmid vector pGLY1162, which comprises an expression cassette that encodes the Trichoderma Reesei mannosidase (TrMNS1) operably linked to the Pichia pastoris AOX1 promoter (PpAOX1-TrMNS1), into the PRO1 locus of yGLY704. This strain has the gene encoding the Pichia pastoris PD1 replaced with the expression cassette encoding the human PDI1, has the PpAOX1-TrMNS1 expression cassette integrated into the PRO1 locus, and is a URA5 prototroph. The PpAOX1 promoter allows overexpression when the cells are grown in the presence of methanol.

Strain yGLY762 was constructed by integrating expression cassettes encoding TrMNS1 and mouse mannosidase IA (MuMNS1A), each operably linked to the Pichia pastoris GAPDH promoter in plasmid vector pGFI207t into strain yGLY733 at the 5′ PRO1 locus UTR in Pichia pastoris genome. This strain has the gene encoding the Pichia pastoris PDI1 replaced with the expression cassette encoding the human PDI, has the PpGAPDH-TrMNS1 and PpGAPDH-MuMNS1A expression cassettes integrated into the PRO1 locus, and is a URA5 prototroph.

Strain yGLY730 is a control strain for strain yGLY733. Strain yGLY730 was generated by transforming pGLY1162, which comprises an expression cassette that encodes the Trichoderma Reesei mannosidase (TrMNS1) operably linked to the Pichia pastoris AOX1 promoter (PpAOX1-TrMNS1), into the PRO1 locus of yGLY24-1. This strain has the Pichia pastoris PDI1, has the PpAOX1-TrMNS1 expression cassette integrated into the PRO1 locus, and is a URA5 prototroph.

Control Strain yGLY760 was constructed by integrating expression cassettes encoding TrMNS1 and mouse mannosidase IA (MuMNS1A), each operably linked to the Pichia pastoris GAPDH promoter in plasmid vector pGFI207t into control strain yGLY730 at the 5′ PRO1 locus UTR in Pichia pastoris genome. This strain has the gene encoding the Pichia pastoris PDI1, has the PpGAPDH-TrMNS1 and PpGAPDH-MuMNS1A expression cassettes integrated into the PRO1 locus, and is a URA5 prototroph.

Strain yGLY2263 was generated by transforming strain yGLY645 with integration/expression plasmid pGLY2260, which targets an expression cassette encoding the anti-DKK1 antibody to the TRP2 locus.

Strain yGLY2674 was generated by counterselecting yGLY733 on 5-FOA plates. This strain has the gene encoding the Pichia pastoris PDI1 replaced with the expression cassette encoding the human PDI, has the PpAOX1-TrMNS1 expression cassette integrated into the PRO1 locus, and is a URA5 auxotroph.

Strain yGLY2677 was generated by counterselecting yGLY762 on 5-FOA plates. This strain has the gene encoding the Pichia pastoris PDI1 replaced with the expression cassette encoding the human PDI, has the PpAOX1-TrMNS1 expression cassette integrated into the PRO1 locus, has the PpGAPH-TrMNS1 and PpGAPDH-MuMNS1A expression cassettes integrated into the PRO1 locus, and is a URA5 auxotroph.

Strains yGLY2690 was generated by integrating plasmid vector pGLY2232, which encodes the human ERO1α protein, into the PRB1 locus. This strain has the gene encoding the Pichia pastoris PDI1 replaced with the expression cassette encoding the human PDI, has the PpAOX1-TrMNS1 expression cassette integrated into the PRO1 locus, the human ERO1α expression cassette integrated into the PRB1 locus, and is a URA5 prototroph.

Strains yGLY2696 was generated by integrating plasmid vector pGLY2233, which encodes the human GRP94 protein, into the PEP4 locus. This strain has the gene encoding the Pichia pastoris PDI1 replaced with the expression cassette encoding the human PDI, has the PpAOX1-TrMNS1 expression cassette integrated into the PRO1 locus, has the PpGAPDH-TrMNS1 and PpGAPDH-MuMNS1A expression cassettes integrated into the PRO1 locus, has the human GRP94 integrated into the PEP4 locus, and is a URA5 prototroph.

Strain yGLY3628 was generated by transforming strain yGLY2696 with integration/expression plasmid pGLY2261, which targets an expression cassette encoding the anti-DKK1 antibody to the TRP2 locus.

Strain yGLY3647 was generated by transforming strain yGLY2690 with integration/expression plasmid pGLY2261, which targets an expression cassette encoding the anti-DKK1 antibody to the TRP2 locus.

The yield of protein produced in a strain, which expresses the human PDI protein in place of the Pichia pastoris PDI1 protein, was compared to the yield of the same protein produced in a strain, which expresses both the human and Pichia pastoris PDI proteins, and a strain, which expresses only the Pichia pastoris PDI1 protein. Strain yGLY733, which expresses the human PDI protein in place of the Pichia pastoris PDI1 protein, strain yGLY714, which expresses both the human and Pichia pastoris PDI1 proteins, and strain yGLY730, which expresses only the Pichia pastoris PDI1 protein were evaluated to determine the effect of replacing the Pichia pastoris PDI1 protein with the human PDI protein on antibody titers produced by the strains. All three yeast strains were transformed with plasmid vector pGLY2261, which encodes the anti-DKK1 antibody.

Titer improvement for culture growth was determined from deep-well plate screening in accordance with the NIH ImageJ software protocol, as described in Rasband, ImageJ, U.S. National Institutes of Health, Bethesda, Md., USA, 1997-2007; and Abramoff, et al., Biophotonics International, 11: 36-42 (2004). Briefly, antibody screening in 96 deep-well plates was performed essentially as follows. Transformants were inoculated to 600 μL BMGY and grown at 24° C. at 840 rpm for two days in a Micro-Plate Shaker. The resulting 50 μL seed culture was transferred to two 96-well plates containing 600 μL fresh BMGY per well and incubated for two days at the same culture condition as above. The two expansion plates were combined to one prior to centrifugation for 5 minutes at 1000 rpm, the cell pellets were induced in 600 μL BMMY per well for two days and then the centrifuged 400 μL clear supernatant was purified using protein A beads. The purified proteins were subjected to SDS-PAGE electrophoresis and the density of protein bands were analyzed using NIH ImageJ software.

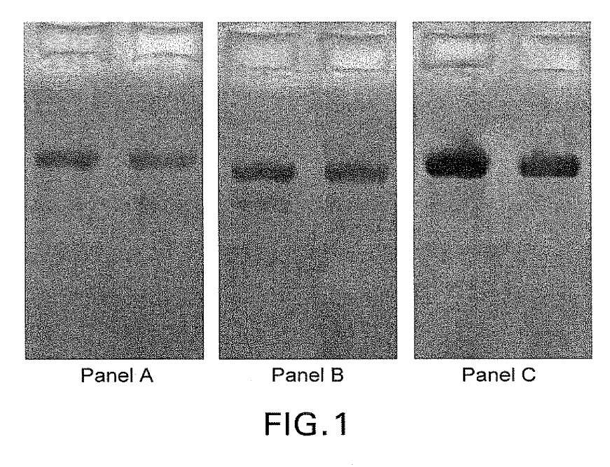

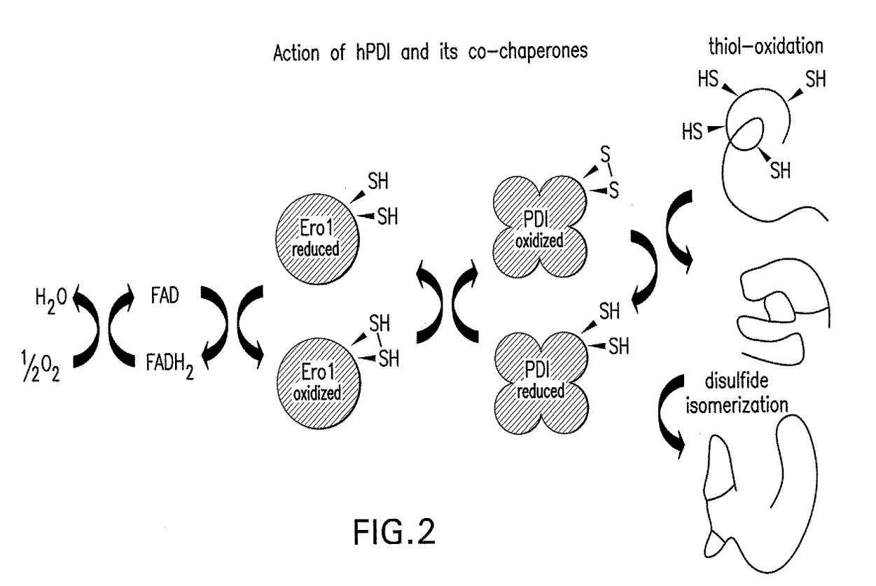

Representative results are shown in FIG. 1. FIG. 1 (Panel B) shows that while yGLY714, which expresses both Pichia pastoris PDI1 and human PDI, improved yield two-fold over the control (yGLY730) (Panel A), a five-fold increase in yield was achieved with strain yGLY733, which expresses only the human PDI (Panel C). The results are also presented in Table 1.

TABLE 1
Replacement of PpPDI1

| | | yGLY714 | |
|---|---|---|---|
| | yGLY730 | (Both Pichia and | yGLY733 |
| | (control) | human PDI) | (human PDI) |
| Pichia pastoris PDI1 | Wild-type | Wild-type | Knockout |
| Human PDI | None | Overexpression | Overexpression |
| Titer improvement | Control | 2-fold | 5-fold |

Strains yGLY730 and yGLY733 were transformed with plasmid vector pGLY2012 which encodes the anti-ADDL antibody. The transformed strains were evaluated by 96 deep well screening as described above and antibody was produced in 500 mL SixFors and 3 L fermentors using the following procedures. Bioreactor Screenings (SIXFORS) were done in 0.5 L vessels (Sixfors multi-fermentation system, ATR Biotech, Laurel, Md.) under the following conditions: pH at 6.5, 24° C., 0.3 SLPM, and an initial stirrer speed of 550 rpm with an initial working volume of 350 mL (330 mL BMGY medium and 20 mL inoculum). IRIS multi-fermenter software (ATR Biotech, Laurel, Md.) was used to linearly increase the stirrer speed from 550 rpm to 1200 rpm over 10 hours, one hour after inoculation. Seed cultures (200 mL of BMGY in a 1 L baffled flask) were inoculated directly from agar plates. The seed flasks were incubated for 72 hours at 24° C. to reach optical densities (OD₆₀₀) between 95 and 100. The fermenters were inoculated with 200 mL stationary phase flask cultures that were concentrated to 20 mL by centrifugation. The batch phase ended on completion of the initial charge glycerol (18-24 h) fermentation and were followed by a second batch phase that was initiated by the addition of 17 mL of glycerol feed solution (50% [w/w] glycerol, 5 mg/L Biotin, 12.5 mL/L PTM1 salts (65 g/L FeSO₄.7H₂O, 20 g/L ZnCl₂, 9 g/L H₂SO₄, 6 g/L CuSO₄.5H₂O, 5 g/L H₂SO₄, 3 g/L MnSO₄.7H₂O, 500 mg/L CoCl₂.6H₂O, 200 mg/L NaMoO₄.2H₂O, 200 mg/L biotin, 80 mg/L NaI, 20 mg/L H₃BO₄)). Upon completion of the second batch phase, as signaled by a spike in dissolved oxygen, the induction phase was initiated by feeding a methanol feed solution (100% MeOH 5 mg/L biotin, 12.5 mL/L PTM1) at 0.6 g/h for 32-40 hours. The cultivation is harvested by centrifugation.

Bioreactor cultivations (3 L) were done in 3 L (Applikon, Foster City, Calif.) and 15 L (Applikon, Foster City, Calif.) glass bioreactors and a 40 L (Applikon, Foster City, Calif.) stainless steel, steam in place bioreactor. Seed cultures were prepared by inoculating BMGY media directly with frozen stock vials at a 1% volumetric ratio. Seed flasks were incubated at 24° C. for 48 hours to obtain an optical density (OD₆₀₀) of 20±5 to ensure that cells are growing exponentially upon transfer. The cultivation medium contained 40 g glycerol, 18.2 g sorbitol, 2.3 g K₂HPO₄, 11.9 g KH₂PO₄, 10 g yeast extract (BD, Franklin Lakes, N.J.), 20 g peptone (BD, Franklin Lakes, N.J.), 4×10⁻³ g biotin and 13.4 g Yeast Nitrogen Base (BD, Franklin Lakes, N.J.) per liter. The bioreactor was inoculated with a 10% volumetric ratio of seed to initial media. Cultivations were done in fed-batch mode under the following conditions: temperature set at 24±0.5° C., pH controlled at to 6.5±0.1 with NH₄OH, dissolved oxygen was maintained at 1.7±0.1 mg/L by cascading agitation rate on the addition of O₂. The airflow rate was maintained at 0.7 vvm. After depletion of the initial charge glycerol (40 g/L), a 50% glycerol solution containing 12.5 mL/L of PTM1 salts was fed exponentially at 50% of the maximum growth rate for eight hours until 250 g/L of wet cell weight was reached. Induction was initiated after a 30 minute starvation phase when methanol was fed exponentially to maintain a specific growth rate of 0.01 h⁻¹. When an oxygen uptake rate of 150 mM/L/h was reached the methanol feed rate was kept constant to avoid oxygen limitation. The results are shown in Table 2, which shows about a three-fold increase in antibody titer.

The antibodies were also analyzed to determine whether replacing the Pichia pastoris PDI1 gene with an expression cassette encoding the human PDI would have an effect on O-glycosylation of the antibodies. In general, O-glycosylation of antibodies intended for use in humans is undesirable.

O-glycan determination was performed using a Dionex-HPLC (HPAEC-PAD) as follows. To measure O-glycosylation reduction, protein was purified from the growth medium using protein A chromatography (Li et al. Nat. Biotechnol. 24(2):210-5 (2006)) and the O-glycans released from and separated from protein by alkaline elimination (beta-elimination) (Harvey, Mass Spectrometry Reviews 18: 349-451 (1999)). This process also reduces the newly formed reducing terminus of the released O-glycan (either oligomannose or mannose) to mannitol. The mannitol group thus serves as a unique indicator of each O-glycan. 0.5 nmole or more of protein, contained within a volume of 100 μL PBS buffer, was required for beta elimination. The sample was treated with 25 μL alkaline borohydride reagent and incubated at 50° C. for 16 hours. About 20 uL arabitol internal standard was added, followed by 10 μL glacial acetic acid. The sample was then centrifuged through a Millipore filter containing both SEPABEADS and AG 50W-X8 resin and washed with water. The samples, including wash, were transferred to plastic autosampler vials and evaporated to dryness in a centrifugal evaporator. 150 μL 1% AcOH/MeOH was added to the samples and the samples evaporated to dryness in a centrifugal evaporator. This last step was repeated five more times. 200 μL of water was added and 100 μL of the sample was analyzed by high pH anion-exchange chromatography coupled with pulsed electrochemical detection-Dionex HPLC (HPAEC-PAD). Average O-glycan occupancy was determined based upon the amount of mannitol recovered.

As shown in Table 2, O-glycosylation was reduced in strains in which the Pichia pastoris PDI1 was replaced with an expression cassette encoding the human PDI. In strain yGLY733, O-glycan occupancy (number of O-glycosylation sites O-glycosylated) was reduced and for those sites occupied, the percent of O-glycans consisting of only one mannose was increased. These results suggest that replacing the Pichia pastoris PDI1 with an expression cassette encoding the human PDI will enable the production of antibodies in Pichia pastoris with reduced O-glycosylation.

TABLE 2
Anti-ADDL antibody: O-Glycan & Titer

| | yGLY730 | yGLY733 |
|---|---|---|
| Pichia PDI1 | Wild-type | Knockout |
| Human PDI | None | Overexpressed |
| O-glycan Occupancy | 7.4 | 4.2 |
| (H2L2) | | |
| O-glycan % | 75.5/24.5 | 82.5/17.5 |
| (Man1/Man2) | | |
| Titer | 12.5 mg/L (SixFors) | 38.3 mg/L (SixFors) |
| | | 93 mg/L (3 L) |

The above three strains (yGLY730, yGLY714, and yGLY733) produce glycoproteins that have Pichia pastoris N-glycosylation patterns. GS 2.0 strains are Pichia pastoris strains that have been genetically engineered to produce glycoproteins having predominantly Man₅GlcNAc₂ N-glycans. The following experiment was performed with GS 2.0 strains that produce glycoproteins that have predominantly Man₅GlcNAc₂ N-glycans to determine the effect of replacing the Pichia pastoris PDI1 protein with the human PDI protein on antibody titers produced by these strains. Strains yGLY2690 and yGLY2696 are GFI 2.0 strains that produce glycoproteins that have predominantly Man₅GlcNAc₂ N-glycans and have the Pichia pastoris PDI1 gene replaced with the expression cassette encoding the human PDI protein (See FIG. 3). These two strains were transformed with plasmid vector pGLY2261, which encodes the anti-DKK1 antibody, to produce strains yGLY3647 and yGLY3628 (See FIG. 3) and the strains evaluated by 96 deep well screening as described above. Antibody was produced in 500 ml SixFors and 3 L fermentors using the parameters described above to determine the effect of replacing the Pichia pastoris PDI1 protein with the human PDI protein on antibody titers produced by the strains. The results are shown in Table 3. Strain yGLY2263 is a control in which plasmid vector pGLY2260 was transformed into strain yGLY645, which produces glycoproteins having predominantly Man₅GlcNAc₂ N-glycans and expresses only the endogenous PDI1 gene.

Table 3 shows that replacing the gene encoding the Pichia pastoris PDI1 with an expression cassette encoding the human PDI in yeast genetically engineered to produce glycoproteins that have predominantly Man₅GlcNAc₂ N-glycans effects an improvement in the titers of antibodies produced by the yeast. Table 3 also shows that O-glycosylation occupancy was still reduced in these strains genetically engineered to produce glycoproteins having predominantly Man₅GlcNAc₂ N-glycans. Additionally, Table 3 shows an increase in the amount of N-glycosylation in the strains with the endogenous PDI1 replaced with the human PDI.

TABLE 3
Anti-DKK1 antibody: Titer, N-glycan & O-glycan

| | yGLY2263 | | |
|---|---|---|---|
| GS2.0 Strain | (control) | yGLY3647 | yGLY3628 |
| Pichia pastoris PDI1 | Wild-type | Knockout | Knockout |
| Human PDI | None | Overexpressed | Overexpressed |
| Human ERO1α | None | Expressed | None |
| Human GRP94 | None | None | Expressed |
| Pichia pastoris PRB1 | Intact | Knockout | Intact |
| Pichia pastoris PEP4 | Intact | Intact | Knockout |
| N-glycan (Man5) | 83.7% | 93.4% | 95.4% |
| O-glycan | 23.7 | 9.2 | 10.0 |
| (Occupancy: H2L2) | | | |
| O-glycan | 55/40 | 88/12 | 87/13 |
| (Man1/Man2) | | | |
| Titer | 27 mg/L | 61 mg/L | 86 mg/L (3 L) |
| | (SixFors) | (SixFors) | |

### EXAMPLE 2

A benefit of the strains shown in Tables 2 and 3 is that making yeast strains that have replaced the endogenous PDI1 gene with an expression cassette that encodes a heterologous PDI not only effects an increase in protein yield but also effects a decrease in both the number of attached O-glycans (occupancy) and a decrease in undesired Man₂ O-glycan structures. Recombinant proteins produced in yeast often display aberrant O-glycosylation structures relative to compositions of the same glycoprotein produced from mammalian cell culture, reflecting the significant differences between the glycosylation machinery of mammalian and yeast cells. These aberrant structures may be immunogenic in humans.

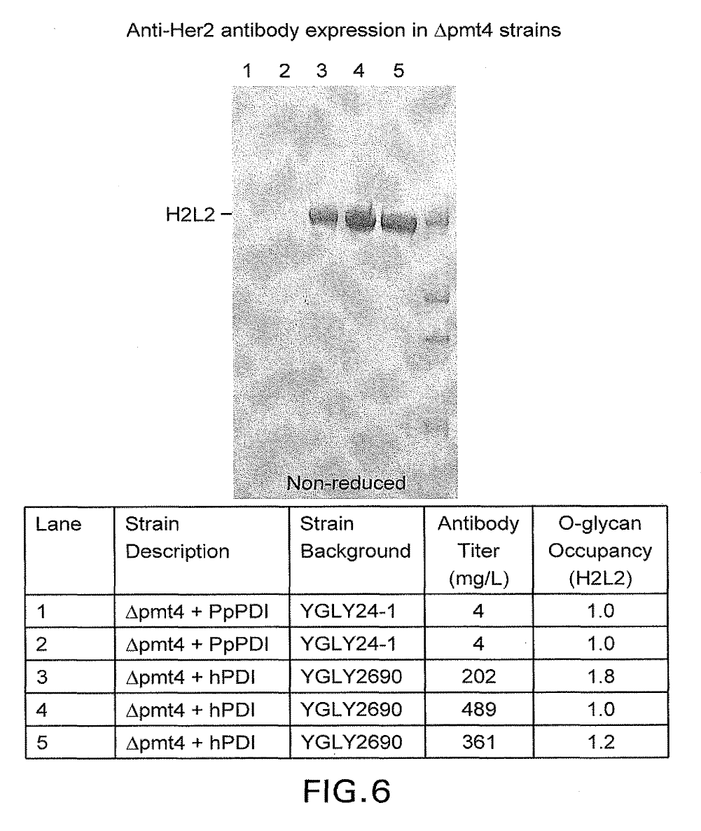

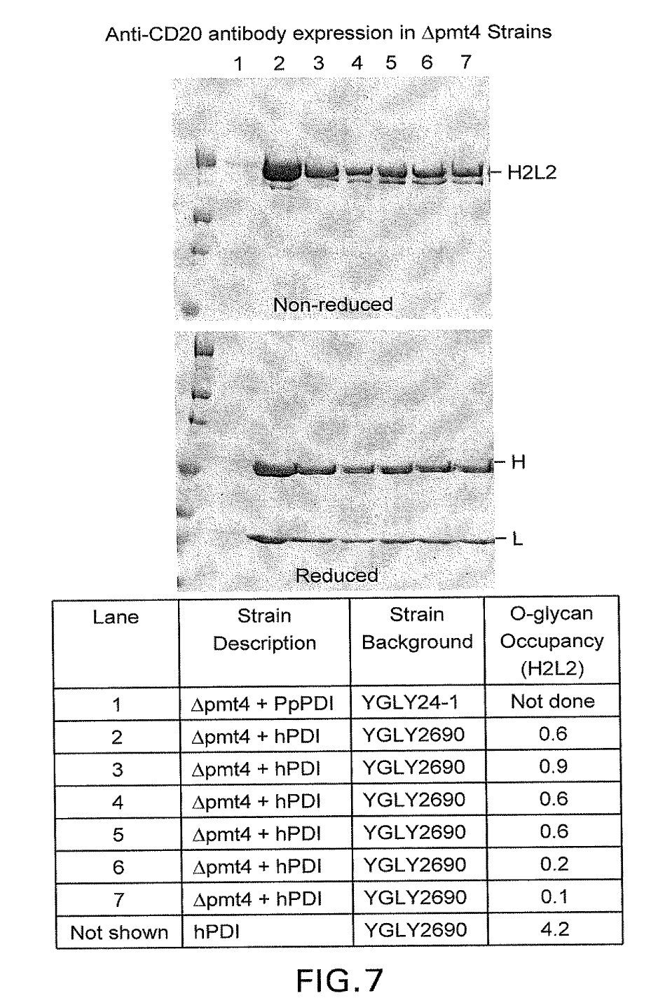

The inventors noted that host cells of Pichia pastoris carrying the human PDI gene in place of the endogenous Pichia pastoris PDI1 gene were strain more resistant to PMT protein inhibitors (See published International Application No. WO2007061631), suggesting that these strains might be better suited to tolerate deletions of various PMT genes. This is because in prior attempts to make ΔPMT knockouts in ΔOCH1/ΔPNO1/ΔPBS2 strains of Pichia pastoris, ΔPMT1 knockouts and ΔPMT2 knockouts could not be obtained; presumably because they are lethal in this genetic background (unpublished results). ΔPMT4 knockouts could be obtained, but they typically exhibited only weak growth and poor protein expression compared to parental strains (See FIGS. 6 and 7). While ΔPMT5 and ΔPMT6 knockouts could be obtained, the deletions exhibited little or no effect on cell growth or protein expression compared to parental strains, suggesting that these PMT genes were not effective in reduction of O-glycosylation.

PMT knockout yeast strains were created in the appropriate Pichia pastoris strains following the procedure outlined for Saccharomyces cerevisiae in Gentzsch and Tanner, EMBO J. 15: 25752-5759 (1996), as described further in Published International Application No. WO 2007061631. The nucleic acid molecules encoding the Pichia pastoris PMT1 and PMT4 are shown in SEQ ID NOs: 47 and 49. The amino acid sequences of the Pichia pastoris PMT1 and PMT4 are shown in SEQ ID NOs: 48 and 50. The primers and DNA templates used for making the PMT deletions using the PCR overlap method are listed below.

To make a PMT1 knockout, the following procedure was followed. Three PCR reactions were set up. PCR reaction A comprised primers PMT1-KO1: 5′-TGAACCCATCT GTAAATAGAATGC-3′ (SEQ ID NO: 17) and PMT1-KO2: 5′-GTGTCACCTAAATCGTA TGTGCCCATTTACTGGA AGCTGCTAACC-3′ (SEQ ID NO: 18) and Pichia pastoris NRRL-Y11430 genomic DNA as the template. PCR reaction B comprised primers PMT1-KO3: 5′-CTCCCTATAGTGAGTCGTATTCATCATTGTACTTT GGTATATTGG-3′ (SEQ ID NO: 19) and PMT1-KO4: 5′-TATTTGTACCTGCGTCCTGTTTGC-3′ (SEQ ID NO: 20) and Pichia pastoris NRRL-Y11430 genomic DNA as the template. PCR reaction C comprised primers PR29: 5′-CACATACGATTTAGGTGACAC-3′ (SEQ ID NO: 21) and PR32: 5′-AATAC GACTCACTATAGGGAG-3′ (SEQ ID NO: 22) and the template was plasmid vector pAG25 (Goldstein and McCusker, Yeast 15: 1541 (1999)). The conditions for all three PCR reactions were one cycle of 98° C. for two minutes, 25 cycles of 98° C. for 10 seconds, 54° C. for 30 seconds, and 72° C. for four minutes, and followed by one cycle of 72° C. for 10 minutes.

Then in a second PCR reaction, primers PMT1-KO1+PMT1-KO4 from above were mixed with the PCR-generated fragments from PCR reactions A, B, and C above. The PCR conditions were one cycle of 98° C. for two minutes, 30 cycles of 98° C. for 10 seconds, 56° C. for 10 seconds, and 72° C. for four minutes, and followed by one cycle of 72° C. for 10 minutes.

The fragment generated in the second PCR reaction was gel-purified and used to transform appropriate strains in which the Pichia pastoris PDI1 gene has been replaced with an expression cassette encoding the human PDI1 protein. Selection of transformants was on rich media plates (YPD) containing 100 μg/mL nourseothricin.

To make a PMT4 knockout, the following procedure was followed. Three PCR reactions were set up. PCR reaction A comprised primers PMT4-KO1: 5′-TGCTCTCCGCGTGCAATAGAAACT-3′ (SEQ ID NO: 23) and PMT4-KO2: 5′-CTCCCTATAGTGAGTCGTATTCACAGTGTACCATCT TTCATCTCC-3′ (SEQ ID NO: 24) and Pichia pastoris NRRL-Y11430 genomic DNA as the template. PCR reaction B comprised primers PMT4-KO3: 5′-GTGTCACCTAAATCGTATGTGAACCTAACTCTAA TTCTTCAAA GC-3′ (SEQ ID NO: 25) and PMT4-KO4: 5′-ACTAGGGTATATAATTCCCAAGGT-3′ (SEQ ID NO: 26) and Pichia pastoris NRRL-Y11430 genomic DNA as the template. PCR reaction C comprised primers PR29: 5′-CACATACGATTTAGGTGACAC-3′ (SEQ ID NO: 21) and PR32: 5′-AATACGACTCACTATAGGGAG-3′ (SEQ ID NO: 22) and plasmid vector pAG25 as the template.

The conditions for all three PCR reactions were one cycle of 98° C. for two minutes, 25 cycles of 98° C. for 10 seconds, 54° C. for 30 seconds, and 72° C. for four minutes, and followed by one cycle of 72° C. for 10 minutes.

Then in a second PCR reaction, primers PMT4-KO1+PMT4-KO4 from above were mixed with the PCR-generated fragments from PCR reactions A, B, and C above. The PCR conditions were one cycle of 98° C. for two minutes, 30 cycles of 98° C. for 10 seconds, 56° C. for 10 seconds, and 72° C. for four minutes, and followed by one cycle of 72° C. for 10 minutes.

The fragment generated in the second PCR reaction was gel-purified and used to transform appropriate strains in which the Pichia pastoris PDI1 gene has been replaced with an expression cassette encoding the human PDI protein. Selection of transformants was on rich media plates (YPD) containing 100 μg/mL nourseothricin.

To test the ability of the strains to produce antibodies with reduced O-glycosylation, expression vectors encoding an anti-Her2 antibody and an anti-CD20 antibody were constructed.

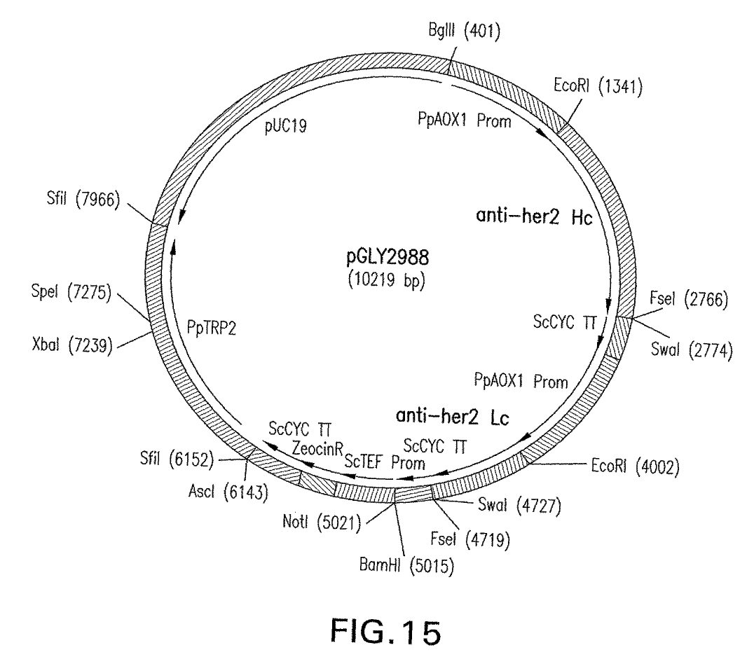

Expression/integration plasmid vector pGLY2988 contains expression cassettes encoding the heavy and light chains of an anti-Her2 antibody. Anti-Her2 heavy (HC) and light (LC) chains fused at the N-terminus to α-MAT pre signal peptide were synthesized by GeneArt AG. Each was synthesized with unique 5′ EcoR1 and 3′ Fse1 sites. The nucleotide and amino acid sequences of the anti-Her2 HC are shown in SEQ ID Nos: 29 and 30, respectively. The nucleotide and amino acid sequences of the anti-Her2 LC are shown in SEQ ID Nos: 31 and 32, respectively. Both nucleic acid molecule fragments encoding the HC and LC fusion proteins were separately subcloned using 5′ EcoR1 and 3′ Fse1 unique sites into an expression plasmid vector pGLY2198 (contains the Pichia pastoris TRP2 targeting nucleic acid molecule and the Zeocin-resistance marker) to form plasmid vector pGLY2987 and pGLY2338, respectively. The LC expression cassette encoding the LC fusion protein under the control of the Pichia pastoris AOX1 promoter and Saccharomyces cerevisiae Cyc terminator was removed from plasmid vector pGLY2338 by digesting with BamHI and NotI and then cloning the DNA fragment into plasmid vector pGLY2987 digested with BamH1 and Not1, thus generating the final expression plasmid vector pGLY2988 (FIG. 15).

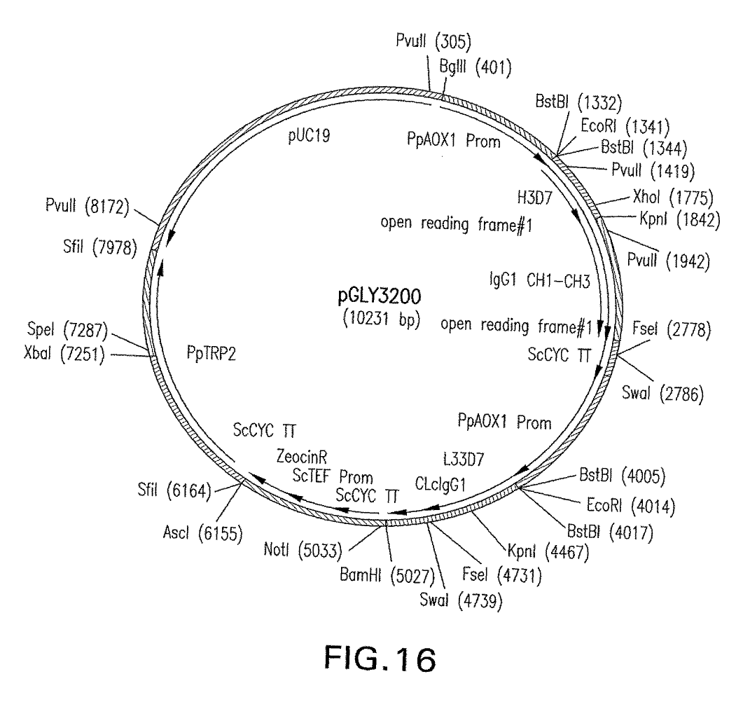

Expression/integration plasmid vector pGLY3200 (map is identical to pGLY2988 except LC and HC are anti-CD20 with α-amylase signal sequences). Anti-CD20 sequences were from GenMab sequence 2C6 except Light chain (LC) framework sequences matched those from VKappa 3 germline. Heavy (HC) and Light (LC) variable sequences fused at the N-terminus to the α-amylase (from Aspergillus niger) signal peptide were synthesized by GeneArt AG. Each was synthesized with unique 5′ EcoR1 and 3′ KpnI sites which allowed for the direct cloning of variable regions into expression vectors containing the IgG1 and V kappa constant regions. The nucleotide and amino acid sequences of the anti-CD20 HC are shown in SEQ ID Nos: 37 and 38, respectively. The nucleotide and amino acid sequences of the anti-CD20 LC are shown in SEQ ID Nos: 35 and 36, respectively. Both HC and LC fusion proteins were subcloned into IgG1 plasmid vector pGLY3184 and V Kappa plasmid vector pGLY2600, respectively, (each plasmid vector contains the Pichia pastoris TRP2 targeting nucleic acid molecule and Zeocin-resistance marker) to form plasmid vectors pGLY3192 and pGLY3196, respectively. The LC expression cassette encoding the LC fusion protein under the control of the Pichia pastoris AOX1 promoter and Saccharomyces cerevisiae Cyc terminator was removed from plasmid vector pGLY3196 by digesting with BamHI and NotI and then cloning the DNA fragment into plasmid vector pGLY3192 digested with BamH1 and Not1, thus generating the final expression plasmid vector pGLY3200 (FIG. 16).

Transformation of appropriate strains disclosed herein with the above anti-Her2 or anti-CD20 antibody expression/integration plasmid vectors was performed essentially as follows. Appropriate Pichia pastoris strains were grown in 50 mL YPD media (yeast extract (1%), peptone (2%), dextrose (2%)) overnight to an OD of between about 0.2 to 6. After incubation on ice for 30 minutes, cells were pelleted by centrifugation at 2500-3000 rpm for 5 minutes. Media was removed and the cells washed three times with ice cold sterile 1M sorbitol before resuspension in 0.5 ml ice cold sterile 1M sorbitol. Ten μL linearized DNA (5-20 ug) and 100 μL cell suspension was combined in an electroporation cuvette and incubated for 5 minutes on ice. Electroporation was in a Bio-Rad GenePulser Xcell following the preset Pichia pastoris protocol (2 kV, 25 μF, 200Ω), immediately followed by the addition of 1 mL YPDS recovery media (YPD media plus 1 M sorbitol). The transformed cells were allowed to recover for four hours to overnight at room temperature (24° C.) before plating the cells on selective media.

Cell Growth conditions of the transformed strains for antibody production was generally as follows. Protein expression for the transformed yeast strains was carried out at in shake flasks at 24° C. with buffered glycerol-complex medium (BMGY) consisting of 1% yeast extract, 2% peptone, 100 mM potassium phosphate buffer pH 6.0, 1.34% yeast nitrogen base, 4×10⁻⁵% biotin, and 1% glycerol. The induction medium for protein expression was buffered methanol-complex medium (BMMY) consisting of 1% methanol instead of glycerol in BMGY. Pmt inhibitor (Pmti-3) in methanol was added to the growth medium to a final concentration of 0.2 μM, 2 μM, or 20 μM at the time the induction medium was added. Cells were harvested and centrifuged at 2,000 rpm for five minutes.

SixFors Fermenter Screening Protocol followed the parameters shown in Table 4.

TABLE 4
SixFors Fermenter Parameters

| Parameter | Set-point | Actuated Element |
|---|---|---|
| pH | 6.5 ± 0.1 | 30% NH₄OH |
| Temperature | 24 ± 0.1 | Cooling Water & Heating Blanket |
| Dissolved O2 | n/a | Initial impeller speed of 550 rpm is |
| | | ramped to 1200 rpm over first 10 hr, then |
| | | fixed at 1200 rpm for remainder of run |

At time of about 18 hours post-inoculation, SixFors vessels containing 350 mL media A (See Table 6 below) plus 4% glycerol were inoculated with strain of interest. A small dose (0.3 mL of 0.2 mg/mL in 100% methanol) of Pmti-3 (5-[[3-(1-Phenyl-2-hydroxy)ethoxy)-4-(2-phenylethoxy)]phenyl]methylene]-4-oxo-2-thioxo-3-thiazolidineacetic Acid) (See Published International Application No. WO 2007061631) was added with inoculum. At time about 20 hour, a bolus of 17 mL 50% glycerol solution (Glycerol Fed-Batch Feed, See Table 7 below) plus a larger dose (0.3 mL of 4 mg/mL) of Pmti-3 was added per vessel. At about 26 hours, when the glycerol was consumed, as indicated by a positive spike in the dissolved oxygen (DO) concentration, a methanol feed (See Table 8 below) was initiated at 0.7 mL/hr continuously. At the same time, another dose of Pmti-3 (0.3 mL of 4 mg/mL stock) was added per vessel. At time about 48 hours, another dose (0.3 mL of 4 mg/mL) of Pmti-3 was added per vessel. Cultures were harvested and processed at time about 60 hours post-inoculation.

TABLE 5
Composition of Media A

| | | |
|---|---|---|
| Martone L-1 | 20 | g/L |
| Yeast Extract | 10 | g/L |
| KH₂PO4 | 11.9 | g/L |
| K₂HPO₄ | 2.3 | g/L |
| Sorbitol | 18.2 | g/L |
| Glycerol | 40 | g/L |
| Antifoam Sigma 204 | 8 | drops/L |
| 10X YNB w/Ammonium Sulfate w/o | 100 | mL/L |
| Amino Acids (134 g/L) | | |
| 250X Biotin (0.4 g/L) | 10 | mL/L |
| 500X Chloramphenicol (50 g/L) | 2 | mL/L |
| 500X Kanamycin (50 g/L) | 2 | mL/L |

TABLE 6
Glycerol Fed-Batch Feed

| | | |
|---|---|---|
| Glycerol | 50 | % m/m |
| PTM1 Salts (see Table IV-E below) | 12.5 | mL/L |
| 250X Biotin (0.4 g/L) | 12.5 | mL/L |

TABLE 7
Methanol Feed

| | | |
|---|---|---|
| Methanol | 100 | % m/m |
| PTM1 Salts | 12.5 | mL/L |
| 250X Biotin (0.4 g/L) | 12.5 | mL/L |

TABLE 8
PTM1 Salts

| | | |
|---|---|---|
| CuSO4—5H2O | 6 | g/L |
| NaI | 80 | mg/L |
| MnSO4—7H2O | 3 | g/L |
| NaMoO4—2H2O | 200 | mg/L |
| H3BO3 | 20 | mg/L |
| CoCl2—6H2O | 500 | mg/L |
| ZnCl2 | 20 | g/L |
| FeSO4—7H2O | 65 | g/L |
| Biotin | 200 | mg/L |
| H2SO4 (98%) | 5 | mL/L |

O-glycan determination was performed using a Dionex-HPLC (HPAEC-PAD) as follows. To measure O-glycosylation reduction, protein was purified from the growth medium using protein A chromatography (Li et al. Nat. Biotechnol. 24(2):210-5 (2006)) and the O-glycans released from and separated from protein by alkaline elimination (beta -elimination) (Harvey, Mass Spectrometry Reviews 18: 349-451 (1999)). This process also reduces the newly formed reducing terminus of the released O-glycan (either oligomannose or mannose) to mannitol. The mannitol group thus serves as a unique indicator of each O-glycan. 0.5 nmole or more of protein, contained within a volume of 100 μL PBS buffer, was required for beta elimination. The sample was treated with 25 μL alkaline borohydride reagent and incubated at 50° C. for 16 hours. About 20 uL arabitol internal standard was added, followed by 10 μL glacial acetic acid. The sample was then centrifuged through a Millipore filter containing both SEPABEADS and AG 50W-X8 resin and washed with water. The samples, including wash, were transferred to plastic autosampler vials and evaporated to dryness in a centrifugal evaporator. 150 μL 1% AcOH/MeOH was added to the samples and the samples evaporated to dryness in a centrifugal evaporator. This last step was repeated five more times. 200 μL of water was added and 100 μL of the sample was analyzed by high pH anion-exchange chromatography coupled with pulsed electrochemical detection-Dionex HPLC (HPAEC-PAD). Average O-glycan occupancy was determined based upon the amount of mannitol recovered.

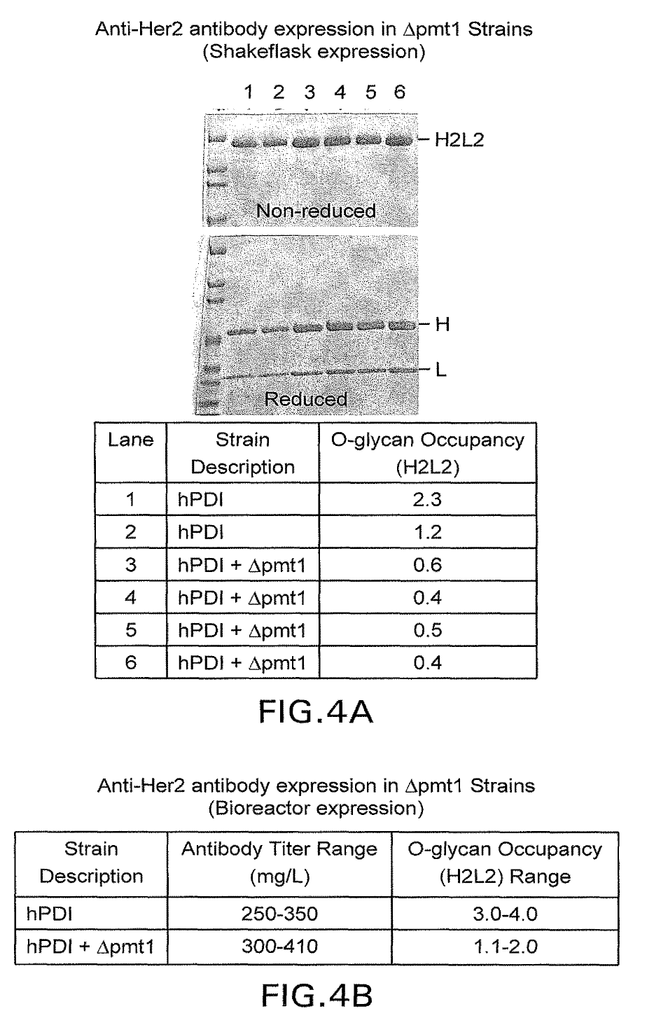

FIGS. 4-7 show that the Pichia pastoris strains in which the endogenous PDI1 is replaced with a heterologous PDI from the same species as the recombinant protein to be produced in the strain and in which native PMT1 or PMT4 genes have been deleted are capable of producing recombinant human antibody at higher titers and with reduced O-glycosylation compared to production of the antibodies in strains that contain the endogenous PDI1 and do not have deletions of the PMT1 or PMT4 genes.

FIGS. 4A and 4B shows representative results from shakeflask (A) and 0.5 L bioreactor (B) expression studies in which human anti-Her2 antibody was produced in Pichia pastoris strains in which the human PDI gene (hPDI) replaced the endogenous PDI1 and strains in which the human PDI replaced the endogenous PDI1 and the PMT1 gene disrupted (hPDI+Δpmt1). Antibodies were recovered and resolved by polyacrylamide gel electrophoresis on non-reducing and reducing polyacrylamide gels. Under non-reducing conditions, the antibodies remained intact whereas under reducing conditions, the antibodies were resolved into HCs and LCs. Lanes 1-2 shows antibodies produced from two clones produced from transformation of strain yGLY2696 with plasmid vector pGLY2988 encoding the anti-Her2 antibody and lanes 3-6 shows the antibodies produced from four clones produced from transformation of strain yGLY2696 in which the PMT1 gene was deleted and with plasmid vector pGLY2988 encoding the anti-Her2 antibody. The Figures showed that the PMT1 deletion improved antibody yield.

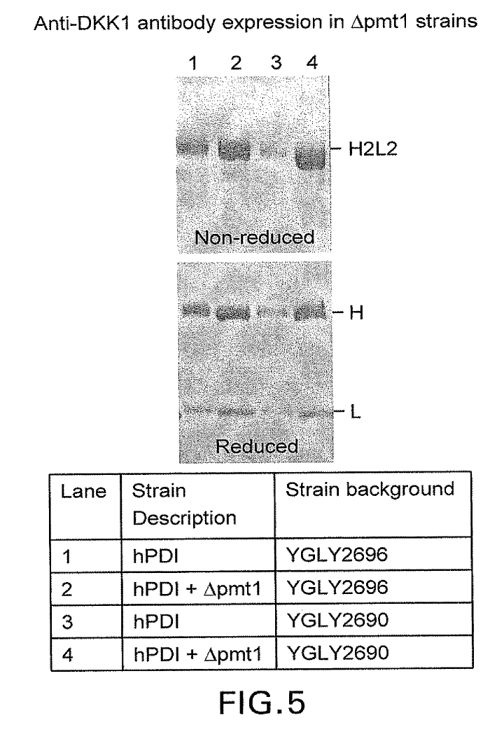

FIG. 5 shows representative results from a shakeflask expression study in which human anti-DKK1 antibody was produced in Pichia pastoris strains in which the human PDI gene (hPDI) replaced the endogenous PDI1 and strains in which the human PDI replaced the endogenous PDI1 and the PMT1 gene is disrupted (hPDI+Δpmt1). Antibodies were recovered and resolved by polyacrylamide gel electrophoresis on non-reducing and reducing polyacrylamide gels. Under non-reducing conditions, the antibodies remained intact whereas under reducing conditions, the antibodies were resolved into HCs and LCs. Lanes 1 and 3 shows antibodies produced from two clones produced from transformation of strains yGLY2696 and yGLY2690 with plasmid vector pGLY2260 encoding the anti-DKK1 antibody and lanes 2 and 4 shows the antibodies produced from two clones produced from transformation of strains yGLY2696 and yGLY2690 in which the PMT1 gene was deleted with plasmid vector pGLY2260 encoding the anti-DKK1 antibody. The figure shows that the PMT1 deletion improved antibody yield.

FIG. 6 shows results from a 0.5 L bioreactor expression study where human anti-Her2 antibody is produced in Pichia pastoris strains in which the human PDI replaced the endogenous PDI1 and the PMT4 gene is disrupted (hPDI+Δpmt4), and strains that express only the endogenous PDI1 but in which the PMT4 gene is disrupted (PpPDI+Δpmt4). Antibodies were recovered and resolved by polyacrylamide gel electrophoresis on non-reducing polyacrylamide gels. Lanes 1 and 2 shows antibodies produced from two clones from transformation of strain yGLY24-1 with plasmid vector pGLY2988 encoding the anti-Her2 antibody and lanes 3-5 show anti-Her2 antibodies produced from three clones produced from transformation of strain yGLY2690 in which the PMT4 gene was deleted. The figure shows that the PMT4 deletion improved antibody yield but in order to have that improvement in yield, the cell must also have the endogenous PDI1 gene replaced with an expression cassette encoding the human PDI.

FIG. 7 shows results from a shakeflask expression study where human anti-CD20 antibody is produced in Pichia pastoris strains in which the human PDI replaced the endogenous PDI1 and the PMT4 gene disrupted (hPDI+Δpmt4) and strains that express only the endogenous PDI1 but in which the PMT4 gene is disrupted (PpPDI+Δpmt4). Antibodies were recovered and resolved by polyacrylamide gel electrophoresis on non-reducing and reducing polyacrylamide gels. Lane 1 shows antibodies produced from strain yGLY24-1 transformed with plasmid vector pGLY3200 encoding the anti-CD20 antibody; lanes 2-7 show anti-CD20 antibodies produced from six clones produced from transformation of strain yGLY2690 in which the PMT4 gene was deleted. The figure shows that the PMT4 deletion improved antibody yield but in order to have that improvement in yield, the cell must also have the endogenous PDI1 gene replaced with an expression cassette encoding the human PDI.

### EXAMPLE 3

This example describes a chimeric BiP gene, in which the human ATPase domain is replaced by the ATPase domain of Pichia pastoris KAR2, fused to the human BiP peptide binding domain, under the control of the KAR2, or other ER-specific promoter from Pichia pastoris. The nucleotide and amino acid sequences of the human BiP are shown in Table 11 as SEQ ID NOs: 55 and 56, respectively. The nucleotide and amino acid sequences of the chimeric BiP are shown in Table 11 as SEQ ID NOs: 57 and 58, respectively. Further improvements in yield may be obtained by combining the replacement of the endogenous PDI1 gene, as described above, with the use of chimeric BiP and human ERdj3 (SEQ D NOs: 76 and 77, respectively).

### EXAMPLE 4

This example demonstrates that occupancy of O-glycans in proteins produced in the above strains expressing the human PDI in place of the Pichia pastoris PDI1 can be significantly reduced when either the Pichia pastoris Golgi Ca²⁺ ATPase (PpPMR1) or the Arabidopsis thaliana ER Ca²⁺ ATPase (AtECA1) is overexpressed in the strains. In this example, the effect is illustrated using glycoengineered Pichia pastoris strains that produce antibodies having predominantly Man₅GlcNAc₂ N-glycans.

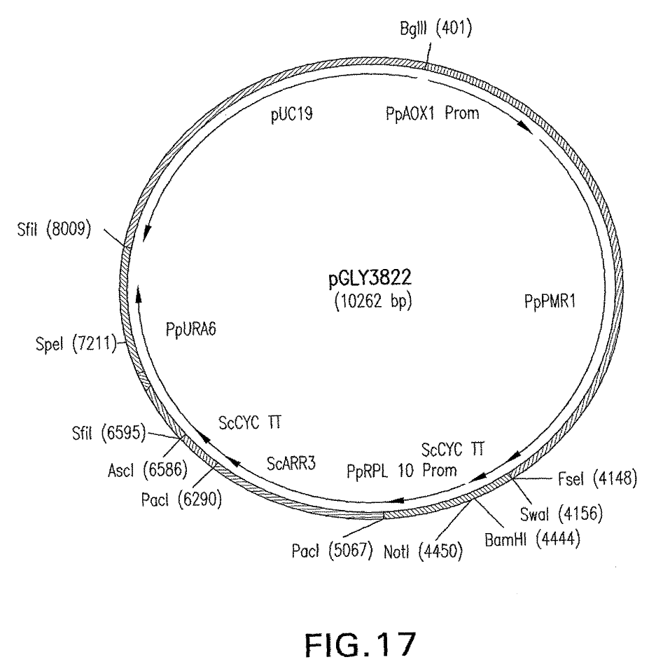

An expression cassette encoding the PpPMR1 gene was constructed as follows. The open reading frame of P. pastoris Golgi Ca²⁺ ATPase (PpPMR1) was PCR amplified from P. pastoris NRRL-Y11430 genomic DNA using the primers (PpPMR1/UP: 5′-GAATTCAT GACAGCTAATGAAAATCCTTTTGAGAATGAG-3′ (SEQ ID NO: 64) and PpPMR1/LP: 5′-GGCCGGCCTCAAACAGCCATGCTGTATCCATTGTATG-3′ (SEQ ID NO: 65). The PCR conditions were one cycle of 95° C. for two minutes; five cycles of 95° C. for 10 seconds, 52° C. for 20 seconds, and 72° C. for 3 minutes; 20 cycles of 95° C. for 10 seconds, 55° C. for 20 seconds, and 72° C. for 3 minutes; followed by 1 cycle of 72° C. for 10 minutes. The resulting PCR product was cloned into pCR2.1 and designated pGLY3811. PpPMR1 was removed from pGLY3811 by digesting with plasmid with PstI and FseI) and the PstI end had been made blunt with T4 DNA polymerase prior to digestion with FseI. The DNA fragment encoding the PpPMR1 was cloned into pGFI30t digested with EcoRI with the ends made blunt with T4 DNA polymerse and FseI to generate pGLY3822 in which the PpPMR1 is operably linked to the AOX1 promoter. Plasmid pGLY3822 targets the Pichia pastoris URA6 locus. Plasmid pGLY3822 is shown in FIG. 17. The DNA sequence of PpPMR1 is set forth in SEQ ID NO: 60 and the amino acid sequence of the PpPMR1 is shown in SEQ ID NO: 61.

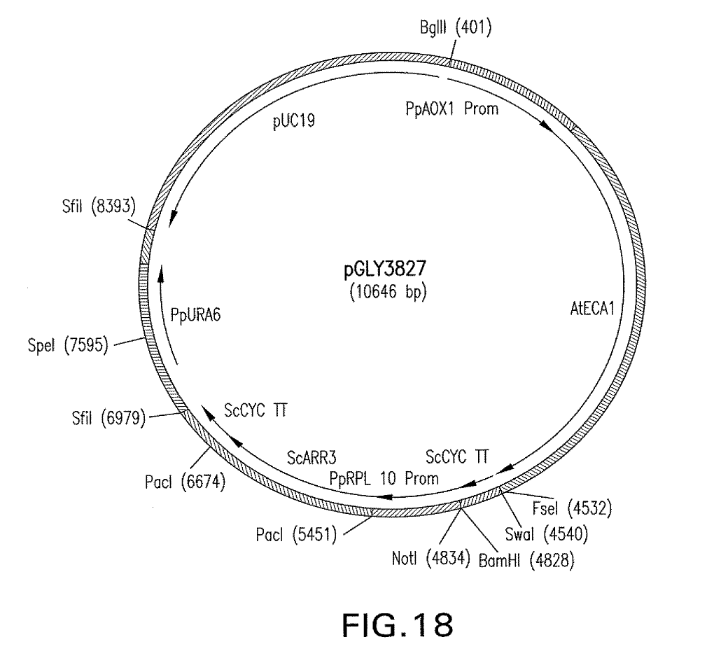

An expression cassette encoding the Arabidopsis thaliana ER Ca²⁺ ATPase (AtECA1) was constructed as follows. A DNA encoding AtECA1 was synthesized from GeneArt AG (Regensburg, Germany) and cloned to make pGLY3306. The synthesized AtECA1 was removed from pGLY3306 by digesting with MlyI and FseI and cloning the DNA fragment encoding the AtECA1 into pGFI30t digested with EcoRI with the ends made blunt with T4 DNA polymerase and FseI to generate integration/expression plasmid pGLY3 827. Plasmid pGLY3827 targets the Pichia pastoris URA6 locus. Plasmid pGLY3827 is shown in FIG. 18. The DNA sequence of the AtECA1 was codon-optimized for expression in Pichia pastoris and is shown in SEQ ID NO: 62. The encoded AtECA1 has the amino acid sequence set forth in SEQ ID NO: 63.

Integration/expression plasmid pGLY3822 (contains expression cassette encoding PpPMR1) or pGLY3827 (contains expression cassette encoding AtECA1) was linearized with SpeI and transformed into Pichia pastoris strain yGLY3647 or yGLY3693 at the URA6 locus. The genomic integration of pGLY3822 or pGLY3827 at URA6 locus was confirmed by colony PCR (cPCR) using primers, 5′AOX1 (5′-GCGACTGGTTCCAATTGACAAGCTT-3′ (SEQ ID NO: 66) and PpPMR1/cLP (5′-GGTTGCTCTCGTCGATACTCAAGTGGGAAG-3′ (SEQ ID NO: 67) for confirming PpPMR1 integration into the URA6 locus, and 5′AOX1 and AtECA1/cLP (5′-GTCGGCTGGAACCTTATCACCAACTCTCAG-3′ (SEQ ID NO: 68) for confirming integration of AtECA1 into the URA6 locus. The PCR conditions were one cycle of 95° C. for 2 minutes, 25 cycles of 95° C. for 10 seconds, 55° C. for 20 seconds, and 72° C. for one minute; followed by one cycle of 72° C. for 10 minutes.

Strain yGLY8238 was generated by transforming strain yGLY3647 with integration/expression plasmid pGLY3833 encoding the PpPMR1 and targeting the URA6 locus. In strain yGLY3647, the Pichia pastoris PDI1 chaperone gene has been replaced with the human PDI gene as described in Example 1 and shown in FIGS. 3A and 3B.

Strain yGLY8240 was generated by transforming strain yGLY3647 with plasmid pGLY3827 encoding the AtECA1 and targeting the URA6 locus. The geneology of the strains is shown in FIGS. 3A and 3B.

The strains were evaluated for the effect the addition of PpPMR1 or AtECA1 to the humanized chaperone strains might have on reducing O-glycosylation of the antibodies produced by the strains. As shown in Table 9 the addition of either PpPMR1 or AtECA1 into strain yGLY3647 effected a significant reduction in O-glycosylation occupancy compared to strain yGLY3647 expressing the human PDI in place of the Pichia pastoris PDI1 or strain yGLY2263 expressing only the endogenous PDI1 but capable of making antibodies with a Man₅GlcNAc₂ glycoform as strain yGLY3647. The results also suggest that yeast strains that express its endogenous PDI1 and not the human PDI and overexpress a Ca²⁺ ATPase will produce glycoproteins with reduced O-glycan occupancy.

TABLE 9

| | | | yGLY3647 + Ca²⁺ ATPase | |
|---|---|---|---|---|
| | | | yGLY8240 | yGLY8238 |
| Strain | yGLY2263 | yGLY3647 | AtECA1 | PpPMR1 |
| O-glycan | 23.7 | 9.2 | 5.54 | 6.28 |
| occupancy | | | | |
| (H2 + L2: | | | | |
| anti-DKK1) | | | | |

O-glycan occupancy was determined by Mannitol assay.

### EXAMPLE 5

A DNA fragment encoding the human calreticulin (hCRT) without its native signal sequence was PCR amplified from a human liver cDNA library (BD Biosciences, San Jose, Calif.) using primers hCRT-BstZ17I-HA/UP: 5′-GTATACCCATACGACGTCCCAGACTA CGCTGAGCCCGCCGTCTACTTCAAGGAGC-3′ (SEQ ID NO: 73) and hCRT-PacI/LP: 5′-TTAATTAACTACAGCTCGTCATGGGCCTGGCCGGGGACATCTTCC-3′ (SEQ ID NO: 74). The PCR conditions were one cycle of 98° C. for two min; 30 cycles of 98° C. for 10 seconds, 55° C. for 30 seconds, and 72° C. for two minutes, and followed by one cycle of 72° C. for 10 minutes. The resulting PCR product was cloned into pCR2.1 Topo vector to make pGLY1224. The DNA encoding the hCRT further included modifications such that the encoded truncated hCRT has an HA tag at its N-terminus and HDEL at its C-terminus. The DNA encoding the hCRT was released from pGLY1224 by digestion with BstZ17I and PacI and the DNA fragment cloned into an expression vector pGLY579, which had been digested with NotI and PacI, along with a DNA fragment encoding the S. cerevisiae alpha-mating factor pre signal sequence having NotI and PacI compatible ends to create pGLY1230. This plasmid is an integration/expression plasmid that encodes the hCRT with the S. cerevisiae alpha-mating factor pre signal sequence and HA tag at the N-terminus and an HDEL sequence at its C-terminus operably linked to the Pichia pastoris GAPDH promoter and targeting the HIS3 locus of Pichia pastoris.

A DNA fragment encoding the human ERp57 (hERp57) was synthesized by GeneArt AG having NotI and PacI compatible ends. The DNA fragment was then cloned into pGLY129 digested with NotI and PacI to produce pGLY1231. This plasmid encodes the hERp57 operably linked to the Pichia pastoris PMA1 promoter.

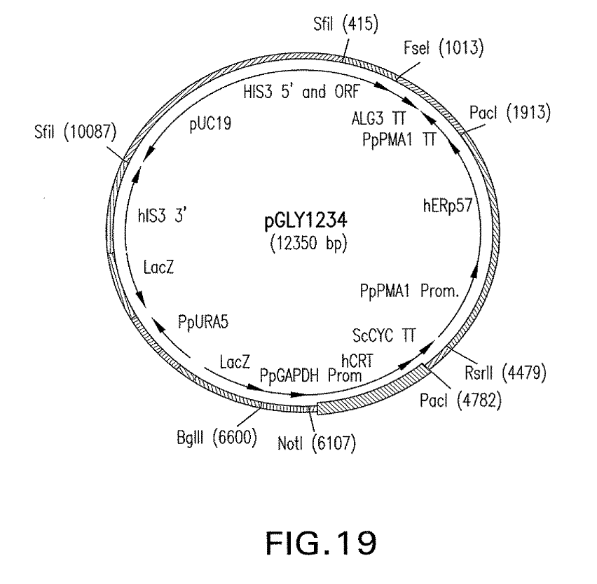

Plasmid pGLY1231 was digested with SwaI and the DNA fragment encoding the hERp57 was cloned into plasmid pGLY1230 digested with PmeI. Thus, integration/expression plasmid pGLY1234 encodes both the hCRT and hERp57. Plasmid pGLY1234 is shown in FIG. 19.

Strain yGLY3642 was generated by counterselecting strain yGLY2690 in the presence of 5′FOA, a URA5 auxotroph.

Strain yGLY3668 was generated by transforming yGLY3642 with integration/expression plasmid pGLY1234 encoding the hCRT and hERp57 and which targets the HIS3 locus.

Strain yGLY3693 was generated by transforming strain yGLY3668 with integration/expression plasmid pGLY2261, which targets an expression cassette encoding the anti-DKK1 antibody to the TRP2 locus.

Strain yGLY8239 was generated by transforming strain yGLY3693 with integration/expression plasmid pGLY3833 encoding the PpPMR1 and targeting the URA6 locus.

Strain yGLY8241 was generated by transforming strain yGLY3693 with integration/expression plasmid pGLY3827 encoding the AtECA1 and targeting the URA6 locus.

The geneology of the strains described in this example are shown in FIGS. 3A and 3B.

The above strains were evaluated to see whether the addition of hCRT and hERp57 to the humanized chaperone strains expressing PpPMR1 or AtECA1 of the previous example might effect a further reduction in O-glycan occupancy of the antibodies produced. As shown in Table 10, in strain yGLY3693 expressing hCRT and hERp57 alone, there was about a 2-fold decrease in O-glycan occupancy, which was further decreased up to a 4-fold in strains that further expressed PpPMR1 or AtECA1. The results also suggest that yeast strains that express its endogenous PDI1 and not the human PDI and overexpress a Ca²⁺ ATPase will produce glycoproteins with reduced O-glycan occupancy.

TABLE 10

| | | | yGLY3693 + Ca²⁺ ATPase | |
|---|---|---|---|---|
| | | | yGLY8241 | yGLY8239 |
| Strain | yGLY2263 | yGLY3693 | AtECA1 | PpPMR1 |
| O-glycan | 23.7 | 10.43 | 5.59 | 7.86 |
| occupancy | | | | |
| (H2 + L2: | | | | |
| anti-DKK1) | | | | |

O-glycan occupancy was determined by Mannitol assay.

TABLE 11
BRIEF DESCRIPTION OF THE SEQUENCES

| SEQ | | |
|---|---|---|
| ID | | |
| NO: | Description | Sequence |
| 1 | PCR primer | AGCGCTGACGCCCCCGAGGAGGAGGACCAC |
| | hPDI/UP1 | |
| 2 | PCR primer | CCTTAATTAATTACAGTTCATCATGCACAGCTTTCTGATCAT |
| | hPDI/LP-PacI | |
| 3 | PCR primer | ATGAATTCAGGC CATATCGGCCATTGTTTACTGTGCG |
| | PB248 | CCCACAGTAG |
| 4 | PCR primer | ATGTTTA AACGTGAGGATTACTGGTGATGAAAGAC |
| | PB249 | |
| 5 | PCR primer | AGACTAGTCTATTTGGAG ACATTGACGGATCCAC |
| | PB250 | |
| 6 | PCR primer | ATCTCGAGAGGCCATGCAGGCCAACCACAAGATGAATCAAAT |
| | PB251 | TTTG |
| 7 | PCR primer | GGTGAGGTTGAGGTCCCAAGTGACTATCAAGGTC |
| | PpPDI/UPi-1 | |
| 8 | PCR primer | GACCTTGATAGTCACTTGGGACCTCAACCTCACC |
| | PpPDI/LPi-1 | |
| 9 | PCR primer | CGCCAATGATGAGGATGCCTCTTCAAAGGTTGTG |
| | PpPDI/UPi-2 | |
| 10 | PCR primer | CACAACCTTTGAAGAGGCATCCTCATCATTGGCG |
| | PpPDI/LPi-2 | |
| 11 | PCR primer | GGCGATTGCATTCGCGAC TGTATC |
| | PpPDI-5′/UP | |
| 12 | PCR primer | CCTAGAGAGCGGTGG CCAAGATG |
| | hPDI-3′/LP | |
| 13 | PCR primer | GTGGCCACACCAGGGGGC ATGGAAC |
| | hPDI/UP | |
| 14 | PCR primer | CCTAGAGAGCGGTGG CCAAGATG |
| | hPDI-3′/LP | |
| 15 | PCR primer | AGCGCTGACGATGAAGTTGATGTGGATGGTACA GTAG |
| | hGRP94/UP1 | |
| 16 | PCR primer | GGCCGGCCTTACAATTCATCATG TTCAGCTGTAGATTC |
| | hGRP94/LP1 | |
| 17 | PCR primer | TGAACCCATCTGTAAATAGAATGC |
| | PMT1-KO1 | |
| 18 | PCR primer | GTGTCACCTAAATCGTATGTGCCCATTTACTGGA |
| | PMT1-KO2 | AGCTGCTAACC |
| 19 | PCR primer | CTCCCTATAGTGAGTCGTATTCATCATTGTACTTT |
| | PMT1-KO3 | GGTATATTGG |
| 20 | PCR primer | TATTTGTACCTGCGTCCTGTTTGC |
| | PMT1-KO4 | |
| 21 | PCR primer | CACATACGATTTAGGTGACAC |
| | PR29 | |
| 22 | PCR primer | AATACGACTCACTATAGGGAG |
| | PR32 | |
| 23 | PCR primer | TGCTCTCCGCGTGCAATAGAAACT |
| | PMT4-KO1 | |
| 24 | PCR primer | CTCCCTATAGTGAGTCGTATTCACAGTGTACCATCT |
| | PMT4-KO2 | TTCATCTCC |
| 25 | PCR primer | GTGTCACCTAAATCGTATGTGAACCTAACTCTAA |
| | PMT4-KO3 | TTCTTCAAAGC |
| 26 | PCR primer | ACTAGGGTATATAATTCCCAAGGT |
| | PMT4-KO4 | |
| 27 | Saccharomyces | ATG AGA TTC CCA TCC ATC TTC ACT GCT GTT TTG TTC GCT |
| | cerevisiae | GCT TCT TCT GCT TTG GCT |
| | mating factor | |
| | pre-signal | |
| | peptide (DNA) | |
| 28 | Saccharomyces | MRFPSIFTAVLFAASSALA |
| | cerevisiae | |
| | mating factor | |
| | pre-signal | |
| | peptide | |
| | (protein) | |
| 29 | Anti-Her2 | GAGGTTCAGTTGGTTGAATCTGGAGGAGGATTGGTTCAACCTG |
| | Heavy chain | GTGGTTCTTTGAGATTGTCCTGTGCTGCTTCCGGTTTCAACATC |
| | (VH + IgG1 | AAGGACACTTACATCCACTGGGTTAGACAAGCTCCAGGAAAG |
| | constant | GGATTGGAGTGGGTTGCTAGAATCTACCCAACTAACGGTTAC |
| | region) (DNA) | ACAAGATACGCTGACTCCGTTAAGGGAAGATTCACTATCTCTG |
| | | CTGACACTTCCAAGAACACTGCTTACTTGCAGATGAACTCCTT |
| | | GAGAGCTGAGGATACTGCTGTTTACTACTGTTCCAGATGGGGT |
| | | GGTGATGGTTTCTACGCTATGGACTACTGGGGTCAAGGAACTT |
| | | TGGTTACTGTTTCCTCCGCTTCTACTAAGGGACCATCTGTTTTC |
| | | CCATTGGCTCCATCTTCTAAGTCTACTTCCGGTGGTACTGCTGC |
| | | TTTGGGATGTTTGGTTAAAGACTACTTCCCAGAGCCAGTTACT |
| | | GTTTCTTGGAACTCCGGTGCTTTGACTTCTGGTGTTCACACTTT |
| | | CCCAGCTGTTTTGCAATCTTCCGGTTTGTACTCTTTGTCCTCCG |
| | | TTGTTACTGTTCCATCCTCTTCCTTGGGTACTCAGACTTACATC |
| | | TGTAACGTTAACCACAAGCCATCCAACACTAAGGTTGACAAG |
| | | AAGGTTGAGCCAAAGTCCTGTGACAAGACTCATACTTGTCCAC |
| | | CATGTCCAGCTCCAGAATTGTTGGGTGGTCCTTCCGTTTTTTTG |
| | | TTCCCACCAAAGCCAAAGGACACTTTGATGATCTCCAGAACTC |
| | | CAGAGGTTACATGTGTTGTTGTTGACGTTTCTCACGAGGACCC |
| | | AGAGGTTAAGTTCAACTGGTACGTTGACGGTGTTGAAGTTCAC |
| | | AACGCTAAGACTAAGCCAAGAGAGGAGCAGTACAACTCCACT |
| | | TACAGAGTTGTTTCCGTTTTGACTGTTTTGCACCAGGATTGGTT |
| | | GAACGGAAAGGAGTACAAGTGTAAGGTTTCCAACAAGGCTTT |
| | | GCCAGCTCCAATCGAAAAGACTATCTCCAAGGCTAAGGGTCA |
| | | ACCAAGAGAGCCACAGGTTTACACTTTGCCACCATCCAGAGA |
| | | TGAGTTGACTAAGAACCAGGTTTCCTTGACTTGTTTGGTTAAG |
| | | GGATTCTACCCATCCGACATTGCTGTTGAATGGGAGTCTAACG |
| | | GTCAACCAGAGAACAACTACAAGACTACTCCACCTGTTTTGG |
| | | ACTCTGACGGTTCCTTTTTCTTGTACTCCAAGTTGACTGTTGAC |
| | | AAGTCCAGATGGCAACAGGGTAACGTTTTCTCCTGTTCCGTTA |
| | | TGCATGAGGCTTTGCACAACCACTACACTCAAAAGTCCTTGTC |
| | | TTTGTCCCCTGGTAAGTAA |
| 30 | Anti-Her2 | EVQLVESGGGLVQPGGSLRLSCAASGFNIKDTYIHWVRQAPGKG |
| | Heavy chain | LEWVARIYPTNGYTRYADSVKGRFTISADTSKNTAYLQMNSLRA |
| | (VH + IgG1 | EDTAVYYCSRWGGDGFYAMDYWGQGTLVTVSSASTKGPSVFPL |
| | constant | APSSKSTSGGTAALGCLVKDYFPEPVTVSWNSGALTSGVHTFPA |
| | region) | VLQSSGLYSLSSVVTVPSSSLGTQTYICNVNHKPSNTKVDKKVEP |
| | (protein) | KSCDKTHTCPPCPAPELLGGPSVFLFPPKPKDTLMISRTPEVTCVV |
| | | VDVSHEDPEVKFNWYVDGVEVHNAKTKPREEQYNSTYRVVSVL |
| | | TVLHQDWLNGKEYKCKVSNKALPAPIEKTISKAKGQPREPQVYT |
| | | LPPSRDELTKNQVSLTCLVKGFYPSDIAVEWESNGQPENNYKTTP |
| | | PVLDSDGSFFLYSKLTVDKSRWQQGNVFSCSVMHEALHNHYTQ |
| | | KSLSLSPGK |
| 31 | Anti-Her2 light | GACATCCAAATGACTCAATCCCCATCTTCTTTGTCTGCTTCCGT |
| | chain (VL + | TGGTGACAGAGTTACTATCACTTGTAGAGCTTCCCAGGACGTT |
| | Kappa constant | AATACTGCTGTTGCTTGGTATCAACAGAAGCCAGGAAAGGCT |
| | region) (DNA) | CCAAAGTTGTTGATCTACTCCGCTTCCTTCTTGTACTCTGGTGT |
| | | TCCATCCAGATTCTCTGGTTCCAGATCCGGTACTGACTTCACTT |
| | | TGACTATCTCCTCCTTGCAACCAGAAGATTTCGCTACTTACTA |
| | | CTGTCAGCAGCACTACACTACTCCACCAACTTTCGGACAGGGT |
| | | ACTAAGGTTGAGATCAAGAGAACTGTTGCTGCTCCATCCGTTT |
| | | TCATTTTCCCACCATCCGACGAACAGTTGAAGTCTGGTACAGC |
| | | TTCCGTTGTTTGTTTGTTGAACAACTTCTACCCAAGAGAGGCT |
| | | AAGGTTCAGTGGAAGGTTGACAACGCTTTGCAATCCGGTAAC |
| | | TCCCAAGAATCCGTTACTGAGCAAGACTCTAAGGAC |
| | | TCCACTTACTCCTTGTCCTCCACTTTGACTTTGTCCAAGGCTGA |
| | | TTACGAGAAGCACAAGGTTTACGCTTGTGAGGTTACACATCA |
| | | GGGTTTGTCCTCCCCAGTTACTAAGTCCTTCAACAGAGGAGAG |
| | | TGTTAA |
| 32 | Anti-Her2 light | DIQMTQSPSSLSASVGDRVTITCRASQDVNTAVAWYQQKPGKAP |
| | chain (VL + | KLLIYSASFLY |
| | Kappa constant | SGVPSRFSGSRSGTDFTLTISSLQPEDFATYYCQQHYTTPPTFGQG |
| | region) | TKVEIKRTVA APSVFIFPPSDEQLKSGTASVVC |
| | | LNNFYPREAKVQWKVDNALQSGNSQESVTEQ |
| | | DSKDSTYSLSSTLTLSKADYEKHKVYACEVTHQGLSSPVTKSFN |
| | | RGEC |
| 33 | Alpha amylase | ATGGTTGCTT GGTGGTCCTT GTTCTTGTAC GGATTGCAAG |
| | signal peptide | TTGCTGCTCC AGCTTTGGCT |
| | (from | |
| | Aspergillus | |
| | niger α- | |
| | amylase) | |
| | (DNA) | |
| 34 | Alpha amylase | MVAWWSLFLY GLQVAAPALA |
| | signal peptide | |
| | (from | |
| | Aspergillus | |
| | niger α- | |
| | amylase) | |
| 35 | Anti-CD20 | GAGATCGTTT TGACACAGTC CCCAGCTACT TTGTCTTTGT |
| | Light chain | CCCCAGGTGA AAGAGCTACA TTGTCCTGTA GAGCTTCCCA |
| | Variable | ATCTGTTTCC TCCTACTTGG CTTGGTATCA ACAAAAGCCA |
| | Region (DNA) | GGACAGGCTC CAAGATTGTT GATCTACGAC GCTTCCAATA |
| | | GAGCTACTGG TATCCCAGCT AGATTCTCTG GTTCTGGTTC |
| | | CGGTACTGAC TTCACTTTGA CTATCTCTTC CTTGGAACCA |
| | | GAGGACTTCG CTGTTTACTA CTGTCAGCAG AGATCCAATT |
| | | GGCCATTGAC TTTCGGTGGT GGTACTAAGG TTGAGATCAA |
| | | GCGTACGGTT GCTGCTCCTT CCGTTTTCAT TTTCCCACCA |
| | | TCCGACGAAC AATTGAAGTC TGGTACCCAA TTCGCCC |
| 36 | Anti-CD20 | EIVLTQSPAT LSLSPGERAT LSCRASQSVS SYLAWYQQKP |
| | Light chain | GQAPRLLIYD ASNRATGIPA RFSGSGSGTD FTLTISSLEP |
| | Variable | EDFAVYYCQQ RSNWPLTFGG GTKVEIKRTV |
| | Region | AAPSVFIFPPSDEQLKSGTQFA |
| 37 | Anti-CD20 | GCTGTTCAGC TGGTTGAATC TGGTGGTGGA TTGGTTCAAC |
| | Heavy chain | CTGGTAGATC CTTGAGATTG TCCTGTGCTG CTTCCGGTTT |
| | Variable | TACTTTCGGT GACTACACTA TGCACTGGGT TAGACAAGCT |
| | Region (DNA) | CCAGGAAAGG GATTGGAATG GGTTTCCGGT ATTTCTTGGA |
| | | ACTCCGGTTC CATTGGTTAC GCTGATTCCG TTAAGGGAAG |
| | | ATTCACTATC TCCAGAGACA ACGCTAAGAA CTCCTTGTAC |
| | | TTGCAGATGA ACTCCTTGAG AGCTGAGGAT ACTGCTTTGT |
| | | ACTACTGTAC TAAGGACAAC CAATACGGTT CTGGTTCCAC |
| | | TTACGGATTG GGAGTTTGGG GACAGGGAAC TTTGGTTACT |
| | | GTCTCGAGTG CTTCTACTAA GGGACCATCC GTTTTTCCAT |
| | | TGGCTCCATC CTCTAAGTCT ACTTCCGGTG GTACCCAATT |
| | | CGCCC |
| 38 | Anti-CD20 | AVQLVESGGG LVQPGRSLRL SCAASGFTFG DYTMHWVRQA |
| | Heavy chain | PGKGLEWVSG ISWNSGSIGY ADSVKGRFTI SRDNAKNSLY |
| | Variable | LQMNSLRAED TALYYCTKDN QYGSGSTYGL GVWGQGTLVT |
| | Region | VSSASTKGPS VFPLAPSSKS TSGGTQFA |
| 39 | human PDI | GACGCCCCCGAGGAGGAGGACCACGTCTTGGTGCTGCGGAAA |
| | Gene (DNA) | AGCAACTTCGCGGAGGCGCTGGCGGCCCACAAGTACCCGCCG |
| | | GTGGAGTTCCATGCCCCCTGGTGTGGCCACTGCAAGGCTCTGG |
| | | CCCCTGAGTATGCCAAAGCCGCTGGGAAGCTGAAGGCAGAAG |
| | | GTTCCGAGATCAGGTTGGCCAAGGTGGACGCCACGGAGGAGT |
| | | CTGACCTAGCCCAGCAGTACGGCGTGCGCGGCTATCCCACCAT |
| | | CAAGTTCTTCAGGAATGGAGACACGGCTTCCCCCAAGGAATA |
| | | TACAGCTGGCAGAGAGGCTGATGACATCGTGAACTGGCTGAA |
| | | GAAGCGCACGGGCCCGGCTGCCACCACCCTGCCTGACGGCGC |
| | | AGCTGCAGAGTCCTTGGTGGAGTCCAGCGAGGTGGCCGTCAT |
| | | CGGCTTCTTCAAGGACGTGGAGTCGGACTCTGCCAAGCAGTTT |
| | | TTGCAGGCAGCAGAGGCCATCGATGACATACCATTTGGGATC |
| | | ACTTCCAACAGTGACGTGTTCTCCAAATACCAGCTCGACAAAG |
| | | ATGGGGTTGTCCTCTTTAAGAAGTTTGATGAAGGCCGGAACA |
| | | ACTTTGAAGGGGAGGTCACCAAGGAGAACCTGCTGGACTTTA |
| | | TCAAACACAACCAGCTGCCCCTTGTCATCGAGTTCACCGAGCA |
| | | GACAGCCCCGAAGATTTTTGGAGGTGAAATCAAGACTCACAT |
| | | CCTGCTGTTCTTGCCCAAGAGTGTGTCTGACTATGACGGCAAA |
| | | CTGAGCAACTTCAAAACAGCAGCCGAGAGCTTCAAGGGCAAG |
| | | ATCCTGTTCATCTTCATCGACAGCGACCACACCGACAACCAGC |
| | | GCATCCTCGAGTTCTTTGGCCTGAAGAAGGAAGAGTGCCCGG |
| | | CCGTGCGCCTCATCACCTTGGAGGAGGAGATGACCAAGTACA |
| | | AGCCCGAATCGGAGGAGCTGACGGCAGAGAGGATCACAGAG |
| | | TTCTGCCACCGCTTCCTGGAGGGCAAAATCAAGCCCCACCTGA |
| | | TGAGCCAGGAGCTGCCGGAGGACTGGGACAAGCAGCCTGTCA |
| | | AGGTGCTTGTTGGGAAGAACTTTGAAGACGTGGCTTTTGATGA |
| | | GAAAAAAAACGTCTTTGTGGAGTTCTATGCCCCATGGTGTGGT |
| | | CACTGCAAACAGTTGGCTCCCATTTGGGATAAACTGGGAGAG |
| | | ACGTACAAGGACCATGAGAACATCGTCATCGCCAAGATGGAC |
| | | TCGACTGCCAACGAGGTGGAGGCCGTCAAAGTGCACGGCTTC |
| | | CCCACACTCGGGTTCTTTCCTGCCAGTGCCGACAGGACGGTCA |
| | | TTGATTACAACGGGGAACGCACGCTGGATGGTTTTAAGAAAT |
| | | TCCTAGAGAGCGGTGGCCAAGATGGGGCAGGGGATGTTGACG |
| | | ACCTCGAGGACCTCGAAGAAGCAGAGGAGCCAGACATGGAG |
| | | GAAGACGATGACCAGAAAGCTGTGAAAGATGAACTGTAA |
| 40 | human PDI | DAPEEEDHVLVLRKSNFAEALAAHKYPPVEFHAPWCGHCKALA |
| | Gene (protein) | PEYAKAAGKLKAEGSEIRLAKVDATEESDLAQQYGVRGYPTIKF |
| | | FRNGDTASPKEYTAGREADDIVNWLKKRTGPAATTLPDGAAAES |
| | | LVESSEVAVIGFFKDVESDSAKQFLQAAEAIDDIPFGITSNSDVFS |
| | | KYQLDKDGVVLFKKFDEGRNNFEGEVTKENLLDFIKHNQLPLVI |
| | | EFTEQTAPKIFGGEIKTHILLFLPKSVSDYDGKLSNFKTAAESFKG |
| | | KILFIFIDSDHTDNQRILEFFGLKKEECPAVRLITLEEEMTKYKPES |
| | | EELTAERITEFCHRFLEGKIKPHLMSQELPEDWDKQPVKVLVGK |
| | | NFEDVAFDEKKNVFVEFYAPWCGHCKQLAPIWDKLGETYKDHE |
| | | NIVIAKMDSTANEVEAVKVHGFPTLGFFPASADRTVIDYNGERTL |
| | | DGFKKFLESGGQDGAGDVDDLEDLEEAEEPDMEEDDDQKAVHD |
| | | EL |
| 41 | Pichia pastoris | ATGCAATTCAACTGGAATATTAAAACTGTGGCAAGTATTTTGT |
| | PDI1 Gene | CCGCTCTCACACTAGCACAAGCAAGTGATCAGGAGGCTATTG |
| | (DNA) | CTCCAGAGGACTCTCATGTCGTCAAATTGACTGAAGCCACTTT |
| | | TGAGTCTTTCATCACCAGTAATCCTCACGTTTTGGCAGAGTTTT |
| | | TTGCCCCTTGGTGTGGTCACTGTAAGAAGTTGGGCCCTGAACT |
| | | TGTTTCTGCTGCCGAGATCTTAAAGGACAATGAGCAGGTTAAG |
| | | ATTGCTCAAATTGATTGTACGGAGGAGAAGGAATTATGTCAA |
| | | GGCTACGAAATTAAAGGGTATCCTACTTTGAAGGTGTTCCATG |
| | | GTGAGGTTGAGGTCCCAAGTGACTATCAAGGTCAAAGACAGA |
| | | GCCAAAGCATTGTCAGCTATATGCTAAAGCAGAGTTTACCCCC |
| | | TGTCAGTGAAATCAATGCAACCAAAGATTTAGACGACACAAT |
| | | CGCCGAGGCAAAAGAGCCCGTGATTGTGCAAGTACTACCGGA |
| | | AGATGCATCCAACTTGGAATCTAACACCACATTTTACGGAGTT |
| | | GCCGGTACTCTCAGAGAGAAATTCACTTTTGTCTCCACTAAGT |
| | | CTACTGATTATGCCAAAAAATACACTAGCGACTCGACTCCTGC |
| | | CTATTTGCTTGTCAGACCTGGCGAGGAACCTAGTGTTTACTCT |
| | | GGTGAGGAGTTAGATGAGACTCATTTGGTGCACTGGATTGAT |
| | | ATTGAGTCCAAACCTCTATTTGGAGACATTGACGGATCCACCT |
| | | TCAAATCATATGCTGAAGCTAACATCCCTTTAGCCTACTATTT |
| | | CTATGAGAACGAAGAACAACGTGCTGCTGCTGCCGATATTATT |
| | | AAACCTTTTGCTAAAGAGCAACGTGGCAAAATTAACTTTGTTG |
| | | GCTTAGATGCCGTTAAATTCGGTAAGCATGCCAAGAACTTAA |
| | | ACATGGATGAAGAGAAACTCCCTCTATTTGTCATTCATGATTT |
| | | GGTGAGCAACAAGAAGTTTGGAGTTCCTCAAGACCAAGAATT |
| | | GACGAACAAAGATGTGACCGAGCTGATTGAGAAATTCATCGC |
| | | AGGAGAGGCAGAACCAATTGTGAAATCAGAGCCAATTCCAGA |
| | | AATTCAAGAAGAGAAAGTCTTCAAGCTAGTCGGAAAGGCCCA |
| | | CGATGAAGTTGTCTTCGATGAATCTAAAGATGTTCTAGTCAAG |
| | | TACTACGCCCCTTGGTGTGGTCACTGTAAGAGAATGGCTCCTG |
| | | CTTATGAGGAATTGGCTACTCTTTACGCCAATGATGAGGATGC |
| | | CTCTTCAAAGGTTGTGATTGCAAAACTTGATCACACTTTGAAC |
| | | GATGTCGACAACGTTGATATTCAAGGTTATCCTACTTTGATCC |
| | | TTTATCCAGCTGGTGATAAATCCAATCCTCAACTGTATGATGG |
| | | ATCTCGTGACCTAGAATCATTGGCTGAGTTTGTAAAGGAGAG |
| | | AGGAACCCACAAAGTGGATGCCCTAGCACTCAGACCAGTCGA |
| | | GGAAGAAAAGGAAGCTGAAGAAGAAGCTGAAAGTGAGGCAG |
| | | ACGCTCACGACGAGCTTTAA |
| 42 | Pichia pastoris | MQFNWNIKTVASILSALTLAQASDQEAIAPEDSHVVKLTEATFES |
| | PDI1 Gene | FITSNPHVLAEFFAPWCGHCKKLGPELVSAAEILKDNEQVKIAQI |
| | (protein) | DCTEEKELCQGYEIKGYPTLKVFHGEVEVPSDYQGQRQSQSIVSY |
| | | MLKQSLPPVSEINATKDLDDTIAEAKEPVIVQVLPEDASNLESNT |
| | | TFYGVAGTLREKFTFVSTKSTDYAKKYTSDSTPAYLLVRPGEEPS |
| | | VYSGEELDETHLVHWIDIESKPLFGDIDGSTFKSYAEANIPLAYYF |
| | | YENEEQRAAAADIIKPFAKEQRGKINFVGLDAVKFGKHAKNLNM |
| | | DEEKLPLFVIHDLVSNKKFGVPQDQELTNKDVTELIEKFIAGEAEP |
| | | IVKSEPIPEIQEEKVFKLVGKAHDEVVFDESKDVLVKYYAPWCG |
| | | HCKRMAPAYEELATLYANDEDASSKVVIAKLDHTLNDVDNVDI |
| | | QGYPTLILYPAGDKSNPQLYDGSRDLESLAEFVKERGTHKVDAL |
| | | ALRPVEEEKEAEEEAESEADAHDEL |
| 43 | human ERO1 α | GAAGAACAACCACCAGAGACTGCTGCTCAGAGATGCTTCTGT |
| | Gene (DNA) | CAGGTTTCCGGTTACTTGGACGACTGTACTTGTGACGTTGAGA |
| | | CTATCGACAGATTCAACAACTACAGATTGTTCCCAAGATTGCA |
| | | GAAGTTGTTGGAGTCCGACTACTTCAGATACTACAAGGTTAAC |
| | | TTGAAGAGACCATGTCCATTCTGGAACGACATTTCCCAGTGTG |
| | | GTAGAAGAGACTGTGCTGTTAAGCCATGTCAATCCGACGAAG |
| | | TTCCAGACGGTATTAAGTCCGCTTCCTACAAGTACTCTGAAGA |
| | | GGCTAACAACTTGATCGAAGAGTGTGAGCAAGCTGAAAGATT |
| | | GGGTGCTGTTGACGAATCTTTGTCCGAGAGACTCAGAAGGCT |
| | | GTTTTGCAGTGGACTAAGCACGATGATTCCTCCGACAACTTCT |
| | | GTGAAGCTGACGACATTCAATCTCCAGAGGCTGAGTACGTTG |
| | | ACTTGTTGTTGAACCCAGAGAGATACACTGGTTACAAGGGTCC |
| | | AGACGCTTGGAAGATTTGGAACGTTATCTACGAAGAGAACTG |
| | | TTTCAAGCCACAGACTATCAAGAGACCATTGAACCCATTGGCT |
| | | TCCGGACAGGGAACTTCTGAAGAGAACACTTTCTACTCTTGGT |
| | | TGGAGGGTTTGTGTGTTGAGAAGAGAGCTTTCTACAGATTGAT |
| | | CTCCGGATTGCACGCTTCTATCAACGTTCACTTGTCCGCTAGA |
| | | TACTTGTTGCAAGAGACTTGGTTGGAAAAGAAGTGGGGTCAC |
| | | AACATTACTGAGTTCCAGCAGAGATTCGACGGTATTTTGACTG |
| | | AAGGTGAAGGTCCAAGAAGATTGAAGAACTTGTACTTTTTGT |
| | | ACTTGATCGAGTTGAGAGCTTTGTCCAAGGTTTTGCCATTCTT |
| | | CGAGAGACCAGACTTCCAATTGTTCACTGGTAACAAGATCCA |
| | | GGACGAAGAGAACAAGATGTTGTTGTTGGAGATTTTGCACGA |
| | | GATCAAGTCCTTTCCATTGCACTTCGACGAGAACTCATTTTTC |
| | | GCTGGTGACAAGAAAGAAGCTCACAAGTTGAAAGAGGACTTC |
| | | AGATTGCACTTCAGAAATATCTCCAGAATCATGGACTGTGTTG |
| | | GTTGTTTCAAGTGTAGATTGTGGGGTAAGTTGCAGACTCAAGG |
| | | ATTGGGTACTGCTTTGAAGATTTTGTTCTCCGAGAAGTTGATC |
| | | GCTAACATGCCTGAATCTGGTCCATCTTACGAGTTCCACTTGA |
| | | CTAGACAAGAGATCGTTTCCTTGTTCAACGCTTTCGGTAGAAT |
| | | CTCCACTTCCGTTAAAGAGTTGGAGAACTTCAGAAACTTGTTG |
| | | CAGAACATCCACTAA |
| 44 | human ERO1 α | EEQPPETAAQRCFCQVSGYLDDCTCDVETIDRFNNYRLFPRLQKL |
| | Gene (protein) | LESDYFRYYKVNLKRPCPFWNDISQCGRRDCAVKPCQSDEVPDG |
| | | IKSASYKYSEEANNLIEECEQAERLGAVDESLSEETQKAVLQWTK |
| | | HDDSSDNFCEADDIQSPEAEYVDLLLNPERYTGYKGPDAWKIWN |
| | | VIYEENCFKPQTIKRPLNPLASGQGTSEENTFYSWLEGLCVEKRA |
| | | FYRLISGLHASINVHLSARYLLQETWLEKKWGHNITEFQQRFDGI |
| | | LTEGEGPRRLKNLYFLYLIELRALSKVLPFFERPDFQLFTGNKIQD |
| | | EENKMLLLEILHEIKSFPLHFDENSFFAGDKKEAHKLKEDFRLHF |
| | | RNISRIMDCVGCFKCRLWGKLQTQGLGTALKILFSEKLIANMPES |
| | | GPSYEFHLTRQEIVSLFNAFGRISTSVKELENFRNLLQNIH |
| 45 | human GRP94 | GATGATGAAGTTGACGTTGACGGTACTGTTGAAGAGGACTTG |
| | Gene (DNA) | GGAAAGTCTAGAGAGGGTTCCAGAACTGACGACGAAGTTGTT |
| | | CAGAGAGAGGAAGAGGCTATTCAGTTGGACGGATTGAACGCT |
| | | TCCCAAATCAGAGAGTTGAGAGAGAAGTCCGAGAAGTTCGCT |
| | | TTCCAAGCTGAGGTTAACAGAATGATGAAATTGATTATCAACT |
| | | CCTTGTACAAGAACAAAGAGATTTTCTTGAGAGAGTTGATCTC |
| | | TAACGCTTCTGACGCTTTGGACAAGATCAGATTGATCTCCTTG |
| | | ACTGACGAAAACGCTTTGTCCGGTAACGAAGAGTTGACTGTT |
| | | AAGATCAAGTGTGACAAAGAGAAGAACTTGTTGCACGTTACT |
| | | GACACTGGTGTTGGAATGACTAGAGAAGAGTTGGTTAAGAAC |
| | | TTGGGTACTATCGCTAAGTCTGGTACTTCCGAGTTCTTGAACA |
| | | AGATGACTGAGGCTCAAGAAGATGGTCAATCCACTTCCGAGT |
| | | TGATTGGTCAGTTCGGTGTTGGTTTCTACTCCGCTTTCTTGGTT |
| | | GCTGACAAGGTTATCGTTACTTCCAAGCACAACAACGACACTC |
| | | AACACATTTGGGAATCCGATTCCAACGAGTTCTCCGTTATTGC |
| | | TGACCCAAGAGGTAACACTTTGGGTAGAGGTACTACTATCACT |
| | | TTGGTTTTGAAAGAAGAGGCTTCCGACTACTTGGAGTTGGACA |
| | | CTATCAAGAACTTGGTTAAGAAGTACTCCCAGTTCATCAACTT |
| | | CCCAATCTATGTTTGGTCCTCCAAGACTGAGAC |
| | | TGTTGAGGAACCAATGGAAGAAGAAGAGGCTGCTAAAGAAG |
| | | AGAAAGAGGAATCTGACGACGAGGCTGCTGTTGAAGAAGAG |
| | | GAAGAAGAAAAGAAGCCAAAGACTAAGAAGGTTGAAAAGAC |
| | | TGTTTGGGACTGGGAGCTTATGAACGACATCAAGCCAATTTGG |
| | | CAGAGACCATCCAAAGAGGTTGAGGAGGACGAGTACAAGGCT |
| | | TTCTACAAGTCCTTCTCCAAAGAATCCGATGACCCAATGGCTT |
| | | ACATCCACTTCACTGCTGAGGGTGAAGTTACTTTCAAGTCCAT |
| | | CTTGTTCGTTCCAACTTCTGCTCCAAGAGGATTGTTCGACGAG |
| | | TACGGTTCTAAGAAGTCCGACTACATCAAACTTTATGTTAGAA |
| | | GAGTTTTCATCACTGACGACTTCCACGATATGATGCCAAAGTA |
| | | CTTGAACTTCGTTAAGGGTGTTGTTGATTCCGATGACTTGCCA |
| | | TTGAACGTTTCCAGAGAGACTTTGCAGCAGCACAAGTTGTTGA |
| | | AGGTTATCAGAAAGAAACTTGTTAGAAAGACTTTGGACATGA |
| | | TCAAGAAGATCGCTGACGACAAGTACAACGACACTTTCTGGA |
| | | AAGAGTTCGGAACTAACATCAAGTTGGGTGTTATTGAGGACC |
| | | ACTCCAACAGAACTAGATTGGCTAAGTTGTTGAGATTCCAGTC |
| | | CTCTCATCACCCAACTGACATCACTTCCTTGGACCAGTACGTT |
| | | GAGAGAATGAAAGAGAAGCAGGACAAAATCTACTTCATGGCT |
| | | GGTTCCTCTAGAAAAGAGGCTGAATCCTCCCCATTCGTTGAGA |
| | | GATTGTTGAAGAAGGGTTACGAGGTTATCTACTTGACTGAGCC |
| | | AGTTGACGAGTACTGTATCCAGGCTTTGCCAGAGTTTGACGGA |
| | | AAGAGATTCCAGAACGTTGCTAAAGAGGGTGTTAAGTTCGAC |
| | | GAATCCGAAAAGACTAAAGAATCCAGAGAGGCTGTTGAGAAA |
| | | GAGTTCGAGCCATTGTTGAACTGGATGAAGGACAAGGCTTTG |
| | | AAGGACAAGATCGAGAAGGCTGTTGTTTCCCAGAGATTGACT |
| | | GAATCCCCATGTGCTTTGGTTGCTTCCCAATACGGATGGAGTG |
| | | GTAACATGGAAAGAATCATGAAGGCTCAGGCTTACCAAACTG |
| | | GAAAGGACATCTCCACTAACTACTACGCTTCCCAGAAGAAAA |
| | | CTTTCGAGATCAACCCAAGACACCCATTGATCAGAGACATGTT |
| | | GAGAAGAATCAAAGAGGACGAGGACGACAAGACTGTTTTGG |
| | | ATTTGGCTGTTGTTTTGTTCGAGACTGCTACTTTGAGATCCGGT |
| | | TACTTGTTGCCAGACACTAAGGCTTACGGTGACAGAATCGAG |
| | | AGAATGTTGAGATTGTCCTTGAACATTGACCCAGACGCTAAG |
| | | GTTGAAGAAGAACCAGAAGAAGAGCCAGAGGAAACTGCTGA |
| | | AGATACTACTGAGGACACTGAACAAGACGAGGACGAAGAGA |
| | | TGGATGTTGGTACTGACGAAGAGGAAGAGACAGCAAAGGAAT |
| | | CCACTGCTGAACACGACGAGTTGTAA |
| 46 | human GRP94 | DDEVDVDGTVEEDLGKSREGSRTDDEVVQREEEAIQLDGLNASQ |
| | Gene (protein) | IRELREKSEKFAFQAEVNRMMKLIINSLYKNKEIFLRELISNASDA |
| | | LDKIRLISLTDENALSGNEELTVKIKCDKEKNLLHVTDTGVGMTR |
| | | EELVKNLGTIAKSGTSEFLNKMTEAQEDGQSTSELIGQFGVGFYS |
| | | AFLVADKVIVTSKHNNDTQHIWESDSNEFSVIADPRGNTLGRGTT |
| | | ITLVLKEEASDYLELDTIKNLVKKYSQFINFPIYVWSSKTETVEEP |
| | | MEEEEAAKEEKEESDDEAAVEEEEEEKKPKTKKVEKTVWDWEL |
| | | MNDIKPIWQRPSKEVEEDEYKAFYKSFSKESDDPMAYIHFTAEGE |
| | | VTFKSILFVPTSAPRGLFDEYGSKKSDYIKLYVRRVFITDDFHDM |
| | | MPKYLNFVKGVVDSDDLPLNVSRETLQQHKLLKVIRKKLVRKTL |
| | | DMIKKIADDKYNDTFWKEFGTNIKLGVIEDHSNRTRLAKLLRFQS |
| | | SHHPTDITSLDQYVERMKEKQDKIYFMAGSSRKEAESSPFVERLL |
| | | KKGYEVIYLTEPVDEYCIQALPEFDGKRFQNVAKEGVKFDESEK |
| | | TKESREAVEKEFEPLLNWMKDKALKDKIEKAVVSQRLTESPCAL |
| | | VASQYGWSGNMERIMKAQAYQTGKDISTNYYASQKKTFEINPR |
| | | HPLIRDMLRRIKEDEDDKTVLDLAVVLFETATLRSGYLLPDTKAY |
| | | GDRIERMLRLSLNIDPDAKVEEEPEEEPEETAEDTTEDTEQDEDE |
| | | EMDVGTDEEEETAKESTAEHDEL |
| 47 | PpPMT1 gene | ACTTTTTCAATTCCTCAGGGTACTCCGTTGGAATTCTGTACTTA |
| | (DNA) | GCAGCATACTGATCTTTGACCACCCAAGGAGCACCAGATCTTT |
| | CDS 3016- | GCGATCTAGTCAACGTCAACTTGAGAAAAGTTTTCACGTACCA |
| | 5385 | CTTAGTGAACGCATTCCTATCACGGGAAACTTGATTTTCGTTC |
| | | ACGGTTACTTCTCCATCAGAGTTTGAGAGGCCAACGCGATAA |
| | | GAGCAGTATCCTTCACGTACGGTACCATCAGGTAAGGTGATG |
| | | GGAGCAAACCGTGCCTTTTCTCTGATGATCCCTTTATATCTGTT |
| | | AGATCCAGCACTTTTAACATTCACTAGATCCCCAGGAAAAAAT |
| | | TCTTTCTTGAAGTGTAAATACACGTCATCGACTAATTGATCTA |
| | | GTCTGGGTATGAGACTGAATTGCACATATCTCAAAATTGGTTC |
| | | TCTGACAGGTTCTGGAAACTTATTTTCAACCGCTTGCATTTCCT |
| | | CCTGTTCGTACTTGAGGGCCTCAAAGAAATCAAACGAGCTGTT |
| | | TCCAGTGATTTCACACGCAAACTTCTTTTGGCTATAATAGTCC |
| | | ATCCTATCAAGGTACTCATCGTAGTTCAAAAACCACTCGCCAG |
| | | TCTGTGGAATGTACCAAATTTCCGTGTCTAGATCATCAGGAAG |
| | | CTGTTGTGGAGGGACAACTTCCACCTGCTTTCTTTTGAAGAGA |
| | | ACCATGGTGTTTGGGGATTAGAAGAAAACAAATATTTGAGCG |
| | | GAACTTGCGAAAAAACGCCCCTAGCGAATGCAAGCTAGACAT |
| | | GTCAGGAAGATAAAATTGATACCGCAGAAGCAGGGGTAGTTG |
| | | GGGAGGGCAATCAAGTACGTTCACAGAGCATGGCTGCGTTAT |
| | | CAACTGACTATTTTATGGCGTGGTTTAGAAGAGAGAGTATCAA |
| | | TTAGGCGTCAACTGGGACCATTATGATTAGACGTTGTAGGTAG |
| | | ATGCAGGTGAAAAATGGACAGACGTAGGCAACAAACACAAA |
| | | CTGTCGGGTAACCTTTAACAGTATTCAATTCCAGGTGTTTCAA |
| | | GACAGCCTTAGATACTAGCAAGCTTCCAGGGAAACCCTATTA |
| | | CTCATGCTCCCACTGTTGGAACTCACAACCAAGAGGCTACATG |
| | | TATGCGTATGCATACAGGTACTGCTCAGTGATAAATTTATTTC |
| | | GCGAGATCGTACTCCAGAAACTTTCATGTAAGCCTTCCTACTT |
| | | CGCTCTGCCCACTATGTTAGCCAGAAAGGTATTAGCTAGACAA |
| | | TGTCTGGTGGTAGCCAGGCTTTGTGCGGGTAGATTTGCCTCCT |
| | | CATTATGCGGGTGCAGTTGTAGAGGTTTGATGAGGCCACCAA |
| | | AATTTAACAGTTCCAAATCTCTTTCGAGATCGATGACCTCATC |
| | | GTCCCTGTTTGAGTCTCCAAATTGTCCTTCCTGTGGTGTGGTTC |
| | | TCCAAACAGAACATCCAGACAAAGATGGGTATTGTCTACTGC |
| | | CCAAAGGTGAAAGGAAAGTTAAAAATTATCAAAATGAACTAA |
| | | AAGAAAAGCTTTTTTTGAATGTGAAAAGGGAAGAACTTGCCG |
| | | ACAGACTGGGCCATGAGGTGGACTCTGAATCACTGATTATAC |
| | | CCAAGGAAATGTACCAAAAGCCCCGTACCCCGAAACGACTGG |
| | | TTTGTCAGAGATGCTTCAAATCGCAAAACTATTCCTTGATCGA |
| | | CCATTCCATTCGTGAAGAAAATCCCGAACACAAGATCCTGGA |
| | | TGAGATCCCTTCAAACGCCAATATCGTCCACGTTTTATCTGCT |
| | | GTTGATTTTCCTCTTGGTCTCAGCAAGGAACTGGTAAACAGAT |
| | | TTAAACCCACTCAGATTACGTACGTTATTACAAAGTCTGACGT |
| | | GTTCTTCCCCGATAAGCTAGGTCTCCAACGGACGGGAGCTGCT |
| | | TATTTTGAAGACAGCTTGGTAAAGCTTGTCGGTGCAGATCCTA |
| | | GAAAGGTAGTATTGGTCTCAGGAAAAAGAAATTGGGGCCTCA |
| | | AACAGCTGCTATCCACTTTACCCAGAGGTCCCAATTACTTTCT |
| | | GGGAATGACGAACACCGGAAAATCAACCCTAATACGATCCAT |
| | | CGTTGGTAAGGATTACTCAAAGAAGCAGACAGAGAATGGCCC |
| | | GGGTGTCTCTCACCTTCCTTCATTCACAAGAAAACCCATGAAG |
| | | TTCAAAATGGACAACAACAGTCTTGAACTCGTAGATCTCCCTG |
| | | GATACACTGCTCCAAATGGAGGTGTTTACAAGTATCTTAAGGA |
| | | AGAGAACTACCGAGACATTTTGAACGTTAAACAGTTAAAGCC |
| | | ATTGACATCCCTCAAGGCATACACAGAAACGTTGCCTTCGAA |
| | | GCCAAAACTATTCAATGGTGTGCGAGTAATATGCATTGGTGGT |
| | | TTAGTGTACATTCGGCCCCCAAAGGGTGTAGTGCTGAAACAGT |
| | | TTAGTCTCGTCAACCTTCCATCCTTCATGTACTCGTCGCTAAAA |
| | | AAGGCCACCAGTGTAATCCAAGCGCCCCCACAAGCCTTGGTG |
| | | AATTGCAGCGTCGTCAAGGAGGACAGTCCAGATGAACTGGTA |
| | | AGATATGTGATCCCTCCATTTTATGGTTTAATTGACCTGGTCAT |
| | | TCAAGGTGTTGGATTTATCAAGCTTCTGCCCACTGGAGCTCGG |
| | | AACACCAGAGAACTGATAGAAATTTTTGCCCCAAAAGACATC |
| | | CAGCTCATGGTGCGTGATTCCATCCTCAAATACGTCTACAAGA |
| | | CCCATGCCGAACACGACTCAACCAATAATCTCCTGCATAAAA |
| | | AGAACATAAAAGCCAGAGGCCAAACCATACTACGAAGACTAC |
| | | CCAAAAAGCCTGTATTCACAAAGCTTTTTCCCGTACCAGCCAA |
| | | CGTACCGTCTCATGAACTGCTCACCATGGTGACGGGAAAGGA |
| | | CGACCTAGCCGAGGAAGACAAAGAATACCGCTACGATATCCA |
| | | GTATCCCAACAGATACTGGGATGAAACCATCTGTAAATAGAA |
| | | TGCTTATGTAATCAAGCACTTTCTGAAATTCCTTAGAGTTTCG |
| | | CGTGTCTCCCCGTCAAAAATCGCGTCTC [symbol] TGCCAGATATTT |
| | | CTCCCGCAAAACGTAACACGTTGTTCTGTTTCCCTTTTGACAA |
| | | TGAGTAAAACAAGTCCTCAAGAGGTGCCAGAAAACACTACTG |
| | | AGCTTAAAATCTCAAAAGGAGAGCTCCGTCCTTTTATTGTGAC |
| | | CTCTCCATCTCCTCAATTGAGCAAGTCTCGTTCTGTGACTTCAA |
| | | CCAAGGAGAAGCTGATATTGGCTAGTTTGTTCATATTTGCAAT |
| | | GGTCATCAGGTTCCACAACGTCGCCCACCCTGACAGCGTTGTG |
| | | TTTGATGAAGTTCACTTTGGGGGGTTTGCCAGAAAGTACATTT |
| | | TGGGAACCTTTTTCATGGATGTTCATCCGCCATTGGCCAAGCT |
| | | ATTATTTGCTGGTGTTGGCAGTCTTGGTGGATACGATGGAGAG |
| | | TTTGAGTTCAAGAAAATTGGTGACGAATTCCCAGAGAATGTTC |
| | | CTTATGTGCTCATGAGATATCTTCCCTCTGGTATGGGAGTTGG |
| | | AACATGTATTATGTTGTATTTGACTCTGAGAGCTTCTGGTTGTC |
| | | AACCAATAGTCTGTGCTCTGACAACCGCTCTTTTGATCATTGA |
| | | GAATGCTAATGTTACAATCTCCAGATTCATTTTGCTGGATTCG |
| | | CCAATGCTGTTTTTTATTGCTTCAACAGTTTACTCTTTCAAGAA |
| | | ATTTCAAATTCAGGAACCGTTTACCTTCCAATGGTACAAGACC |
| | | CTTATTGCTACTGGTGTTTCTTTAGGGTTAGCAGCTTCCAGTAA |
| | | ATGGGTTGGTTTGTTCACCGTTGCCTGGATTGGATTGATAACA |
| | | ATTTGGGACTTATGGTTCATCATTGGTGATTTGACTGTTTCTGT |
| | | AAAGAAAATTTTCGGCCATTTTATCACCAGAGCTGTAGCTTTC |
| | | TTAGTCGTCCCCACTCTGATCTACCTCACTTTCTTTGCCATCCA |
| | | TTTGCAAGTCTTAACCAAGGAAGGTGATGGTGGTGCTTTCATG |
| | | TCTTCCGTCTTCAGATCGACCTTAGAAGGTAATGCTGTTCCAA |
| | | AACAGTCGCTGGCCAACGTTGGTTTGGGCTCTTTAGTCACTAT |
| | | CCGTCATTTGAACACCAGAGGTGGTTACTTACACTCTCACAAT |
| | | CATCTTTACGAGGGTGGTTCTGGTCAACAGCAGGTCACCTTGT |
| | | ACCCACACATTGATTCTAATAATCAATGGATTGTACAGGATTA |
| | | CAACGCGACTGAGGAGCCAACTGAATTTGTTCCATTGAAAGA |
| | | CGGTGTCAAAATCAGATTAAACCACAAATTGACTTCCCGAAG |
| | | ATTGCACTCTCATAACCTCAGACCTCCTGTGACTGAACAAGAT |
| | | TGGCAAAATGAGGTATCTGCTTATGGACATGAGGGCTTTGGC |
| | | GGTGATGCCAATGATGACTTTGTTGTGGAGATTGCCAAGGATC |
| | | TTTCAACTACTGAAGAAGCTAAGGAAAACGTTAGGGCCATTC |
| | | AAACTGTTTTTAGATTGAGACATGCGATGACTGGTTGTTACTT |
| | | GTTCTCCCACGAAGTCAAGCTTCCCAAGTGGGCATATGAGCA |
| | | ACAAGAGGTTACTTGTGCTACTCAAGGTATCAAACCACTATCT |
| | | TACTGGTACGTTGAGACCAACGAAAACCCATTCTTGGATAAA |
| | | GAGGTTGATGAAATAGTTAGCTATCCTGTTCCGACTTTCTTTC |
| | | AAAAGGTTGCCGAGCTACACGCCAGAATGTGGAAGATCAACA |
| | | AGGGCTTAACTGATCATCATGTCTATGAATCCAGTCCAGATTC |
| | | TTGGCCCTTCCTGCTCAGAGGTATAAGCTACTGGTCAAAAAAT |
| | | CACTCACAAATTTATTTCATAGGTAATGCTGTCACTTGGTGGA |
| | | CAGTCACCGCAAGTATTGCTTTGTTCTCTGTCTTTTTGGTTTTC |
| | | TCTATTCTGAGATGGCAAAGAGGTTTTGGGTTCAGCGTTGACC |
| | | CAACTGTGTTCAACTTCAATGTTCAAATGCTTCATTACATCCT |
| | | AGGATGGGTACTGCATTACTTGCCATCTTTCCTTATGGCCCGT |
| | | CAGCTATTTTTGCACCACTATCTACCATCATTGTACTTTGGTAT |
| | | ATTGGCTCTCGGACATGTGTTTGAGATTATTCACTCTTATGTCT |
| | | TCAAAAACAAACAGGTTGTGTCTTACTCCATATTCGTTCTCTTT |
| | | TTTGCCGTTGCGCTTTCTTTCTTCCAAAGATATTCTCCATTGAT |
| | | CTATGCAGGACGATGGACCAAGGACCAATGCAACGAATCCAA |
| | | GATACTCAAGTGGGACTTTGACTGTAACACCTTCCCCAGTCAC |
| | | ACATCTCAGTATGAAATATGGGCATCCCCTGTACAAACTTCCA |
| | | CTCCTAAAGAAGGAACCCACTCAGAATCTACCGTCGGAGAAC |
| | | CTGACGTTGAGAAGCTGGGAGAGACAGTC [symbol] GCTGTGTTTA |
| | | TATAGCCCTGTACGTAAAATCTATGACACAAGTTTATGGTTAT |
| | | TTGTCTTATGTAAGCAATATTTGGATTGATGTCTCGAGACCAT |
| | | CAACTCCATCACTGATAAGTTGATCGGATTTGTATTTCTGTCC |
| | | CCTATTTACTAATTCCCTTTCCAGAAATAGATCATGAATGAGG |
| | | CAGAATATAAGTGCCAAAGATGCCGGCTGCCGTTGACCATAG |
| | | ACGGATCTCTGGAAGACCTTAGCATATCACAGGCCAATCTTTT |
| | | GACGGGACGAAATGGGAACTTTACAAAGAACACAATCCCCTT |
| | | GGAGGATGCCGTGGAAGAAGATTTACCCAAGGTGCCTCAGAG |
| | | CCGACTTAACCTCTTTAAAGAGGTCTACCAGAAGATGGATCAC |
| | | GATTTTACCAATGCCAGAGATGAATTTGTTGTGTTGAACAAGC |
| | | ACAATGATAACAGCGACGTCAATGTGGAGTATGATTACGAAG |
| | | AAAACAACACTATCAGTCGTAGAATCAACACAATGACGAATA |
| | | TCTTCAATATCCTCAGCAACAAGTACGAAATTGATTTTCCGGT |
| | | TTGCTACGAATGCGCCACATTGCTGATGGAGGAATTGAAGAA |
| | | TGAGTACGAAAGGGTCAATGCTGATAAAGAAGTTTACGCAAA |
| | | GTTTCTATCCAAGCTTCGCAAACAGGACGCAGGTACAAATAT |
| | | GAAAGAAAGAACTGCTCAACTACTGGAGCAATTGGAGAAAAC |
| | | TAAGCAAGAAGAGAGAGATAAAGAAAAGAAGCTCCAAGGCC |
| | | TATATGATGAAAGAGATAGTTTGGAAAAGGTATTAGCTTCTTT |
| | | AGAGAATGAAATGGAACAGTTGAATATTGAAGAGCAGCAAAT |
| | | TTTTGAATTAGAGAACAAATATGAATATGAGTTAATGGAGTTC |
| | | AAGAATGAGCAAAGCAGAATGGAAGCAATGTATGAGGATGG |
| | | TTTGACGCAATTAGATAATTTAAGAAAAGTGAACGTCTTTAAT |
| | | GACGCTTTCAATATCTCGCATGATGGTCAATTCGGCACTATAA |
| | | ATGGGCTCAGGTTGGGCACGTTAGACAGTAAGAGGGTTTCTT |
| | | GGTATGAAATAAATGCTGCGTTGGGTCAAGTTGTTTTGTTACT |
| | | CTTCACGTTATTGAGCAGACTTGAGCTTGAGCTCAAACATTAC |
| | | AAGATTTTTCCCATTGGCTCGACTTCCAAGATTGAATACCAAG |
| | | TTGACCCAGATTCCAAACCTGTTACTATTAACTGCTTTTCTTCG |
| | | GGAGAACAGTTACTGGATAAGCTTTTTCATTCTAATAAACTAG |
| | | ATCCTGCTATGAACGCAATCCTAGAAATCACTATTCAAATTGC |
| | | AGATCATTTCACAAAACAAGATCCAACAAACGAATTGCCCTA |
| | | CAAAATGGAGAACGAAACAATATCAAACTTGAATATCAAACC |
| | | TTCCAAACGTAAATCCAACGAGGAATGGACTTTGGCATGCAA |
| | | ACATCTGTTGACCAATCTCAAATGGATAATTGCCTTCAGTAGT |
| | | TCAACGTGAACTAGTGTATTAAAAAAAAGAAACAGAAACTTT |
| | | ATTGGATTATAAAACTATTTATCAAGTTCAAATTAACATAGCG |
| | | ACGAAGAGACCAGCTGCGGCTAAGACTGAACTACCTAGTACC |
| | | GCTTGGGCACCGTTACCAGTTTCTGTACCTGTGCCAGTGGTAC |
| | | CAGTACCAGTACCGGTTCCAGTGCCAGTTCCTGTGCCTGTGCC |
| | | TGTGCCTGTGCCGGTTCCGGTTCCAGTGCCTGTGCCTGTGCCC |
| | | GTGCCTGTTGCAGTGGTATTAGTGAAACCTCCTGTGCCAGTTG |
| | | CAGTGGTATTAGTAAATCCTCCTGTTCCTGTGGTGTTTGTGAG |
| | | TCCTCCAGTTTCGGTCAAGTTTCCAGGAACACTAACATCAGGG |
| | | GTTGAAGTGATCTCTGGTGGCACCGTGGGGACTGTGACATTGA |
| | | CATCATTTGTGAAGATTGGCTCCAACTCAGTTGTAGCCTTAAC |
| | | AACGCTTAATGCGAGAGTTGCACCGATCAAACTTTTGAATTGC |
| | | ATTTTACTTTTGTTACTTCTAAAATGAGATGAGGAAAGAAAGA |
| | | AGAGAGAAGTGGAAGCACTGAAAGTGTGGTGTTATATCTGAA |
| | | AAATTCATTACCAATCAAAACGTCAGACGATGATATGTCTAA |
| | | GCCCGTGCAGAAACGTCTAGATCTTTTCAAACGTAAAGTACTT |
| | | CCCCTTTTGGCACATCGTGGACTTGCTATTCCAAATATAGACG |
| | | GGGACCTTTTTTAGAGTATCCCCGGGCGCCTCGAATTCTGGGG |
| | | TATTTTTTTGCTATAGCATGAATTGGCAATAGGGATTGGGGAC |
| | | AACGTGTTTGACAGAAGACGTGTGTGTCCTGCCAAAAAGGGG |
| | | TAAAGGTGCATTTGCCAAGGCCTGTGAATGATCTGAACACTA |
| | | GAGGAAAGCAAGAAGGCTGTGTCGTAGTCTGTATTGGCTGTG |
| | | TTGTCGCTGTGTCGGTTGCTTCAAAACTTTATTCGAGTCCGGT |
| | | ACGCGTCAATGGGTATTTTTCAAAAAGTTTCTAACTCCCTCAA |
| | | TCAACTTTGGTTTTGGCCGGATATGGCATGCCAGAAAAGGAA |
| | | GTTTTACTCCTGGCGATGATGTTTACAAATCAAGCTTAGAGGG |
| | | AGTAACCAATGCAGATAAGTTTGCGATGGCGCTGATCTTTATG |
| | | CTCTCAACACCTTCTCGACTATTCAGGGTCATTTCGTGGCTTTG |
| | | TATTTCGGGCACAACTGATCACCGAGGATCAATGAAATTTTCA |
| | | TGCACATCACTGATCCAGTTTCTGTCGAATTTGCAATTCCAGT |
| | | TGATTGCAGGACCCGCGTTCTGCCTACACATTTTCTCGTGATT |
| | | GTGGAAGTAATTCTAATTGACAGTCGATCACCACAATGACAA |
| | | TCTTAGTTGACCTTAGATTCCAGTGGAATGCAGTTGAATTGTC |
| | | TTTTCGTTTAATTAGAGGAGAGTAACGGACCAGGGCTCCTTTA |
| | | TTGTATATAATAATTATAATTTTTTTCACTATTTCACCTTTTCG |
| | | CTTGGAATATAAAATTCTAATTATAATTCAACAGGAAATATTG |
| | | TCCAAACCACATGAAGTTGTCATG |
| 48 | PpPMT1 gene | MCQIFLPQNVTRCSVSLLTMSKTSPQEVPENTTELKISKGELRPFI |
| | (protein) | VTSPSPQLSKSRSVTSTKEKLILASLFIFAMVIRFHNVAHPDSVVF |
| | | DEVHFGGFARKYILGTFFMDVHPPLAKLLFAGVGSLGGYDGEFE |
| | | FKKIGDEFPENVPYVLMRYLPSGMGVGTCIMLYLTLRASGCQPIV |
| | | CALTTALLIIENANVTISRFILLDSPMLFFIASTVYSFKKFQIQEPFT |
| | | FQWYKTLIATGVSLGLAASSKWVGLFTVAWIGLITIWDLWFIIGD |
| | | LTVSVKKIFGHFITRAVAFLVVPTLIYLTFFAIHLQVLTKEGDGGA |
| | | FMSSVFRSTLEGNAVPKQSLANVGLGSLVTIRHLNTRGGYLHSH |
| | | NHLYEGGSGQQQVTLYPHIDSNNQWIVQDYNATEEPTEFVPLKD |
| | | GVKIRLNHKLTSRRLHSHNLRPPVTEQDWQNEVSAYGHEGFGG |
| | | DANDDFVVEIAKDLSTTEEAKENVRAIQTVFRLRHAMTGCYLFS |
| | | HEVKLPKWAYEQQEVTCATQGIKPLSYWYVETNENPFLDKEVD |
| | | EIVSYPVPTFFQKVAELHARMWKINKGLTDHHVYESSPDSWPFL |
| | | LRGISYWSKNHSQIYFIGNAVTWWTVTASIALFSVFLVFSILRWQ |
| | | RGFGFSVDPTVFNFNVQMLHYILGWVLHYLPSFLMARQLFLHHY |
| | | LPSLYFGILALGHVFEIIHSYVFKNKQVVSYSIFVLFFAVALSFFQR |
| | | YSPLIYAGRWTKDQCNESKILKWDFDCNTFPSHTSQYEIWASPV |
| | | QTSTPKEGTHSESTVGEPDVEKLGETV |
| 49 | PpPMT4 | TAGTAAAGAAATCTTGCAGTTTAATTCTTCCTCTTGTGTTTTTA |
| | (DNA) | GCGATGAGACATCGGCACTCAGAGTTAAGTTTGCTTGCATCTG |
| | CDS 3168- | CTCTGATAACTTTTGCTGTGACTCTGTTGCAATGCTTTTGGTAA |
| | 5394 | CGGTCAATTCGTCTATGGTTTGTTGATACTTTGACTTTAAGGC |
| | | AGTAATATTGTCCTGTAGTTTATCATTATATGCTTCCAATGTTT |
| | | TGACCTTTGATGAAATGTTTTTTCGATTAACAGTTAGTTCATCG |
| | | AAGGAGAGCTCCAACTCTGATACTTGCATTCTTAAATTATTTA |
| | | TAATGGTATCCTTAACTTCTAGTGATTTCGAGTGGCTTGCCTG |
| | | GGCACTCTTAAGTTCTTTTCTCAACTGTGCTATGGATGGCTCA |
| | | AGCACTAGAATTTGTTTCTCTGAATCGAATAATTTTATTTCTAG |
| | | CTTCTGAGCAAGCTCACAGGCGCTTACTTTTTCGGAAGTTAGA |
| | | AACTTTGCTTCGTTATTCATGGCAGACAGTTCTATTCTTAATTG |
| | | CTTATTTTCTTTCCTAACTTCCAAAATCTCCGATTCCAGGGGTT |
| | | CATATCTACGGGAGGAAACCTGATTGCATGACTTTTCGAACGT |
| | | TTTTTGATCAGAAAGTTGACAGATTGTGCCATCAGTTGACGAG |
| | | ACAGCCTCAAACTGAGTTGCTTCCATGTTGCACAAATTATCAT |
| | | TGAATTCAGCCACTTCTTTCTCCAAATCTCCGTTTACCAGCTCC |
| | | TTCTTCTTTCCTGAAGCAATAGATGATGATCGATGAATATAGT |
| | | CCTCTTTCAATGGGTTTTGGATCTCTTTGTCCCATTGACAAGAA |
| | | GCTATGCTCCTTGAATCCTTCATTGACATTGGGTATGAAATTTT |
| | | GCTACCATCTACCTTTGCACTAATTTCTGTGGGCGAATTGTGT |
| | | GTTTTCAGTAGATCTTCAAGTGCTTTCTTTTCGTTTTCTATCTT |
| | | CATAAGAGATATTCTCAATTTATTTACGGTGTCAGTGGCAGAC |
| | | AAATTGTTTAATGAAACGGTATCCAGAGACTCCTGCTCCAGGT |
| | | ACTCTGACTGAGCTACAGGGAAGGGTTTCTTGTCGTGTATGGA |
| | | GAACTTCTGCTCAAGTTGGGCTTTCAAGGAATTCACTTGGTGA |
| | | TGCAAAAGCTCATTTTCCTCATTCAAAGAAGTATTCATATTTTT |
| | | AAGTTCCTCCAGCTCAAACGTACTGCGGCCTTCCAAAGTGCAG |
| | | ATTTTATCCTTAAGACTCAATTTCTCATTCTTCAAACTTTCCAT |
| | | CTCTCTATTGAGCGTTTCAACCTGGTCAGTTTTCAACTTGAGTT |
| | | CTTCTGAAAATTTGATACTTGAACTTTTAGCAAGGGAAGCTTC |
| | | ATCAAAAGTATCTTGTAGCTTGGTTTCTAAAGACCAGTTAGAA |
| | | TCAAGTAGCCTTTGCTGCTCTTGTTCCAACTTTTTGGAAAAAA |
| | | TTGCCTGGTGAGTGATCTTTTCAGCGAGATCCTCATTTTCTTTA |
| | | ACTTTTTGCTTCAATAGAGATTTGAGTTTTTGATTATCAGAGTT |
| | | CAGCTTCCGACATTCGACTAAAAGGTTGTCACTAATACCTGTT |
| | | AAAAAATCTATTTTGTTCGTCTTTTTTTTGCTTGGCGACTTTAG |
| | | AGGTAATGCAGGAAGGGAAGGATGAAATTCTGACTCCTGGTG |
| | | CCGATTTCTCAGCTTTCTCGGAAGTGGGGGTAGCTGAAATGGT |
| | | ACATTGTGGTTCGTATCACCAAGATCCATTTTTATGTCTTTGTC |
| | | CATTAGAAAATTCAAGAATCTTTCAAAAAAAATAGAAACAGA |
| | | AGATTTAGTAAACTTAGGTGAGGTGATATAAACCTAATTGCCT |
| | | GTTTTATTTTGATCATGTATGTAAATTGTGAAAGGTAAATACG |
| | | CGAAACTTATGTATGTATTGCAAAGATGCACAAGACACACAA |
| | | GGATTAATGGGCTATTTGCTCTACATTCGCAAAAAATAGCCAG |
| | | CATTTATTTTTTGAATGGATACTCAATAAGCCCATCCCTACGC |
| | | TTCCATATCTTTTTTTTCTTTTTGGTAGTAACATGCTCCACGAA |
| | | TACCTCTTCACAAGTAGATTTTTTAAATGAGCGGATAAAGCGG |
| | | GGGTCCCATAGTTCACTAGCAACTCCTAAGTCTTTGCAGCATC |
| | | TCATTAAAGCATTGCTCTTACAGCCTTCAGTAGCAGTAGGAAT |
| | | TCCCTTCTCTGAAAAAAAATCTTGCTCTCCGCGTGCAATAGAA |
| | | ACTAGTCGGCCCTGTACAATTAAAGCATACTCCCTGGTTAAAG |
| | | TACCTCCTCCGAACTTGCTCTTGTTGATCAAAGTTTCTGACCTT |
| | | GGGGCCAGTCCCCAGCCACCAGGGCCAAACGCTTTATTGAGG |
| | | ATACGACGATACTTAATCTCTGGAAGATAAAGTAGTCCATCTG |
| | | GTGTGATTTCGACATCTTCGTTGCTAATTGGTTGACATAATAT |
| | | GTTACTACTTTCATTACTGAAGGAGCAAATACCTAGTCCATGG |
| | | AACGAATCCGACCAATTGATTCCATCGCCACTTGTATTAGAGA |
| | | TTGGGGTGTCGTTTAACTGTGAAGTTCCAAACAAAATTGATAA |
| | | ACTGCTCTCGTTCTTAGCTTGGCCACTTTTTGGAGTCTCAATAG |
| | | TAGCGTTTTGGCTCTCGTGAATTTTCTGCACAGAGTCGGATGA |
| | | AGAAGGTGCAAATGCTTCTAGCATTGTAGAGTCGACCACATA |
| | | GAACCTTTTTAAAGAGTTATGAAAATAACTCTTGGTAGGGCCA |
| | | AATACAACCCGATATCGTCTTAGCATAAGAGCTGCTTCTTTGG |
| | | AATATCGTTTCTTGTAAGTAATTACGTGTTGGCTAAACACTTA |
| | | GAAGTCAGTCGCGCATGCGGCCAAAAACAGACTAGGGATAGA |
| | | AGATGAACTGACAAAAACATCAAGAAGGTGAAGACATTCATT |
| | | CTATGAAAACTAGTTTTTATATAAAATTATGGTCTGCATTTAG |
| | | AGAGCAATGATGTAATCAAACATCAATAAGTGCTTGTCGCAT |
| | | CAATATTTAATAGGTAATCATGGAGTATTCTAGTCTACCGCCT |
| | | TAAAAAAAGCTCACTCGATCTAGTGCAGCTTGATTGTGTACTT |
| | | CAATAGTATTCCAACGACCTTAACATCTTAACACCATGTAAAT |
| | | TTAAGATCCACGTATACGATACAATTTCTTTCAATATCAATTC |
| | | TCGTTCAAGCCAACTG [symbol] ATAAAATCAAGAAAGAGATCGAG |
| | | AAAAGTTTCTTTGAACACTGAAAAGGAGCTGAAAAATAGCCA |
| | | TATTTCTCTTGGAGATGAAAGATGGTACACTGTGGGTCTTCTC |
| | | TTGGTGACAATCACAGCTTTCTGTACTCGATTCTATGCTATCA |
| | | ACTATCCAGATGAGGTTGTTTTTGACGAAGTTCATTTCGGAAA |
| | | ATTTGCTAGCTACTATCTAGAGCGTACTTATTTTTTTGATCTGC |
| | | ACCCTCCGTTTGCCAAGCTCCTGATTGCGTTTGTCGGCTTTTTA |
| | | GCTGGGTACAATGGTGAGTTCAAGTTTACAACTATTGGTGAAT |
| | | CTTATATCAAAAACGAGGTTCCCTACGTAGTTTACAGATCATT |
| | | GAGCGCTGTGCAAGGATCTTTAACGGTGCCAATTGTTTATTTG |
| | | TGTCTCAAAGAATGCGGATATACAGTTTTGACTTGTGTTTTTG |
| | | GTGCATGTATCATATTGTTTGATGGGGCCCACGTTGCTGAGAC |
| | | TAGACTAATCTTGCTGGATGCCACGTTGATTTTTTTCGTTTCAT |
| | | TGTCCATCTATAGCTATATCAAATTCACAAAACAAAGATCAGA |
| | | ACCATTCGGCCAAAAGTGGTGGAAGTGGCTGTTCTTTACAGG |
| | | GGTGTCTTTATCTTGCGTCATAAGTACCAAGTATGTGGGGGTG |
| | | TTCACCTATCTTACAATAGGCTGTGGTGTCCTGTTTGACTTATG |
| | | GAGTTTACTGGATTATAAAAAGGGACATTCCTTGGCATATGTT |
| | | GGTAAACACTTTGCTGCACGATTTTTCCTTCTAATACTGGTCCC |
| | | TTTCTTGATATATCTCAATTGGTTTTATGTTCATTTCGCTATTC |
| | | TAAGCAAGTCTGGCCCAGGAGACAGTTTTATGAGCTCTGAATT |
| | | CCAGGAGACTCTCGGAGATTCTCCTCTTGCAGCTTTCGCAAAG |
| | | GAAGTTCACTTTAACGACATAATCACAATAAAGCATAAAGAG |
| | | ACTGATGCCATGTTGCACTCACACTTGGCAAACTACCCCCTCC |
| | | GTTACGAGGACGGGAGGGTATCATCTCAAGGTCAACAAGTTA |
| | | CAGCATACTCTGGAGAGGACCCAAACAATAATTGGCAGATTA |
| | | TTTCTCCCGAAGGACTTACTGGCGTTGTAACTCAGGGCGATGT |
| | | CGTTAGACTGAGACACGTTGGGACAGATGGCTATCTACTGAC |
| | | GCATGATGTTGCGTCTCCTTTCTATCCAACTAACGAGGAGTTT |
| | | ACTGTAGTGGGACAGGAGAAAGCTACTCAACGCTGGAACGAA |
| | | ACACTTTTTAGAATTGATCCCTATGACAAGAAGAAAACCCGTC |
| | | CTTTGAAGTCGAAAGCTTCATTTTTCAAACTCATTCATGTTCCT |
| | | ACGGTTGTGGCCATGTGGACTCATAATGACCAGCTTCTTCCTG |
| | | ATTGGGGTTTCAACCAACAAGAAGTCAATGGTAATAAGAAGC |
| | | TTGCTGATGAATCAAACTTATGGGTTGTAGACAATATCGTCGA |
| | | TATTGCAGAGGACGATCCAAGGAAACACTACGTTCCAAAGGA |
| | | AGTGAAAAATTTGCCATTTTTGACCAAGTGGTTGGAATTACAA |
| | | AGACTTATGTTTATTCAGAATAACAAGTTGAGCTCAGATCATC |
| | | CATTTGCGTCTGACCCTATATCTTGGCCTTTTTCACTTAGTGGG |
| | | GTTTCATTTTGGACAAACAACGAGTCACGCAAACAGATCTATT |
| | | TTGTCGGAAATATTCCTGGATGGTGGATGGAGGTTGCAGCATT |
| | | GGGATCCTTTCTAGGACTCGTGTTTGCAGATCAGTTCACGAGA |
| | | AGAAGAAACAGTCTTGTTTTGACCAATAGCGCCAGGTCTCGGT |
| | | TATACAATAATTTGGGGTTCTTCTTTGTAGGCTGGTGTTGTCAT |
| | | TACCTACCCTTTTTCCTAATGAGCCGTCAAAAATTTTTGCACC |
| | | ATTACTTACCTGCACATTTAATAGCAGCCATGTTCACTGCTGG |
| | | TTTCTTGGAATTTATTTTTACTGACAACAGAACTGAAGAATTC |
| | | AAGGATCAGAAAACTTCATGTGAACCTAACTCTAATTCTTCAA |
| | | AGCCGAAAGAGCAATTGATTCTGTGGTTAAGTTTCTCGTCCTT |
| | | TGTCGCTTTGCTACTAAGCATCATTGTTTGGACTTTCTTCTTTT |
| | | TTGCTCCTCTAACATATGGTAATACTGCGCTTTCGGCGGAGGA |
| | | GGTTCAGCAGCGACAATGGTTAGATATGAAGCTCCAATTCGC |
| | | CAAG [symbol] GAGTATACAATGTGTAGTTCAACGCAAAGGAAATT |
| | | CTAACTTTCTGTGCAATCTGGTGACAATTTCTAAATAACTATC |
| | | ACAATTGGAAGAAGAGATTATCCCAAATCTTATCAAAAAATC |
| | | GATGATTGCCAGTGCACAATTAGGCTTGAATTTTTCTTGCAGC |
| | | AACGAAGAGATTACTTCAGTGATGTTCATTAGCCTGAAATCTT |
| | | CACTTTCGTGGTCTATCGGATTAGGAATTAGACCTTGTTTCAT |
| | | CGGCAGGTCGTATATGTATTCCACTTCTGGTTGAATAAAATCT |
| | | TCGGGTGGTTTGTTTCTGAACATATATGAGATGGCTCCCACTG |
| | | GACTGATATATTGCGAAACATAGTCCTCATTCAACCCTGCCTC |
| | | CTCGTAACATTCTTTCAGGCAAGTTTGCAAAGTGCCATTAGGA |
| | | TATTCCAAGCCTCCTGCCACAGTATTATCTAACATACCGGGAA |
| | | ATGTTGGTTTGTGTCTGCTTCTCCTAGGTATCCAAAGTTGAAT |
| | | ACTGTTAGGATCGGCAGAATTTTGCAAATATCCATTGATATGA |
| | | ACTCCATAAGTAACAACTCCCAAAATATTAGAAAAAGCCCTTT |
| | | CCACCAACATGTACATCTTATGGTTATCGCAGTAAACTGCAAA |
| | | AAGCTCATTTCTCCAACCGCTAAGGGTTTCAAAGAGACGCTGA |
| | | TCTCTCCAACGCTGAGCTATCTTTGCAAACATCTGCGTTCTTTT |
| | | ATTTTCGGTATCCAGACTAGGAATTATCTTGACTTCGTGTTTTT |
| | | CATTATTTACTATCACAGCCTGTGTTTCGAACTCAAATTGTTTT |
| | | GCCACCTTGGGAATTATATACCCTAGTAAGATCCCATCATGCG |
| | | ATAAGAATTTATACACAGATACTTCAAATTCATGAAAAGATG |
| | | GCTCATCTTTATGAGGAACAGAATCAACAGATCTGACTAGATC |
| | | AATATATGGCATTGGTTGATTTTATTCAATGGTTATCTATCTCA |
| | | AACATGCTATAAAAATAAGGTAATTCCTTTATGGTGTTAGGGT |
| | | GTTATAGTTTTTGCGTAGAAAATAATTGTCATCATTTTTGGGC |
| | | AACCTATGAAACAACTACTCAGAGAAGTTGAGACATCTCTTTT |
| | | GACAAATGAAACCGAAATATCCCCTGCCCTTAAGCTATTAATT |
| | | ACTCAGTTAAATAAATCAACCCATGAAGATAAATCAACAGAA |
| | | AGAAAAACGTTTTGGCTAGCATTAGACAATTTAAGGCAAAAA |
| | | ATCGGTCTACAATCCCAATCACATGTCCTTTTCTTTCTACATCT |
| | | TTTTGAAGAGCTAGCTCCAACTTTAGAAAATGAGAAAATATTT |
| | | TTAACCTGGATTACTTCTTTTTTGAAGTTAGCAATTAATAGTGC |
| | | AGGGGTACCACATTGTGTGGTGAACGAGTCAAGGAGAATTAT |
| | | AATGAATTTATTATTGCCCTCAAAAGCTACAAACACCGAATAC |
| | | AATTTGTTAAAGAATTCTGCTGCAGGCATTCAATTACTTGTGC |
| | | AAGTGTATTTGCTAAAAACTGATTTAGTTGTTGATTCCACTTCT |
| | | AGTAGTCCCCAGGAGTATGAAGAGAGGGTTAGATTCATAAAG |
| | | AAAAACTGCAGGGATTTACTACAAGGTCTTGATTTAAATAATC |
| | | AAGTACTAGAGGCTATCAGCAAAGAATTTACGGATCCTCACT |
| | | ACCGCTTCGAGTGCTTCGTACTTTTGTCCTCATTAATGTCGTCA |
| | | TCAGCCTTGTTGTACCAGATAATGCAAACAACTTTGTGGCATA |
| | | ATATACTTTTGTCTATATTGATAGATAAAAGTAACAGTGTGGT |
| | | TGAGTCAGGAATCAAGGTTCTCAGTATGGTTTTGCCCCACGTC |
| | | TGTGATGTAATAGCGGATTATCTACCGACCATTATGGCGATTT |
| | | TAAGTAAAGGTCTGGGGGGTGTTGAAATTGATGATGAGTCAC |
| | | CATTACCATCAAATTGGAAAGTATTGAATGATCAGGATCCTGA |
| | | AATTATTGGTCCAGCATTTGTTAGCTATAAACAACTGTTCACT |
| | | GTATTATACGGCCTGTTCCCTCTTAGTTTAACATCATTTATTCG |
| | | CAGTCCATCTACATATATCGACTCTAACAAGATTATAGACGAT |
| | | CTCAAGCTTCAGTTGCTTGAAACTAAAGTGAAGTCAAAGTGTC |
| | | AGGACTTGCTAAAGTGTTTTATTGTTCATCCAAATTATTTTATA |
| | | TATTCTTCCCAGGAGGAAGAAATTTTTGATACTTCAAGGTGGG |
| | | ACAAAATGCACTCCCCGAACGAGATAGCAGCATTTTGTTATCA |
| | | ATTGGAATTCCGTGGGACATCGAAGGAGAATGCCTTTGATAT |
| | | GAGGGTAGATGACCTTTTGGAAGGTCATCGATATCTATATTTG |
| | | AAAGATATGAAGGATGCGCAGAAAGAGAGGGCTAAAAAATG |
| | | TGAAAATTCTATTATCTCACTCGAAAGTTCATCTGATAGTAAG |
| | | TCAGTTTCACAATACGACGAAGACTCGACGAAAGAAACCACT |
| | | TGCAGGCATGTTTCGTTTTATTTAAGAGAGATCCTTTTGGCAA |
| | | AAAATGAATTGGACTTCACGCTACATATCAATCAGGTACTTGG |
| | | AGCCGAGTGTGAGCTTTTGAAAAAAAAATTGAACGAAATGGA |
| | | TACCCTACGAGATCAAAACAGGTTTTTAGCTGACATAAACGA |
| | | AGGTTTACGAATACAGCAATCTAAGGCGAGTGAGCAAATTAC |
| | | GGAATTGCTCAAAGAAAAAGAGCGTTCTCAAAATGATTTCAA |
| | | CTCTCTGGTTACTCATATGCTTAAACAATCTAACGAATTAAAA |
| | | GAAAGGGAGTCGAAACTAGTCGAGATTCATCAATCAAATGAT |
| | | GCAGAGATAGGAGATTTAAATTATAGGTTGGAAAAACTGTGC |
| | | AACCTTATACAACCCAAAGAATTAGAAGTGGAACTGCTCAAG |
| | | AAGAAGTTGCGTGTAGCATCGATCCTTTTTTCGCAAGATAAAT |
| | | CAAAATCTTCAAGCAAGACATCTCTAGCACATTTGCACCAGGC |
| | | AGGCGACGCAACT |
| 50 | PpPMT4 | MIKSRKRSRKVSLNTEKELKNSHISLGDERWYTVGLLLVTITAFC |
| | (protein) | TRFYAINYPDEVVFDEVHFGKFASYYLERTYFFDLHPPFAKLLIA |
| | | FVGFLAGYNGEFKFTTIGESYIKNEVPYVVYRSLSAVQGSLTVPIV |
| | | YLCLKECGYTVLTCVFGACIILFDGAHVAETRLILLDATLIFFVSL |
| | | SIYSYIKFTKQRSEPFGQKWWKWLFFTGVSLSCVISTKYVGVFTY |
| | | LTIGCGVLFDLWSLLDYKKGHSLAYVGKHFAARFFLLILVPFLIY |
| | | LNWFYVHFAILSKSGPGDSFMSSEFQETLGDSPLAAFAKEVHFND |
| | | IITIKHKETDAMLHSHLANYPLRYEDGRVSSQGQQVTAYSGEDP |
| | | NNNWQIISPEGLTGVVTQGDVVRLRHVGTDGYLLTHDVASPFYP |
| | | TNEEFTVVGQEKATQRWNETLFRIDPYDKKKTRPLKSKASFFKLI |
| | | HVPTVVAMWTHNDQLLPDWGFNQQEVNGNKKLADESNLWVV |
| | | DNIVDIAEDDPRKHYVPKEVKNLPFLTKWLELQRLMFIQNNKLSS |
| | | DHPFASDPISWPFSLSGVSFWTNNESRKQIYFVGNIPGWWMEVA |
| | | ALGSFLGLVFADQFTRRRNSLVLTNSARSRLYNNLGFFFVGWCC |
| | | HYLPFFLMSRQKFLHHYLPAHLIAAMFTAGFLEFIFTDNRTEEFK |
| | | DQKTSCEPNSNSSKPKEQLILWLSFSSFVALLLSIIVWTFFFFAPLT |
| | | YGNTALSAEEVQQRQWLDMKLQFAK |
| 51 | anti-DKK1 | ACGATGGTCGCTTGGTGGTCTTTGTTTCTGTACGGTCTTCAGGT |
| | Heavy chain | CGCTGCACCTGCTTTGGCTGAGGTTCAGTTGGTTCAATCTGGT |
| | (VH + | GCTGAGGTTAAGAAACCTGGTGCTTCCGTTAAGGTTTCCTGTA |
| | IgG2m4) (α- | AGGCTTCCGGTTACACTTTCACTGACTACTACATCCACTGGGT |
| | amylase | TAGACAAGCTCCAGGTCAAGGATTGGAATGGATGGGATGGAT |
| | encoding | TCACTCTAACTCCGGTGCTACTACTTACGCTCAGAAGTTCCAG |
| | sequences | GCTAGAGTTACTATGTCCAGAGACACTTCTTCTTCCACTGCTT |
| | underlined) | ACATGGAATTGTCCAGATTGGAATCCGATGACACTGCTATGTA |
| | (DNA) | CTTTTGTTCCAGAGAGGACTACTGGGGACAGGGAACTTTGGTT |
| | | ACTGTTTCCTCCGCTTCTACTAAAGGGCCCTCTGTTTTTCCATT |
| | | GGCTCCATGTTCTAGATCCACTTCCGAATCCACTGCTGCTTTG |
| | | GGATGTTTGGTTAAGGACTACTTCCCAGAGCCAGTTACTGTTT |
| | | CTTGGAACTCCGGTGCTTTGACTTCTGGTGTTCACACTTTCCCA |
| | | GCTGTTTTGCAATCTTCCGGTTTGTACTCCTTGTCCTCCGTTGT |
| | | TACTGTTACTTCCTCCAACTTCGGTACTCAGACTTACACTTGTA |
| | | ACGTTGACCACAAGCCATCCAACACTAAGGTTGACAAGACTG |
| | | TTGAGAGAAAGTGTTGTGTTGAGTGTCCACCATGTCCAGCTCC |
| | | ACCAGTTGCTGGTCCATCCGTTTTTTTGTTCCCACCAAAGCCA |
| | | AAGGACACTTTGATGATCTCCAGAACTCCAGAGGTTACATGTG |
| | | TTGTTGTTGACGTTTCCCAAGAGGACCCAGAGGTTCAATTCAA |
| | | CTGGTACGTTGACGGTGTTGAAGTTCACAACGCTAAGACTAA |
| | | GCCAAGAGAAGAGCAGTTCAACTCCACTTTCAGAGTTGTTTCC |
| | | GTTTTGACTGTTTTGCACCAGGATTGGTTGAACGGTAAAGAAT |
| | | ACAAGTGTAAGGTTTCCAACAAGGGATTGCCATCCTCCATCGA |
| | | AAAGACTATCTCCAAGACTAAGGGACAACCAAGAGAGCCACA |
| | | GGTTTACACTTTGCCACCATCCAGAGAAGAGATGACTAAGAA |
| | | CCAGGTTTCCTTGACTTGTTTGGTTAAAGGATTCTACCCATCC |
| | | GACATTGCTGTTGAGTGGGAATCTAACGGTCAACCAGAGAAC |
| | | AACTACAAGACTACTCCACCAATGTTGGATTCTGACGGTTCCT |
| | | TCTTCTTGTACTCCAAGTTGACTGTTGACAAGTCCAGATGGCA |
| | | ACAGGGTAACGTTTTCTCCTGTTCCGTTATGCATGAGGCTTTG |
| | | CACAACCACTACACTCAAAAGTCCTTGTCTTTGTCCCCTGGTA |
| | | AGTAA |
| 52 | anti-DKK1 | EVQLVQSGAEVKKPGASVKVSCKASGYTFTDYYIHWVRQAPGQ |
| | Heavy chain | GLEWMGWIHSNSGATTYAQKFQARVTMSRDTSSSTAYMELSRL |
| | (VH + | ESDDTAMYFCSREDYWGQGTLVTVSSASTKGPSVFPLAPCSRST |
| | IgG2m4) | SESTAALGCLVKDYFPEPVTVSWNSGALTSGVHTFPAVLQSSGL |
| | (protein) | YSLSSVVTVTSSNFGTQTYTCNVDHKPSNTKVDKTVERKCCVEC |
| | | PPCPAPPVAGPSVFLFPPKPKDTLMISRTPEVTCVVVDVSQEDPEV |
| | | QFNWYVDGVEVHNAKTKPREEQFNSTFRVVSVLTVLHQDWLN |
| | | GKEYKCKVSNKGLPSSIEKTISKTKGQPREPQVYTLPPSREEMTK |
| | | NQVSLTCLVKGFYPSDIAVEWESNGQPENNYKTTPPMLDSDGSF |
| | | FLYSKLTVDKSRWQQGNVFSCSVMHEALHNHYTQKSLSLSPGK |
| 53 | anti-DKK1 | ACGATGGTCGCTTGGTGGTCTTTGTTTCTGTACGGTCTTCAGGT |
| | Light chain | CGCTGCACCTGCTTTGGCTCAGTCCGTTTTGACACAACCACCA |
| | (VL + lambda | TCTGTTTCTGGTGCTCCAGGACAGAGAGTTACTATCTCCTGTA |
| | constant | CTGGTTCCTCTTCCAACATTGGTGCTGGTTACGATGTTCACTG |
| | regions) (α- | GTATCAACAGTTGCCAGGTACTGCTCCAAAGTTGTTGATCTAC |
| | amylase | GGTTACTCCAACAGACCATCTGGTGTTCCAGACAGATTCTCTG |
| | encoding | GTTCTAAGTCTGGTGCTTCTGCTTCCTTGGCTATCACTGGATTG |
| | sequences | AGACCAGATGACGAGGCTGACTACTACTGTCAATCCTACGAC |
| | underlined) | AACTCCTTGTCCTCTTACGTTTTCGGTGGTGGTACTCAGTTGAC |
| | (DNA) | TGTTTTGTCCCAGCCAAAGGCTAATCCAACTGTTACTTTGTTCC |
| | | CACCATCTTCCGAAGAACTGCAGGCTAATAAGGCTACTTTGGT |
| | | TTGTTTGATCTCCGACTTCTACCCAGGTGCTGTTACTGTTGCTT |
| | | GGAAGGCTGATGGTTCTCCAGTTAAGGCTGGTGTTGAGACTAC |
| | | TAAGCCATCCAAGCAGTCCAATAACAAGTACGCTGCTAGCTCT |
| | | TACTTGTCCTTGACACCAGAACAATGGAAGTCCCACAGATCCT |
| | | ACTCTTGTCAGGTTACACACGAGGGTTCTACTGTTGAAAAGAC |
| | | TGTTGCTCCAACTGAGTGTTCCTAA |
| 54 | anti-DKK1 | QSVLTQPPSVSGAPGQRVTISCTGSSSNIGAGYDVHWYQQLPGTA |
| | Light chain | PKLLIYGYSNRPSGVPDRFSGSKSGASASLAITGLRPDDEADYYC |
| | (VL + lambda | QSYDNSLSSYVFGGGTQLTVLSQPKANPTVTLFPPSSEELQANKA |
| | constant | TLVCLISDFYPGAVTVAWKADGSPVKAGVETTKPSKQSNNKYA |
| | regions) | ASSYLSLTPEQWKSHRSYSCQVTHEGSTVEKTVAPTECS |
| | (protein) | |
| 55 | Human BiP | GAGGAAGAGGACAAGAAAGAGGATGTTGGTACTGTTGTCGGT |
| | (DNA) | ATCGACTTGGGTACTACCTACTCCTGTGTCGGTGTTTTCAAGA |
| | | ACGGTAGAGTGGAGATTATCGCCAACGACCAGGGTAACAGAA |
| | | TTACTCCATCCTACGTTGCTTTTACCCCAGAAGGAGAGAGATT |
| | | GATCGGAGACGCTGCTAAGAACCAATTGACCTCCAACCCAGA |
| | | GAACACTGTTTTCGACGCCAAGAGACTGATTGGTAGAACTTG |
| | | GAACGACCCATCCGTTCAACAAGACATCAAGTTCTTGCCCTTC |
| | | AAGGTCGTCGAGAAGAAAACCAAGCCATACATCCAGGTTGAC |
| | | ATCGGTGGTGGTCAAACTAAGACTTTCGCTCCAGAGGAAATCT |
| | | CCGCTATGGTCCTGACTAAGATGAAAGAGACTGCCGAGGCTT |
| | | ACTTGGGTAAAAAGGTTACCCACGCTGTTGTTACTGTTCCAGC |
| | | TTACTTCAACGACGCTCAGAGACAAGCTACTAAGGACGCTGG |
| | | TACTATCGCTGGACTGAACGTGATGAGAATCATCAACGAGCC |
| | | AACTGCTGCTGCTATTGCCTACGGATTGGACAAGAGAGAGGG |
| | | AGAGAAGAACATCTTGGTTTTCGACTTGGGTGGTGGTACTTTC |
| | | GACGTTTCCTTGTTGACCATCGACAACGGTGTTTTCGAAGTTG |
| | | TTGCTACCAACGGTGATACTCACTTGGGTGGAGAGGACTTCGA |
| | | TCAGAGAGTGATGGAACACTTCATCAAGCTGTACAAGAAGAA |
| | | AACCGGAAAGGACGTTAGAAAGGACAACAGAGCCGTTCAGA |
| | | AGTTGAGAAGAGAGGTTGAGAAGGCTAAGGCTTTGTCCTCCC |
| | | AACACCAAGCTAGAATCGAGATCGAATCCTTCTACGAGGGTG |
| | | AAGATTTCTCCGAGACCTTGACTAGAGCCAAGTTCGAAGAGC |
| | | TGAACATGGACCTGTTCAGATCCACTATGAAGCCAGTTCAGA |
| | | AGGTTTTGGAGGATTCCGACTTGAAGAAGTCCGACATCGACG |
| | | AGATTGTTTTGGTTGGTGGTTCCACCAGAATCCCAAAGATCCA |
| | | GCAGCTGGTCAAAGAGTTCTTCAACGGTAAAGAGCCATCCAG |
| | | AGGTATTAACCCAGATGAGGCTGTTGCTTACGGTGCTGCTGTT |
| | | CAAGCTGGTGTTTTGTCTGGTGACCAGGACACTGGTGACTTGG |
| | | TTTTGTTGCATGTTTGCCCATTGACTTTGGGTATCGAGACTGTT |
| | | GGTGGTGTTATGACCAAGTTGATCCCATCCAACACTGTTGTTC |
| | | CCACCAAGAACTCCCAAATTTTCTCCACTGCTTCCGACAACCA |
| | | GCCAACCGTTACTATTAAGGTCTACGAAGGTGAAAGACCATT |
| | | GACCAAGGACAACCACTTGTTGGGAACTTTCGACTTGACTGGT |
| | | ATTCCACCTGCTCCAAGAGGTGTTCCACAAATCGAGGTTACCT |
| | | TCGAGATCGACGTCAACGGTATCTTGAGAGTTACTGCCGAGG |
| | | ATAAGGGAACCGGTAACAAGAACAAGATCACCATCACCAACG |
| | | ACCAAAACAGATTGACCCCCGAAGAGATCGAAAGAATGGTCA |
| | | ACGATGCTGAGAAGTTCGCCGAAGAGGATAAGAAGCTGAAAG |
| | | AGAGAATCGACACCAGAAACGAGTTGGAATCCTACGCTTACT |
| | | CCTTGAAGAACCAGATCGGTGACAAAGAAAAGTTGGGTGGAA |
| | | AGCTGTCATCCGAAGATAAAGAAACTATGGAAAAGGCCGTCG |
| | | AAGAAAAGATTGAGTGGCTGGAATCTCACCAAGATGCTGACA |
| | | TCGAGGACTTCAAGGCCAAGAAGAAAGAGTTGGAAGAGATCG |
| | | TCCAGCCAATCATTTCTAAGTTGTACGGTTCTGCTGGTCCACC |
| | | ACCAACTGGTGAAGAAGATACTGCCGAGCACGACGAGTTGTA |
| | | G |
| 56 | Human BiP | EEEDKKEDVGTVVGIDLGTTYSCVGVFKNGRVEIIANDQGNRITP |
| | (protein) | SYVAFTPEGERLIGDAAKNQLTSNPENTVFDAKRLIGRTWNDPSV |
| | ATPase | QQDIKFLPFKVVEKKTKPYIQVDIGGGQTKTFAPEEISAMVLTKM |
| | domain | KETAEAYLGKKVTHAVVTVPAYFNDAQRQATKDAGTIAGLNV |
| | underlined | MRIINEPTAAAIAYGLDKREGEKNILVFDLGGGTFDVSLLTIDNG |
| | | VFEVVATNGDTHLGGEDFDQRVMEHFIKLYKKKTGKDVRKDNR |
| | | AVQKLRREVEKAKALSSQHQARIEIESFYEGEDFSETLTRAKFEE |
| | | LNMDLFRSTMKPVQKVLEDSDLKKSDIDEIVLVGGSTRIPKIQQL |
| | | VKEFFNGKEPSRGINPDEAVAYGAAVQAGVLSGDQDTGDLVLL |
| | | HVCPLTLGIETVGGVMTKLIPSNTVVPTKNSQIFSTASDNQPTVTI |
| | | KVYEGERPLTKDNHLLGTFDLTGIPPAPRGVPQIEVTFEIDVNGIL |
| | | RVTAEDKGTGNKNKITITNDQNRLTPEEIERMVNDAEKFAEEDK |
| | | KLKERIDTRNELESYAYSLKNQIGDKEKLGGKLSSEDKETMEKA |
| | | VEEKIEWLESHQDADIEDFKAKKKELEEIVQPIISKLYGSAGPPPT |
| | | GEEDTAEHDEL |
| 57 | Chimeric BiP | GACGATGTCGAATCTTATGGAACAGTGATTGGTATCGATTTGG |
| | (DNA) | GTACCACGTACTCTTGTGTCGGTGTGATGAAGTCGGGTCGTGT |
| | | AGAAATTCTTGCTAATGACCAAGGTAACAGAATCACTCCTTCC |
| | | TACGTTAGTTTCACTGAAGACGAGAGACTGGTTGGTGATGCTG |
| | | CTAAGAACTTAGCTGCTTCTAACCCAAAAAACACCATCTTTGA |
| | | TATTAAGAGATTGATCGGTATGAAGTATGATGCCCCAGAGGT |
| | | CCAAAGAGACTTGAAGCGTCTTCCTTACACTGTCAAGAGCAA |
| | | GAACGGCCAACCTGTCGTTTCTGTCGAGTACAAGGGTGAGGA |
| | | GAAGTCTTTCACTCCTGAGGAGATTTCCGCCATGGTCTTGGGT |
| | | AAGATGAAGTTGATCGCTGAGGACTACTTAGGAAAGAAAGTC |
| | | ACTCATGCTGTCGTTACCGTTCCAGCCTACTTCAACGACGCTC |
| | | AACGTCAAGCCACTAAGGATGCCGGTCTCATCGCCGGTTTGAC |
| | | TGTTCTGAGAATTGTGAACGAGCCTACCGCCGCTGCCCTTGCT |
| | | TACGGTTTGGACAAGACTGGTGAGGAAAGACAGATCATCGTC |
| | | TACGACTTGGGTGGAGGAACCTTCGATGTTTCTCTGCTTTCTA |
| | | TTGAGGGTGGTGCTTTCGAGGTTCTTGCTACCGCCGGTGACAC |
| | | CCACTTGGGTGGTGAGGACTTTGACTACAGAGTTGTTCGCCAC |
| | | TTCGTTAAGATTTTCAAGAAGAAGCATAACATTGACATCAGCA |
| | | ACAATGATAAGGCTTTAGGTAAGCTGAAGAGAGAGGTCGAAA |
| | | AGGCCAAGCGTACTTTGTCTTCCCAGATGACTACCAGAATTGA |
| | | GATTGACTCTTTCGTCGACGGTATCGACTTCTCTGAGCAACTG |
| | | TCTAGAGCTAAGTTTGAGGAGATCAACATTGAATTATTCAAGA |
| | | AGACACTGAAACCAGTTGAACAAGTCCTCAAAGACGCTGGTG |
| | | TCAAGAAATCTGAAATTGATGACATTGTCTTGGTTGGTGGTTC |
| | | TACCAGAATTCCAAAGGTTCAACAATTATTGGAGGATTACTTT |
| | | GACGGAAAGAAGGCTTCTAAGGGAATTAACCCAGATGAAGCT |
| | | GTCGCATACGGTGCTGCTGTTCAGGCTGGTGTTTTGTCTGGTG |
| | | ATCAAGATACAGGTGACCTGGTACTGCTTGATGTATGTCCCCT |
| | | TACACTTGGTATTGAAACTGTGGGAGGTGTCATGACCAAACTG |
| | | ATTCCAAGGAACACAGTGGTGCCTACCAAGAAGTCTCAGATC |
| | | TTTTCTACAGCTTCTGATAATCAACCAACTGTTACAATCAAGG |
| | | TCTATGAAGGTGAAAGACCCCTGACAAAAGACAATCATCTTC |
| | | TGGGTACATTTGATCTGACTGGAATTCCTCCTGCTCCTCGTGG |
| | | GGTCCCACAGATTGAAGTCACCTTTGAGATAGATGTGAATGGT |
| | | ATTCTTCGAGTGACAGCTGAAGACAAGGGTACAGGGAACAAA |
| | | AATAAGATCACAATCACCAATGACCAGAATCGCCTGACACCT |
| | | GAAGAAATCGAAAGGATGGTTAATGATGCTGAGAAGTTTGCT |
| | | GAGGAAGACAAAAAGCTCAAGGAGCGCATTGATACTAGAAAT |
| | | GAGTTGGAAAGCTATGCCTATTCTCTAAAGAATCAGATTGGA |
| | | GATAAAGAAAAGCTGGGAGGTAAACTTTCCTCTGAAGATAAG |
| | | GAGACCATGGAAAAAGCTGTAGAAGAAAAGATTGAATGGCTG |
| | | GAAAGCCACCAAGATGCTGACATTGAAGACTTCAAAGCTAAG |
| | | AAGAAGGAACTGGAAGAAATTGTTCAACCAATTATCAGCAAA |
| | | CTCTATGGAAGTGCAGGCCCTCCCCCAACTGGTGAAGAGGAT |
| | | ACAGCAGAACATGATGAGTTGTAG |
| 58 | Chimeric BiP | DDVESYGTVIGIDLGTTYSCVGVMKSGRVEILANDQGNRITPSYV |
| | (protein) | SFTEDERLVGDAAKNLAASNPKNTIFDIKRLIGMKYDAPEVQRDL |
| | ATPase | KRLPYTVKSKNGQPVVSVEYKGEEKSFTPEEISAMVLGKMKLIA |
| | domain | EDYLGKKVTHAVVTVPAYFNDAQRQATKDAGLIAGLTVLRIVN |
| | underlined | EPTAAALAYGLDKTGEERQIIVYDLGGGTFDVSLLSIEGGAFEVL |
| | | ATAGDTHLGGEDFDYRVVRHFVKIFKKKHNIDISNNDKALGKLK |
| | | REVEKAKRTLSSQMTTRIEIDSFVDGIDFSEQLSRAKFEEINIELFK |
| | | KTLKPVEQVLKDAGVKKSEIDDIVLVGGSTRIPKVQQLLEDYFDG |
| | | KKASKGINPDEAVAYGAAVQAGVLSGDQDTGDLVLLDVCPLTL |
| | | GIETVGGVMTKLIPRNTVVPTKKSQIFSTASDNQPTVTIKVYEGER |
| | | PLTKDNHLLGTFDLTGIPPAPRGVPQIEVTFEIDVNGILRVTAEDK |
| | | GTGNKNKITITNDQNRLTPEEIERMVNDAEKFAEEDKKLKERIDT |
| | | RNELESYAYSLKNQIGDKEKLGGKLSSEDKETMEKAVEEKIEWL |
| | | ESHQDADIEDFKAKKKELEEIVQPIISKLYGSAGPPPTGEEDTAEH |
| | | DEL |
| 59 | PpPDI1 | AACACGAACACTGTAAATAGAATAAAAGAAAACTTGGATAGT |
| | promoter | AGAACTTCAATGTAGTGTTTCTATTGTCTTACGCGGCT |
| | | CTTTAGATTGCAATCCCCAGAATGGAATCGTCCATCTTTCTCA |
| | | ACCCACTCAAAGATAATCTACCAGACATACCTACGCC |
| | | CTCCATCCCAGCACCACGTCGCGATCACCCCTAAAACTTCAAT |
| | | AATTGAACACGTACTGATTTCCAAACCTTCTTCTTCT |
| | | TCCTATCTATAAGA |
| 60 | PpPMR1 | ATGACAGCTAATGAAAATCCTTTTGAGAATGAGCTGACAGGA |
| | | TCTTCTGAATCTGCCCCCCCTGCATTGGAATCGAAGACTGGAG |
| | | AGTCTCTTAAGTATTGCAAATATACCGTGGATCAGGTCATAGA |
| | | AGAGTTTCAAACGGATGGTCTCAAAGGATTGTGCAATTCCCA |
| | | GGACATCGTATATCGGAGGTCTGTTCATGGGCCAAATGAAAT |
| | | GGAAGTCGAAGAGGAAGAGAGTCTTTTTTCGAAATTCTTGTCA |
| | | AGTTTCTACAGCGATCCATTGATTCTGTTACTGATGGGTTCCG |
| | | CTGTGATTAGCTTTTTGATGTCTAACATTGATGATGCGATATCT |
| | | ATCACTATGGCAATTACGATCGTTGTCACAGTTGGATTTGTTC |
| | | AAGAGTATCGATCCGAGAAATCATTGGAGGCATTGAACAAGT |
| | | TAGTCCCTGCCGAAGCTCATCTAACTAGGAATGGGAACACTG |
| | | AAACTGTTCTTGCTGCCAACCTAGTCCCAGGAGACTTGGTGGA |
| | | TTTTTCGGTTGGTGACAGAATTCCGGCTGATGTGAGAATTATT |
| | | CACGCTTCCCACTTGAGTATCGACGAGAGCAACCTAACTGGTG |
| | | AAAATGAACCAGTTTCTAAAGACAGCAAACCTGTTGAAAGTG |
| | | ATGACCCAAACATTCCCTTGAACAGCCGTTCATGTATTGGGTA |
| | | TATGGGCACTTTAGTTCGTGATGGTAATGGCAAAGGTATTGTC |
| | | ATCGGAACAGCCAAAAACACAGCTTTTGGCTCTGTTTTCGAAA |
| | | TGATGAGCTCTATTGAGAAACCAAAGACTCCTCTTCAACAGGC |
| | | TATGGATAAACTTGGTAAGGATTTGTCTGCTTTTTCCTTCGGA |
| | | ATCATCGGCCTTATTTGCTTGGTTGGTGTTTTTCAAGGTAGACC |
| | | CTGGTTGGAAATGTTCCAGATCTCTGTATCCTTGGCTGTTGCT |
| | | GCGATTCCAGAAGGTCTTCCTATTATTGTGACTGTGACTCTTG |
| | | CTCTTGGTGTGTTGCGTATGGCTAAACAGAGGGCCATCGTCAA |
| | | AAGACTGCCTAGTGTTGAAACTTTGGGATCCGTCAATGTTATC |
| | | TGTAGTGATAAGACGGGAACATTGACCCAAAATCATATGACC |
| | | GTTAACAGATTATGGACTGTGGATATGGGCGATGAATTCTTGA |
| | | AAATTGAACAAGGGGAGTCCTATGCCAATTATCTCAAACCCG |
| | | ATACGCTAAAAGTTCTGCAAACTGGTAATATAGTCAACAATG |
| | | CCAAATATTCAAATGAAAAGGAAAAATACCTCGGAAACCCAA |
| | | CTGATATTGCAATTATTGAATCTTTAGAAAAATTTGATTTGCA |
| | | GGACATTAGAGCAACAAAGGAAAGAATGTTGGAGATTCCATT |
| | | TTCTTCGTCCAAGAAATATCAGGCCGTCAGTGTTCACTCTGGA |
| | | GACAAAAGCAAATCTGAAATTTTTGTTAAAGGCGCTCTGAAC |
| | | AAAGTTTTGGAAAGATGTTCAAGATATTACAATGCTGAAGGT |
| | | ATCGCCACTCCACTCACAGATGAAATTAGAAGAAAATCCTTG |
| | | CAAATGGCCGATACGTTAGCATCTTCAGGATTGAGAATACTGT |
| | | CGTTTGCTTACGACAAAGGCAATTTTGAAGAAACTGGCGATG |
| | | GACCATCGGATATGATCTTTTGTGGTCTTTTAGGTATGAACGA |
| | | TCCTCCTAGACCATCTGTAAGTAAATCAATTTTGAAATTCATG |
| | | AGAGGTGGGGTTCACATTATTATGATTACAGGAGATTCAGAA |
| | | TCCACGGCCGTAGCCGTTGCCAAACAGGTCGGAATGGTAATT |
| | | GACAATTCAAAATATGCTGTCCTCAGTGGAGACGATATAGAT |
| | | GCTATGAGTACAGAGCAACTGTCTCAGGCGATCTCACATTGTT |
| | | CTGTATTTGCCCGGACTACTCCAAAACATAAGGTGTCCATTGT |
| | | AAGAGCACTACAGGCCAGAGGAGATATTGTTGCAATGACTGG |
| | | TGACGGTGTCAATGATGCCCCAGCTCTAAAACTGGCCGACATC |
| | | GGAATTGCCATGGGTAATATGGGGACCGATGTTGCCAAAGAG |
| | | GCAGCCGACATGGTTTTGACTGATGATGACTTTTCTACAATCT |
| | | TATCTGCAATCCAGGAGGGTAAAGGTATTTTCTACAACATCCA |
| | | GAACTTTTTAACGTTCCAACTTTCTACTTCAATTGCTGCTCTTT |
| | | CGTTAATTGCTCTGAGTACTGCTTTCAACCTGCCAAATCCATT |
| | | GAATGCCATGCAGATTTTGTGGATCAATATTATCATGGATGGA |
| | | CCTCCAGCTCAGTCTTTGGGTGTTGAGCCAGTTGATAAAGCTG |
| | | TGATGAACAAACCACCAAGAAAGCGAAATGATAAAATTCTGA |
| | | CAGGTAAGGTGATTCAAAGGGTAGTACAAAGTAGTTTTATCA |
| | | TTGTTTGTGGTACTCTGTACGTATACATGCATGAGATCAAAGA |
| | | TAATGAGGTCACAGCAAGAGACACTACGATGACCTTTACATG |
| | | CTTTGTATTCTTTGACATGTTCAACGCATTAACGACAAGACAC |
| | | CATTCTAAAAGTATTGCAGAACTTGGATGGAATAATACTATGT |
| | | TCAACTTTTCCGTTGCAGCTTCTATTTTGGGTCAACTAGGAGCT |
| | | ATTTACATTCCATTTTTGCAGTCTATTTTCCAGACTGAACCTCT |
| | | GAGCCTCAAAGATTTGGTCCATTTATTGTTGTTATCGAGTTCA |
| | | GTATGGATTGTAGACGAGCTTCGAAAACTCTACGTCAGGAGA |
| | | CGTGACGCATCCCCATACAATGGATACAGCATGGCTGTTTGA |
| 61 | PpPMR1 | MTANENPFENELTGSSESAPPALESKTGESLKYCKYTVDQVIEEF |
| | | QTDGLKGLCNSQDIVYRRSVHGPNEMEVEEEESLFSKFLSSFYSD |
| | | PLILLLMGSAVISFLMSNIDDAISITMAITIVVTVGFVQEYRSEKSL |
| | | EALNKLVPAEAHLTRNGNTETVLAANLVPGDLVDFSVGDRIPAD |
| | | VRIIHASHLSIDESNLTGENEPVSKDSKPVESDDPNIPLNSRSCIGY |
| | | MGTLVRDGNGKGIVIGTAKNTAFGSVFEMMSSIEKPKTPLQQAM |
| | | DKLGKDLSAFSFGIIGLICLVGVFQGRPWLEMFQISVSLAVAAIPE |
| | | GLPIIVTVTLALGVLRMAKQRAIVKRLPSVETLGSVNVICSDKTG |
| | | TLTQNHMTVNRLWTVDMGDEFLKIEQGESYANYLKPDTLKVLQ |
| | | TGNIVNNAKYSNEKEKYLGNPTDIAIIESLEKFDLQDIRATKERML |
| | | EIPFSSSKKYQAVSVHSGDKSKSEIFVKGALNKVLERCSRYYNAE |
| | | GIATPLTDEIRRKSLQMADTLASSGLRILSFAYDKGNFEETGDGPS |
| | | DMIFCGLLGMNDPPRPSVSKSILKFMRGGVHIIMITGDSESTAVA |
| | | VAKQVGMVIDNSKYAVLSGDDIDAMSTEQLSQAISHCSVFARTT |
| | | PKHKVSIVRALQARGDIVAMTGDGVNDAPALKLADIGIAMGNM |
| | | GTDVAKEAADMVLTDDDFSTILSAIQEGKGIFYNIQNFLTFQLSTS |
| | | IAALSLIALSTAFNLPNPLNAMQILWINIIMDGPPAQSLGVEPVDK |
| | | AVMNKPPRKRNDKILTGKVIQRVVQSSFIIVCGTLYVYMHEIKDN |
| | | EVTARDTTMTFTCFVFFDMFNALTTRHHSKSIAELGWNNTMFNF |
| | | SVAASILGQLGAIYIPFLQSIFQTEPLSLKDLVHLLLLSSSVWIVDE |
| | | LRKLYVRRRDASPYNGYSMAV |
| 62 | Arabidopsis | ATGGGAAAGGGTTCCGAGGACCTGGTTAAGAAAGAATCCCTG |
| | Thaliana | AACTCCACTCCAGTTAACTCTGACACTTTCCCAGCTTGGGCTA |
| | AtECA1 | AGGATGTTGCTGAGTGCGAAGAGCACTTCGTTGTTTCCAGAGA |
| | (codon | GAAGGGTTTGTCCTCCGACGAAGTCTTGAAGAGACACCAAAT |
| | optimized for | CTACGGACTGAACGAGTTGGAAAAGCCAGAGGGAACCTCCAT |
| | Pichia | CTTCAAGCTGATCTTGGAGCAGTTCAACGACACCCTTGTCAGA |
| | pastoris) | ATTTTGTTGGCTGCCGCTGTTATTTCCTTCGTCCTGGCTTTTTTT |
| | | GATGGTGACGAGGGTGGTGAAATGGGTATCACTGCCTTCGTT |
| | | GAGCCTTTGGTCATCTTCCTGATCTTGATCGTTAACGCCATCGT |
| | | TGGTATCTGGCAAGAGACTAACGCTGAAAAGGCTTTGGAGGC |
| | | CTTGAAAGAGATTCAATCCCAGCAGGCTACCGTTATGAGAGA |
| | | TGGTACTAAGGTTTCCTCCTTGCCAGCTAAAGAATTGGTTCCA |
| | | GGTGACATCGTTGAGCTGAGAGTTGGTGATAAGGTTCCAGCC |
| | | GACATGAGAGTTGTTGCTTTGATCTCCTCCACCTTGAGAGTTG |
| | | AACAAGGTTCCCTGACTGGTGAATCTGAGGCTGTTTCCAAGAC |
| | | TACTAAGCACGTTGACGAGAACGCTGACATCCAGGGTAAAAA |
| | | GTGCATGGTTTTCGCCGGTACTACCGTTGTTAACGGTAACTGC |
| | | ATCTGTTTGGTCACTGACACTGGAATGAACACCGAGATCGGTA |
| | | GAGTTCACTCCCAAATCCAAGAAGCTGCTCAACACGAAGAGG |
| | | ACACCCCATTGAAGAAGAAGCTGAACGAGTTCGGAGAGGTCT |
| | | TGACCATGATCATCGGATTGATCTGTGCCCTGGTCTGGTTGAT |
| | | CAACGTCAAGTACTTCTTGTCCTGGGAATACGTTGATGGATGG |
| | | CCAAGAAACTTCAAGTTCTCCTTCGAGAAGTGCACCTACTACT |
| | | TCGAGATCGCTGTTGCTTTGGCTGTTGCTGCTATTCCAGAGGG |
| | | ATTGCCAGCTGTTATCACCACTTGCTTGGCCTTGGGTACTAGA |
| | | AAGATGGCTCAGAAGAACGCCCTTGTTAGAAAGTTGCCATCC |
| | | GTTGAGACTTTGGGTTGTACTACCGTCATCTGTTCCGACAAGA |
| | | CTGGTACTTTGACTACCAACCAGATGGCCGTTTCCAAATTGGT |
| | | TGCCATGGGTTCCAGAATCGGTACTCTGAGATCCTTCAACGTC |
| | | GAGGGAACTTCTTTTGACCCAAGAGATGGAAAGATTGAGGAC |
| | | TGGCCAATGGGTAGAATGGACGCCAACTTGCAGATGATTGCT |
| | | AAGATCGCCGCTATCTGTAACGACGCTAACGTTGAGCAATCC |
| | | GACCAACAGTTCGTTTCCAGAGGAATGCCAACTGAGGCTGCC |
| | | TTGAAGGTTTTGGTCGAGAAGATGGGTTTCCCAGAAGGATTG |
| | | AACGAGGCTTCTTCCGATGGTGACGTCTTGAGATGTTGCAGAC |
| | | TGTGGAGTGAGTTGGAGCAGAGAATCGCTACTTTGGAGTTCG |
| | | ACAGAGATAGAAAGTCCATGGGTGTCATGGTTGATTCTTCCTC |
| | | CGGTAACAAGTTGTTGTTGGTCAAAGGAGCAGTTGAAAACGT |
| | | TTTGGAGAGATCCACCCACATTCAATTGCTGGACGGTTCCAAG |
| | | AGAGAATTGGACCAGTACTCCAGAGACTTGATCTTGCAGTCCT |
| | | TGAGAGACATGTCCTTGTCCGCCTTGAGATGTTTGGGTTTCGC |
| | | TTACTCTGACGTTCCATCCGATTTCGCTACTTACGATGGTTCTG |
| | | AGGATCATCCAGCTCACCAACAGTTGCTGAACCCATCCAACTA |
| | | CTCCTCCATCGAATCCAACCTGATCTTCGTTGGTTTCGTCGGTC |
| | | TTAGAGACCCACCAAGAAAAGAAGTTAGACAGGCCATCGCTG |
| | | ATTGTAGAACCGCCGGTATCAGAGTTATGGTCATCACCGGAG |
| | | ATAACAAGTCCACTGCCGAGGCTATTTGTAGAGAGATCGGAG |
| | | TTTTCGAGGCTGACGAGGACATTTCTTCCAGATCCCTGACCGG |
| | | TATTGAGTTCATGGACGTCCAAGACCAGAAGAACCACTTGAG |
| | | ACAGACCGGTGGTTTGTTGTTCTCCAGAGCCGAACCAAAGCA |
| | | CAAGCAAGAGATTGTCAGACTGCTGAAAGAGGACGGAGAAGT |
| | | TGTTGCTATGACCGGTGATGGTGTTAATGACGCCCCAGCTTTG |
| | | AAGTTGGCTGACATCGGTGTTGCTATGGGAATTTCCGGTACTG |
| | | AAGTTGCTAAGGAAGCCTCCGATATGGTTTTGGCTGACGACA |
| | | ACTTTTCAACTATCGTTGCTGCTGTCGGAGAAGGTAGAAGTAT |
| | | CTACAACAACATGAAAGCCTTTATCAGATACATGATTTCCTCC |
| | | AACATCGGTGAAGTTGCCTCCATTTTCTTGACTGCTGCCTTGG |
| | | GTATTCCTGAGGGAATGATCCCAGTTCAGTTGTTGTGGGTTAA |
| | | CTTGGTTACTGACGGTCCACCTGCTACTGCTTTGGGTTTCAAC |
| | | CCACCAGACAAAGACATTATGAAGAAGCCACCAAGAAGATCC |
| | | GACGATTCCTTGATCACCGCCTGGATCTTGTTCAGATACATGG |
| | | TCATCGGTCTTTATGTTGGTGTTGCCACCGTCGGTGTTTTCATC |
| | | ATCTGGTACACCCACTCTTCCTTCATGGGTATTGACTTGTCTCA |
| | | AGATGGTCATTCTTTGGTTTCCTACTCCCAATTGGCTCATTGGG |
| | | GACAATGTTCTTCCTGGGAGGGTTTCAAGGTTTCCCCATTCAC |
| | | TGCTGGTTCCCAGACTTTCTCCTTCGATTCCAACCCATGTGACT |
| | | ACTTCCAGCAGGGAAAGATCAAGGCTTCCACCTTGTCTTTGTC |
| | | CGTTTTGGTCGCCATTGAGATGTTCAACTCCCTGAACGCTTTG |
| | | TCTGAGGACGGTTCCTTGGTTACTATGCCACCTTGGGTGAACC |
| | | CATGGTTGTTGTTGGCTATGGCTGTTTCCTTCGGATTGCACTTC |
| | | GTCATCCTGTACGTTCCATTCTTGGCCCAGGTTTTCGGTATTGT |
| | | TCCACTGTCCTTGAACGAGTGGTTGTTGGTCTTGGCCGTTTCTT |
| | | TGCCAGTTATCCTGATCGACGAGGTTTTGAAGTTCGTTGGTAG |
| | | ATGCACCTCTGGTTACAGATACTCCCCAAGAACTCTGTCCACC |
| | | AAGCAGAAAGAAGAGTAA |
| 63 | AtECA1 | MGKGSEDLVKKESLNSTPVNSDTFPAWAKDVAECEEHFVVSRE |
| | | KGLSSDEVLKRHQIYGLNELEKPEGTSIFKLILEQFNDTLVRILLA |
| | | AAVISFVLAFFDGDEGGEMGITAFVEPLVIFLILIVNAIVGIWQETN |
| | | AEKALEALKEIQSQQATVMRDGTKVSSLPAKELVPGDIVELRVG |
| | | DKVPADMRVVALISSTLRVEQGSLTGESEAVSKTTKHVDENADI |
| | | QGKKCMVFAGTTVVNGNCICLVTDTGMNTEIGRVHSQIQEAAQ |
| | | HEEDTPLKKKLNEFGEVLTMIIGLICALVWLINVKYFLSWEYVDG |
| | | WPRNFKFSFEKCTYYFEIAVALAVAAIPEGLPAVITTCLALGTRK |
| | | MAQKNALVRKLPSVETLGCTTVICSDKTGTLTTNQMAVSKLVA |
| | | MGSRIGTLRSFNVEGTSFDPRDGKIEDWPMGRMDANLQMIAKIA |
| | | AICNDANVEQSDQQFVSRGMPTEAALKVLVEKMGFPEGLNEASS |
| | | DGDVLRCCRLWSELEQRIATLEFDRDRKSMGVMVDSSSGNKLL |
| | | LVKGAVENVLERSTHIQLLDGSKRELDQYSRDLILQSLRDMSLSA |
| | | LRCLGFAYSDVPSDFATYDGSEDHPAHQQLLNPSNYSSIESNLIFV |
| | | GFVGLRDPPRKEVRQAIADCRTAGIRVMVITGDNKSTAEAICREI |
| | | GVFEADEDISSRSLTGIEFMDVQDQKNHLRQTGGLLFSRAEPKHK |
| | | QEIVRLLKEDGEVVAMTGDGVNDAPALKLADIGVAMGISGTEV |
| | | AKEASDMVLADDNFSTIVAAVGEGRSIYNNMKAFIRYMISSNIGE |
| | | VASIFLTAALGIPEGMIPVQLLWVNLVTDGPPATALGFNPPDKDI |
| | | MKKPPRRSDDSLITAWILFRYMVIGLYVGVATVGVFIIWYTHSSF |
| | | MGIDLSQDGHSLVSYSQLAHWGQCSSWEGFKVSPFTAGSQTFSF |
| | | DSNPCDYFQQGKIKASTLSLSVLVAIEMFNSLNALSEDGSLVTMP |
| | | PWVNPWLLLAMAVSFGLHFVILYVPFLAQVFGIVPLSLNEWLLV |
| | | LAVSLPVILIDEVLKFV |
| | | GRCTSGYRYSPRTLSTKQKEE |
| 64 | PpPMR1/UP | GAATTCATGACAGCTAATGAAAATCCTTTTGAGAATGAG |
| 65 | PpPMR1/LP | GGCCGGCCTCAAACAGCCATGCTGTATCCATTGTATG |
| 66 | 5′AOX1 | GCGACTGGTTCCAATTGACAAGCTT |
| 67 | PpPMR1/cLP | GGTTGCTCTCGTCGATACTCAAGTGGGAAG |
| 68 | AtECA1/cLP | GTCGGCTGGAACCTTATCACCAACTCTCAG |
| 69 | Human | ATGAGATTTCCTTCAATTTTTACTGCTGTTTTATTCGCAGCATC |
| | calreticulin | CTCCGCATTAGCTTACCCATACGACGTCCCAGACTACGCTTAC |
| | (hCRT)-DNA | CCATACGACGTCCCAGACTACGCTGAGCCCGCCGTCTACTTCA |
| | | AGGAGCAGTTTCTGGACGGAGACGGGTGGACTTCCCGCTGGA |
| | | TCGAATCCAAACACAAGTCAGATTTTGGCAAATTCGTTCTCAG |
| | | TTCCGGCAAGTTCTACGGTGACGAGGAGAAAGATAAAGGTTT |
| | | GCAGACAAGCCAGGATGCACGCTTTTATGCTCTGTCGGCCAGT |
| | | TTCGAGCCTTTCAGCAACAAAGGCCAGACGCTGGTGGTGCAG |
| | | TTCACGGTGAAACATGAGCAGAACATCGACTGTGGGGGCGGC |
| | | TATGTGAAGCTGTTTCCTAATAGTTTGGACCAGACAGACATGC |
| | | ACGGAGACTCAGAATACAACATCATGTTTGGTCCCGACATCTG |
| | | TGGCCCTGGCACCAAGAAGGTTCATGTCATCTTCAACTACAAG |
| | | GGCAAGAACGTGCTGATCAACAAGGACATCCGTTGCAAGGAT |
| | | GATGAGTTTACACACCTGTACACACTGATTGTGCGGCCAGACA |
| | | ACACCTATGAGGTGAAGATTGACAACAGCCAGGTGGAGTCCG |
| | | GCTCCTTGGAAGACGATTGGGACTTCCTGCCACCCAAGAAGA |
| | | TAAAGGATCCTGATGCTTCAAAACCGGAAGACTGGGATGAGC |
| | | GGGCCAAGATCGATGATCCCACAGACTCCAAGCCTGAGGACT |
| | | GGGACAAGCCCGAGCATATCCCTGACCCTGATGCTAAGAAGC |
| | | CCGAGGACTGGGATGAAGAGATGGACGGAGAGTGGGAACCC |
| | | CCAGTGATTCAGAACCCTGAGTACAAGGGTGAGTGGAAGCCC |
| | | CGGCAGATCGACAACCCAGATTACAAGGGCACTTGGATCCAC |
| | | CCAGAAATTGACAACCCCGAGTATTCTCCCGATCCCAGTATCT |
| | | ATGCCTATGATAACTTTGGCGTGCTGGGCCTGGACCTCTGGCA |
| | | GGTCAAGTCTGGCACCATCTTTGACAACTTCCTCATCACCAAC |
| | | GATGAGGCATACGCTGAGGAGTTTGGCAACGAGACGTGGGGC |
| | | GTAACAAAGGCAGCAGAGAAACAAATGAAGGACAAACAGGA |
| | | CGAGGAGCAGAGGCTTAAGGAGGAGGAAGAAGACAAGAAAC |
| | | GCAAAGAGGAGGAGGAGGCAGAGGACAAGGAGGATGATGAG |
| | | GACAAAGATGAGGATGAGGAGGATGAGGAGGACAAGGAGGA |
| | | AGATGAGGAGGAAGATGTCCCCGGCCAGGCCCATGACGAGCT |
| | | GTAG |
| 70 | Human | MRFPSIFTAVLFAASSALAYPYDVPDYAYPYDVPDYAEPAVYFK |
| | calreticulin | EQFLDGDGWTSRWIESKHKSDFGKFVLSSGKFYGDEEKDKGLQT |
| | (hCRT)-protein | SQDARFYALSASFEPFSNKGQTLVVQFTVKHEQNIDCGGGYVKL |
| | | FPNSLDQTDMHGDSEYNIMFGPDICGPGTKKVHVIFNYKGKNVLI |
| | | NKDIRCKDDEFTHLYTLIVRPDNTYEVKIDNSQVESGSLEDDWDF |
| | | LPPKKIKDPDASKPEDWDERAKIDDPTDSKPEDWDKPEHIPDPDA |
| | | KKPEDWDEEMDGEWEPPVIQNPEYKGEWKPRQIDNPDYKGTWI |
| | | HPEIDNPEYSPDPSIYAYDNFGVLGLDLWQVKSGTIFDNFLITNDE |
| | | AYAEEFGNETWGVTKAAEKQMKDKQDEEQRLKEEEEDKKRKE |
| | | EEEAEDKEDDEDKDEDEEDEEDKEEDEEEDVPGQAHDEL |
| 71 | Human ERp57 | ATGCAATTCAACTGGAACATCAAGACTGTTGCTTCCATCTTGT |
| | (DNA) | CCGCTTTGACTTTGGCTCAAGCTTCTGACGTTTTGGAGTTGACT |
| | | GACGACAACTTCGAGTCCAGAATTTCTGACACTGGTTCCGCTG |
| | | GATTGATGTTGGTTGAGTTCTTCGCTCCATGGTGTGGTCATTGT |
| | | AAGAGATTGGCTCCAGAATACGAAGCTGCTGCTACTAGATTG |
| | | AAGGGTATCGTTCCATTGGCTAAGGTTGACTGTACTGCTAACA |
| | | CTAACACTTGTAACAAGTACGGTGTTTCCGGTTACCCAACTTT |
| | | GAAGATCTTCAGAGATGGTGAAGAAGCTGGAGCTTACGACGG |
| | | TCCAAGAACTGCTGACGGTATCGTTTCCCACTTGAAGAAGCAA |
| | | GCTGGTCCAGCTTCTGTTCCATTGAGAACTGAGGAGGAGTTCA |
| | | AGAAGTTCATCTCCGACAAGGACGCTTCTATCGTTGGTTTCTT |
| | | CGACGATTCTTTCTCTGAAGCTCACTCCGAATTCTTGAAGGCT |
| | | GCTTCCAACTTGAGAGACAACTACAGATTCGCTCACACTAACG |
| | | TTGAGTCCTTGGTTAACGAGTACGACGATAACGGTGAAGGTA |
| | | TCATCTTGTTCAGACCATCCCACTTGACTAACAAGTTCGAGGA |
| | | CAAGACAGTTGCTTACACTGAGCAGAAGATGACTTCCGGAAA |
| | | GATCAAGAAGTTTATCCAAGAGAACATCTTCGGTATCTGTCCA |
| | | CACATGACTGAGGACAACAAGGACTTGATTCAGGGAAAGGAC |
| | | TTGTTGATCGCTTACTACGACGTTGACTACGAGAAGAACGCTA |
| | | AGGGTTCCAACTACTGGAGAAACAGAGTTATGATGGTTGCTA |
| | | AGAAGTTCTTGGACGCTGGTCACAAGTTGAACTTCGCTGTTGC |
| | | TTCTAGAAAGACTTTCTCCCACGAGTTGTCTGATTTCGGATTG |
| | | GAATCCACTGCTGGAGAGATTCCAGTTGTTGCTATCAGAACTG |
| | | CTAAGGGAGAGAAGTTCGTTATGCAAGAGGAGTTCTCCAGAG |
| | | ATGGAAAGGCTTTGGAGAGATTCTTGCAGGATTACTTCGACG |
| | | GTAACTTGAAGAGATACTTGAAGTCCGAGCCAATTCCAGAAT |
| | | CTAACGACGGTCCAGTTAAAGTTGTTGTTGCTGAGAACTTCGA |
| | | CGAGATCGTTAACAACGAGAACAAGGACGTTTTGATCGAGTT |
| | | TTACGCTCCTTGGTGTGGACACTGTAAAAACTTGGAGCCAAAG |
| | | TACAAGGAATTGGGTGAAAAGTTGTCCAAGGACCCAAACATC |
| | | GTTATCGCTAAGATGGACGCTACTGCTAACGATGTTCCATCCC |
| | | CATACGAAGTTAGAGGTTTCCCAACTATCTACTTCTCCCCAGC |
| | | TAACAAGAAGTTGAACCCAAAGAAGTACGAGGGAGGTAGAG |
| | | AATTGTCCGACTTCATCTCCTACTTGCAGAGAGAGGCTACTAA |
| | | TCCACCAGTTATCCAAGAGGAGAAGCCAAAGAAGAAGAAGA |
| | | AAGCTCACGACGAGTTGTAG |
| 72 | Human ERp57 | MQFNWNIKTVASILSALTLAQASDVLELTDDNFESRISDTGSAGL |
| | (protein) | MLVEFFAPWCGHCKRLAPEYEAAATRLKGIVPLAKVDCTANTN |
| | | TCNKYGVSGYPTLKIFRDGEEAGAYDGPRTADGIVSHLKKQAGP |
| | | ASVPLRTEEEFKKFISDKDASIVGFFDDSFSEAHSEFLKAASNLRD |
| | | NYRFAHTNVESLVNEYDDNGEGIILFRPSHLTNKFEDKTVAYTEQ |
| | | KMTSGKIKKFIQENIFGICPHMTEDNKDLIQGKDLLIAYYDVDYE |
| | | KNAKGSNYWRNRVMMVAKKFLDAGHKLNFAVASRKTFSHELS |
| | | DFGLESTAGEIPVVAIRTAKGEKFVMQEEFSRDGKALERFLQDYF |
| | | DGNLKRYLKSEPIPESNDGPVKVVVAENFDEIVNNENKDVLIEFY |
| | | APWCGHCKNLEPKYKELGEKLSKDPNIVIAKMDATANDVPSPYE |
| | | VRGFPTIYFSPANKKLNPKKYEGGRELSDFISYLQREATNPPVIQE |
| | | EKPKKKKKAHDEL |
| 73 | hCRT- | GTATACCCATACGACGTCCCAGACTACGCTGAGCCCGCCGTCT |
| | BstZ17I- | ACTTCAAGGAGC |
| | HA/UP | |
| 74 | hCRT-PacI/LP | TTAATTAACTACAGCTCGTCATGGGCCTGGCCGGGGACATCTT |
| | | CC |
| 75 | Synthetic | KLGFFKR |
| | peptide that | |
| | binds CRT | |
| 76 | hERdj3 | ATGAGATTTCCTTCAATTTTTACTGCTGTTTTATTCGCAGCATC |
| | (DNA) | CTCCGCATTAGCTGGTAGAGACTTCTACAAGATTTTGGGTGTT |
| | | CCAAGATCCGCTTCCATCAAGGACATCAAGAAGGCTTACAGA |
| | | AAGTTGGCTTTGCAATTGCACCCAGACAGAAACCCAGATGAC |
| | | CCACAAGCTCAAGAGAAGTTCCAAGACTTGGGTGCTGCTTAC |
| | | GAAGTTTTGTCCGATTCCGAGAAGAGAAAGCAGTACGACACT |
| | | TACGGTGAAGAAGGATTGAAGGACGGTCACCAATCTTCTCAC |
| | | GGTGACATCTTCTCCCACTTTTTCGGTGACTTCGGTTTCATGTT |
| | | CGGTGGTACTCCAAGACAACAGGACAGAAACATCCCAAGAGG |
| | | TTCCGACATTATCGTTGACTTGGAGGTTACATTGGAAGAGGTT |
| | | TACGCTGGTAACTTCGTTGAAGTTGTTAGAAACAAGCCAGTTG |
| | | CTAGACAAGCTCCAGGTAAAAGAAAGTGTAACTGTAGACAAG |
| | | AGATGAGAACTACTCAGTTGGGTCCTGGTAGATTCCAAATGA |
| | | CACAGGAAGTTGTTTGCGACGAGTGTCCAAACGTTAAGTTGGT |
| | | TAACGAAGAGAGAACTTTGGAGGTTGAGATCGAGCCAGGTGT |
| | | TAGAGATGGAATGGAATACCCATTCATCGGTGAAGGTGAACC |
| | | ACATGTTGATGGTGAACCTGGTGACTTGAGATTCAGAATCAA |
| | | AGTTGTTAAGCACCCAATCTTCGAGAGAAGAGGTGACGACTT |
| | | GTACACTAACGTTACTATTTCCTTGGTTGAATCCTTGGTTGGTT |
| | | TCGAGATGGACATCACTCATTTGGACGGTCACAAGGTTCACAT |
| | | TTCCAGAGACAAGATCACTAGACCAGGTGCTAAGTTGTGGAA |
| | | GAAGGGTGAAGGATTGCCAAACTTCGACAACAACAACATCAA |
| | | GGGATCTTTGATCATCACTTTCGACGTTGACTTCCCAAAAGAG |
| | | CAGTTGACTGAAGAAGCTAGAGAGGGTATCAAGCAGTTGTTG |
| | | AAGCAAGGTTCCGTTCAGAAGGTTTACAACGGATTGCAGGGA |
| | | TACTAA |
| 77 | hERdj3 | MRFPSIFTAVLFAASSALAGRDFYKILGVPRSASIKDIKKAYRKLA |
| | (protein) | LQLHPDRNPDDPQAQEKFQDLGAAYEVLSDSEKRKQYDTYGEE |
| | | GLKDGHQSSHGDIFSHFFGDFGFMFGGTPRQQDRNIPRGSDIIVDL |
| | | EVTLEEVYAGNFVEVVRNKPVARQAPGKRKCNCRQEMRTTQLG |
| | | PGRFQMTQEVVCDECPNVKLVNEERTLEVEIEPGVRDGMEYPFI |
| | | GEGEPHVDGEPGDLRFRIKVVKHPIFERRGDDLYTNVTISLVESL |
| | | VGFEMDITHLDGHKVHISRDKITRPGAKLWKKGEGLPNFDNNNI |
| | | KGSLIITFDVDFPKEQLTEEAREGIKQLLKQGSVQKVYNGLQGY |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. A Pichia pastoris host cell comprising a deletion or disruption of an endogenous gene encoding a Protein Disulphide Isomerase (PDI) and nucleic acid molecules encoding a human PDI, a recombinant human protein, and optionally, a human ERO1α protein.

2-4. (canceled)

5. The Pichia pastoris host cell of claim 1, wherein at least one endogenous gene encoding a protein O-mannosyltransferase (PMT) protein has been disrupted or deleted.

6. The Pichia pastoris host cell of claim 1, wherein the host cell further includes a nucleic acid molecule encoding an endogenous or heterologous Ca²⁺ ATPase.

7. The Pichia pastoris host cell of claim 1, wherein the host cell further includes a nucleic acid molecule encoding an ERp57 protein and a nucleic acid molecule encoding a calreticulin protein.

8-13. (canceled)

14. A method for producing a recombinant human protein comprising:
(a) providing a Pichia pastoris host cell in which an endogenous gene encoding a Protein Disulphide Isomerase (PDI) has been disrupted or deleted and the host cell expresses a human PDI and optionally, a human ERO1α protein are;
(b) introducing a nucleic acid molecule encoding the recombinant human protein into the host cell; and
(c) growing the host cell under conditions suitable for producing the recombinant human protein.

15-16. (canceled)

17. The method of claim 14, wherein at least one endogenous gene encoding a protein O-mannosyltransferase (PMT) protein has been disrupted or deleted.

18. The method of claim 14, wherein the host cell further includes a nucleic acid molecule encoding an endogenous or heterologous Ca²⁺ ATPase.

19. The method of claim 14, wherein the host cell further includes a nucleic acid molecule encoding an ERp57 protein and a nucleic acid molecule encoding a calreticulin protein.

20. A method for reducing O-glycosylation of a recombinant human protein produced in a Pichia pastoris host comprising:
(a) providing a Pichia pastoris host cell in which an endogenous gene encoding a Protein Disulphide Isomerase (PDI) has been disrupted or deleted and the host cell expresses a human PDI and optionally, a human ERO1α protein;
(b) introducing a nucleic acid molecule encoding the recombinant human protein into the host cell; and
(c) growing the host cell under conditions suitable for producing the human recombinant protein wherein the O-glycosylation of the recombinant protein is reduced compared to the O-glycosylation of the recombinant protein produced in a host cell that does not contain the human PDI.

21-22. (canceled)

23. The method of claim 20, wherein at least one endogenous gene encoding a protein O-mannosyltransferase (PMT) protein has been disrupted or deleted.

24. The method of claim 20, wherein the host cell further includes a nucleic acid molecule encoding an endogenous or heterologous Ca²⁺ ATPase.

25. The method of claim 20, wherein the host cell further includes a nucleic acid molecule encoding an ERp57 protein and a nucleic acid molecule encoding a calreticulin protein.

26. The method of claim 20, wherein the recombinant protein is selected from the group consisting of mammalian or human enzymes, cytokines, growth factors, hormones, vaccines, antibodies, and fusion proteins.

27. The method of claim 14, wherein the recombinant protein is selected from the group consisting of mammalian or human enzymes, cytokines, growth factors, hormones, vaccines, antibodies, and fusion proteins.

28. The lower eukaryote host cell of claim 1, wherein the recombinant protein is selected from the group consisting of mammalian or human enzymes, cytokines, growth factors, hormones, vaccines, antibodies, and fusion proteins.

29. The method of claim 20, wherein the host cell has been genetically modified to express glycoproteins in which the glycosylation pattern is human-like or humanized.

30. The method of claim 14, wherein the host cell has been genetically modified to express glycoproteins in which the glycosylation pattern is human-like or humanized.

31. The Pichia pastoris host cell of claim 1, wherein the host cell has been genetically modified to express glycoproteins in which the glycosylation pattern is human-like or humanized.
